SEQUENCE LISTING (1289 sequences)

SEQ ID NO: 1 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcagtggtagagcgcgtgcttagcatgcacgaggtcctgggttcgatccccagtacctcca

SEQ ID NO: 2 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggggaattagctcaagtggtagagcgcttgcttagcacgcaagaggtagtgggatcgatgcccacattctcca

SEQ ID NO: 3 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggggaattagctcaaatggtagagcgctcgcttagcatgcgagaggtagcgggatcgatgcccgcattctcca

SEQ ID NO: 4 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggggaattagctcaagtggtagagcgcttgcttagcatgcaagaggtagtgggatcgatgcccacattctcca

SEQ ID NO: 5 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggggaattagctcaagcggtagagcgcttgcttagcatgcaagaggtagtgggatcgatgcccacattctcca

SEQ ID NO: 6 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggggaattagctcaagtggtagagcgcttgcttagcatgcaagaggtagtgggatcaatgcccacattctcca

SEQ ID NO: 7 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggggaattagctcaagtggtagagcgctcgcttagcatgcgagaggtagtgggatcgatgcccgcattctcca

SEQ ID NO: 8 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggggaattagcccaagtggtagagcgcttgcttagcatgcaagaggtagtgggatcgatgcccacattctcca

SEQ ID NO: 9 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtgtagctcagtggtagagcgcgtgcttagcatgcacgaggccccgggttcaatccccggcacctcca

SEQ ID NO: 10 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtgtagctcagtggtagagcgcgtgcttagcatgtacgaggtcccgggttcaatccccggcacctcca

SEQ ID NO: 11 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggggatgtagctcagtggtagagcgcatgcttagcatgcatgaggtcccgggttcgatccccagcatctcca

SEQ ID NO: 12 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtgtagctcagtggtagagcgcgtgcttagcatgcacgaggccctgggttcaatccccagcacctcca

SEQ ID NO: 13 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcagcggtagagcgcgtgcttagcatgcacgaggtcctgggttcaatccccaatacctcca

SEQ ID NO: 14 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtgtagctcagtggtagagcgcgtgcttagcatgcacgaggccccgggttcaatccctggcacctcca

SEQ ID NO: 15 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gggggattagctcaaatggtagagcgctcgcttagcatgcgagaggtagcgggatcgatgcccgcatcctcca

SEQ ID NO: 16 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggggaattagctcaggcggtagagcgctcgcttagcatgcgagaggtagcgggatcgacgcccgcattctcca

SEQ ID NO: 17 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggggatgtagctcagtggtagagcgcatgcttcgcatgtatgaggtcccgggttcgatccccggcatctcca

SEQ ID NO: 18 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggggatgtagctcagtggtagagcgcatgcttcgcatgtatgaggccccgggttcgatccccggcatctcca

SEQ ID NO: 19 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggggatgtagctcagtggtagagcgcgcgcttcgcatgtgtgaggtcccgggttcaatccccggcatctcca

SEQ ID NO: 20 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtgtagctcagtggtagagcgcgtgcttcgcatgtacgaggccccgggttcgacccccggctcctcca

SEQ ID NO: 21 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtgtagctcagtggtagagcgcatgctttgcatgtatgaggtcccgggttcgatccccggcacctcca

SEQ ID NO: 22 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggggatgtagctcagtggtagagcgcatgctttgcatgtatgaggtcccgggttcgatccccggcatctcca

SEQ ID NO: 23 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggggatgtagctcagtggtagagcgcatgctttgcatgtatgaggccccgggttcgatccccggcatctcca

SEQ ID NO: 24 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggggatgtagctcagtggtagagcgcatgctttgcacgtatgaggccccgggttcaatccccggcatctcca

SEQ ID NO: 25 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtgtagctcagtggtagagcgcatgctttgcatgtatgaggcctcgggttcgatccccgacacctcca

SEQ ID NO: 26 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtgtagctcagtggtagagcacatgctttgcatgtgtgaggccccgggttcgatccccggcacctcca

SEQ ID NO: 27 - DNA, 71 bp, synthetic construct
  misc_feature 1..71: note = Description of sequence: transcripted tRNA sequence; tRNA name: Ala_AGC_chr6:28763741-28763812 (-)
  source 1..71: mol_type = other DNA
gggggtgtagctcagtggtagagcgcatgctttgcatgtatgaggcctcggttcgatccccgacacctcca

SEQ ID NO: 28 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gggccagtggcgcaatggataacgcgtctgactacggatcagaagattccaggttcgactcctggctggctcg

SEQ ID NO: 29 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gggccagtggcgcaatggataacgcgtctgactacggatcagaagattctaggttcgactcctggctggctcg

SEQ ID NO: 30 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggccgcgtggcctaatggataaggcgtctgattccggatcagaagattgagggttcgagtcccttcgtggtcg

SEQ ID NO: 31 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gacccagtggcctaatggataaggcatcagcctccggagctggggattgtgggttcgagtcccatctgggtcg

SEQ ID NO: 32 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccccagtggcctaatggataaggcactggcctcctaagccagggattgtgggttcgagtcccacctggggta

SEQ ID NO: 33 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccccagtggcctaatggataaggcactggcctcctaagccagggattgtgggttcgagtcccacctggggtg

SEQ ID NO: 34 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccccggtggcctaatggataaggcattggcctcctaagccagggattgtgggttcgagtcccacccggggta

SEQ ID NO: 35 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccccagtggcctaatggataaggcattggcctcctaagccagggattgtgggttcgagtcccatctggggtg

SEQ ID NO: 36 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccccagtggcctgatggataaggtactggcctcctaagccagggattgtgggttcgagttccacctggggta

SEQ ID NO: 37 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggccgcgtggcctaatggataaggcgtctgacttcggatcagaagattgcaggttcgagtcctgccgcggtcg

SEQ ID NO: 38 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gaccacgtggcctaatggataaggcgtctgacttcggatcagaagattgagggttcgaatccctccgtggtta

SEQ ID NO: 39 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gaccgcgtggcctaatggataaggcgtctgacttcggatcagaagattgagggttcgagtcccttcgtggtcg

SEQ ID NO: 40 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gaccacgtggcctaatggataaggcgtctgacttcggatcagaagattgagggttcgaatcccttcgtggtta

SEQ ID NO: 41 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gaccacgtggcctaatggataaggcgtctgacttcggatcagaagattgagggttcgaatcccttcgtggttg

SEQ ID NO: 42 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggccgtgtggcctaatggataaggcgtctgacttcggatcaaaagattgcaggtttgagttctgccacggtcg

SEQ ID NO: 43 - DNA, 85 bp, synthetic construct
  misc_feature 1..85: note = Description of sequence: transcripted tRNA sequence; tRNA name: Ala_AGC_chr6:28763741-28763812 (-)
  source 1..85: mol_type = other DNA
ggctccgtggcgcaatggatagcgcattggacttctagaggctgaaggcattcaaaggttccgggttcgagtcccggcggagtcg

SEQ ID NO: 44 - DNA, 88 bp, synthetic construct
  misc_feature 1..88: note = Description of sequence: transcripted tRNA sequence; tRNA name: Ala_AGC_chr6:28763741-28763812 (-)
  source 1..88: mol_type = other DNA
ggctctgtggcgcaatggatagcgcattggacttctagtgacgaatagagcaattcaaaggttgtgggttcgaatcccaccagagtcg

SEQ ID NO: 45 - DNA, 91 bp, synthetic construct
  misc_feature 1..91: note = Description of sequence: transcripted tRNA sequence; tRNA name: Ala_AGC_chr6:28763741-28763812 (-)
  source 1..91: mol_type = other DNA
ggctctgtggcgcaatggatagcgcattggacttctagctgagcctagtgtggtcattcaaaggttgtgggttcgagtcccaccagagtcg

SEQ ID NO: 46 - DNA, 86 bp, synthetic construct
  misc_feature 1..86: note = Description of sequence: transcripted tRNA sequence; tRNA name: Ala_AGC_chr6:28763741-28763812 (-)
  source 1..86: mol_type = other DNA
ggctctgtggcgcaatggatagcgcattggacttctagatagttagagaaattcaaaggttgtgggttcgagtcccaccagagtcg

SEQ ID NO: 47 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggcgcaatggacgagcgcgctggacttctaatccagaggttccgggttcgagtcccggcagagatg

SEQ ID NO: 48 - DNA, 87 bp, synthetic construct
  misc_feature 1..87: note = Description of sequence: transcripted tRNA sequence; tRNA name: Ala_AGC_chr6:28763741-28763812 (-)
  source 1..87: mol_type = other DNA
ggctctgtggcgcaatggatagcgcattggacttctagcctaaatcaagagattcaaaggttgcgggttcgagtccctccagagtcg

SEQ ID NO: 49 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggcgcaatcggttagcgcgttcggctgttaaccgaaaggttggtggttcgatcccacccagggacg

SEQ ID NO: 50 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggcgcaatcggctagcgcgtttggctgttaactaaaaggttggcggttcgaacccacccagaggcg

SEQ ID NO: 51 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggtgcaatcggttagcgcgttccgctgttaaccgaaagcttggtggttcgagcccacccagggatg

SEQ ID NO: 52 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggcgcaatcggctagcgcgtttggctgttaactaaaaagttggtggttcgaacacacccagaggcg

SEQ ID NO: 53 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggcgcaatcggttagcgcgttcggctgttaaccgaaaggttggtggttcgagcccacccagggacg

SEQ ID NO: 54 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggcgcaatcggttagcgcattcggctgttaaccgaaaggttggtggttcgagcccacccagggacg

SEQ ID NO: 55 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggcgcaatcggttagcgcgttcggctgttaaccgaaagattggtggttcgagcccacccagggacg

SEQ ID NO: 56 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggcgcaatcggttagcgcgttcggctgttaactgaaaggttggtggttcgagcccacccagggacg

SEQ ID NO: 57 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggcgcaatgggttagcgcgttcggctgttaaccgaaaggttggtggttcgagcccatccagggacg

SEQ ID NO: 58 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggcgtagtcggttagcgcgttcggctgttaaccgaaaagttggtggttcgagcccacccaggaacg

SEQ ID NO: 59 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggcgcaatcggctagcgcgtttggctgttaactaaaaggttggtggttcgaacccacccagaggcg

SEQ ID NO: 60 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtctctgtggcgcaatcggttagcgcgttcggctgttaactgaaaggttagtggttcgagcccacccggggacg

SEQ ID NO: 61 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
tcctcgttagtatagtggttagtatccccgcctgtcacgcgggagaccggggttcaattccccgacggggag

SEQ ID NO: 62 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
tcctcgttagtatagtggtgagtatccccgcctgtcacgcgggagaccggggttcgattccccgacggggag

SEQ ID NO: 63 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
tcctcgttagtatagtggtgagtgtccccgtctgtcacgcgggagaccggggttcgattccccgacggggag

SEQ ID NO: 64 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggcatagctcagtggtagagcatttgactgcagatcaagaggtccctggttcaaatccaggtgccccct

SEQ ID NO: 65 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcaggggtagagcatttgactgcagatcaagaggtccctggttcaaatccaggtgcccccc

SEQ ID NO: 66 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagcttagcggtagagcatttgactgcagatcaagaggtccccggttcaaatccgggtgccccct

SEQ ID NO: 67 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagcttaggggtagagcatttgactgcagatcaaaaggtccctggttcaaatccaggtgcccctt

SEQ ID NO: 68 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcaggggtagagcatttgactgcagatcaagaggtccccagttcaaatctgggtgccccct

SEQ ID NO: 69 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcaggggtagagcatttgactgcagatcaagaagtccccggttcaaatccgggtgccccct

SEQ ID NO: 70 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcaggggtagagcatttgactgcagatcaagaggtctctggttcaaatccaggtgccccct

SEQ ID NO: 71 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcaggggtagagcacttgactgcagatcaagaagtccttggttcaaatccaggtgccccct

SEQ ID NO: 72 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggggatatagctcaggggtagagcatttgactgcagatcaagaggtccccggttcaaatccgggtgcccccc

SEQ ID NO: 73 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagttcaggggtagagcatttgactgcagatcaagaggtccctggttcaaatccaggtgccccct

SEQ ID NO: 74 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcaggggtagagcatttgactgcaaatcaagaggtccctgattcaaatccaggtgccccct

SEQ ID NO: 75 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcagtggtagagcatttgactgcagatcaagaggtccccggttcaaatccgggtgccccct

SEQ ID NO: 76 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggcgtatagctcaggggtagagcatttgactgcagatcaagaggtccccagttcaaatctgggtgccccct

SEQ ID NO: 77 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcacaggtagagcatttgactgcagatcaagaggtccccggttcaaatctgggtgccccct

SEQ ID NO: 78 - DNA, 70 bp, synthetic construct
  misc_feature 1..70: note = Description of sequence: transcripted tRNA sequence; tRNA name: Ala_AGC_chr6:28763741-28763812 (-)
  source 1..70: mol_type = other DNA
gggcgtatagctcaggggtagagcatttgactgcagatcaagaggtccccagttcaaatctgggtgccca

SEQ ID NO: 79 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcacaggtagagcatttgactgcagatcaagaggtccccggttcaaatccggttactccct

SEQ ID NO: 80 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcaggggtagagcacttgactgcagatcaagaggtccctggttcaaatccaggtgccccct

SEQ ID NO: 81 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcagtggtagagcatttgactgcagatcaagaggtccctggttcaaatccgggtgccccct

SEQ ID NO: 82 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gggggtatagctcagtgggtagagcatttgactgcagatcaagaggtccccggttcaaatccgggtgccccct

SEQ ID NO: 83 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtgtagctcagtggtagagcatttgactgcagatcaagaggtccctggttcaaatccaggtgccccct

SEQ ID NO: 84 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gggggtatagctcaggtggtagagcatttgactgcagatcaagaggtccccggttcaaatccgggtgccccct

SEQ ID NO: 85 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcaggggtagagcatttgactgcagatcaagaggtccccggttcaaatccgggtgccccct

SEQ ID NO: 86 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtatagctcaggggtagagcatttgactgcagatcaagaggtccctggttcaaatccaggtgccccct

SEQ ID NO: 87 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggttccatggtgtaatggttagcactctggactctgaatccagcgatccgagttcaaatctcggtggaacct

SEQ ID NO: 88 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggttccatggtgtaatggttagcactctggactctgaatccagcgatccgagttcaagtctcggtggaacct

SEQ ID NO: 89 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggttccatggtgtaatggtgagcactctggactctgaatccagcgatccgagttcgagtctcggtggaacct

SEQ ID NO: 90 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggttccatggtgtaatggtaagcactctggactctgaatccagcgatccgagttcgagtctcggtggaacct

SEQ ID NO: 91 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggttccatggtgtaatggttagcactctggactctgaatccggtaatccgagttcaaatctcggtggaacct

SEQ ID NO: 92 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggccccatggtgtaatggtcagcactctggactctgaatccagcgatccgagttcaaatctcggtgggaccc

SEQ ID NO: 93 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggttccatggtgtaatggtaagcactctggactctgaatccagccatctgagttcgagtctctgtggaacct

SEQ ID NO: 94 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggtcccatggtgtaatggttagcactctggactttgaatccagcgatccgagttcaaatctcggtgggacct

SEQ ID NO: 95 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggtcccatggtgtaatggttagcactctggactttgaatccagcaatccgagttcgaatctcggtgggacct

SEQ ID NO: 96 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggccccatggtgtaatggttagcactctggactttgaatccagcgatccgagttcaaatctcggtgggacct

SEQ ID NO: 97 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggtcccatggtgtaatggttagcactctgggctttgaatccagcaatccgagttcgaatcttggtgggacct

SEQ ID NO: 98 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
tccctggtggtctagtggttaggattcggcgctctcaccgccgcggcccgggttcgattcccggtcagggaa

SEQ ID NO: 99 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
tccctggtggtctagtggttaggattcggcgctctcaccgccgcggcccgggttcgattcccggtcaggaaa

SEQ ID NO: 100 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
tcccatatggtctagcggttaggattcctggttttcacccaggtggcccgggttcgactcccggtatgggaa

SEQ ID NO: 101 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
tcccacatggtctagcggttaggattcctggttttcacccaggcggcccgggttcgactcccggtgtgggaa

SEQ ID NO: 102 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
tccctggtggtctagtggctaggattcggcgctttcaccgccgcggcccgggttcgattcccggccagggaa

SEQ ID NO: 103 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
tccctggtggtctagtggctaggattcggcgctttcaccgccgcggcccgggttcgattcccggtcagggaa

SEQ ID NO: 104 - DNA, 71 bp, synthetic construct
  misc_feature 1..71: note = Description of sequence: transcripted tRNA sequence; tRNA name: Ala_AGC_chr6:28763741-28763812 (-)
  source 1..71: mol_type = other DNA
gcattggtggttcagtggtagaattctcgcctcccacgcgggagacccgggttcaattcccggccaatgca

SEQ ID NO: 105 - DNA, 71 bp, synthetic construct
  misc_feature 1..71: note = Description of sequence: transcripted tRNA sequence; tRNA name: Ala_AGC_chr6:28763741-28763812 (-)
  source 1..71: mol_type = other DNA
gcgccgctggtgtagtggtatcatgcaagattcccattcttgcgacccgggttcgattcccgggcggcgca

SEQ ID NO: 106 - DNA, 71 bp, synthetic construct
  misc_feature 1..71: note = Description of sequence: transcripted tRNA sequence; tRNA name: Ala_AGC_chr6:28763741-28763812 (-)
  source 1..71: mol_type = other DNA
gcattggtggttcaatggtagaattctcgcctcccacgcaggagacccaggttcgattcctggccaatgca

SEQ ID NO: 107 - DNA, 71 bp, synthetic construct
  misc_feature 1..71: note = Description of sequence: transcripted tRNA sequence; tRNA name: Ala_AGC_chr6:28763741-28763812 (-)
  source 1..71: mol_type = other DNA
gcatgggtggttcagtggtagaattctcgcctgccacgcgggaggcccgggttcgattcccggcccatgca

SEQ ID NO: 108 - DNA, 71 bp, synthetic construct
  misc_feature 1..71: note = Description of sequence: transcripted tRNA sequence; tRNA name: Ala_AGC_chr6:28763741-28763812 (-)
  source 1..71: mol_type = other DNA
gcattggtggttcagtggtagaattctcgcctgccacgcgggaggcccgggttcgattcccggccaatgca

SEQ ID NO: 109 - DNA, 71 bp, synthetic construct
  misc_feature 1..71: note = Description of sequence: transcripted tRNA sequence; tRNA name: Ala_AGC_chr6:28763741-28763812 (-)
  source 1..71: mol_type = other DNA
gcattggtggttcagtggtagaattctcgcctgccacgcgggaggcccgggtttgattcccggccagtgca

SEQ ID NO: 110 - DNA, 71 bp, synthetic construct
  misc_feature 1..71: note = Description of sequence: transcripted tRNA sequence; tRNA name: Ala_AGC_chr6:28763741-28763812 (-)
  source 1..71: mol_type = other DNA
gcataggtggttcagtggtagaattcttgcctgccacgcaggaggcccaggtttgattcctggcccatgca

SEQ ID NO: 111 - DNA, 71 bp, synthetic construct
  misc_feature 1..71: note = Description of sequence: transcripted tRNA sequence; tRNA name: Ala_AGC_chr6:28763741-28763812 (-)
  source 1..71: mol_type = other DNA
gcattggtggttcagtggtagaattctcgcctgccatgcgggcggccgggcttcgattcctggccaatgca

SEQ ID NO: 112 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gcgttggtggtatagtggttagcatagctgccttccaagcagttgacccgggttcgattcccggccaacgca

SEQ ID NO: 113 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gcgttggtggtatagtggtgagcatagctgccttccaagcagttgacccgggttcgattcccggccaacgca

SEQ ID NO: 114 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gcgttggtggtatagtggtaagcatagctgccttccaagcagttgacccgggttcgattcccggccaacgca

SEQ ID NO: 115 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gcgttggtggtatagtggtgagcatagttgccttccaagcagttgacccgggctcgattcccgcccaacgca

SEQ ID NO: 116 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gccgtgatcgtatagtggttagtactctgcgttgtggccgcagcaacctcggttcgaatccgagtcacggca

SEQ ID NO: 117 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gccatgatcgtatagtggttagtactctgcgctgtggccgcagcaacctcggttcgaatccgagtcacggca

SEQ ID NO: 118 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggccggttagctcagttggttagagcgtggcgctaataacgccaaggtcgcgggttcgatccccgtacgggcca

SEQ ID NO: 119 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggccggttagctcagttggttagagcgtggtgctaataacgccaaggtcgcgggttcgatccccgtactggcca

SEQ ID NO: 120 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggctggttagctcagttggttagagcgtggtgctaataacgccaaggtcgcgggttcgatccccgtactggcca

SEQ ID NO: 121 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggccggttagctcagttggttagagcgtggtgctaataacgccaaggtcgcgggttcgaaccccgtacgggcca

SEQ ID NO: 122 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggccggttagctcagttggttagagcgtggtgctaataacgccaaggtcgcgggttcgatccccgtacgggcca

SEQ ID NO: 123 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggccggttagctcagttggttagagcgtggtgctaataacgctaaggtcgcgggttcgatccccgtactggcca

SEQ ID NO: 124 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggccggttagctcagttggtcagagcgtggtgctaataacgccaaggtcgcgggttcgatccccgtacgggcca

SEQ ID NO: 125 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggccggttagctcagtcggctagagcgtggtgctaataacgccaaggtcgcgggttcgatccccgtacgggcca

SEQ ID NO: 126 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggctggttagttcagttggttagagcgtggtgctaataacgccaaggtcgtgggttcgatccccatatcggcca

SEQ ID NO: 127 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggccggttagctcagttggtaagagcgtggtgctgataacaccaaggtcgcgggctcgactcccgcaccggcca

SEQ ID NO: 128 - DNA, 93 bp, synthetic construct
  misc_feature 1..93: note = Description of sequence: transcripted tRNA sequence; tRNA name: Ala_AGC_chr6:28763741-28763812 (-)
  source 1..93: mol_type = other DNA
gctccagtggcgcaatcggttagcgcgcggtacttatatgacagtgcgagcggagcaatgccgaggttgtgagttcgatcctcacctggagca

SEQ ID NO: 129 - DNA, 93 bp, synthetic construct
  misc_feature 1..93: note = Description of sequence: transcripted tRNA sequence; tRNA name: Ala_AGC_chr6:28763741-28763812 (-)
  source 1..93: mol_type = other DNA
gctccagtggcgcaatcggttagcgcgcggtacttatacagcagtacatgcagagcaatgccgaggttgtgagttcgagcctcacctggagca

SEQ ID NO: 130 - DNA, 94 bp, synthetic construct
  misc_feature 1..94: note = Description of sequence: transcripted tRNA sequence; tRNA name: Ala_AGC_chr6:28763741-28763812 (-)
  source 1..94: mol_type = other DNA
gctccagtggcgcaatcggttagcgcgcggtacttatatggcagtatgtgtgcgagtgatgccgaggttgtgagttcgagcctcacctggagca

SEQ ID NO: 131 - DNA, 94 bp, synthetic construct
  misc_feature 1..94: note = Description of sequence: transcripted tRNA sequence; tRNA name: Ala_AGC_chr6:28763741-28763812 (-)
  source 1..94: mol_type = other DNA
gctccagtggcgcaatcggttagcgcgcggtacttatacaacagtatatgtgcgggtgatgccgaggttgtgagttcgagcctcacctggagca

SEQ ID NO: 132 - DNA, 94 bp, synthetic construct
  misc_feature 1..94: note = Description of sequence: transcripted tRNA sequence; tRNA name: Ala_AGC_chr6:28763741-28763812 (-)
  source 1..94: mol_type = other DNA
gctccagtggcgcaatcggttagcgcgcggtacttataagacagtgcacctgtgagcaatgccgaggttgtgagttcaagcctcacctggagca

SEQ ID NO: 133 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
ggtagcgtggccgagcggtctaaggcgctggattaaggctccagtctcttcggaggcgtgggttcgaatcccaccgctgcca

SEQ ID NO: 134 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
ggtagcgtggccgagcggtctaaggcgctggattaaggctccagtctcttcgggggcgtgggttcgaatcccaccgctgcca

SEQ ID NO: 135 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
ggtagcgtggccgagcggtctaaggcgctggattaaggctccagtctcttcgggggcgtgggttcaaatcccaccgctgcca

SEQ ID NO: 136 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
ggtagcgtggccgagtggtctaagacgctggattaaggctccagtctcttcgggggcgtgggtttgaatcccaccgctgcca

SEQ ID NO: 137 - DNA, 106 bp, synthetic construct
  misc_feature 1..106: note = Description of sequence: transcripted tRNA sequence; tRNA name: Ala_AGC_chr6:28763741-28763812 (-)
  source 1..106: mol_type = other DNA
gtcaggatggccgagtggtctaaggcgccagactcaagctaagcttcctccgcggtggggattctggtctccaatggaggcgtgggttcgaatcccacttctgaca

SEQ ID NO: 138 - DNA, 105 bp, synthetic construct
  misc_feature 1..105: note = Description of sequence: transcripted tRNA sequence; tRNA name: Ala_AGC_chr6:28763741-28763812 (-)
  source 1..105: mol_type = other DNA
gtcaggatggccgagtggtctaaggcgccagactcaagcttggcttcctcgtgttgaggattctggtctccaatggaggcgtgggttcgaatcccacttctgaca

SEQ ID NO: 139 - DNA, 108 bp, synthetic construct
  misc_feature 1..108: note = Description of sequence: transcripted tRNA sequence; tRNA name: Ala_AGC_chr6:28763741-28763812 (-)
  source 1..108: mol_type = other DNA
gtcaggatggccgagtggtctaaggcgccagactcaagcttactgcttcctgtgttcgggtcttctggtctccgtatggaggcgtgggttcgaatcccacttctgaca

SEQ ID NO: 140 - DNA, 107 bp, synthetic construct
  misc_feature 1..107: note = Description of sequence: transcripted tRNA sequence; tRNA name: Ala_AGC_chr6:28763741-28763812 (-)
  source 1..107: mol_type = other DNA
gtcaggatggccgagtggtctaaggcgccagactcaagttgctacttcccaggtttggggcttctggtctccgcatggaggcgtgggttcgaatcccacttctgaca

SEQ ID NO: 141 - DNA, 106 bp, synthetic construct
  misc_feature 1..106: note = Description of sequence: transcripted tRNA sequence; tRNA name: Ala_AGC_chr6:28763741-28763812 (-)
  source 1..106: mol_type = other DNA
gtcaggatggccgagtggtctaaggcgccagactcaaggtaagcaccttgcctgcgggctttctggtctccggatggaggcgtgggttcgaatcccacttctgaca

SEQ ID NO: 142 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gcctccttagtgcagtaggtagcgcatcagtctcaaaatctgaatggtcctgagttcaagcctcagagggggca

SEQ ID NO: 143 - DNA, 84 bp, synthetic construct
  misc_feature 1..84: note = Description of sequence: transcripted tRNA sequence; tRNA name: Ala_AGC_chr6:28763741-28763812 (-)
  source 1..84: mol_type = other DNA
gtcaggatggccgagcagtcttaaggcgctgcgttcaaatcgcaccctccgctggaggcgtgggttcgaatcccacttttgaca

SEQ ID NO: 144 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
gtcaggatggccgagcggtctaaggcgctgcgttcaggtcgcagtctcccctggaggcgtgggttcgaatcccactcctgaca

SEQ ID NO: 145 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
gtcaggatggccgagcggtctaaggcgctgcgttcaggtcgcagtctcccctggaggcgtgggttcgaatcccacttctgaca

SEQ ID NO: 146 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
accaggatggccgagtggttaaggcgttggacttaagatccaatggacatatgtccgcgtgggttcgaaccccactcctggta

SEQ ID NO: 147 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
accgggatggccgagtggttaaggcgttggacttaagatccaatgggctggtgcccgcgtgggttcgaaccccactctcggta

SEQ ID NO: 148 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
accagaatggccgagtggttaaggcgttggacttaagatccaatggattcatatccgcgtgggttcgaaccccacttctggta

SEQ ID NO: 149 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
accgggatggctgagtggttaaggcgttggacttaagatccaatggacaggtgtccgcgtgggttcgagccccactcccggta

SEQ ID NO: 150 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
ggtagcgtggccgagcggtctaaggcgctggatttaggctccagtctcttcggaggcgtgggttcgaatcccaccgctgcca

SEQ ID NO: 151 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
ggtagtgtggccgagcggtctaaggcgctggatttaggctccagtctcttcgggggcgtgggttcgaatcccaccactgcca

SEQ ID NO: 152 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
ggtagcgtggccgagtggtctaaggcgctggatttaggctccagtcatttcgatggcgtgggttcgaatcccaccgctgcca

SEQ ID NO: 153 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcccggctagctcagtcggtagagcatgggactcttaatcccagggtcgtgggttcgagccccacgttgggcg

SEQ ID NO: 154 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcccagctagctcagtcggtagagcataagactcttaatctcagggttgtggattcgtgccccatgctgggtg

SEQ ID NO: 155 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gcagctagctcagtcggtagagcatgagactcttaatctcagggtcatgggttcgtgccccatgttgggtgcca

SEQ ID NO: 156 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcccggctagctcagtcggtagagcatgagactcttaatctcagggtcgtgggttcgagccccacgttgggcg

SEQ ID NO: 157 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcccggctagctcagtcggtagagcatgagacccttaatctcagggtcgtgggttcgagccccacgttgggcg

SEQ ID NO: 158 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcccggctagctcagtcggtagagcatgggactcttaatctcagggtcgtgggttcgagccccacgttgggcg

SEQ ID NO: 159 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcccggctagctcagtcgatagagcatgagactcttaatctcagggtcgtgggttcgagccgcacgttgggcg

SEQ ID NO: 160 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcccagctagctcagtcggtagagcatgagactcttaatctcagggtcatgggtttgagccccacgtttggtg

SEQ ID NO: 161 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcctggctagctcagtcggcaaagcatgagactcttaatctcagggtcgtgggctcgagctccatgttgggcg

SEQ ID NO: 162 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcccgactacctcagtcggtggagcatgggactcttcatcccagggttgtgggttcgagccccacattgggca

SEQ ID NO: 163 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcctggatagctcagttggtagagcatcagacttttaatctgagggtccagggttcaagtccctgttcaggca

SEQ ID NO: 164 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
acccagatagctcagtcagtagagcatcagacttttaatctgagggtccaaggttcatgtccctttttgggtg

SEQ ID NO: 165 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcctggatagctcagttggtagagcatcagacttttaatctgagggtccagggttcaagtccctgttcaggcg

SEQ ID NO: 166 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcccggatagctcagtcggtagagcatcagacttttaatctgagggtccagggttcaagtccctgttcgggcg

SEQ ID NO: 167 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcctggatagctcagtcggtagagcatcagacttttaatctgagggtccagggttcaagtccctgttcaggcg

SEQ ID NO: 168 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcccggatagctcagtcggtagagcatcagacttttaatctgagggtccggggttcaagtccctgttcgggcg

SEQ ID NO: 169 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcctgggtagctcagtcggtagagcatcagacttttaatctgagggtccagggttcaagtccctgtccaggcg

SEQ ID NO: 170 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcctggatagctcagttggtagaacatcagacttttaatctgacggtgcagggttcaagtccctgttcaggcg

SEQ ID NO: 171 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcctcgttagcgcagtaggtagcgcgtcagtctcataatctgaaggtcgtgagttcgatcctcacacggggca

SEQ ID NO: 172 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccctcttagcgcagtgggcagcgcgtcagtctcataatctgaaggtcctgagttcgagcctcagagagggca

SEQ ID NO: 173 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcctccttagcgcagtaggcagcgcgtcagtctcataatctgaaggtcctgagttcgaacctcagagggggca

SEQ ID NO: 174 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccctcttagcgcagcgggcagcgcgtcagtctcataatctgaaggtcctgagttcgagcctcagagagggca

SEQ ID NO: 175 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccctcttagcgcagctggcagcgcgtcagtctcataatctgaaggtcctgagttcaagcctcagagagggca

SEQ ID NO: 176 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcctcgttagcgcagtaggcagcgcgtcagtctcataatctgaaggtcgtgagttcgagcctcacacggggca

SEQ ID NO: 177 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccctcttagtgcagctggcagcgcgtcagtttcataatctgaaagtcctgagttcaagcctcagagagggca

SEQ ID NO: 178 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccgaaatagctcagttgggagagcgttagactgaagatctaaaggtccctggttcgatcccgggtttcggca

SEQ ID NO: 179 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccgaaatagctcagttgggagagcgttagactgaagatctaaaggtccctggttcaatcccgggtttcggca

SEQ ID NO: 180 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccgagatagctcagttgggagagcgttagactgaagatctaaaggtccctggttcaatcccgggtttcggca

SEQ ID NO: 181 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gccgaaatagctcagttgggagagcgttagaccgaagatcttaaaggtccctggttcaatcccgggtttcggca

SEQ ID NO: 182 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gctgaaatagctcagttgggagagcgttagactgaagatcttaaagttccctggttcaaccctgggtttcagcc

SEQ ID NO: 183 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggctcgttggtctaggggtatgattctcgcttaggatgcgagaggtcccgggttcaaatcccggacgagccc

SEQ ID NO: 184 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggctcgttggtctaggggtatgattctcgcttagggtgcgagaggtcccgggttcaaatcccggacgagccc

SEQ ID NO: 185 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggctcgttggtctaggggtatgattctcgcttcgggtgcgagaggtcccgggttcaaatcccggacgagccc

SEQ ID NO: 186 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggctcgttggtctaggggtatgattctcgcttcgggtgtgagaggtcccgggttcaaatcccggacgagccc

SEQ ID NO: 187 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggctcgttggtctagtggtatgattctcgctttgggtgcgagaggtcccgggttcaaatcccggacgagccc

SEQ ID NO: 188 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggctcgttggtctaggggtatgattctcggtttgggtccgagaggtcccgggttcaaatcccggacgagccc

SEQ ID NO: 189 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggctcgttggtctaggggtatgattctcgctttgggtgcgagaggtcccgggttcaaatcccggacgagccc

SEQ ID NO: 190 - DNA, 87 bp, synthetic construct
  misc_feature 1..87: note = Description of sequence: transcripted tRNA sequence; tRNA name: Ala_AGC_chr6:28763741-28763812 (-)
  source 1..87: mol_type = other DNA
gcccggatgatcctcagtggtctggggtgcaggcttcaaacctgtagctgtctagcgacagagtggttcaattccacctttcgggcg

SEQ ID NO: 191 - DNA, 84 bp, synthetic construct
  misc_feature 1..84: note = Description of sequence: transcripted tRNA sequence; tRNA name: Ala_AGC_chr6:28763741-28763812 (-)
  source 1..84: mol_type = other DNA
gctcggatgatcctcagtggtctggggtgcaggcttcaaacctgtagctgtctagtgacagagtggttcaattccacctttgta

SEQ ID NO: 192 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gtagtcgtggccgagtggttaaggcgatggactagaaatccattggggtttccccgcgcaggttcgaatcctgccgactacg

SEQ ID NO: 193 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gtagtcgtggccgagtggttaaggcgatggactagaaatccattggggtctccccgcgcaggttcgaatcctgccgactacg

SEQ ID NO: 194 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gtagtcgtggccgagtggttaaggcgatggactagaaatccattggggtttccccacgcaggttcgaatcctgccgactacg

SEQ ID NO: 195 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gtagtcgtggccgagtggttaaggtgatggactagaaacccattggggtctccccgcgcaggttcgaatcctgccgactacg

SEQ ID NO: 196 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gctgtgatggccgagtggttaaggcgttggactcgaaatccaatggggtctccccgcgcaggttcgaatcctgctcacagcg

SEQ ID NO: 197 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gctgtgatggccgagtggttaaggcgttggactcgaaatccaatggggtctccccgcgcaggttcaaatcctgctcacagcg

SEQ ID NO: 198 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gctgtgatggccgagtggttaaggtgttggactcgaaatccaatgggggttccccgcgcaggttcaaatcctgctcacagcg

SEQ ID NO: 199 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gtcacggtggccgagtggttaaggcgttggactcgaaatccaatggggtttccccgcacaggttcgaatcctgttcgtgacg

SEQ ID NO: 200 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gacgaggtggccgagtggttaaggcgatggactgctaatccattgtgctctgcacgcgtgggttcgaatcccaccctcgtcg

SEQ ID NO: 201 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gacgaggtggccgagtggttaaggcgatggactgctaatccattgtgctctgcacgcgtgggttcgaatcccaccttcgtcg

SEQ ID NO: 202 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gacgaggtggccgagtggttaaggcgatggactgctaatccattgtgctttgcacgcgtgggttcgaatcccatcctcgtcg

SEQ ID NO: 203 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gacgaggtggccgagtggttaaggcgatggactgctaatccattgtgctctgcacgcgtgggttcgaatcccatcctcgtcg

SEQ ID NO: 204 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gacgaggtggccgagtggttaaggcgatggactgctaatccattgtgctctgcacacgtgggttcgaatcccatcctcgtcg

SEQ ID NO: 205 - DNA, 84 bp, synthetic construct
  misc_feature 1..84: note = Description of sequence: transcripted tRNA sequence; tRNA name: Ala_AGC_chr6:28763741-28763812 (-)
  source 1..84: mol_type = other DNA
ggagaggcctggccgagtggttaaggcgatggactgctaatccattgtgctctgcacgcgtgggttcgaatcccatcctcgtcg

SEQ ID NO: 206 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gcagcgatggccgagtggttaaggcgttggacttgaaatccaatggggtctccccgcgcaggttcgaaccctgctcgctgcg

SEQ ID NO: 207 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gtagtcgtggccgagtggttaaggcgatggacttgaaatccattggggtttccccgcgcaggttcgaatcctgccgactacg

SEQ ID NO: 208 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gtagtcgtggccgagtggttaaggcgatggacttgaaatccattggggtctccccgcgcaggttcgaatcctgccgactacg

SEQ ID NO: 209 - DNA, 82 bp, synthetic construct
  misc_feature 1..82: note = Description of sequence: transcripted tRNA sequence; tRNA name: Ala_AGC_chr6:28763741-28763812 (-)
  source 1..82: mol_type = other DNA
gtagtcgtggccgagtggttaaggcgatggacttgaaatccattggggtttccccgcgcaggttcgaatcctgtcggctacg

SEQ ID NO: 210 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggcgccgtggcttagttggttaaagcgcctgtctagtaaacaggagatcctgggttcgaatcccagcggtgcct

SEQ ID NO: 211 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggctccgtggcttagctggttaaagcgcctgtctagtaaacaggagatcctgggttcgaatcccagcggggcct

SEQ ID NO: 212 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggctccgtagcttagttggttaaagcgcctgtctagtaaacaggagatcctgggttcgactcccagcggggcct

SEQ ID NO: 213 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggcttcgtggcttagctggttaaagcgcctgtctagtaaacaggagatcctgggttcgaatcccagcgaggcct

SEQ ID NO: 214 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggcgccgtggcttagctggttaaagcgcctgtctagtaaacaggagatcctgggttcgaatcccagcggtgcct

SEQ ID NO: 215 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggccctgtggcttagctggtcaaagcgcctgtctagtaaacaggagatcctgggttcgaatcccagcggggcct

SEQ ID NO: 216 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggctctatggcttagttggttaaagcgcctgtctcgtaaacaggagatcctgggttcgactcccagtggggcct

SEQ ID NO: 217 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggcgcggtggccaagtggtaaggcgtcggtctcgtaaaccgaagatcacgggttcgaaccccgtccgtgcct

SEQ ID NO: 218 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggctctgtggcttagttggctaaagcgcctgtctcgtaaacaggagatcctgggttcgaatcccagcggggcct

SEQ ID NO: 219 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggcgcggtggccaagtggtaaggcgtcggtctcgtaaaccgaagatcgcgggttcgaaccccgtccgtgcct

SEQ ID NO: 220 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggccctgtagctcagcggttggagcgctggtctcgtaaacctaggggtcgtgagttcaaatctcaccagggcct

SEQ ID NO: 221 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggctctatggcttagttggttaaagcgcctgtcttgtaaacaggagatcctgggttcgaatcccagtagagcct

SEQ ID NO: 222 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggctccatagctcagtggttagagcactggtcttgtaaaccaggggtcgcgagttcgatcctcgctggggcct

SEQ ID NO: 223 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggctccatagctcaggggttagagcgctggtcttgtaaaccaggggtcgcgagttcaattctcgctggggcct

SEQ ID NO: 224 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggctccatagctcaggggttagagcactggtcttgtaaaccaggggtcgcgagttcaaatctcgctggggcct

SEQ ID NO: 225 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggccctatagctcaggggttagagcactggtcttgtaaaccaggggtcgcgagttcaaatctcgctggggcct

SEQ ID NO: 226 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggctccatagctcaggggttagagcactggtcttgtaaaccagggtcgcgagttcaaatctcgctggggcct

SEQ ID NO: 227 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggcctcgtggcgcaacggtagcgcgtctgactccagatcagaaggttgcgtgttcaaatcacgtcggggtca

SEQ ID NO: 228 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gacctcgtggcgcaatggtagcgcgtctgactccagatcagaaggttgcgtgttcaagtcacgtcggggtca

SEQ ID NO: 229 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gacctcgtggcgcaacggtagcgcgtctgactccagatcagaaggttgcgtgttcaaatcacgtcggggtca

SEQ ID NO: 230 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gacctcgtggcgcaacggtagcgcgtctgactccagatcagaaggctgcgtgttcgaatcacgtcggggtca

SEQ ID NO: 231 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gacctcgtggcgcaacggcagcgcgtctgactccagatcagaaggttgcgtgttcaaatcacgtcggggtca

SEQ ID NO: 232 - DNA, 93 bp, synthetic construct
  misc_feature 1..93: note = Description of sequence: transcripted tRNA sequence; tRNA name: Ala_AGC_chr6:28763741-28763812 (-)
  source 1..93: mol_type = other DNA
ccttcaatagttcagctggtagagcagaggactatagctacttcctcagtaggagacgtccttaggttgctggttcgattccagcttgaagga

SEQ ID NO: 233 - DNA, 91 bp, synthetic construct
  misc_feature 1..91: note = Description of sequence: transcripted tRNA sequence; tRNA name: Ala_AGC_chr6:28763741-28763812 (-)
  source 1..91: mol_type = other DNA
ccttcgatagctcagttggtagagcggaggactgtagttggctgtgtccttagacatccttaggtcgctggttcgaatccggctcgaagga

SEQ ID NO: 234 - DNA, 89 bp, synthetic construct
  misc_feature 1..89: note = Description of sequence: transcripted tRNA sequence; tRNA name: Ala_AGC_chr6:28763741-28763812 (-)
  source 1..89: mol_type = other DNA
ccttcgatagctcagttggtagagcggaggactgtagtggatagggcgtggcaatccttaggtcgctggttcgattccggctcgaagga

SEQ ID NO: 235 - DNA, 89 bp, synthetic construct
  misc_feature 1..89: note = Description of sequence: transcripted tRNA sequence; tRNA name: Ala_AGC_chr6:28763741-28763812 (-)
  source 1..89: mol_type = other DNA
ccttcgatagctcagttggtagagcggaggactgtaggctcattaagcaaggtatccttaggtcgctggttcgaatccggctcggagga

SEQ ID NO: 236 - DNA, 94 bp, synthetic construct
  misc_feature 1..94: note = Description of sequence: transcripted tRNA sequence; tRNA name: Ala_AGC_chr6:28763741-28763812 (-)
  source 1..94: mol_type = other DNA
ccttcgatagctcagctggtagagcggaggactgtagattgtatagacatttgcggacatccttaggtcgctggttcgattccagctcgaagga

SEQ ID NO: 237 - DNA, 93 bp, synthetic construct
  misc_feature 1..93: note = Description of sequence: transcripted tRNA sequence; tRNA name: Ala_AGC_chr6:28763741-28763812 (-)
  source 1..93: mol_type = other DNA
ccttcgatagctcagctggtagagcggaggactgtagctacttcctcagcaggagacatccttaggtcgctggttcgattccggctcgaagga

SEQ ID NO: 238 - DNA, 89 bp, synthetic construct
  misc_feature 1..89: note = Description of sequence: transcripted tRNA sequence; tRNA name: Ala_AGC_chr6:28763741-28763812 (-)
  source 1..89: mol_type = other DNA
ccttcgatagctcagctggtagagcggaggactgtaggcgcgcgcccgtggccatccttaggtcgctggttcgattccggctcgaagga

SEQ ID NO: 239 - DNA, 94 bp, synthetic construct
  misc_feature 1..94: note = Description of sequence: transcripted tRNA sequence; tRNA name: Ala_AGC_chr6:28763741-28763812 (-)
  source 1..94: mol_type = other DNA
ccttcgatagctcagctggtagagcggaggactgtagcctgtagaaacatttgtggacatccttaggtcgctggttcgattccggctcgaagga

SEQ ID NO: 240 - DNA, 94 bp, synthetic construct
  misc_feature 1..94: note = Description of sequence: transcripted tRNA sequence; tRNA name: Ala_AGC_chr6:28763741-28763812 (-)
  source 1..94: mol_type = other DNA
ccttcgatagctcagctggtagagcggaggactgtagattgtacagacatttgcggacatccttaggtcgctggttcgattccggctcgaagga

SEQ ID NO: 241 - DNA, 89 bp, synthetic construct
  misc_feature 1..89: note = Description of sequence: transcripted tRNA sequence; tRNA name: Ala_AGC_chr6:28763741-28763812 (-)
  source 1..89: mol_type = other DNA
ccttcgatagctcagctggtagagcggaggactgtagtacttaatgtgtggtcatccttaggtcgctggttcgattccggctcgaagga

SEQ ID NO: 242 - DNA, 89 bp, synthetic construct
  misc_feature 1..89: note = Description of sequence: transcripted tRNA sequence; tRNA name: Ala_AGC_chr6:28763741-28763812 (-)
  source 1..89: mol_type = other DNA
ccttcgatagctcagctggtagagcggaggactgtaggggtttgaatgtggtcatccttaggtcgctggttcgaatccggctcggagga

SEQ ID NO: 243 - DNA, 94 bp, synthetic construct
  misc_feature 1..94: note = Description of sequence: transcripted tRNA sequence; tRNA name: Ala_AGC_chr6:28763741-28763812 (-)
  source 1..94: mol_type = other DNA
ccttcgatagctcagctggtagagcggaggactgtagactgcggaaacgtttgtggacatccttaggtcgctggttcaattccggctcgaagga

SEQ ID NO: 244 - DNA, 90 bp, synthetic construct
  misc_feature 1..90: note = Description of sequence: transcripted tRNA sequence; tRNA name: Ala_AGC_chr6:28763741-28763812 (-)
  source 1..90: mol_type = other DNA
ctttcgatagctcagttggtagagcggaggactgtaggttcattaaactaaggcatccttaggtcgctggttcgaatccggctcgaagga

SEQ ID NO: 245 - DNA, 88 bp, synthetic construct
  misc_feature 1..88: note = Description of sequence: transcripted tRNA sequence; tRNA name: Ala_AGC_chr6:28763741-28763812 (-)
  source 1..88: mol_type = other DNA
tcttcaatagctcagctggtagagcggaggactgtaggtgcacgcccgtggccattcttaggtgctggtttgattccgacttggagag

SEQ ID NO: 246 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gtttccgtagtgtagtggttatcacgttcgcctaacacgcgaaaggtccccggttcgaaaccgggcggaaaca

SEQ ID NO: 247 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gtttccgtagtgtagtggtcatcacgttcgcctaacacgcgaaaggtccccggttcgaaaccgggcggaaaca

SEQ ID NO: 248 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gtttccgtagtgtagtggttatcacgttcgcctaacacgcgaaaggtccctggatcaaaaccaggcggaaaca

SEQ ID NO: 249 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gtttccgtagtgtagtggttatcacgttcgcctaacacgcgaaaggtccgcggttcgaaaccgggcggaaaca

SEQ ID NO: 250 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gtttccgtagtgtagtggttatcacgtttgcctaacacgcgaaaggtccccggttcgaaaccgggcagaaaca

SEQ ID NO: 251 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gggggtgtagctcagtggtagagcgtatgcttaacattcatgaggctctgggttcgatccccagcacttcca

SEQ ID NO: 252 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gtttccgtagtgtagtggttatcacgttcgcctcacacgcgaaaggtccccggttcgaaaccgggcggaaaca

SEQ ID NO: 253 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcttctgtagtgtagtggttatcacgttcgcctcacacgcgaaaggtccccggttcgaaaccgggcagaagca

SEQ ID NO: 254 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gtttccgtagtgtagcggttatcacattcgcctcacacgcgaaaggtccccggttcgatcccgggcggaaaca

SEQ ID NO: 255 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gtttccgtagtgtagtggttatcacgttcgcctcacacgcgaaaggtccccggttcgaaactgggcggaaaca

SEQ ID NO: 256 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtttccgtagtgtagtggttatcacgttcgcctcacacgcgtaaaggtccccggttcgaaaccgggcggaaaca

SEQ ID NO: 257 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gtttccgtagtggagtggttatcacgttcgcctcacacgcgaaaggtccccggtttgaaaccaggcggaaaca

SEQ ID NO: 258 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggttccatagtgtagtggttatcacgtctgctttacacgcagaaggtcctgggttcgagccccagtggaacca

SEQ ID NO: 259 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggttccatagtgtagcggttatcacgtctgctttacacgcagaaggtcctgggttcgagccccagtggaacca

SEQ ID NO: 260 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggttccatagtgtagtggttatcacatctgctttacacgcagaaggtcctgggttcaagccccagtggaacca

SEQ ID NO: 261 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gtttccgtggtgtagtggttatcacattcgccttacacgcgaaaggtcctcgggtcgaaaccgagcggaaaca

SEQ ID NO: 262 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
agcagagtggcgcagcggaagcgtgctgggcccataacccagaggtcgatggatcgaaaccatcctctgcta

SEQ ID NO: 263 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
agcagagtggcgcagcggaagcgtgctgggcccataacccagaggtcgatggatctaaaccatcctctgcta

SEQ ID NO: 264 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tccctggtggtctagtggctaggattcggcgctttcaccgccgcggcccgggttcgattcccggtcagggaat

SEQ ID NO: 265 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gcgttggtggtttagtggtagaattctcgcctcccatgcgggagacccgggttcaattcccggccactgcac

SEQ ID NO: 266 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
ggccttggtggtgcagtggtagaattctcgcctcccacgtgggagacccgggttcaattcccggccaatgca

SEQ ID NO: 267 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gtccctggtggtctagtggctaggattcggcgctttcaccgccgcggcccgggttcgattcccggccagggaa

SEQ ID NO: 268 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
tgtctctgtggcgcaatcggttagcgcgttcggctgttaaccgaaagattggtggttcgagcccacccagggacg

SEQ ID NO: 269 - DNA, 86 bp, synthetic construct
  misc_feature 1..86: note = Description of sequence: transcripted tRNA sequence; tRNA name: Ala_AGC_chr6:28763741-28763812 (-)
  source 1..86: mol_type = other DNA
tggctccgtggcgcaatggatagcgcattggacttctagaggctgaaggcattcaaaggttccgggttcgagtcccggcggagtcg

SEQ ID NO: 270 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gcccggctagctcagtcggtagagcatgagactcttaatctcagggtcgtgggttcgagccccacgttgggcgc

SEQ ID NO: 271 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccgtgatcgtatagtggttagtactctgcgttgtggccgcagcaacctcggttcgaatccgagtcacggcag

SEQ ID NO: 272 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gcgttggtggtatagtggtgagcatagctgccttccaagcagttgacccgggttcgattcccggccaacgcag

SEQ ID NO: 273 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tccctggtggtctagtggttaggattcggcgctctcaccgccgcggcccgggttcgattcccggtcagggaaa

SEQ ID NO: 274 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
aggttccatggtgtaatggtgagcactctggactctgaatccagcgatccgagttcgagtctcggtggaacct

SEQ ID NO: 275 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
tgtctctgtggcgtagtcggttagcgcgttcggctgttaaccgaaaagttggtggttcgagcccacccaggaacg

SEQ ID NO: 276 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
tgtctctgtggcgcaatcggttagcgcgttcggctgttaaccgaaaggttggtggttcgagcccacccagggacg

SEQ ID NO: 277 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
gtctctgtggcgcaatcggttagcgcattcggctgttaaccgaaaggttggtggttcgagcccacccagggacgc

SEQ ID NO: 278 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
gtctctgtggcgcaatgggttagcgcgttcggctgttaaccgaaaggttggtggttcgagcccatccagggacgc

SEQ ID NO: 279 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gcactggtggttcagtggtagaattctcgcctcacacgcgggacacccgggttcaattcccggtcaaggcaa

SEQ ID NO: 280 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtttccgtagtgtagtggttatcacgttcgcctcacacgcgaaaggtccccggttcgaaactgggcggaaacag

SEQ ID NO: 281 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gcactggtggttcagtggtagaattctcgcctcccacgcgggagacccgggtttaattcccggtcaagataa

SEQ ID NO: 282 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
gtttccgtagtgtagtggttatcacgttcgcctcacacgcgtaaaggtccccggttcgaaaccgggcggaaacat

SEQ ID NO: 283 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tagcagagtggcgcagcggaagcgtgctgggcccataacccagaggtcgatggatcgaaaccatcctctgcta

SEQ ID NO: 284 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtttccgtagtgtagtggttatcacgttcgcctcacacgcgaaaggtccccggttcgaaaccgggcggaaacaa

SEQ ID NO: 285 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tcctcgttagtatagtggtgagtatccccgcctgtcacgcgggagaccggggttcgattccccgacggggagg

SEQ ID NO: 286 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
tgcatgggtggttcagtggtagaattctcgcctgccacgcgggaggcccgggttcgattcccggcccatgca

SEQ ID NO: 287 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tccctggtggtctagtggttaggattcggcgctctcaccgccgcggcccgggttcgattcccggtcagggaag

SEQ ID NO: 288 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
atccttgttactatagtggtgagtatctctgcctgtcatgcgtgagagagggggtcgattccccgacggggag

SEQ ID NO: 289 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gcattggtggttcagtggtagaattctcgcctgccacgcgggaggcccgggttcgattcccggccaatgcac

SEQ ID NO: 290 - DNA, 84 bp, synthetic construct
  misc_feature 1..84: note = Description of sequence: transcripted tRNA sequence; tRNA name: Ala_AGC_chr6:28763741-28763812 (-)
  source 1..84: mol_type = other DNA
gtcaggatggccgagcggtctaaggcgctgcgttcaggtcgcagtctcccctggaggcgtgggttcgaatcccactcctgacaa

SEQ ID NO: 291 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
cgcgttggtggtatagtggtgagcatagctgccttccaagcagttgacccgggttcgattcccggccaacgca

SEQ ID NO: 292 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
cgtctctgtggcgcaatcggttagcgcgttcggctgttaaccgaaaggttggtggttcgatcccacccagggacg

SEQ ID NO: 293 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
cgcgttggtggtgtagtggtgagcacagctgcctttcaagcagttaacgcgggttcgattcccgggtaacgaa

SEQ ID NO: 294 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
cggctcgttggtctaggggtatgattctcgcttcgggtgcgagaggtcccgggttcaaatcccggacgagccc

SEQ ID NO: 295 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggctcgttggtctaggggtatgattctcgcttagggtgcgagaggtcccgggttcaaatcccggacgagccct

SEQ ID NO: 296 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
cgcccggatagctcagtcggtagagcatcagacttttaatctgagggtccagggttcaagtccctgttcgggcg

SEQ ID NO: 297 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gcccggatagctcagtcggtagagcatcagacttttaatctgagggtccagggttcaagtccctgttcgggcgt

SEQ ID NO: 298 - DNA, 107 bp, synthetic construct
  misc_feature 1..107: note = Description of sequence: transcripted tRNA sequence; tRNA name: Ala_AGC_chr6:28763741-28763812 (-)
  source 1..107: mol_type = other DNA
tgtcaggatggccgagtggtctaaggcgccagactcaaggtaagcaccttgcctgcgggctttctggtctccggatggaggcgtgggttcgaatcccacttctgaca

SEQ ID NO: 299 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ttccctggtggtctagtggttaggattcggcgctctcaccgccgcggcccgggttcgattcccggtcaggaaa

SEQ ID NO: 300 - DNA, 90 bp, synthetic construct
  misc_feature 1..90: note = Description of sequence: transcripted tRNA sequence; tRNA name: Ala_AGC_chr6:28763741-28763812 (-)
  source 1..90: mol_type = other DNA
gccttcgatagctcagttggtagagcggaggactgtagtggatagggcgtggcaatccttaggtcgctggttcgattccggctcgaagga

SEQ ID NO: 301 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
cgggggattagctcaaatggtagagcgctcgcttagcatgcgagaggtagcgggatcgatgcccgcatcctcca

SEQ ID NO: 302 - DNA, 94 bp, synthetic construct
  misc_feature 1..94: note = Description of sequence: transcripted tRNA sequence; tRNA name: Ala_AGC_chr6:28763741-28763812 (-)
  source 1..94: mol_type = other DNA
agctccagtggcgcaatcggttagcgcgcggtacttatacagcagtacatgcagagcaatgccgaggttgtgagttcgagcctcacctggagca

SEQ ID NO: 303 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gcgccgctggtgtagtggtatcatgcaagattcccattcttgcgacccgggttcgattcccgggcggcgcat

SEQ ID NO: 304 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tcccatatggtctagcggttaggattcctggttttcacccaggtggcccgggttcgactcccggtatgggaac

SEQ ID NO: 305 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gggggatgtagctcagtggtagagcgcgcgcttcgcatgtgtgaggtcccgggttcaatccccggcatctcca

SEQ ID NO: 306 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gcattggtggttcagtggtagaattctcgcctgccacgcgggaggcccgggttcgattcccggccaatgcaa

SEQ ID NO: 307 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gggccagtggcgcaatggataacgcgtctgactacggatcagaagattctaggttcgactcctggctggctcgc

SEQ ID NO: 308 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggtttccgtagtgtagtggttatcacgttcgcctaacacgcgaaaggtccccggttcgaaaccgggcggaaaca

SEQ ID NO: 309 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
agtttccgtagtgtagtggttatcacgttcgcctaacacgcgaaaggtccccggttcgaaaccgggcggaaaca

SEQ ID NO: 310 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
aggtagcgtggccgagcggtctaaggcgctggattaaggctccagtctcttcgggggcgtgggttcgaatcccaccgctgcca

SEQ ID NO: 311 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtttccgtagtgtagtggtcatcacgttcgcctaacacgcgaaaggtccccggttcgaaaccgggcggaaacat

SEQ ID NO: 312 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggctcgttggtctaggggtatgattctcgctttgggtgcgagaggtcccgggttcaaatcccggacgagccca

SEQ ID NO: 313 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggctccatagctcaggggttagagcactggtcttgtaaaccagggtcgcgagttcaaatctcgctggggcctg

SEQ ID NO: 314 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tggggatgtagctcagtggtagagcgcatgctttgcatgtatgaggccccgggttcgatccccggcatctcca

SEQ ID NO: 315 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
cgcccggctagctcagtcggtagagcatgagactcttaatctcagggtcgtgggttcgagccccacgttgggcg

SEQ ID NO: 316 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtttccgtagtgtagtggttatcacgttcgcctaacacgcgaaaggtccccggttcgaaaccgggcggaaacaa

SEQ ID NO: 317 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gcccggctagctcagtcggtagagcatgagactcttaatctcagggtcgtgggttcgagccccacgttgggcgt

SEQ ID NO: 318 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtttccgtagtgtagtggttatcacgttcgcctcacacgcgaaaggtccccggttcgaaaccgggcggaaacac

SEQ ID NO: 319 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
cagcagagtggcgcagcggaagcgtgctgggcccataacccagaggtcgatggatcgaaaccatcctctgcta

SEQ ID NO: 320 - DNA, 85 bp, synthetic construct
  misc_feature 1..85: note = Description of sequence: transcripted tRNA sequence; tRNA name: Ala_AGC_chr6:28763741-28763812 (-)
  source 1..85: mol_type = other DNA
ggagaggcctggccgagtggttaaggcgatggactgctaatccattgtgctctgcacgcgtgggttcgaatcccatcctcgtcgc

SEQ ID NO: 321 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggccccatggtgtaatggttagcactctggactttgaatccagcgatccgagttcaaatctcggtgggacctg

SEQ ID NO: 322 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggccccatggtgtaatggttagcactctggactttgaatccagcgatccgagttcaaatctcggtgggaccta

SEQ ID NO: 323 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
gtagtcgtggccgagtggttaaggcgatggacttgaaatccattggggtctccccgcgcaggttcgaatcctgccgactacgg

SEQ ID NO: 324 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
agcagagtggcgcagcggaagcgtgctgggcccataacccagaggtcgatggatcgaaaccatcctctgctat

SEQ ID NO: 325 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggaccacgtggcctaatggataaggcgtctgacttcggatcagaagattgagggttcgaatccctccgtggtta

SEQ ID NO: 326 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
tgtagtcgtggccgagtggttaaggcgatggactagaaatccattggggtctccccgcgcaggttcgaatcctgccgactacg

SEQ ID NO: 327 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
agcagagtggcgcagcggaagcgtgctgggcccataacccagaggtcgatggatcgaaaccatcctctgctag

SEQ ID NO: 328 - DNA, 84 bp, synthetic construct
  misc_feature 1..84: note = Description of sequence: transcripted tRNA sequence; tRNA name: Ala_AGC_chr6:28763741-28763812 (-)
  source 1..84: mol_type = other DNA
cgtcaggatggccgagcggtctaaggcgctgcgttcaggtcgcagtctcccctggaggcgtgggttcgaatcccactcctgaca

SEQ ID NO: 329 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
ggctccgtggcttagctggttaaagcgcctgtctagtaaacaggagatcctgggttcgaatcccagcggggcctg

SEQ ID NO: 330 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
agggccagtggcgcaatggataacgcgtctgactacggatcagaagattccaggttcgactcctggctggctcg

SEQ ID NO: 331 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggtttccgtagtgtagtggttatcacgttcgcctcacacgcgaaaggtccccggttcgaaaccgggcggaaaca

SEQ ID NO: 332 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
aggggatgtagctcagtggtagagcgcatgcttcgcatgtatgaggtcccgggttcgatccccggcatctcca

SEQ ID NO: 333 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
tggccggttagctcagttggttagagcgtggtgctaataacgccaaggtcgcgggttcgatccccgtacgggcca

SEQ ID NO: 334 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
cggctcgttggtctaggggtatgattctcgcttagggtgcgagaggtcccgggttcaaatcccggacgagccc

SEQ ID NO: 335 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
agcccggctagctcagtcggtagagcatgagactcttaatctcagggtcgtgggttcgagccccacgttgggcg

SEQ ID NO: 336 - DNA, 92 bp, synthetic construct
  misc_feature 1..92: note = Description of sequence: transcripted tRNA sequence; tRNA name: Ala_AGC_chr6:28763741-28763812 (-)
  source 1..92: mol_type = other DNA
tccttcgatagctcagttggtagagcggaggactgtagttggctgtgtccttagacatccttaggtcgctggttcgaatccggctcgaagga

SEQ ID NO: 337 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggggaattagctcaaatggtagagcgctcgcttagcatgcgagaggtagcgggatcgatgcccgcattctccag

SEQ ID NO: 338 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
cgccctcttagcgcagcgggcagcgcgtcagtctcataatctgaaggtcctgagttcgagcctcagagagggca

SEQ ID NO: 339 - DNA, 95 bp, synthetic construct
  misc_feature 1..95: note = Description of sequence: transcripted tRNA sequence; tRNA name: Ala_AGC_chr6:28763741-28763812 (-)
  source 1..95: mol_type = other DNA
tgctccagtggcgcaatcggttagcgcgcggtacttatatggcagtatgtgtgcgagtgatgccgaggttgtgagttcgagcctcacctggagca

SEQ ID NO: 340 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tgccgtgatcgtatagtggttagtactctgcgttgtggccgcagcaacctcggttcgaatccgagtcacggca

SEQ ID NO: 341 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
ggccggttagctcagttggttagagcgtggtgctaataacgccaaggtcgcgggttcgatccccgtacgggccac

SEQ ID NO: 342 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
agtttccgtagtgtagtggttatcacgtttgcctaacacgcgaaaggtccccggttcgaaaccgggcagaaaca

SEQ ID NO: 343 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gcttctgtagtgtagtggttatcacgttcgcctcacacgcgaaaggtccccggttcgaaaccgggcagaagcaa

SEQ ID NO: 344 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ttcctcgttagtatagtggtgagtatccccgcctgtcacgcgggagaccggggttcgattccccgacggggag

SEQ ID NO: 345 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
gtagtcgtggccgagtggttaaggcgatggacttgaaatccattggggtttccccgcgcaggttcgaatcctgtcggctacgg

SEQ ID NO: 346 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
aggttccatggtgtaatggttagcactctggactctgaatccagcgatccgagttcaaatctcggtggaacct

SEQ ID NO: 347 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tcctcgttagtatagtggtgagtgtccccgtctgtcacgcgggagaccggggttcgattccccgacggggaga

SEQ ID NO: 348 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtttccgtagtgtagtggttatcacgttcgcctaacacgcgaaaggtccctggatcaaaaccaggcggaaacaa

SEQ ID NO: 349 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
cggccggttagctcagttggttagagcgtggtgctaataacgccaaggtcgcgggttcgatccccgtactggcca

SEQ ID NO: 350 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggccccatggtgtaatggtcagcactctggactctgaatccagcgatccgagttcaaatctcggtgggaccca

SEQ ID NO: 351 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggccccatggtgtaatggttagcactctggactttgaatccagcgatccgagttcaaatctcggtgggacctt

SEQ ID NO: 352 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tgggggtgtagctcagtggtagagcgcgtgcttagcatgtacgaggtcccgggttcaatccccggcacctcca

SEQ ID NO: 353 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggggatgtagctcagtggtagagcgcatgcttagcatgcatgaggtcccgggttcgatccccagcatctccag

SEQ ID NO: 354 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
agggggtgtagctcagtggtagagcgcgtgcttcgcatgtacgaggccccgggttcgacccccggctcctcca

SEQ ID NO: 355 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gggggtgtagctcagtggtagagcgcgtgcttagcatgcacgaggccccgggttcaatccccggcacctccat

SEQ ID NO: 356 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gggggtgtagctcagtggtagagcgcgtgcttagcatgcacgaggccccgggttcaatccccggcacctccag

SEQ ID NO: 357 - DNA, 107 bp, synthetic construct
  misc_feature 1..107: note = Description of sequence: transcripted tRNA sequence; tRNA name: Ala_AGC_chr6:28763741-28763812 (-)
  source 1..107: mol_type = other DNA
gtcaggatggccgagtggtctaaggcgccagactcaagctaagcttcctccgcggtggggattctggtctccaatggaggcgtgggttcgaatcccacttctgacac

SEQ ID NO: 358 - DNA, 106 bp, synthetic construct
  misc_feature 1..106: note = Description of sequence: transcripted tRNA sequence; tRNA name: Ala_AGC_chr6:28763741-28763812 (-)
  source 1..106: mol_type = other DNA
tgtcaggatggccgagtggtctaaggcgccagactcaagcttggcttcctcgtgttgaggattctggtctccaatggaggcgtgggttcgaatcccacttctgaca

SEQ ID NO: 359 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggttccatggtgtaatggttagcactctggactctgaatccagcgatccgagttcaaatctcggtggaacctt

SEQ ID NO: 360 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
ggtagcgtggccgagcggtctaaggcgctggattaaggctccagtctcttcgggggcgtgggttcgaatcccaccgctgccag

SEQ ID NO: 361 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
tgcctccttagcgcagtaggcagcgcgtcagtctcataatctgaaggtcctgagttcgaacctcagagggggca

SEQ ID NO: 362 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
agcccggatagctcagtcggtagagcatcagacttttaatctgagggtccagggttcaagtccctgttcgggcg

SEQ ID NO: 363 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gcctccttagcgcagtaggcagcgcgtcagtctcataatctgaaggtcctgagttcgaacctcagagggggcag

SEQ ID NO: 364 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ttccctggtggtctagtggttaggattcggcgctctcaccgccgcggcccgggttcgattcccggtcagggaa

SEQ ID NO: 365 - DNA, 84 bp, synthetic construct
  misc_feature 1..84: note = Description of sequence: transcripted tRNA sequence; tRNA name: Ala_AGC_chr6:28763741-28763812 (-)
  source 1..84: mol_type = other DNA
caccaggatggccgagtggttaaggcgttggacttaagatccaatggacatatgtccgcgtgggttcgaaccccactcctggta

SEQ ID NO: 366 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tggctcgttggtctaggggtatgattctcgcttagggtgcgagaggtcccgggttcaaatcccggacgagccc

SEQ ID NO: 367 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
agccccagtggcctaatggataaggcattggcctcctaagccagggattgtgggttcgagtcccatctggggtg

SEQ ID NO: 368 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggggatatagctcaggggtagagcatttgactgcagatcaagaggtccccggttcaaatccgggtgccccccc

SEQ ID NO: 369 - DNA, 94 bp, synthetic construct
  misc_feature 1..94: note = Description of sequence: transcripted tRNA sequence; tRNA name: Ala_AGC_chr6:28763741-28763812 (-)
  source 1..94: mol_type = other DNA
cccttcgatagctcagctggtagagcggaggactgtagctacttcctcagcaggagacatccttaggtcgctggttcgattccggctcgaagga

SEQ ID NO: 370 - DNA, 90 bp, synthetic construct
  misc_feature 1..90: note = Description of sequence: transcripted tRNA sequence; tRNA name: Ala_AGC_chr6:28763741-28763812 (-)
  source 1..90: mol_type = other DNA
cccttcgatagctcagctggtagagcggaggactgtaggcgcgcgcccgtggccatccttaggtcgctggttcgattccggctcgaagga

SEQ ID NO: 371 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
tgggggattagctcaaatggtagagcgctcgcttagcatgcgagaggtagcgggatcgatgcccgcatcctcca

SEQ ID NO: 372 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
gtagtcgtggccgagtggttaaggcgatggactagaaatccattggggtctccccgcgcaggttcgaatcctgccgactacgg

SEQ ID NO: 373 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gcctcgttagcgcagtaggtagcgcgtcagtctcataatctgaaggtcgtgagttcgatcctcacacggggcac

SEQ ID NO: 374 - DNA, 92 bp, synthetic construct
  misc_feature 1..92: note = Description of sequence: transcripted tRNA sequence; tRNA name: Ala_AGC_chr6:28763741-28763812 (-)
  source 1..92: mol_type = other DNA
ggctctgtggcgcaatggatagcgcattggacttctagctgagcctagtgtggtcattcaaaggttgtgggttcgagtcccaccagagtcga

SEQ ID NO: 375 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
gtctctgtggcgcaatcggttagcgcgttcggctgttaaccgaaaggttggtggttcgagcccacccagggacgc

SEQ ID NO: 376 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
ggcagcgatggccgagtggttaaggcgttggacttgaaatccaatggggtctccccgcgcaggttcgaaccctgctcgctgcg

SEQ ID NO: 377 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggttccatagtgtagtggttatcacgtctgctttacacgcagaaggtcctgggttcgagccccagtggaaccat

SEQ ID NO: 378 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggttccatagtgtagcggttatcacgtctgctttacacgcagaaggtcctgggttcgagccccagtggaaccac

SEQ ID NO: 379 - DNA, 87 bp, synthetic construct
  misc_feature 1..87: note = Description of sequence: transcripted tRNA sequence; tRNA name: Ala_AGC_chr6:28763741-28763812 (-)
  source 1..87: mol_type = other DNA
tggctctgtggcgcaatggatagcgcattggacttctagatagttagagaaattcaaaggttgtgggttcgagtcccaccagagtcg

SEQ ID NO: 380 - DNA, 84 bp, synthetic construct
  misc_feature 1..84: note = Description of sequence: transcripted tRNA sequence; tRNA name: Ala_AGC_chr6:28763741-28763812 (-)
  source 1..84: mol_type = other DNA
taccagaatggccgagtggttaaggcgttggacttaagatccaatggattcatatccgcgtgggttcgaaccccacttctggta

SEQ ID NO: 381 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggcccggatagctcagtcggtagagcatcagacttttaatctgagggtccggggttcaagtccctgttcgggcg

SEQ ID NO: 382 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gccgaaatagctcagttgggagagcgttagactgaagatctaaaggtccctggttcgatcccgggtttcggcag

SEQ ID NO: 383 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gcccggatagctcagtcggtagagcatcagacttttaatctgagggtccagggttcaagtccctgttcgggcgg

SEQ ID NO: 384 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gccgaaatagctcagttgggagagcgttagactgaagatctaaaggtccctggttcaatcccgggtttcggcag

SEQ ID NO: 385 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
ggacgaggtggccgagtggttaaggcgatggactgctaatccattgtgctttgcacgcgtgggttcgaatcccatcctcgtcg

SEQ ID NO: 386 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggctcgttggtctaggggtatgattctcggtttgggtccgagaggtcccgggttcaaatcccggacgagcccc

SEQ ID NO: 387 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
agtcacggtggccgagtggttaaggcgttggactcgaaatccaatggggtttccccgcacaggttcgaatcctgttcgtgacg

SEQ ID NO: 388 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ctcctcgttagtatagtggttagtatccccgcctgtcacgcgggagaccggggttcaattccccgacggggag

SEQ ID NO: 389 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggacctcgtggcgcaacggtagcgcgtctgactccagatcagaaggctgcgtgttcgaatcacgtcggggtca

SEQ ID NO: 390 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggggatgtagctcagtggtagagcgcatgctttgcatgtatgaggccccgggttcgatccccggcatctccat

SEQ ID NO: 391 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gccgaaatagctcagttgggagagcgttagactgaagatctaaaggtccctggttcgatcccgggtttcggcac

SEQ ID NO: 392 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
aggggatgtagctcagtggtagagcgcatgctttgcacgtatgaggccccgggttcaatccccggcatctcca

SEQ ID NO: 393 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
gtctctgtggcgcaatcggttagcgcgttcggctgttaaccgaaaggttggtggttcgagcccacccagggacgg

SEQ ID NO: 394 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tcccacatggtctagcggttaggattcctggttttcacccaggcggcccgggttcgactcccggtgtgggaac

SEQ ID NO: 395 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggctccatagctcaggggttagagcgctggtcttgtaaaccaggggtcgcgagttcaattctcgctggggcctg

SEQ ID NO: 396 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
tggtagtgtggccgagcggtctaaggcgctggatttaggctccagtctcttcgggggcgtgggttcgaatcccaccactgcca

SEQ ID NO: 397 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
ggctccatagctcaggggttagagcactggtcttgtaaaccaggggtcgcgagttcaaatctcgctggggcctc

SEQ ID NO: 398 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tggctcgttggtctagtggtatgattctcgctttgggtgcgagaggtcccgggttcaaatcccggacgagccc

SEQ ID NO: 399 - DNA, 95 bp, synthetic construct
  misc_feature 1..95: note = Description of sequence: transcripted tRNA sequence; tRNA name: Ala_AGC_chr6:28763741-28763812 (-)
  source 1..95: mol_type = other DNA
ccttcgatagctcagctggtagagcggaggactgtagattgtacagacatttgcggacatccttaggtcgctggttcgattccggctcgaaggaa

SEQ ID NO: 400 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
aggccctatagctcaggggttagagcactggtcttgtaaaccaggggtcgcgagttcaaatctcgctggggcct

SEQ ID NO: 401 - DNA, 90 bp, synthetic construct
  misc_feature 1..90: note = Description of sequence: transcripted tRNA sequence; tRNA name: Ala_AGC_chr6:28763741-28763812 (-)
  source 1..90: mol_type = other DNA
tccttcgatagctcagctggtagagcggaggactgtagtacttaatgtgtggtcatccttaggtcgctggttcgattccggctcgaagga

SEQ ID NO: 402 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tggctcgttggtctaggggtatgattctcgctttgggtgcgagaggtcccgggttcaaatcccggacgagccc

SEQ ID NO: 403 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gcccggctagctcagtcggtagagcatgggactcttaatcccagggtcgtgggttcgagccccacgttgggcgc

SEQ ID NO: 404 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
cggccggttagctcagttggttagagcgtggtgctaataacgccaaggtcgcgggttcgatccccgtacgggcca

SEQ ID NO: 405 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tcccacatggtctagcggttaggattcctggttttcacccaggcggcccgggttcgactcccggtgtgggaat

SEQ ID NO: 406 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
gacgaggtggccgagtggttaaggcgatggactgctaatccattgtgctctgcacgcgtgggttcgaatcccatcctcgtcga

SEQ ID NO: 407 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gccgtgatcgtatagtggttagtactctgcgttgtggccgcagcaacctcggttcgaatccgagtcacggcat

SEQ ID NO: 408 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
cgccgtgatcgtatagtggttagtactctgcgttgtggccgcagcaacctcggttcgaatccgagtcacggca

SEQ ID NO: 409 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggttccatggtgtaatggttagcactctggactctgaatccagcgatccgagttcaaatctcggtggaacctg

SEQ ID NO: 410 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
tgcccggctagctcagtcggtagagcatgggactcttaatcccagggtcgtgggttcgagccccacgttgggcg

SEQ ID NO: 411 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gggccgcgtggcctaatggataaggcgtctgacttcggatcagaagattgcaggttcgagtcctgccgcggtcg

SEQ ID NO: 412 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gcgccgctggtgtagtggtatcatgcaagattcccattcttgcgacccgggttcgattcccgggcggcgcac

SEQ ID NO: 413 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gggccgcgtggcctaatggataaggcgtctgattccggatcagaagattgagggttcgagtcccttcgtggtcg

SEQ ID NO: 414 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
cgccccggtggcctaatggataaggcattggcctcctaagccagggattgtgggttcgagtcccacccggggta

SEQ ID NO: 415 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gcccggctagctcagtcggtagagcatgagacccttaatctcagggtcgtgggttcgagccccacgttgggcgt

SEQ ID NO: 416 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
aggcgcggtggccaagtggtaaggcgtcggtctcgtaaaccgaagatcacgggttcgaaccccgtccgtgcct

SEQ ID NO: 417 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
ggtagcgtggccgagtggtctaaggcgctggatttaggctccagtcatttcgatggcgtgggttcgaatcccaccgctgccac

SEQ ID NO: 418 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
gggtagcgtggccgagcggtctaaggcgctggattaaggctccagtctcttcgggggcgtgggttcgaatcccaccgctgcca

SEQ ID NO: 419 - DNA, 84 bp, synthetic construct
  misc_feature 1..84: note = Description of sequence: transcripted tRNA sequence; tRNA name: Ala_AGC_chr6:28763741-28763812 (-)
  source 1..84: mol_type = other DNA
agtcaggatggccgagcggtctaaggcgctgcgttcaggtcgcagtctcccctggaggcgtgggttcgaatcccacttctgaca

SEQ ID NO: 420 - DNA, 84 bp, synthetic construct
  misc_feature 1..84: note = Description of sequence: transcripted tRNA sequence; tRNA name: Ala_AGC_chr6:28763741-28763812 (-)
  source 1..84: mol_type = other DNA
gtcaggatggccgagcggtctaaggcgctgcgttcaggtcgcagtctcccctggaggcgtgggttcgaatcccacttctgacag

SEQ ID NO: 421 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gcctcgttagcgcagtaggcagcgcgtcagtctcataatctgaaggtcgtgagttcgagcctcacacggggcag

SEQ ID NO: 422 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
ggtagcgtggccgagcggtctaaggcgctggatttaggctccagtctcttcggaggcgtgggttcgaatcccaccgctgccag

SEQ ID NO: 423 - DNA, 89 bp, synthetic construct
  misc_feature 1..89: note = Description of sequence: transcripted tRNA sequence; tRNA name: Ala_AGC_chr6:28763741-28763812 (-)
  source 1..89: mol_type = other DNA
tggctctgtggcgcaatggatagcgcattggacttctagtgacgaatagagcaattcaaaggttgtgggttcgaatcccaccagagtcg

SEQ ID NO: 424 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
cgcattggtggttcagtggtagaattctcgcctgccacgcgggaggcccgggttcgattcccggccaatgca

SEQ ID NO: 425 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
gctgtgatggccgagtggttaaggcgttggactcgaaatccaatggggtctccccgcgcaggttcgaatcctgctcacagcgt

SEQ ID NO: 426 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
ggcgccgtggcttagctggttaaagcgcctgtctagtaaacaggagatcctgggttcgaatcccagcggtgcctg

SEQ ID NO: 427 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
cgacctcgtggcgcaacggtagcgcgtctgactccagatcagaaggttgcgtgttcaaatcacgtcggggtca

SEQ ID NO: 428 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
agacgaggtggccgagtggttaaggcgatggactgctaatccattgtgctctgcacgcgtgggttcgaatcccatcctcgtcg

SEQ ID NO: 429 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
cggcgccgtggcttagttggttaaagcgcctgtctagtaaacaggagatcctgggttcgaatcccagcggtgcct

SEQ ID NO: 430 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggcctcgtggcgcaacggtagcgcgtctgactccagatcagaaggttgcgtgttcaaatcacgtcggggtcaa

SEQ ID NO: 431 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
agcgttggtggtatagtggtaagcatagctgccttccaagcagttgacccgggttcgattcccggccaacgca

SEQ ID NO: 432 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tcctcgttagtatagtggtgagtatccccgcctgtcacgcgggagaccggggttcgattccccgacggggaga

SEQ ID NO: 433 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggctcgttggtctaggggtatgattctcgcttcgggtgcgagaggtcccgggttcaaatcccggacgagccct

SEQ ID NO: 434 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
ggcgccgtggcttagttggttaaagcgcctgtctagtaaacaggagatcctgggttcgaatcccagcggtgcctt

SEQ ID NO: 435 - DNA, 83 bp, synthetic construct
  misc_feature 1..83: note = Description of sequence: transcripted tRNA sequence; tRNA name: Ala_AGC_chr6:28763741-28763812 (-)
  source 1..83: mol_type = other DNA
gtagtcgtggccgagtggttaaggcgatggactagaaatccattggggtctccccgcgcaggttcgaatcctgccgactacgt

SEQ ID NO: 436 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
tgacctcgtggcgcaatggtagcgcgtctgactccagatcagaaggttgcgtgttcaagtcacgtcggggtca

SEQ ID NO: 437 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
aggcgcggtggccaagtggtaaggcgtcggtctcgtaaaccgaagatcgcgggttcgaaccccgtccgtgcct

SEQ ID NO: 438 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
agggggtatagctcagtggtagagcatttgactgcagatcaagaggtccccggttcaaatccgggtgccccct

SEQ ID NO: 439 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gggggtatagctcagtggtagagcatttgactgcagatcaagaggtccctggttcaaatccgggtgccccctc

SEQ ID NO: 440 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
gggggtatagctcagtggtagagcatttgactgcagatcaagaggtccccggttcaaatccgggtgccccctc

SEQ ID NO: 441 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
aggtcccatggtgtaatggttagcactctggactttgaatccagcgatccgagttcaaatctcggtgggacct

SEQ ID NO: 442 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gacccagtggcctaatggataaggcatcagcctccggagctggggattgtgggttcgagtcccatctgggtcgc

SEQ ID NO: 443 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
agccccagtggcctaatggataaggcactggcctcctaagccagggattgtgggttcgagtcccacctggggta

SEQ ID NO: 444 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gccccagtggcctaatggataaggcactggcctcctaagccagggattgtgggttcgagtcccacctggggtgt

SEQ ID NO: 445 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
agaccgcgtggcctaatggataaggcgtctgacttcggatcagaagattgagggttcgagtcccttcgtggtcg

SEQ ID NO: 446 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
cgtctctgtggcgcaatcggttagcgcgttcggctgttaaccgaaaggttggtggttcgagcccacccagggacg

SEQ ID NO: 447 - DNA, 73 bp, synthetic construct
  misc_feature 1..73: note = Description of sequence: transcripted tRNA sequence; tRNA name: Ala_AGC_chr6:28763741-28763812 (-)
  source 1..73: mol_type = other DNA
ggcgttggtggtatagtggttagcatagctgccttccaagcagttgacccgggttcgattcccggccaacgca

SEQ ID NO: 448 - DNA, 74 bp, synthetic construct
  misc_feature 1..74: note = Description of sequence: transcripted tRNA sequence; tRNA name: Ala_AGC_chr6:28763741-28763812 (-)
  source 1..74: mol_type = other DNA
gtttccgtagtgtagcggttatcacattcgcctcacacgcgaaaggtccccggttcgatcccgggcggaaacag

SEQ ID NO: 449 - DNA, 75 bp, synthetic construct
  misc_feature 1..75: note = Description of sequence: transcripted tRNA sequence; tRNA name: Ala_AGC_chr6:28763741-28763812 (-)
  source 1..75: mol_type = other DNA
tggcgccgtggcttagttggttaaagcgcctgtctagtaaacaggagatcctgggttcgaatcccagcggtgcct

SEQ ID NO: 450 - DNA, 94 bp, synthetic construct
  misc_feature 1..94: note = Description of sequence: transcripted tRNA sequence; tRNA name: Ala_AGC_chr6:28763741-28763812 (-)
  source 1..94: mol_type = other DNA
gctccagtggcgcaatcggttagcgcgcggtacttatatgacagtgcgagcggagcaatgccgaggttgtgagttcgatcctcacctggagcac

SEQ ID NO: 451 - DNA, 72 bp, synthetic construct
  misc_feature 1..72: note = Description of sequence: transcripted tRNA sequence; tRNA name: Ala_AGC_chr6:28763741-28763812 (-)
  source 1..72: mol_type = other DNA
gcatgggtggttcagtggtagaattctcgcctgccacgcgggaggcccgggttcgattcccggcccatgcag

SEQ ID NO: 452 - DNA, 18 bp, synthetic construct
  misc_feature 1..18: note = Description of sequence: Exemplary variable region sequence
  source 1..18: mol_type = other DNA
aaaatataaatatatttc

SEQ ID NO: 453-454 - skipped (2 entries)

SEQ ID NO: 455 - DNA, 15 bp, synthetic construct
  misc_feature 1..15: note = Description of sequence: Exemplary variable region sequence
  source 1..15: mol_type = other DNA
aattcttcggaatgt

SEQ ID NO: 456-460 - skipped (5 entries)

SEQ ID NO: 461 - DNA, 20 bp, synthetic construct
  misc_feature 1..20: note = Description of sequence: Exemplary variable region sequence
  source 1..20: mol_type = other DNA
caggcgggttctgcccgcgc

SEQ ID NO: 462 - DNA, 18 bp, synthetic construct
  misc_feature 1..18: note = Description of sequence: Exemplary variable region sequence
  source 1..18: mol_type = other DNA
catacctgcaagggtatc

SEQ ID NO: 463 - DNA, 15 bp, synthetic construct
  misc_feature 1..15: note = Description of sequence: Exemplary variable region sequence
  source 1..15: mol_type = other DNA
cgaccgcaaggttgt

SEQ ID NO: 464 - DNA, 15 bp, synthetic construct
  misc_feature 1..15: note = Description of sequence: Exemplary variable region sequence
  source 1..15: mol_type = other DNA
cgaccttgcggtcat

SEQ ID NO: 465 - DNA, 18 bp, synthetic construct
  misc_feature 1..18: note = Description of sequence: Exemplary variable region sequence
  source 1..18: mol_type = other DNA
cgatgctaatcacatcgt

SEQ ID NO: 466 - DNA, 15 bp, synthetic construct
  misc_feature 1..15: note = Description of sequence: Exemplary variable region sequence
  source 1..15: mol_type = other DNA
cgatggtgacatcat

SEQ ID NO: 467 - DNA, 16 bp, synthetic construct
  misc_feature 1..16: note = Description of sequence: Exemplary variable region sequence
  source 1..16: mol_type = other DNA
cgatggtttacatcgt

SEQ ID NO: 468 - DNA, 13 bp, synthetic construct
  misc_feature 1..13: note = Description of sequence: Exemplary variable region sequence
  source 1..13: mol_type = other DNA
cgccgtaaggtgt

SEQ ID NO: 469 - DNA, 12 bp, synthetic construct
  misc_feature 1..12: note = Description of sequence: Exemplary variable region sequence
  source 1..12: mol_type = other DNA
cgccttaggtgt

SEQ ID NO: 470 - DNA, 15 bp, synthetic construct
  misc_feature 1..15: note = Description of sequence: Exemplary variable region sequence
  source 1..15: mol_type = other DNA
cgcctttcgacgcgt

SEQ ID NO: 471 - DNA, 13 bp, synthetic construct
  misc_feature 1..13: note = Description of sequence: Exemplary variable region sequence
  source 1..13: mol_type = other DNA
cgcttcacggcgt

SEQ ID NO: 472 - DNA, 15 bp, synthetic construct
  misc_feature 1..15: note = Description of sequence: Exemplary variable region sequence
  source 1..15: mol_type = other DNA
cggcagcaatgctgt

SEQ ID NO: 473 - DNA, 13 bp, synthetic construct
  misc_feature 1..13: note = Description of sequence: Exemplary variable region sequence
  source 1..13: mol_type = other DNA
cggctccgccttc

SEQ ID NO: 474 - DNA, 16 bp, synthetic construct
  misc_feature 1..16: note = Description of sequence: Exemplary variable region sequence
  source 1..16: mol_type = other DNA
cgggtatcacagggtc

SEQ ID NO: 475 - DNA, 17 bp, synthetic construct
  misc_feature 1..17: note = Description of sequence: Exemplary variable region sequence
  source 1..17: mol_type = other DNA
cggtgcgcaagcgctgt

SEQ ID NO: 476 - DNA, 18 bp, synthetic construct
  misc_feature 1..18: note = Description of sequence: Exemplary variable region sequence
  source 1..18: mol_type = other DNA
cgtacgggtgaccgtacc

SEQ ID NO: 477 - DNA, 13 bp, synthetic construct
  misc_feature 1..13: note = Description of sequence: Exemplary variable region sequence
  source 1..13: mol_type = other DNA
cgtcaaagacttc

SEQ ID NO: 478 - DNA, 13 bp, synthetic construct
  misc_feature 1..13: note = Description of sequence: Exemplary variable region sequence
  source 1..13: mol_type = other DNA
cgtcgtaagactt

SEQ ID NO: 479 - DNA, 14 bp, synthetic construct
  misc_feature 1..14: note = Description of sequence: Exemplary variable region sequence
  source 1..14: mol_type = other DNA
cgttgaataaacgt

SEQ ID NO: 480-486 - skipped (7 entries)

SEQ ID NO: 487 - DNA, 15 bp, synthetic construct
  misc_feature 1..15: note = Description of sequence: Exemplary variable region sequence
  source 1..15: mol_type = other DNA
tactcgtatgggtgc

SEQ ID NO: 488 - DNA, 13 bp, synthetic construct
  misc_feature 1..13: note = Description of sequence: Exemplary variable region sequence
  source 1..13: mol_type = other DNA
tactttgcggtgt

SEQ ID NO: 489 - DNA, 19 bp, synthetic construct
  misc_feature 1..19: note = Description of sequence: Exemplary variable region sequence
  source 1..19: mol_type = other DNA
taggcgagtaacatcgtgc

SEQ ID NO: 490 - DNA, 19 bp, synthetic construct
  misc_feature 1..19: note = Description of sequence: Exemplary variable region sequence
  source 1..19: mol_type = other DNA
taggcgtgaatagcgcctc

SEQ ID NO: 491 - DNA, 19 bp, synthetic construct
  misc_feature 1..19: note = Description of sequence: Exemplary variable region sequence
  source 1..19: mol_type = other DNA
taggtcgcgagagcggcgc

SEQ ID NO: 492 - DNA, 19 bp, synthetic construct
  misc_feature 1..19: note = Description of sequence: Exemplary variable region sequence
  source 1..19: mol_type = other DNA
taggtcgcgtaagcggcgc

SEQ ID NO: 493 - DNA, 17 bp, synthetic construct
  misc_feature 1..17: note = Description of sequence: Exemplary variable region sequence
  source 1..17: mol_type = other DNA
taggtggttatccacgc

SEQ ID NO: 494-495 - skipped (2 entries)

SEQ ID NO: 496 - DNA, 18 bp, synthetic construct
  misc_feature 1..18: note = Description of sequence: Exemplary variable region sequence
  source 1..18: mol_type = other DNA
tatacgtgaaagcgtatc

SEQ ID NO: 497 - DNA, 21 bp, synthetic construct
  misc_feature 1..21: note = Description of sequence: Exemplary variable region sequence
  source 1..21: mol_type = other DNA
tatagggtcaaaaactctatc

SEQ ID NO: 498 - DNA, 20 bp, synthetic construct
  misc_feature 1..20: note = Description of sequence: Exemplary variable region sequence
  source 1..20: mol_type = other DNA
tatgcagaaatacctgcatc

SEQ ID NO: 499 - DNA, 15 bp, synthetic construct
  misc_feature 1..15: note = Description of sequence: Exemplary variable region sequence
  source 1..15: mol_type = other DNA
tccccatacgggggc

SEQ ID NO: 500 - DNA, 14 bp, synthetic construct
  misc_feature 1..14: note = Description of sequence: Exemplary variable region sequence
  source 1..14: mol_type = other DNA
tcccgaaggggttc

SEQ ID NO: 501 - DNA, 15 bp, synthetic construct
  misc_feature 1..15: note = Description of sequence: Exemplary variable region sequence
  source 1..15: mol_type = other DNA
tctacgtatgtgggc

SEQ ID NO: 502 - DNA, 14 bp, synthetic construct
  misc_feature 1..14: note = Description of sequence: Exemplary variable region sequence
  source 1..14: mol_type = other DNA
tctcataggagttc

SEQ ID NO: 503 - DNA, 14 bp, synthetic construct
  misc_feature 1..14: note = Description of sequence: Exemplary variable region sequence
  source 1..14: mol_type = other DNA
tctcctctggaggc

SEQ ID NO: 504 - DNA, 15 bp, synthetic construct
  misc_feature 1..15: note = Description of sequence: Exemplary variable region sequence
  source 1..15: mol_type = other DNA
tcttagcaataaggt

SEQ ID NO: 505 - DNA, 14 bp, synthetic construct
  misc_feature 1..14: note = Description of sequence: Exemplary variable region sequence
  source 1..14: mol_type = other DNA
tcttgtaggagttc

SEQ ID NO: 506 - DNA, 15 bp, synthetic construct
  misc_feature 1..15: note = Description of sequence: Exemplary variable region sequence
  source 1..15: mol_type = other DNA
tgaacgtaagttcgc

SEQ ID NO: 507 - DNA, 16 bp, synthetic construct
  misc_feature 1..16: note = Description of sequence: Exemplary variable region sequence
  source 1..16: mol_type = other DNA
tgaactgcgaggttcc

SEQ ID NO: 508 - skipped

SEQ ID NO: 509 - DNA, 15 bp, synthetic construct
  misc_feature 1..15: note = Description of sequence: Exemplary variable region sequence
  source 1..15: mol_type = other DNA
tgaccgaaaggtcgt

SEQ ID NO: 510 - DNA, 15 bp, synthetic construct
  misc_feature 1..15: note = Description of sequence: Exemplary variable region sequence
  source 1..15: mol_type = other DNA
tgaccgcaaggtcgt

SEQ ID NO: 511 - DNA, 14 bp, synthetic construct
  misc_feature 1..14: note = Description of sequence: Exemplary variable region sequence
  source 1..14: mol_type = other DNA
tgagctctgctctc

SEQ ID NO: 512 - DNA, 18 bp, synthetic construct
  misc_feature 1..18: note = Description of sequence: Exemplary variable region sequence
  source 1..18: mol_type = other DNA
tgaggcctcacggcctac

SEQ ID NO: 513 - DNA, 15 bp, synthetic construct
  misc_feature 1..15: note = Description of sequence: Exemplary variable region sequence
  source 1..15: mol_type = other DNA
tgagggcaacttcgt

SEQ ID NO: 514 - DNA, 16 bp, synthetic construct
  misc_feature 1..16: note = Description of sequence: Exemplary variable region sequence
  source 1..16: mol_type = other DNA
tgagggtcatacctcc

SEQ ID NO: 515 - DNA, 18 bp, synthetic construct
  misc_feature 1..18: note = Description of sequence: Exemplary variable region sequence
  source 1..18: mol_type = other DNA
tgagggtgcaaatcctcc

SEQ ID NO: 516 - DNA, 13 bp, synthetic construct
  misc_feature 1..13: note = Description of sequence: Exemplary variable region sequence
  source 1..13: mol_type = other DNA
tgccgaaaggcgt

SEQ ID NO: 517 - DNA, 13 bp, synthetic construct
  misc_feature 1..13: note = Description of sequence: Exemplary variable region sequence
  source 1..13: mol_type = other DNA
tgccgtaaggcgt

SEQ ID NO: 518 - DNA, 14 bp, synthetic construct
  misc_feature 1..14: note = Description of sequence: Exemplary variable region sequence
  source 1..14: mol_type = other DNA
tgcggtctccgcgc

SEQ ID NO: 519 - DNA, 11 bp, synthetic construct
  misc_feature 1..11: note = Description of sequence: Exemplary variable region sequence
  source 1..11: mol_type = other DNA
tgctagagcat

SEQ ID NO: 520 - DNA, 16 bp, synthetic construct
  misc_feature 1..16: note = Description of sequence: Exemplary variable region sequence
  source 1..16: mol_type = other DNA
tgctcgtatagagctc

SEQ ID NO: 521 - DNA, 15 bp, synthetic construct
  misc_feature 1..15: note = Description of sequence: Exemplary variable region sequence
  source 1..15: mol_type = other DNA
tggacaattgtctgc

SEQ ID NO: 522 - DNA, 15 bp, synthetic construct
  misc_feature 1..15: note = Description of sequence: Exemplary variable region sequence
  source 1..15: mol_type = other DNA
tggacagatgtccgt

SEQ ID NO: 523 - DNA, 15 bp, synthetic construct
  misc_feature 1..15: note = Description of sequence: Exemplary variable region sequence
  source 1..15: mol_type = other DNA
tggacaggtgtccgc

SEQ ID NO: 524 - DNA, 15 bp, synthetic construct
  misc_feature 1..15: note = Description of sequence: Exemplary variable region sequence
  source 1..15: mol_type = other DNA
tggacggttgtccgc

SEQ ID NO: 525 - DNA, 13 bp, synthetic construct
  misc_feature 1..13: note = Description of sequence: Exemplary variable region sequence
  source 1..13: mol_type = other DNA
tggacttgtggtc

SEQ ID NO: 526 - DNA, 16 bp, synthetic construct
  misc_feature 1..16: note = Description of sequence: Exemplary variable region sequence
  source 1..16: mol_type = other DNA
tggagattctctccgc

SEQ ID NO: 527 - DNA, 14 bp, synthetic construct
  misc_feature 1..14: note = Description of sequence: Exemplary variable region sequence
  source 1..14: mol_type = other DNA
tggcataggcctgc

SEQ ID NO: 528 - DNA, 14 bp, synthetic construct
  misc_feature 1..14: note = Description of sequence: Exemplary variable region sequence
  source 1..14: mol_type = other DNA
tggcttatgtctac

SEQ ID NO: 529 - DNA, 16 bp, synthetic construct
  misc_feature 1..16: note = Description of sequence: Exemplary variable region sequence
  source 1..16: mol_type = other DNA
tgggagttaatcccgt

SEQ ID NO: 530 - DNA, 14 bp, synthetic construct
  misc_feature 1..14: note = Description of sequence: Exemplary variable region sequence
  source 1..14: mol_type = other DNA
tgggatcttcccgc

SEQ ID NO: 531 - DNA, 16 bp, synthetic construct
  misc_feature 1..16: note = Description of sequence: Exemplary variable region sequence
  source 1..16: mol_type = other DNA
tgggcagaaatgtctc

SEQ ID NO: 532 - DNA, 15 bp, synthetic construct
  misc_feature 1..15: note = Description of sequence: Exemplary variable region sequence
  source 1..15: mol_type = other DNA
tgggcgttcgcccgc

SEQ ID NO: 533 - DNA, 14 bp, synthetic construct
  misc_feature 1..14: note = Description of sequence: Exemplary variable region sequence
  source 1..14: mol_type = other DNA
tgggcttcgcccgc

SEQ ID NO: 534 - DNA, 16 bp, synthetic construct
  misc_feature 1..16: note = Description of sequence: Exemplary variable region sequence
  source 1..16: mol_type = other DNA
tgggggataaccccgt

SEQ ID NO: 535 - DNA, 15 bp, synthetic construct
  misc_feature 1..15: note = Description of sequence: Exemplary variable region sequence
  source 1..15: mol_type = other DNA
tgggggtttccccgt

SEQ ID NO: 536 - skipped

SEQ ID NO: 537 - DNA, 15 bp, synthetic construct
  misc_feature 1..15: note = Description of sequence: Exemplary variable region sequence
  source 1..15: mol_type = other DNA
tggtggcaacaccgt

SEQ ID NO: 538 - DNA, 14 bp, synthetic construct
  misc_feature 1..14: note = Description of sequence: Exemplary variable region sequence
  source 1..14: mol_type = other DNA
tggtttatagccgt

SEQ ID NO: 539 - DNA, 19 bp, synthetic construct
  misc_feature 1..19: note = Description of sequence: Exemplary variable region sequence
  source 1..19: mol_type = other DNA
tgtacggtaataccgtacc

SEQ ID NO: 540 - DNA, 15 bp, synthetic construct
  misc_feature 1..15: note = Description of sequence: Exemplary variable region sequence
  source 1..15: mol_type = other DNA
tgtccgcaaggacgt

SEQ ID NO: 541 - DNA, 15 bp, synthetic construct
  misc_feature 1..15: note = Description of sequence: Exemplary variable region sequence
  source 1..15: mol_type = other DNA
tgtcctaacggacgt

SEQ ID NO: 542 - DNA, 18 bp, synthetic construct
  misc_feature 1..18: note = Description of sequence: Exemplary variable region sequence
  source 1..18: mol_type = other DNA
tgtcctattaacggacgt

SEQ ID NO: 543 - DNA, 16 bp, synthetic construct
  misc_feature 1..16: note = Description of sequence: Exemplary variable region sequence
  source 1..16: mol_type = other DNA
tgtccttcacgggcgt

SEQ ID NO: 544 - DNA, 13 bp, synthetic construct
  misc_feature 1..13: note = Description of sequence: Exemplary variable region sequence
  source 1..13: mol_type = other DNA
tgtcttaggacgt

SEQ ID NO: 545 - DNA, 18 bp, synthetic construct
  misc_feature 1..18: note = Description of sequence: Exemplary variable region sequence
  source 1..18: mol_type = other DNA
tgtgcgttaacgcgtacc

SEQ ID NO: 546 - DNA, 16 bp, synthetic construct
  misc_feature 1..16: note = Description of sequence: Exemplary variable region sequence
  source 1..16: mol_type = other DNA
tgtgtcgcaaggcacc

SEQ ID NO: 547 - DNA, 15 bp, synthetic construct
  misc_feature 1..15: note = Description of sequence: Exemplary variable region sequence
  source 1..15: mol_type = other DNA
tgttcgtaaggactt

SEQ ID NO: 548 - DNA, 16 bp, synthetic construct
  misc_feature 1..16: note = Description of sequence: Exemplary variable region sequence
  source 1..16: mol_type = other DNA
ttcacagaaatgtgtc

SEQ ID NO: 549 - DNA, 14 bp, synthetic construct
  misc_feature 1..14: note = Description of sequence: Exemplary variable region sequence
  source 1..14: mol_type = other DNA
ttccctcgtggagt

SEQ ID NO: 550 - DNA, 14 bp, synthetic construct
  misc_feature 1..14: note = Description of sequence: Exemplary variable region sequence
  source 1..14: mol_type = other DNA
ttccctctgggagc

SEQ ID NO: 551 - DNA, 14 bp, synthetic construct
  misc_feature 1..14: note = Description of sequence: Exemplary variable region sequence
  source 1..14: mol_type = other DNA
ttcccttgtggatc

SEQ ID NO: 552 - DNA, 13 bp, synthetic construct
  misc_feature 1..13: note = Description of sequence: Exemplary variable region sequence
  source 1..13: mol_type = other DNA
ttccttcgggagc

SEQ ID NO: 553 - DNA, 15 bp, synthetic construct
  misc_feature 1..15: note = Description of sequence: Exemplary variable region sequence
  source 1..15: mol_type = other DNA
ttctagcaatagagt

SEQ ID NO: 554 - DNA, 16 bp, synthetic construct
  misc_feature 1..16: note = Description of sequence: Exemplary variable region sequence
  source 1..16: mol_type = other DNA
ttctccactggggagc

SEQ ID NO: 555 - DNA, 15 bp, synthetic construct
  misc_feature 1..15: note = Description of sequence: Exemplary variable region sequence
  source 1..15: mol_type = other DNA
ttctcgagagggagc

SEQ ID NO: 556 - DNA, 15 bp, synthetic construct
  misc_feature 1..15: note = Description of sequence: Exemplary variable region sequence
  source 1..15: mol_type = other DNA
ttctcgtatgagagc

SEQ ID NO: 557 - DNA, 19 bp, synthetic construct
  misc_feature 1..19: note = Description of sequence: Exemplary variable region sequence
  source 1..19: mol_type = other DNA
tttaaggttttcccttaac

SEQ ID NO: 558 - DNA, 14 bp, synthetic construct
  misc_feature 1..14: note = Description of sequence: Exemplary variable region sequence
  source 1..14: mol_type = other DNA
tttcattgtggagt

SEQ ID NO: 559 - DNA, 14 bp, synthetic construct
  misc_feature 1..14: note = Description of sequence: Exemplary variable region sequence
  source 1..14: mol_type = other DNA
tttcgaaggaatcc

SEQ ID NO: 560 - DNA, 13 bp, synthetic construct
  misc_feature 1..13: note = Description of sequence: Exemplary variable region sequence
  source 1..13: mol_type = other DNA
tttcttcggaagc

SEQ ID NO: 561 - DNA, 16 bp, synthetic construct
  misc_feature 1..16: note = Description of sequence: Exemplary variable region sequence
  source 1..16: mol_type = other DNA
tttggggcaactcaac

SEQ ID NO: 562-624 - skipped (63 entries)

SEQ ID NO: 625 - RNA, 76 nt, synthetic construct
  misc_feature 1..76: note = Description of sequence: tRNA-based effector molecule; TREM No: 1
  source 1..76: mol_type = other RNA
gggggattagctcaaatggtagagcgctcgcttctgatgcgagaggtagcgggatcgatgcccgcatcctccacca

SEQ ID NO: 626 - RNA, 75 nt, synthetic construct
  misc_feature 1..75: note = Description of sequence: tRNA-based effector molecule; TREM No: 2
  source 1..75: mol_type = other RNA
ggggatgtagctcagtggtagagcgcatgcttctgatgtatgaggccccgggttcgatccccggcatctccacca

SEQ ID NO: 627 - RNA, 85 nt, synthetic construct
  misc_feature 1..85: note = Description of sequence: tRNA-based effector molecule; TREM No: 3
  source 1..85: mol_type = other RNA
ggtagcgtggccgagcggtctaaggcgctggattctggctccagtctcttcgggggcgtgggttcgaatcccaccgctgccacca

SEQ ID NO: 628 - RNA, 86 nt, synthetic construct
  misc_feature 1..86: note = Description of sequence: tRNA-based effector molecule; TREM No: 4
  source 1..86: mol_type = other RNA
gtcaggatggccgagtggtctaaggcgccagactctggttctggtctccaatggaggcgtgggttcgaatcccacttctgacacca

SEQ ID NO: 629 - RNA, 86 nt, synthetic construct
  misc_feature 1..86: note = Description of sequence: tRNA-based effector molecule; TREM No: 5
  source 1..86: mol_type = other RNA
gtcaggatggccgagcggtctaaggcgctgcgttctggtcgcagtctcccctggaggcgtgggttcgaatcccactcctgacacca

SEQ ID NO: 630 - RNA, 87 nt, synthetic construct
  misc_feature 1..87: note = Description of sequence: tRNA-based effector molecule; TREM No: 6
  source 1..87: mol_type = other RNA
gaccaggatggccgagtggttaaggcgttggactctggatccaatggacatatgtccgcgtgggttcgaaccccactcctggtacca

SEQ ID NO: 631 - RNA, 85 nt, synthetic construct
  misc_feature 1..85: note = Description of sequence: tRNA-based effector molecule; TREM No: 7
  source 1..85: mol_type = other RNA
ggtagcgtggccgagcggtctaaggcgctggattctggctccagtctcttcggaggcgtgggttcgaatcccaccgctgccacca

SEQ ID NO: 632 - RNA, 85 nt, synthetic construct
  misc_feature 1..85: note = Description of sequence: tRNA-based effector molecule; TREM No: 8
  source 1..85: mol_type = other RNA
gtagtcgtggccgagtggttaaggcgatggactctgaatccattggggtttccccacgcaggttcgaatcctgccgactacgcca

SEQ ID NO: 633 - RNA, 85 nt, synthetic construct
  misc_feature 1..85: note = Description of sequence: tRNA-based effector molecule; TREM No: 9
  source 1..85: mol_type = other RNA
gctgtgatggccgagtggttaaggcgttggactctgaatccaatggggtctccccgcgcaggttcgaatcctgctcacagcgcca

SEQ ID NO: 634 - RNA, 85 nt, synthetic construct
  misc_feature 1..85: note = Description of sequence: tRNA-based effector molecule; TREM No: 10
  source 1..85: mol_type = other RNA
gacgaggtggccgagtggttaaggcgatggactctgaatccattgtgctttgcacgcgtgggttcgaatcccatcctcgtcgcca

SEQ ID NO: 635 - RNA, 85 nt, synthetic construct
  misc_feature 1..85: note = Description of sequence: tRNA-based effector molecule; TREM No: 11
  source 1..85: mol_type = other RNA
gcagcgatggccgagtggttaaggcgttggactctgaatccaatggggtctccccgcgcaggttcgaaccctgctcgctgcgcca

SEQ ID NO: 636 - RNA, 75 nt, synthetic construct
  misc_feature 1..75: note = Description of sequence: tRNA-based effector molecule; TREM No: 12
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 46: mod_base = OTHER; note = t is r-thymine at this position
  modified_base 49: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 65: mod_base = OTHER; note = t is r-thymine at this position
  modified_base 69: mod_base = OTHER; note = t is r-thymine at this position
gggggtatagctcagtggtagagcgcgtgcttctgatgcacgaggtcctgggttcgatccccagtacctccacca

SEQ ID NO: 637 - RNA, 75 nt, synthetic construct
  misc_feature 1..75: note = Description of sequence: tRNA-based effector molecule; TREM No: 13
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 39: mod_base = OTHER; note = t is r-thymine at this position
  modified_base 46: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 69: mod_base = OTHER; note = t is r-thymine at this position
gggggtgtagctcagtggtagagcgcgtgcttctgatgtacgaggtcccgggttcaatccccggcacctccacca

SEQ ID NO: 638 - RNA, 75 nt, synthetic construct
  misc_feature 1..75: note = Description of sequence: tRNA-based effector molecule; TREM No: 14
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 49: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 69: mod_base = OTHER; note = t is r-thymine at this position
gggggtgtagctcagtggtagagcgcgtgcttctgatgcacgaggccctgggttcaatccccagcacctccacca

SEQ ID NO: 639 - RNA, 75 nt, synthetic construct
  misc_feature 1..75: note = Description of sequence: tRNA-based effector molecule; TREM No: 15
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 69: mod_base = OTHER; note = t is r-thymine at this position
gggggtgtagctcagtggtagagcgcgtgcttctgatgcacgaggccccgggttcaatccccggcacctccacca

SEQ ID NO: 640 - RNA, 75 nt, synthetic construct
  misc_feature 1..75: note = Description of sequence: tRNA-based effector molecule; TREM No: 16
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 41: mod_base = OTHER; note = t is r-thymine at this position
  modified_base 46: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 67: mod_base = OTHER; note = t is r-thymine at this position
  modified_base 69: mod_base = OTHER; note = t is r-thymine at this position
ggggatgtagctcagtggtagagcgcatgcttctgatgcatgaggtcccgggttcgatccccagcatctccacca

SEQ ID NO: 641 - RNA, 75 nt, synthetic construct
  misc_feature 1..75: note = Description of sequence: tRNA-based effector molecule; TREM No: 17
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 46: mod_base = OTHER; note = t is r-thymine at this position
  modified_base 49: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 65: mod_base = OTHER; note = t is r-thymine at this position
  modified_base 69: mod_base = OTHER; note = t is r-thymine at this position
gggggtatagctcagcggtagagcgcgtgcttctgatgcacgaggtcctgggttcaatccccaatacctccacca

SEQ ID NO: 642 - RNA, 76 nt, synthetic construct
  misc_feature 1..76: note = Description of sequence: tRNA-based effector molecule; TREM No: 18
  source 1..76: mol_type = other RNA
  modified_base 7: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 17: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 29: mod_base = OTHER; note = t is r-thymine at this position
  modified_base 32: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 38: mod_base = OTHER; note = t is r-thymine at this position
  modified_base 47: mod_base = OTHER; note = t is r-thymine at this position
  modified_base 50: mod_base = OTHER; note = t is r-thymine at this position
  modified_base 55: mod_base = OTHER; note = t is r-thymine at this position
  modified_base 59: mod_base = OTHER; note = t is r-thymine at this position
  modified_base 67: mod_base = OTHER; note = t is r-thymine at this position
  modified_base 68: mod_base = OTHER; note = t is r-thymine at this position
  modified_base 70: mod_base = OTHER; note = t is r-thymine at this position
ggggaattagctcaagtggtagagcgcttgcttctgatgcaagaggtagtgggatcgatgcccacattctccacca

SEQ ID NO: 643 - RNA, 76 nt, synthetic construct
  misc_feature 1..76: note = Description of sequence: tRNA-based effector molecule; TREM No: 19
  source 1..76: mol_type = other RNA
  modified_base 7: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 17: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 29: mod_base = OTHER; note = t is r-thymine at this position
  modified_base 32: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 38: mod_base = OTHER; note = t is r-thymine at this position
  modified_base 47: mod_base = OTHER; note = t is r-thymine at this position
  modified_base 50: mod_base = OTHER; note = t is r-thymine at this position
  modified_base 55: mod_base = OTHER; note = t is r-thymine at this position
  modified_base 59: mod_base = OTHER; note = t is r-thymine at this position
  modified_base 67: mod_base = OTHER; note = t is r-thymine at this position
  modified_base 68: mod_base = OTHER; note = t is r-thymine at this position
  modified_base 70: mod_base = OTHER; note = t is r-thymine at this position
ggggaattagctcaagtggtagagcgcttgcttctgatgcaagaggtagtgggatcaatgcccacattctccacca

SEQ ID NO: 644 - RNA, 76 nt, synthetic construct; identical to SEQ ID NO: 625
  misc_feature 1..76: note = Description of sequence: tRNA-based effector molecule; TREM No: 20
  source 1..76: mol_type = other RNA
  modified_base 7: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 17: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 32: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 38: mod_base = OTHER; note = t is r-thymine at this position
  modified_base 47: mod_base = OTHER; note = t is r-thymine at this position
  modified_base 55: mod_base = OTHER; note = t is r-thymine at this position
  modified_base 59: mod_base = OTHER; note = t is r-thymine at this position
  modified_base 67: mod_base = OTHER; note = t is r-thymine at this position
  modified_base 70: mod_base = OTHER; note = t is r-thymine at this position

SEQ ID NO: 645 - RNA, 76 nt, synthetic construct
  misc_feature 1..76: note = Description of sequence: tRNA-based effector molecule; TREM No: 21
  source 1..76: mol_type = other RNA
  modified_base 7: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 17: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 32: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 38: mod_base = OTHER; note = t is r-thymine at this position
  modified_base 47: mod_base = OTHER; note = t is r-thymine at this position
  modified_base 55: mod_base = OTHER; note = t is r-thymine at this position
  modified_base 59: mod_base = OTHER; note = t is r-thymine at this position
  modified_base 67: mod_base = OTHER; note = t is r-thymine at this position
  modified_base 68: mod_base = OTHER; note = t is r-thymine at this position
  modified_base 70: mod_base = OTHER; note = t is r-thymine at this position
ggggaattagctcaaatggtagagcgctcgcttctgatgcgagaggtagcgggatcgatgcccgcattctccacca

SEQ ID NO: 646 - RNA, 76 nt, synthetic construct
  misc_feature 1..76: note = Description of sequence: tRNA-based effector molecule; TREM No: 22
  source 1..76: mol_type = other RNA
  modified_base 7: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 29: mod_base = OTHER; note = t is r-thymine at this position
  modified_base 32: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 38: mod_base = OTHER; note = t is r-thymine at this position
  modified_base 47: mod_base = OTHER; note = t is r-thymine at this position
  modified_base 50: mod_base = OTHER; note = t is r-thymine at this position
  modified_base 55: mod_base = OTHER; note = t is r-thymine at this position
  modified_base 59: mod_base = OTHER; note = t is r-thymine at this position
  modified_base 67: mod_base = OTHER; note = t is r-thymine at this position
  modified_base 68: mod_base = OTHER; note = t is r-thymine at this position
  modified_base 70: mod_base = OTHER; note = t is r-thymine at this position
ggggaattagctcaagcggtagagcgcttgcttctgatgcaagaggtagtgggatcgatgcccacattctccacca

SEQ ID NO: 647 - RNA, 76 nt, synthetic construct
  misc_feature 1..76: note = Description of sequence: tRNA-based effector molecule; TREM No: 23
  source 1..76: mol_type = other RNA
  modified_base 7: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 32: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 38: mod_base = OTHER; note = t is r-thymine at this position
  modified_base 47: mod_base = OTHER; note = t is r-thymine at this position
  modified_base 55: mod_base = OTHER; note = t is r-thymine at this position
  modified_base 67: mod_base = OTHER; note = t is r-thymine at this position
  modified_base 68: mod_base = OTHER; note = t is r-thymine at this position
  modified_base 70: mod_base = OTHER; note = t is r-thymine at this position
ggggaattagctcaggcggtagagcgctcgcttctgatgcgagaggtagcgggatcgacgcccgcattctccacca

SEQ ID NO: 648 - RNA, 76 nt, synthetic construct
  misc_feature 1..76: note = Description of sequence: tRNA-based effector molecule; TREM No: 24
  source 1..76: mol_type = other RNA
  modified_base 7: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 17: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 32: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 38: mod_base = OTHER; note = t is r-thymine at this position
  modified_base 47: mod_base = OTHER; note = t is r-thymine at this position
  modified_base 50: mod_base = OTHER; note = t is r-thymine at this position
  modified_base 55: mod_base = OTHER; note = t is r-thymine at this position
  modified_base 59: mod_base = OTHER; note = t is r-thymine at this position
  modified_base 67: mod_base = OTHER; note = t is r-thymine at this position
  modified_base 68: mod_base = OTHER; note = t is r-thymine at this position
  modified_base 70: mod_base = OTHER; note = t is r-thymine at this position
ggggaattagctcaagtggtagagcgctcgcttctgatgcgagaggtagtgggatcgatgcccgcattctccacca

SEQ ID NO: 649 - RNA, 76 nt, synthetic construct
  misc_feature 1..76: note = Description of sequence: tRNA-based effector molecule; TREM No: 25
  source 1..76: mol_type = other RNA
  modified_base 7: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 17: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 29: mod_base = OTHER; note = t is r-thymine at this position
  modified_base 32: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 47: mod_base = OTHER; note = t is r-thymine at this position
  modified_base 50: mod_base = OTHER; note = t is r-thymine at this position
  modified_base 55: mod_base = OTHER; note = t is r-thymine at this position
  modified_base 59: mod_base = OTHER; note = t is r-thymine at this position
  modified_base 67: mod_base = OTHER; note = t is r-thymine at this position
  modified_base 68: mod_base = OTHER; note = t is r-thymine at this position
  modified_base 70: mod_base = OTHER; note = t is r-thymine at this position
ggggaattagctcaagtggtagagcgcttgcttctgacgcaagaggtagtgggatcgatgcccacattctccacca

SEQ ID NO: 650 - RNA, 76 nt, synthetic construct
  misc_feature 1..76: note = Description of sequence: tRNA-based effector molecule; TREM No: 26
  source 1..76: mol_type = other RNA
  modified_base 7: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 29: mod_base = OTHER; note = t is r-thymine at this position
  modified_base 32: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 38: mod_base = OTHER; note = t is r-thymine at this position
  modified_base 47: mod_base = OTHER; note = t is r-thymine at this position
  modified_base 55: mod_base = OTHER; note = t is r-thymine at this position
  modified_base 59: mod_base = OTHER; note = t is r-thymine at this position
  modified_base 63: mod_base = OTHER; note = t is r-thymine at this position
  modified_base 67: mod_base = OTHER; note = t is r-thymine at this position
  modified_base 68: mod_base = OTHER; note = t is r-thymine at this position
  modified_base 70: mod_base = OTHER; note = t is r-thymine at this position
ggggaattagctcaagcggtagagcgcttgcttctgatgcaagaggtagcaggatcgatgcctgcattctccacca

SEQ ID NO: 651 - RNA, 76 nt, synthetic construct
  misc_feature 1..76: note = Description of sequence: tRNA-based effector molecule; TREM No: 27
  source 1..76: mol_type = other RNA
  modified_base 7: mod_base = OTHER; note = t is r-thymine at this position
  modified_base 8: mod_base = OTHER; note = t is r-thymine at this position
  modified_base 17: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 29: mod_base = OTHER; note = t is r-thymine at this position
  modified_base 32: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 38: mod_base = OTHER; note = t is r-thymine at this position
  modified_base 47: mod_base = OTHER; note = t is r-thymine at this position
  modified_base 50: mod_base = OTHER; note = t is r-thymine at this position
  modified_base 55: mod_base = OTHER; note = t is r-thymine at this position
  modified_base 59: mod_base = OTHER; note = t is r-thymine at this position
  modified_base 67: mod_base = OTHER; note = t is r-thymine at this position
  modified_base 68: mod_base = OTHER; note = t is r-thymine at this position
  modified_base 70: mod_base = OTHER; note = t is r-thymine at this position
ggggaattagcccaagtggtagagcgcttgcttctgatgcaagaggtagtgggatcgatgcccacattctccacca

SEQ ID NO: 652 - RNA, 75 nt, synthetic construct
  misc_feature 1..75: note = Description of sequence: tRNA-based effector molecule; TREM No: 28
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 39: mod_base = OTHER; note = t is r-thymine at this position
  modified_base 41: mod_base = OTHER; note = t is r-thymine at this position
  modified_base 46: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 67: mod_base = OTHER; note = t is r-thymine at this position
  modified_base 69: mod_base = OTHER; note = t is r-thymine at this position
ggggatgtagctcagtggtagagcgcatgcttctgatgtatgaggtcccgggttcgatccccggcatctccacca

SEQ ID NO: 653 - RNA, 75 nt, synthetic construct; identical to SEQ ID NO: 626
  misc_feature 1..75: note = Description of sequence: tRNA-based effector molecule; TREM No: 29
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 39: mod_base = OTHER; note = t is r-thymine at this position
  modified_base 41: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 67: mod_base = OTHER; note = t is r-thymine at this position
  modified_base 69: mod_base = OTHER; note = t is r-thymine at this position

SEQ ID NO: 654 - RNA, 75 nt, synthetic construct
  misc_feature 1..75: note = Description of sequence: tRNA-based effector molecule; TREM No: 30
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 39: mod_base = OTHER; note = t is r-thymine at this position
  modified_base 41: mod_base = OTHER; note = t is r-thymine at this position
  modified_base 46: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 67: mod_base = OTHER; note = t is r-thymine at this position
  modified_base 69: mod_base = OTHER; note = t is r-thymine at this position
ggggatgtagctcagtggtagagcgcgcgcttctgatgtgtgaggtcccgggttcaatccccggcatctccacca

SEQ ID NO: 655 - RNA, 75 nt, synthetic construct
  misc_feature 1..75: note = Description of sequence: tRNA-based effector molecule; TREM No: 31
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 39: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 66: mod_base = OTHER; note = t is r-thymine at this position
  modified_base 69: mod_base = OTHER; note = t is r-thymine at this position
gggggtgtagctcagtggtagagcgcgtgcttctgatgtacgaggccccgggttcgacccccggctcctccacca

SEQ ID NO: 656 - RNA, 75 nt, synthetic construct
  misc_feature 1..75: note = Description of sequence: tRNA-based effector molecule; TREM No: 32
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 39: mod_base = OTHER; note = t is r-thymine at this position
  modified_base 41: mod_base = OTHER; note = t is r-thymine at this position
  modified_base 46: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 69: mod_base = OTHER; note = t is r-thymine at this position
gggggtgtagctcagtggtagagcgcatgcttctgatgtatgaggtcccgggttcgatccccggcacctccacca

SEQ ID NO: 657 - RNA, 75 nt, synthetic construct
  misc_feature 1..75: note = Description of sequence: tRNA-based effector molecule; TREM No: 33
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 39: mod_base = OTHER; note = t is r-thymine at this position
  modified_base 41: mod_base = OTHER; note = t is r-thymine at this position
  modified_base 48: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 69: mod_base = OTHER; note = t is r-thymine at this position
gggggtgtagctcagtggtagagcgcatgcttctgatgtatgaggcctcgggttcgatccccgacacctccacca

SEQ ID NO: 658 - RNA, 75 nt, synthetic construct
  misc_feature 1..75: note = Description of sequence: tRNA-based effector molecule; TREM No: 34
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 39: mod_base = OTHER; note = t is r-thymine at this position
  modified_base 41: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 67: mod_base = OTHER; note = t is r-thymine at this position
  modified_base 69: mod_base = OTHER; note = t is r-thymine at this position
ggggatgtagctcagtggtagagcgcatgcttctgacgtatgaggccccgggttcaatccccggcatctccacca

SEQ ID NO: 659 - RNA, 75 nt, synthetic construct
  misc_feature 1..75: note = Description of sequence: tRNA-based effector molecule; TREM No: 35
  source 1..75: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 39: mod_base = OTHER; note = t is r-thymine at this position
  modified_base 41: mod_base = OTHER; note = t is r-thymine at this position
  modified_base 53: mod_base = OTHER; note = t is r-thymine at this position
  modified_base 54: mod_base = OTHER; note = t is r-thymine at this position
  modified_base 58: mod_base = OTHER; note = t is r-thymine at this position
  modified_base 69: mod_base = OTHER; note = t is r-thymine at this position
gggggtgtagctcagtggtagagcacatgcttctgatgtgtgaggccccgggttcgatccccggcacctccacca

SEQ ID NO: 660 - RNA, 74 nt, synthetic construct
  misc_feature 1..74: note = Description of sequence: tRNA-based effector molecule; TREM No: 36
  source 1..74: mol_type = other RNA
  modified_base 6: mod_base = OTHER; note = t is r-thymine at this position
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 28: mod_base = OTHER; note = t is r-thymine at this position
  modified_base 31: mod_base = OTHER; note = t is r-thymine at this position
  modified_base 32: mod_base = OTHER; note = t is r-thymine at this position
  modified_base 34: mod_base = OTHER; note = t is r-thymine at this position
  modified_base 37: mod_base = OTHER; note = t is r-thymine at this position
  modified_base 39: mod_base = OTHER; note = t is r-thymine at this position
  modified_base 41: mod_base = OTHER; note = t is r-thymine at this position
  modified_base 48: mod_base = OTHER; note = t is r-thymine at this position
  modified_base 52: mod_base = OTHER; note = t is r-thymine at this position
  modified_base 53: mod_base = OTHER; note = t is r-thymine at this position
  modified_base 57: mod_base = OTHER; note = t is r-thymine at this position
  modified_base 68: mod_base = OTHER; note = t is r-thymine at this position
gggggtgtagctcagtggtagagcgcatgcttctgatgtatgaggcctcggttcgatccccgacacctccacca

SEQ ID NO: 661 - RNA, 85 nt, synthetic construct; identical to SEQ ID NO: 631
  misc_feature 1..85: note = Description of sequence: tRNA-based effector molecule; TREM No: 37
  source 1..85: mol_type = other RNA
  modified_base 3: mod_base = OTHER; note = t is r-thymine at this position
  modified_base 8: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 29: mod_base = OTHER; note = t is r-thymine at this position
  modified_base 33: mod_base = OTHER; note = t is r-thymine at this position
  modified_base 34: mod_base = OTHER; note = t is r-thymine at this position
  modified_base 36: mod_base = OTHER; note = t is r-thymine at this position
  modified_base 40: mod_base = OTHER; note = t is r-thymine at this position
  modified_base 45: mod_base = OTHER; note = t is r-thymine at this position
  modified_base 47: mod_base = OTHER; note = t is r-thymine at this position
  modified_base 49: mod_base = OTHER; note = t is r-thymine at this position
  modified_base 50: mod_base = OTHER; note = t is r-thymine at this position
  modified_base 59: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position

SEQ ID NO: 662 - RNA, 85 nt, synthetic construct; identical to SEQ ID NO: 627
  misc_feature 1..85: note = Description of sequence: tRNA-based effector molecule; TREM No: 38
  source 1..85: mol_type = other RNA
  modified_base 3: mod_base = OTHER; note = t is r-thymine at this position
  modified_base 8: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 29: mod_base = OTHER; note = t is r-thymine at this position
  modified_base 33: mod_base = OTHER; note = t is r-thymine at this position
  modified_base 34: mod_base = OTHER; note = t is r-thymine at this position
  modified_base 36: mod_base = OTHER; note = t is r-thymine at this position
  modified_base 40: mod_base = OTHER; note = t is r-thymine at this position
  modified_base 45: mod_base = OTHER; note = t is r-thymine at this position
  modified_base 47: mod_base = OTHER; note = t is r-thymine at this position
  modified_base 49: mod_base = OTHER; note = t is r-thymine at this position
  modified_base 50: mod_base = OTHER; note = t is r-thymine at this position
  modified_base 59: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position

SEQ ID NO: 663 - RNA, 85 nt, synthetic construct
  misc_feature 1..85: note = Description of sequence: tRNA-based effector molecule; TREM No: 39
  source 1..85: mol_type = other RNA
  modified_base 3: mod_base = OTHER; note = t is r-thymine at this position
  modified_base 8: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 29: mod_base = OTHER; note = t is r-thymine at this position
  modified_base 33: mod_base = OTHER; note = t is r-thymine at this position
  modified_base 34: mod_base = OTHER; note = t is r-thymine at this position
  modified_base 36: mod_base = OTHER; note = t is r-thymine at this position
  modified_base 40: mod_base = OTHER; note = t is r-thymine at this position
  modified_base 45: mod_base = OTHER; note = t is r-thymine at this position
  modified_base 47: mod_base = OTHER; note = t is r-thymine at this position
  modified_base 49: mod_base = OTHER; note = t is r-thymine at this position
  modified_base 50: mod_base = OTHER; note = t is r-thymine at this position
  modified_base 59: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
ggtagcgtggccgagcggtctaaggcgctggattctggctccagtctcttcgggggcgtgggttcaaatcccaccgctgccacca

SEQ ID NO: 664 - RNA, 85 nt, synthetic construct
  misc_feature 1..85: note = Description of sequence: tRNA-based effector molecule; TREM No: 40
  source 1..85: mol_type = other RNA
  modified_base 3: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 29: mod_base = OTHER; note = t is r-thymine at this position
  modified_base 33: mod_base = OTHER; note = t is r-thymine at this position
  modified_base 34: mod_base = OTHER; note = t is r-thymine at this position
  modified_base 36: mod_base = OTHER; note = t is r-thymine at this position
  modified_base 40: mod_base = OTHER; note = t is r-thymine at this position
  modified_base 45: mod_base = OTHER; note = t is r-thymine at this position
  modified_base 47: mod_base = OTHER; note = t is r-thymine at this position
  modified_base 49: mod_base = OTHER; note = t is r-thymine at this position
  modified_base 50: mod_base = OTHER; note = t is r-thymine at this position
  modified_base 59: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 65: mod_base = OTHER; note = t is r-thymine at this position
  modified_base 69: mod_base = OTHER; note = t is r-thymine at this position
ggtagcgtggccgagtggtctaagacgctggattctggctccagtctcttcgggggcgtgggtttgaatcccaccgctgccacca

SEQ ID NO: 665 - RNA, 87 nt, synthetic construct
  misc_feature 1..87: note = Description of sequence: tRNA-based effector molecule; TREM No: 41
  source 1..87: mol_type = other RNA
  modified_base 2: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 34: mod_base = OTHER; note = t is r-thymine at this position
  modified_base 36: mod_base = OTHER; note = t is r-thymine at this position
  modified_base 39: mod_base = OTHER; note = t is r-thymine at this position
  modified_base 40: mod_base = OTHER; note = t is r-thymine at this position
  modified_base 42: mod_base = OTHER; note = t is r-thymine at this position
  modified_base 45: mod_base = OTHER; note = t is r-thymine at this position
  modified_base 47: mod_base = OTHER; note = t is r-thymine at this position
  modified_base 51: mod_base = OTHER; note = t is r-thymine at this position
  modified_base 53: mod_base = OTHER; note = t is r-thymine at this position
  modified_base 61: mod_base = OTHER; note = t is r-thymine at this position
  modified_base 65: mod_base = OTHER; note = t is r-thymine at this position
  modified_base 66: mod_base = OTHER; note = t is r-thymine at this position
  modified_base 71: mod_base = OTHER; note = t is r-thymine at this position
gtcaggatggccgagtggtctaaggcgccagactctggttctggtctccgtatggaggcgtgggttcgaatcccacttctgacacca

SEQ ID NO: 666 - RNA, 87 nt, synthetic construct
  misc_feature 1..87: note = Description of sequence: tRNA-based effector molecule; TREM No: 42
  source 1..87: mol_type = other RNA
  modified_base 2: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 34: mod_base = OTHER; note = t is r-thymine at this position
  modified_base 36: mod_base = OTHER; note = t is r-thymine at this position
  modified_base 39: mod_base = OTHER; note = t is r-thymine at this position
  modified_base 40: mod_base = OTHER; note = t is r-thymine at this position
  modified_base 42: mod_base = OTHER; note = t is r-thymine at this position
  modified_base 45: mod_base = OTHER; note = t is r-thymine at this position
  modified_base 47: mod_base = OTHER; note = t is r-thymine at this position
  modified_base 53: mod_base = OTHER; note = t is r-thymine at this position
  modified_base 61: mod_base = OTHER; note = t is r-thymine at this position
  modified_base 65: mod_base = OTHER; note = t is r-thymine at this position
  modified_base 66: mod_base = OTHER; note = t is r-thymine at this position
  modified_base 71: mod_base = OTHER; note = t is r-thymine at this position
gtcaggatggccgagtggtctaaggcgccagactctggttctggtctccgcatggaggcgtgggttcgaatcccacttctgacacca

SEQ ID NO: 667 - RNA, 87 nt, synthetic construct
  misc_feature 1..87: note = Description of sequence: tRNA-based effector molecule; TREM No: 43
  source 1..87: mol_type = other RNA
  modified_base 2: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 34: mod_base = OTHER; note = t is r-thymine at this position
  modified_base 36: mod_base = OTHER; note = t is r-thymine at this position
  modified_base 39: mod_base = OTHER; note = t is r-thymine at this position
  modified_base 40: mod_base = OTHER; note = t is r-thymine at this position
  modified_base 42: mod_base = OTHER; note = t is r-thymine at this position
  modified_base 45: mod_base = OTHER; note = t is r-thymine at this position
  modified_base 47: mod_base = OTHER; note = t is r-thymine at this position
  modified_base 53: mod_base = OTHER; note = t is r-thymine at this position
  modified_base 61: mod_base = OTHER; note = t is r-thymine at this position
  modified_base 65: mod_base = OTHER; note = t is r-thymine at this position
  modified_base 66: mod_base = OTHER; note = t is r-thymine at this position
  modified_base 71: mod_base = OTHER; note = t is r-thymine at this position
gtcaggatggccgagtggtctaaggcgccagactctggttctggtctccggatggaggcgtgggttcgaatcccacttctgacacca

SEQ ID NO: 668 - RNA, 86 nt, synthetic construct; identical to SEQ ID NO: 628
  misc_feature 1..86: note = Description of sequence: tRNA-based effector molecule; TREM No: 44
  source 1..86: mol_type = other RNA
  modified_base 2: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 34: mod_base = OTHER; note = t is r-thymine at this position
  modified_base 36: mod_base = OTHER; note = t is r-thymine at this position
  modified_base 39: mod_base = OTHER; note = t is r-thymine at this position
  modified_base 40: mod_base = OTHER; note = t is r-thymine at this position
  modified_base 42: mod_base = OTHER; note = t is r-thymine at this position
  modified_base 45: mod_base = OTHER; note = t is r-thymine at this position
  modified_base 47: mod_base = OTHER; note = t is r-thymine at this position
  modified_base 52: mod_base = OTHER; note = t is r-thymine at this position
  modified_base 60: mod_base = OTHER; note = t is r-thymine at this position
  modified_base 64: mod_base = OTHER; note = t is r-thymine at this position
  modified_base 65: mod_base = OTHER; note = t is r-thymine at this position
  modified_base 70: mod_base = OTHER; note = t is r-thymine at this position

SEQ ID NO: 669 - RNA, 87 nt, synthetic construct
  misc_feature 1..87: note = Description of sequence: tRNA-based effector molecule; TREM No: 45
  source 1..87: mol_type = other RNA
  modified_base 2: mod_base = OTHER; note = t is r-thymine at this position
  modified_base 8: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 22: mod_base = OTHER; note = t is r-thymine at this position
  modified_base 30: mod_base = OTHER; note = t is r-thymine at this position
  modified_base 34: mod_base = OTHER; note = t is r-thymine at this position
  modified_base 35: mod_base = OTHER; note = t is r-thymine at this position
  modified_base 37: mod_base = OTHER; note = t is r-thymine at this position
  modified_base 40: mod_base = OTHER; note = t is r-thymine at this position
  modified_base 48: mod_base = OTHER; note = t is r-thymine at this position
  modified_base 53: mod_base = OTHER; note = t is r-thymine at this position
  modified_base 61: mod_base = OTHER; note = t is r-thymine at this position
  modified_base 65: mod_base = OTHER; note = t is r-thymine at this position
  modified_base 66: mod_base = OTHER; note = t is r-thymine at this position
  modified_base 71: mod_base = OTHER; note = t is r-thymine at this position
gtcaggatggccgagcagtcttaaggcgctgcgttctgatcgcaccctccgctggaggcgtgggttcgaatcccacttttgacacca

SEQ ID NO: 670 - RNA, 86 nt, synthetic construct; identical to SEQ ID NO: 629
  misc_feature 1..86: note = Description of sequence: tRNA-based effector molecule; TREM No: 46
  source 1..86: mol_type = other RNA
  modified_base 2: mod_base = OTHER; note = t is r-thymine at this position
  modified_base 8: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 29: mod_base = OTHER; note = t is r-thymine at this position
  modified_base 33: mod_base = OTHER; note = t is r-thymine at this position
  modified_base 34: mod_base = OTHER; note = t is r-thymine at this position
  modified_base 36: mod_base = OTHER; note = t is r-thymine at this position
  modified_base 39: mod_base = OTHER; note = t is r-thymine at this position
  modified_base 45: mod_base = OTHER; note = t is r-thymine at this position
  modified_base 47: mod_base = OTHER; note = t is r-thymine at this position
  modified_base 52: mod_base = OTHER; note = t is r-thymine at this position
  modified_base 60: mod_base = OTHER; note = t is r-thymine at this position
  modified_base 64: mod_base = OTHER; note = t is r-thymine at this position
  modified_base 65: mod_base = OTHER; note = t is r-thymine at this position
  modified_base 70: mod_base = OTHER; note = t is r-thymine at this position

SEQ ID NO: 671 - RNA, 86 nt, synthetic construct
  misc_feature 1..86: note = Description of sequence: tRNA-based effector molecule; TREM No: 47
  source 1..86: mol_type = other RNA
  modified_base 2: mod_base = OTHER; note = t is r-thymine at this position
  modified_base 8: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 29: mod_base = OTHER; note = t is r-thymine at this position
  modified_base 33: mod_base = OTHER; note = t is r-thymine at this position
  modified_base 34: mod_base = OTHER; note = t is r-thymine at this position
  modified_base 36: mod_base = OTHER; note = t is r-thymine at this position
  modified_base 39: mod_base = OTHER; note = t is r-thymine at this position
  modified_base 45: mod_base = OTHER; note = t is r-thymine at this position
  modified_base 47: mod_base = OTHER; note = t is r-thymine at this position
  modified_base 52: mod_base = OTHER; note = t is r-thymine at this position
  modified_base 60: mod_base = OTHER; note = t is r-thymine at this position
  modified_base 64: mod_base = OTHER; note = t is r-thymine at this position
  modified_base 65: mod_base = OTHER; note = t is r-thymine at this position
  modified_base 70: mod_base = OTHER; note = t is r-thymine at this position
gtcaggatggccgagcggtctaaggcgctgcgttctggtcgcagtctcccctggaggcgtgggttcgaatcccacttctgacacca

SEQ ID NO: 672 - RNA, 86 nt, synthetic construct
  misc_feature 1..86: note = Description of sequence: tRNA-based effector molecule; TREM No: 48
  source 1..86: mol_type = other RNA
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 27: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 44: mod_base = OTHER; note = t is r-thymine at this position
  modified_base 50: mod_base = OTHER; note = t is r-thymine at this position
  modified_base 52: mod_base = OTHER; note = t is r-thymine at this position
  modified_base 54: mod_base = OTHER; note = t is r-thymine at this position
  modified_base 60: mod_base = OTHER; note = t is r-thymine at this position
  modified_base 64: mod_base = OTHER; note = t is r-thymine at this position
  modified_base 65: mod_base = OTHER; note = t is r-thymine at this position
accaggatggccgagtggttaaggcgttggactctggatccaatggacatatgtccgcgtgggttcgaaccccactcctggtacca

SEQ ID NO: 673 - RNA, 86 nt, synthetic construct
  misc_feature 1..86: note = Description of sequence: tRNA-based effector molecule; TREM No: 49
  source 1..86: mol_type = other RNA
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 27: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 44: mod_base = OTHER; note = t is r-thymine at this position
  modified_base 48: mod_base = OTHER; note = t is r-thymine at this position
  modified_base 49: mod_base = OTHER; note = t is r-thymine at this position
  modified_base 52: mod_base = OTHER; note = t is r-thymine at this position
  modified_base 54: mod_base = OTHER; note = t is r-thymine at this position
  modified_base 60: mod_base = OTHER; note = t is r-thymine at this position
  modified_base 64: mod_base = OTHER; note = t is r-thymine at this position
  modified_base 65: mod_base = OTHER; note = t is r-thymine at this position
accagaatggccgagtggttaaggcgttggactctggatccaatggattcatatccgcgtgggttcgaaccccacttctggtacca

SEQ ID NO: 674 - RNA, 86 nt, synthetic construct
  misc_feature 1..86: note = Description of sequence: tRNA-based effector molecule; TREM No: 50
  source 1..86: mol_type = other RNA
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 27: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 44: mod_base = OTHER; note = t is r-thymine at this position
  modified_base 49: mod_base = OTHER; note = t is r-thymine at this position
  modified_base 52: mod_base = OTHER; note = t is r-thymine at this position
  modified_base 60: mod_base = OTHER; note = t is r-thymine at this position
  modified_base 64: mod_base = OTHER; note = t is r-thymine at this position
  modified_base 65: mod_base = OTHER; note = t is r-thymine at this position
accgggatggccgagtggttaaggcgttggactctggatccaatgggctggtgcccgcgtgggttcgaaccccactctcggtacca

SEQ ID NO: 675 - RNA, 86 nt, synthetic construct
  misc_feature 1..86: note = Description of sequence: tRNA-based effector molecule; TREM No: 51
  source 1..86: mol_type = other RNA
  modified_base 8: mod_base = OTHER; note = t is r-thymine at this position
  modified_base 12: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 27: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 44: mod_base = OTHER; note = t is r-thymine at this position
  modified_base 52: mod_base = OTHER; note = t is r-thymine at this position
  modified_base 54: mod_base = OTHER; note = t is r-thymine at this position
  modified_base 60: mod_base = OTHER; note = t is r-thymine at this position
  modified_base 64: mod_base = OTHER; note = t is r-thymine at this position
  modified_base 65: mod_base = OTHER; note = t is r-thymine at this position
accgggatggctgagtggttaaggcgttggactctggatccaatggacaggtgtccgcgtgggttcgagccccactcccggtacca

SEQ ID NO: 676 - RNA, 85 nt, synthetic construct
  misc_feature 1..85: note = Description of sequence: tRNA-based effector molecule; TREM No: 52
  source 1..85: mol_type = other RNA
  modified_base 3: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 29: mod_base = OTHER; note = t is r-thymine at this position
  modified_base 33: mod_base = OTHER; note = t is r-thymine at this position
  modified_base 34: mod_base = OTHER; note = t is r-thymine at this position
  modified_base 36: mod_base = OTHER; note = t is r-thymine at this position
  modified_base 40: mod_base = OTHER; note = t is r-thymine at this position
  modified_base 45: mod_base = OTHER; note = t is r-thymine at this position
  modified_base 48: mod_base = OTHER; note = t is r-thymine at this position
  modified_base 49: mod_base = OTHER; note = t is r-thymine at this position
  modified_base 50: mod_base = OTHER; note = t is r-thymine at this position
  modified_base 54: mod_base = OTHER; note = t is r-thymine at this position
  modified_base 59: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
ggtagcgtggccgagtggtctaaggcgctggattctggctccagtcatttcgatggcgtgggttcgaatcccaccgctgccacca

SEQ ID NO: 677 - RNA, 85 nt, synthetic construct
  misc_feature 1..85: note = Description of sequence: tRNA-based effector molecule; TREM No: 53
  source 1..85: mol_type = other RNA
  modified_base 3: mod_base = OTHER; note = t is r-thymine at this position
  modified_base 6: mod_base = OTHER; note = t is r-thymine at this position
  modified_base 8: mod_base = OTHER; note = t is r-thymine at this position
  modified_base 19: mod_base = OTHER; note = t is r-thymine at this position
  modified_base 21: mod_base = OTHER; note = t is r-thymine at this position
  modified_base 29: mod_base = OTHER; note = t is r-thymine at this position
  modified_base 33: mod_base = OTHER; note = t is r-thymine at this position
  modified_base 34: mod_base = OTHER; note = t is r-thymine at this position
  modified_base 36: mod_base = OTHER; note = t is r-thymine at this position
  modified_base 40: mod_base = OTHER; note = t is r-thymine at this position
  modified_base 45: mod_base = OTHER; note = t is r-thymine at this position
  modified_base 47: mod_base = OTHER; note = t is r-thymine at this position
  modified_base 49: mod_base = OTHER; note = t is r-thymine at this position
  modified_base 50: mod_base = OTHER; note = t is r-thymine at this position
  modified_base 59: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
ggtagtgtggccgagcggtctaaggcgctggattctggctccagtctcttcgggggcgtgggttcgaatcccaccactgccacca

SEQ ID NO: 678 - RNA, 85 nt, synthetic construct
  misc_feature 1..85: note = Description of sequence: tRNA-based effector molecule; TREM No: 54
  source 1..85: mol_type = other RNA
  modified_base 2: mod_base = OTHER; note = t is r-thymine at this position
  modified_base 5: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 43: mod_base = OTHER; note = t is r-thymine at this position
  modified_base 44: mod_base = OTHER; note = t is r-thymine at this position
  modified_base 49: mod_base = OTHER; note = t is r-thymine at this position
  modified_base 50: mod_base = OTHER; note = t is r-thymine at this position
  modified_base 51: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
  modified_base 72: mod_base = OTHER; note = t is r-thymine at this position
gtagtcgtggccgagtggttaaggcgatggactctgaatccattggggtttccccgcgcaggttcgaatcctgccgactacgcca

SEQ ID NO: 679 - RNA, 85 nt, synthetic construct
  misc_feature 1..85: note = Description of sequence: tRNA-based effector molecule; TREM No: 55
  source 1..85: mol_type = other RNA
  modified_base 2: mod_base = OTHER; note = t is r-thymine at this position
  modified_base 5: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 43: mod_base = OTHER; note = t is r-thymine at this position
  modified_base 44: mod_base = OTHER; note = t is r-thymine at this position
  modified_base 49: mod_base = OTHER; note = t is r-thymine at this position
  modified_base 51: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
  modified_base 72: mod_base = OTHER; note = t is r-thymine at this position
gtagtcgtggccgagtggttaaggcgatggactctgaatccattggggtctccccgcgcaggttcgaatcctgccgactacgcca

SEQ ID NO: 680 - RNA, 85 nt, synthetic construct; identical to SEQ ID NO: 632
  misc_feature 1..85: note = Description of sequence: tRNA-based effector molecule; TREM No: 56
  source 1..85: mol_type = other RNA
  modified_base 2: mod_base = OTHER; note = t is r-thymine at this position
  modified_base 5: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 43: mod_base = OTHER; note = t is r-thymine at this position
  modified_base 44: mod_base = OTHER; note = t is r-thymine at this position
  modified_base 49: mod_base = OTHER; note = t is r-thymine at this position
  modified_base 50: mod_base = OTHER; note = t is r-thymine at this position
  modified_base 51: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
  modified_base 72: mod_base = OTHER; note = t is r-thymine at this position

SEQ ID NO: 681 - RNA, 85 nt, synthetic construct
  misc_feature 1..85: note = Description of sequence: tRNA-based effector molecule; TREM No: 57
  source 1..85: mol_type = other RNA
  modified_base 2: mod_base = OTHER; note = t is r-thymine at this position
  modified_base 5: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 25: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 43: mod_base = OTHER; note = t is r-thymine at this position
  modified_base 44: mod_base = OTHER; note = t is r-thymine at this position
  modified_base 49: mod_base = OTHER; note = t is r-thymine at this position
  modified_base 51: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
  modified_base 72: mod_base = OTHER; note = t is r-thymine at this position
gtagtcgtggccgagtggttaaggtgatggactctgaacccattggggtctccccgcgcaggttcgaatcctgccgactacgcca

SEQ ID NO: 682 - RNA, 85 nt, synthetic construct; identical to SEQ ID NO: 633
  misc_feature 1..85: note = Description of sequence: tRNA-based effector molecule; TREM No: 58
  source 1..85: mol_type = other RNA
  modified_base 3: mod_base = OTHER; note = t is r-thymine at this position
  modified_base 5: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 27: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 44: mod_base = OTHER; note = t is r-thymine at this position
  modified_base 49: mod_base = OTHER; note = t is r-thymine at this position
  modified_base 51: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
  modified_base 72: mod_base = OTHER; note = t is r-thymine at this position
  modified_base 75: mod_base = OTHER; note = t is r-thymine at this position

SEQ ID NO: 683 - RNA, 85 nt, synthetic construct
  misc_feature 1..85: note = Description of sequence: tRNA-based effector molecule; TREM No: 59
  source 1..85: mol_type = other RNA
  modified_base 3: mod_base = OTHER; note = t is r-thymine at this position
  modified_base 5: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 27: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 44: mod_base = OTHER; note = t is r-thymine at this position
  modified_base 49: mod_base = OTHER; note = t is r-thymine at this position
  modified_base 51: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
  modified_base 72: mod_base = OTHER; note = t is r-thymine at this position
  modified_base 75: mod_base = OTHER; note = t is r-thymine at this position
gctgtgatggccgagtggttaaggcgttggactctgaatccaatggggtctccccgcgcaggttcaaatcctgctcacagcgcca

SEQ ID NO: 684 - RNA, 85 nt, synthetic construct
  misc_feature 1..85: note = Description of sequence: tRNA-based effector molecule; TREM No: 60
  source 1..85: mol_type = other RNA
  modified_base 2: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 27: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 44: mod_base = OTHER; note = t is r-thymine at this position
  modified_base 49: mod_base = OTHER; note = t is r-thymine at this position
  modified_base 50: mod_base = OTHER; note = t is r-thymine at this position
  modified_base 51: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
  modified_base 72: mod_base = OTHER; note = t is r-thymine at this position
  modified_base 74: mod_base = OTHER; note = t is r-thymine at this position
  modified_base 75: mod_base = OTHER; note = t is r-thymine at this position
gtcacggtggccgagtggttaaggcgttggactctgaatccaatggggtttccccgcacaggttcgaatcctgttcgtgacgcca

SEQ ID NO: 685 - RNA, 85 nt, synthetic construct
  misc_feature 1..85: note = Description of sequence: tRNA-based effector molecule; TREM No: 61
  source 1..85: mol_type = other RNA
  modified_base 3: mod_base = OTHER; note = t is r-thymine at this position
  modified_base 5: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 25: mod_base = OTHER; note = t is r-thymine at this position
  modified_base 27: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 44: mod_base = OTHER; note = t is r-thymine at this position
  modified_base 50: mod_base = OTHER; note = t is r-thymine at this position
  modified_base 51: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
  modified_base 72: mod_base = OTHER; note = t is r-thymine at this position
  modified_base 75: mod_base = OTHER; note = t is r-thymine at this position
gctgtgatggccgagtggttaaggtgttggactctgaatccaatgggggttccccgcgcaggttcaaatcctgctcacagcgcca

SEQ ID NO: 686 - RNA, 85 nt, synthetic construct
  misc_feature 1..85: note = Description of sequence: tRNA-based effector molecule; TREM No: 62
  source 1..85: mol_type = other RNA
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 43: mod_base = OTHER; note = t is r-thymine at this position
  modified_base 44: mod_base = OTHER; note = t is r-thymine at this position
  modified_base 46: mod_base = OTHER; note = t is r-thymine at this position
  modified_base 49: mod_base = OTHER; note = t is r-thymine at this position
  modified_base 51: mod_base = OTHER; note = t is r-thymine at this position
  modified_base 59: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
gacgaggtggccgagtggttaaggcgatggactctgaatccattgtgctctgcacgcgtgggttcgaatcccaccctcgtcgcca

SEQ ID NO: 687 - RNA, 85 nt, synthetic construct; identical to SEQ ID NO: 634
  misc_feature 1..85: note = Description of sequence: tRNA-based effector molecule; TREM No: 63
  source 1..85: mol_type = other RNA
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 43: mod_base = OTHER; note = t is r-thymine at this position
  modified_base 44: mod_base = OTHER; note = t is r-thymine at this position
  modified_base 46: mod_base = OTHER; note = t is r-thymine at this position
  modified_base 49: mod_base = OTHER; note = t is r-thymine at this position
  modified_base 50: mod_base = OTHER; note = t is r-thymine at this position
  modified_base 51: mod_base = OTHER; note = t is r-thymine at this position
  modified_base 59: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
  modified_base 74: mod_base = OTHER; note = t is r-thymine at this position

SEQ ID NO: 688 - RNA, 85 nt, synthetic construct
  misc_feature 1..85: note = Description of sequence: tRNA-based effector molecule; TREM No: 64
  source 1..85: mol_type = other RNA
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 43: mod_base = OTHER; note = t is r-thymine at this position
  modified_base 44: mod_base = OTHER; note = t is r-thymine at this position
  modified_base 46: mod_base = OTHER; note = t is r-thymine at this position
  modified_base 49: mod_base = OTHER; note = t is r-thymine at this position
  modified_base 51: mod_base = OTHER; note = t is r-thymine at this position
  modified_base 59: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
  modified_base 74: mod_base = OTHER; note = t is r-thymine at this position
gacgaggtggccgagtggttaaggcgatggactctgaatccattgtgctctgcacgcgtgggttcgaatcccatcctcgtcgcca

SEQ ID NO: 689 - RNA, 85 nt, synthetic construct
  misc_feature 1..85: note = Description of sequence: tRNA-based effector molecule; TREM No: 65
  source 1..85: mol_type = other RNA
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 43: mod_base = OTHER; note = t is r-thymine at this position
  modified_base 44: mod_base = OTHER; note = t is r-thymine at this position
  modified_base 46: mod_base = OTHER; note = t is r-thymine at this position
  modified_base 49: mod_base = OTHER; note = t is r-thymine at this position
  modified_base 51: mod_base = OTHER; note = t is r-thymine at this position
  modified_base 59: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
gacgaggtggccgagtggttaaggcgatggactctgaatccattgtgctctgcacgcgtgggttcgaatcccaccttcgtcgcca

SEQ ID NO: 690 - RNA, 85 nt, synthetic construct
  misc_feature 1..85: note = Description of sequence: tRNA-based effector molecule; TREM No: 66
  source 1..85: mol_type = other RNA
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 43: mod_base = OTHER; note = t is r-thymine at this position
  modified_base 44: mod_base = OTHER; note = t is r-thymine at this position
  modified_base 46: mod_base = OTHER; note = t is r-thymine at this position
  modified_base 49: mod_base = OTHER; note = t is r-thymine at this position
  modified_base 51: mod_base = OTHER; note = t is r-thymine at this position
  modified_base 59: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
  modified_base 74: mod_base = OTHER; note = t is r-thymine at this position
gacgaggtggccgagtggttaaggcgatggactctgaatccattgtgctctgcacacgtgggttcgaatcccatcctcgtcgcca

SEQ ID NO: 691 - RNA, 87 nt, synthetic construct
  misc_feature 1..87: note = Description of sequence: tRNA-based effector molecule; TREM No: 67
  source 1..87: mol_type = other RNA
  modified_base 10: mod_base = OTHER; note = t is r-thymine at this position
  modified_base 18: mod_base = OTHER; note = t is r-thymine at this position
  modified_base 21: mod_base = OTHER; note = t is r-thymine at this position
  modified_base 22: mod_base = OTHER; note = t is r-thymine at this position
  modified_base 30: mod_base = OTHER; note = t is r-thymine at this position
  modified_base 35: mod_base = OTHER; note = t is r-thymine at this position
  modified_base 37: mod_base = OTHER; note = t is r-thymine at this position
  modified_base 41: mod_base = OTHER; note = t is r-thymine at this position
  modified_base 45: mod_base = OTHER; note = t is r-thymine at this position
  modified_base 46: mod_base = OTHER; note = t is r-thymine at this position
  modified_base 48: mod_base = OTHER; note = t is r-thymine at this position
  modified_base 51: mod_base = OTHER; note = t is r-thymine at this position
  modified_base 53: mod_base = OTHER; note = t is r-thymine at this position
  modified_base 61: mod_base = OTHER; note = t is r-thymine at this position
  modified_base 65: mod_base = OTHER; note = t is r-thymine at this position
  modified_base 66: mod_base = OTHER; note = t is r-thymine at this position
  modified_base 71: mod_base = OTHER; note = t is r-thymine at this position
ggagaggcctggccgagtggttaaggcgatggactctgaatccattgtgctctgcacgcgtgggttcgaatcccatcctcgtcgcca

SEQ ID NO: 692 - RNA, 85 nt, synthetic construct
  misc_feature 1..85: note = Description of sequence: tRNA-based effector molecule; TREM No: 68
  source 1..85: mol_type = other RNA
  modified_base 2: mod_base = OTHER; note = t is r-thymine at this position
  modified_base 5: mod_base = OTHER; note = t is r-thymine at this position
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 43: mod_base = OTHER; note = t is r-thymine at this position
  modified_base 44: mod_base = OTHER; note = t is r-thymine at this position
  modified_base 49: mod_base = OTHER; note = t is r-thymine at this position
  modified_base 50: mod_base = OTHER; note = t is r-thymine at this position
  modified_base 51: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 69: mod_base = OTHER; note = t is r-thymine at this position
  modified_base 72: mod_base = OTHER; note = t is r-thymine at this position
  modified_base 74: mod_base = OTHER; note = t is r-thymine at this position
gtagtcgtggccgagtggttaaggcgatggactctgaatccattggggtttccccgcgcaggttcgaatcctgtcggctacgcca

SEQ ID NO: 693 - RNA, 85 nt, synthetic construct; identical to SEQ ID NO: 635
  misc_feature 1..85: note = Description of sequence: tRNA-based effector molecule; TREM No: 69
  source 1..85: mol_type = other RNA
  modified_base 8: mod_base = OTHER; note = t is r-thymine at this position
  modified_base 16: mod_base = OTHER; note = t is r-thymine at this position
  modified_base 19: mod_base = OTHER; note = t is r-thymine at this position
  modified_base 20: mod_base = OTHER; note = t is r-thymine at this position
  modified_base 27: mod_base = OTHER; note = t is r-thymine at this position
  modified_base 28: mod_base = OTHER; note = t is r-thymine at this position
  modified_base 33: mod_base = OTHER; note = t is r-thymine at this position
  modified_base 35: mod_base = OTHER; note = t is r-thymine at this position
  modified_base 39: mod_base = OTHER; note = t is r-thymine at this position
  modified_base 44: mod_base = OTHER; note = t is r-thymine at this position
  modified_base 49: mod_base = OTHER; note = t is r-thymine at this position
  modified_base 51: mod_base = OTHER; note = t is r-thymine at this position
  modified_base 63: mod_base = OTHER; note = t is r-thymine at this position
  modified_base 64: mod_base = OTHER; note = t is r-thymine at this position
  modified_base 72: mod_base = OTHER; note = t is r-thymine at this position
  modified_base 75: mod_base = OTHER; note = t is r-thymine at this position

SEQ ID NO: 694 - RNA, 76 nt, synthetic construct
  misc_feature 1..76: note = Description of sequence: tRNA-based effector molecule; TREM No: 70
  source 1..76: mol_type = other RNA
gggccagtggcgcaatggataacgcgtctgactctggatcagaagattctaggttcgactcctggctggctcgcca

SEQ ID NO: 695 - RNA, 76 nt, synthetic construct
  misc_feature 1..76: note = Description of sequence: tRNA-based effector molecule; TREM No: 71
  source 1..76: mol_type = other RNA
ggccgcgtggcctaatggataaggcgtctgattctggatcagaagattgagggttcgagtcccttcgtggtcgcca

SEQ ID NO: 696 - RNA, 76 nt, synthetic construct
  misc_feature 1..76: note = Description of sequence: tRNA-based effector molecule; TREM No: 72
  source 1..76: mol_type = other RNA
gccccagtggcctaatggataaggcattggcctctgaagccagggattgtgggttcgagtcccatctggggtgcca

SEQ ID NO: 697 - RNA, 76 nt, synthetic construct
  misc_feature 1..76: note = Description of sequence: tRNA-based effector molecule; TREM No: 73
  source 1..76: mol_type = other RNA
ggccgcgtggcctaatggataaggcgtctgactctggatcagaagattgcaggttcgagtcctgccgcggtcgcca

SEQ ID NO: 698 - RNA, 88 nt, synthetic construct
  misc_feature 1..88: note = Description of sequence: tRNA-based effector molecule; TREM No: 74
  source 1..88: mol_type = other RNA
ggctccgtggcgcaatggatagcgcattggactctgagaggctgaaggcattcaaaggttccgggttcgagtcccggcggagtcgcca

SEQ ID NO: 699 - RNA, 75 nt, synthetic construct
  misc_feature 1..75: note = Description of sequence: tRNA-based effector molecule; TREM No: 75
  source 1..75: mol_type = other RNA
ggctcgttggtctaggggtatgattctcgcttctgatgcgagaggtcccgggttcaaatcccggacgagccccca

SEQ ID NO: 700 - RNA, 75 nt, synthetic construct
  misc_feature 1..75: note = Description of sequence: tRNA-based effector molecule; TREM No: 76
  source 1..75: mol_type = other RNA
ggctcgttggtctaggggtatgattctcgcttctggtgcgagaggtcccgggttcaaatcccggacgagccccca

SEQ ID NO: 701-1199 - skipped (499 entries)

SEQ ID NO: 1200 - DNA, 73 bp, synthetic construct; identical to SEQ ID NO: 4
  misc_feature 1..73: note = Description of sequence: Consensus sequence
  source 1..73: mol_type = other DNA

SEQ ID NO: 1201 - DNA, 72 bp, synthetic construct; identical to SEQ ID NO: 17
  misc_feature 1..72: note = Description of sequence: Consensus sequence
  source 1..72: mol_type = other DNA

SEQ ID NO: 1202 - DNA, 72 bp, synthetic construct
  misc_feature 1..72: note = Description of sequence: Consensus sequence
  source 1..72: mol_type = other DNA
gggggtgtagctcagtggtagagcgcatgctttgcatgtatgaggccccgggttcgatccccggcacctcca

SEQ ID NO: 1203 - DNA, 73 bp, synthetic construct; identical to SEQ ID NO: 28
  misc_feature 1..73: note = Description of sequence: Consensus sequence
  source 1..73: mol_type = other DNA

SEQ ID NO: 1204 - DNA, 73 bp, synthetic construct; identical to SEQ ID NO: 30
  misc_feature 1..73: note = Description of sequence: Consensus sequence
  source 1..73: mol_type = other DNA

SEQ ID NO: 1205 - DNA, 73 bp, synthetic construct; identical to SEQ ID NO: 32
  misc_feature 1..73: note = Description of sequence: Consensus sequence
  source 1..73: mol_type = other DNA

SEQ ID NO: 1206 - DNA, 73 bp, synthetic construct
  misc_feature 1..73: note = Description of sequence: Consensus sequence
  source 1..73: mol_type = other DNA
gaccgcgtggcctaatggataaggcgtctgacttcggatcagaagattgagggttcgagtccctccgtggtcg

SEQ ID NO: 1207 - DNA, 74 bp, synthetic construct
  misc_feature 1..74: note = Description of sequence: Consensus sequence
  source 1..74: mol_type = other DNA
  variation 21: note = n is a, c, g, or t
  variation 65: note = n is a, c, g, or t
ggctctgtggcgcaatggatnagcgcattggacttctaattcaaaggttgcgggttcgagtcccnccagagtcg

SEQ ID NO: 1208 - DNA, 75 bp, synthetic construct
  misc_feature 1..75: note = Description of sequence: Consensus sequence
  source 1..75: mol_type = other DNA
  variation 43: note = n is a, c, g, or t
gtctctgtggcgcaatcggttagcgcgttcggctgttaaccgnaaaggttggtggttcgagcccacccagggacg

SEQ ID NO: 1209 - DNA, 72 bp, synthetic construct; identical to SEQ ID NO: 62
  misc_feature 1..72: note = Description of sequence: Consensus sequence
  source 1..72: mol_type = other DNA

SEQ ID NO: 1210 - DNA, 73 bp, synthetic construct
  misc_feature 1..73: note = Description of sequence: Consensus sequence
  source 1..73: mol_type = other DNA
  variation 16: note = n is a, c, g, or t
gggggtatagctcagngggtagagcatttgactgcagatcaagaggtccccggttcaaatccgggtgccccct

SEQ ID NO: 1211 - DNA, 72 bp, synthetic construct
  misc_feature 1..72: note = Description of sequence: Consensus sequence
  source 1..72: mol_type = other DNA
  variation 20: note = n is a, c, g, or t
ggttccatggtgtaatggtnagcactctggactctgaatccagcgatccgagttcaagtctcggtggaacct

SEQ ID NO: 1212 - DNA, 72 bp, synthetic construct; identical to SEQ ID NO: 94
  misc_feature 1..72: note = Description of sequence: Consensus sequence
  source 1..72: mol_type = other DNA

SEQ ID NO: 1213 - DNA, 72 bp, synthetic construct; identical to SEQ ID NO: 98
  misc_feature 1..72: note = Description of sequence: Consensus sequence
  source 1..72: mol_type = other DNA

SEQ ID NO: 1214 - DNA, 72 bp, synthetic construct
  misc_feature 1..72: note = Description of sequence: Consensus sequence
  source 1..72: mol_type = other DNA
  variation 42: note = n is a, c, g, or t
tccctggtggtctagtggctaggattcggcgctttcaccgcngcggcccgggttcgattcccggtcagggaa

SEQ ID NO: 1215 - DNA, 71 bp, synthetic construct
  misc_feature 1..71: note = Description of sequence: Consensus sequence
  source 1..71: mol_type = other DNA
  variation 40: note = n is a, c, g, or t
gcattggtggttcagtggtagaattctcgcctcccacgcnggagacccgggttcgattcccggccaatgca

SEQ ID NO: 1216 - DNA, 71 bp, synthetic construct; identical to SEQ ID NO: 108
  misc_feature 1..71: note = Description of sequence: Consensus sequence
  source 1..71: mol_type = other DNA

SEQ ID NO: 1217 - DNA, 72 bp, synthetic construct; identical to SEQ ID NO: 113
  misc_feature 1..72: note = Description of sequence: Consensus sequence
  source 1..72: mol_type = other DNA

SEQ ID NO: 1218 - DNA, 74 bp, synthetic construct; identical to SEQ ID NO: 122
  misc_feature 1..74: note = Description of sequence: Consensus sequence
  source 1..74: mol_type = other DNA

SEQ ID NO: 1219 - DNA, 74 bp, synthetic construct
  misc_feature 1..74: note = Description of sequence: Consensus sequence
  source 1..74: mol_type = other DNA
gctccagtggcgcaatcggttagcgcgcggtacttataatgccgaggttgtgagttcgagcctcacctggagca

SEQ ID NO: 1220 - DNA, 82 bp, synthetic construct; identical to SEQ ID NO: 134
  misc_feature 1..82: note = Description of sequence: Consensus sequence
  source 1..82: mol_type = other DNA

SEQ ID NO: 1221 - DNA, 85 bp, synthetic construct
  misc_feature 1..85: note = Description of sequence: Consensus sequence
  source 1..85: mol_type = other DNA
  variation 21: note = n is a, c, g, or t
  variation 52: note = n is a, c, g, or t
gtcaggatggccgagtggtcntaaggcgccagactcaagttctggtctccgnatggaggcgtgggttcgaatcccacttctgaca

SEQ ID NO: 1222 - DNA, 83 bp, synthetic construct; identical to SEQ ID NO: 144
  misc_feature 1..83: note = Description of sequence: Consensus sequence
  source 1..83: mol_type = other DNA

SEQ ID NO: 1223 - DNA, 83 bp, synthetic construct
  misc_feature 1..83: note = Description of sequence: Consensus sequence
  source 1..83: mol_type = other DNA
accaggatggccgagtggttaaggcgttggacttaagatccaatggacagatgtccgcgtgggttcgaaccccactcctggta

SEQ ID NO: 1224 - DNA, 82 bp, synthetic construct
  misc_feature 1..82: note = Description of sequence: Consensus sequence
  source 1..82: mol_type = other DNA
  variation 54: note = n is a, c, g, or t
ggtagcgtggccgagcggtctaaggcgctggatttaggctccagtctcttcggnggcgtgggttcgaatcccaccgctgcca

SEQ ID NO: 1225 - DNA, 76 bp, synthetic construct
  misc_feature 1..76: note = Description of sequence: Consensus sequence
  source 1..76: mol_type = other DNA
  variation 74: note = n is a, c, g, or t
  variation 75: note = n is a, c, g, or t
  variation 76: note = n is a, c, g, or t
gcccggctagctcagtcggtagagcatgagactcttaatctcagggtcgtgggttcgagccccacgttgggcgnnn

SEQ ID NO: 1226 - DNA, 73 bp, synthetic construct; identical to SEQ ID NO: 167
  misc_feature 1..73: note = Description of sequence: Consensus sequence
  source 1..73: mol_type = other DNA

SEQ ID NO: 1227 - DNA, 73 bp, synthetic construct
  misc_feature 1..73: note = Description of sequence: Consensus sequence
  source 1..73: mol_type = other DNA
  variation 17: note = n is a, c, g, or t
gccctcttagcgcagtnggcagcgcgtcagtctcataatctgaaggtcctgagttcgagcctcagagagggca

SEQ ID NO: 1228 - DNA, 74 bp, synthetic construct
  misc_feature 1..74: note = Description of sequence: Consensus sequence
  source 1..74: mol_type = other DNA
  variation 41: note = n is a, c, g, or t
gccgaaatagctcagttgggagagcgttagactgaagatcntaaaggtccctggttcaatcccgggtttcggca

SEQ ID NO: 1229 - DNA, 72 bp, synthetic construct; identical to SEQ ID NO: 183
  misc_feature 1..72: note = Description of sequence: Consensus sequence
  source 1..72: mol_type = other DNA

SEQ ID NO: 1230 - DNA, 72 bp, synthetic construct; identical to SEQ ID NO: 185
  misc_feature 1..72: note = Description of sequence: Consensus sequence
  source 1..72: mol_type = other DNA

SEQ ID NO: 1231 - DNA, 72 bp, synthetic construct; identical to SEQ ID NO: 189
  misc_feature 1..72: note = Description of sequence: Consensus sequence
  source 1..72: mol_type = other DNA

SEQ ID NO: 1232 - DNA, 82 bp, synthetic construct; identical to SEQ ID NO: 192
  misc_feature 1..82: note = Description of sequence: Consensus sequence
  source 1..82: mol_type = other DNA

SEQ ID NO: 1233 - DNA, 82 bp, synthetic construct; identical to SEQ ID NO: 196
  misc_feature 1..82: note = Description of sequence: Consensus sequence
  source 1..82: mol_type = other DNA

SEQ ID NO: 1234 - DNA, 84 bp, synthetic construct
  misc_feature 1..84: note = Description of sequence: Consensus sequence
  source 1..84: mol_type = other DNA
  variation 8: note = n is a, c, g, or t
  variation 9: note = n is a, c, g, or t
gacgaggnntggccgagtggttaaggcgatggactgctaatccattgtgctctgcacgcgtgggttcgaatcccatcctcgtcg

SEQ ID NO: 1235 - DNA, 82 bp, synthetic construct
  misc_feature 1..82: note = Description of sequence: Consensus sequence
  source 1..82: mol_type = other DNA
gtagtcgtggccgagtggttaaggcgatggacttgaaatccattggggtctccccgcgcaggttcgaatcctgccggctacg

SEQ ID NO: 1236 - DNA, 74 bp, synthetic construct; identical to SEQ ID NO: 211
  misc_feature 1..74: note = Description of sequence: Consensus sequence
  source 1..74: mol_type = other DNA

SEQ ID NO: 1237 - DNA, 75 bp, synthetic construct
  misc_feature 1..75: note = Description of sequence: Consensus sequence
  source 1..75: mol_type = other DNA
  variation 4: note = n is a, c, g, or t
  variation 13: note = n is a, c, g, or t
  variation 17: note = n is a, c, g, or t
  variation 20: note = n is a, c, g, or t
  variation 43: note = n is a, c, g, or t
  variation 51: note = n is a, c, g, or t
  variation 67: note = n is a, c, g, or t
  variation 69: note = n is a, c, g, or t
ggcnctgtggctnagtnggntaaagcgccggtctcgtaaaccnggagatcntgggttcgaatcccancngggcct

SEQ ID NO: 1238 - DNA, 74 bp, synthetic construct
  misc_feature 1..74: note = Description of sequence: Consensus sequence
  source 1..74: mol_type = other DNA
  variation 16: note = n is a, c, g, or t
ggctccatagctcagngggttagagcactggtcttgtaaaccaggggtcgcgagttcaaatctcgctggggcct

SEQ ID NO: 1239 - DNA, 72 bp, synthetic construct; identical to SEQ ID NO: 229
  misc_feature 1..72: note = Description of sequence: Consensus sequence
  source 1..72: mol_type = other DNA

SEQ ID NO: 1240 - DNA, 73 bp, synthetic construct
  misc_feature 1..73: note = Description of sequence: Consensus sequence
  source 1..73: mol_type = other DNA
ccttcgatagctcagctggtagagcggaggactgtagatccttaggtcgctggttcgattccggctcgaagga

SEQ ID NO: 1241 - DNA, 73 bp, synthetic construct; identical to SEQ ID NO: 246
  misc_feature 1..73: note = Description of sequence: Consensus sequence
  source 1..73: mol_type = other DNA

SEQ ID NO: 1242 - DNA, 73 bp, synthetic construct; identical to SEQ ID NO: 252
  misc_feature 1..73: note = Description of sequence: Consensus sequence
  source 1..73: mol_type = other DNA

SEQ ID NO: 1243 - DNA, 73 bp, synthetic construct; identical to SEQ ID NO: 258
  misc_feature 1..73: note = Description of sequence: Consensus sequence
  source 1..73: mol_type = other DNA

SEQ ID NO: 1244 - DNA, 72 bp, synthetic construct; identical to SEQ ID NO: 262
  misc_feature 1..72: note = Description of sequence: Consensus sequence
  source 1..72: mol_type = other DNA

SEQ ID NO: 1245 - DNA, 76 bp, synthetic construct
  misc_feature 1..76: note = Description of sequence: Consensus sequence
  source 1..76: mol_type = other DNA
  variation 74: note = n is a, c, g, or t
  variation 75: note = n is a, c, g, or t
  variation 76: note = n is a, c, g, or t
ggggaattagctcaagtggtagagcgcttgcttagcatgcaagaggtagtgggatcgatgcccacattctccannn

SEQ ID NO: 1246 - DNA, 75 bp, synthetic construct
  misc_feature 1..75: note = Description of sequence: Consensus sequence
  source 1..75: mol_type = other DNA
  variation 73: note = n is a, c, g, or t
  variation 74: note = n is a, c, g, or t
  variation 75: note = n is a, c, g, or t
ggggatgtagctcagtggtagagcgcatgcttcgcatgtatgaggtcccgggttcgatccccggcatctccannn

SEQ ID NO: 1247 - DNA, 75 bp, synthetic construct
  misc_feature 1..75: note = Description of sequence: Consensus sequence
  source 1..75: mol_type = other DNA
  variation 73: note = n is a, c, g, or t
  variation 74: note = n is a, c, g, or t
  variation 75: note = n is a, c, g, or t
gggggtgtagctcagtggtagagcgcatgctttgcatgtatgaggccccgggttcgatccccggcacctccannn

SEQ ID NO: 1248 - DNA, 76 bp, synthetic construct
  misc_feature 1..76: note = Description of sequence: Consensus sequence
  source 1..76: mol_type = other DNA
  variation 74: note = n is a, c, g, or t
  variation 75: note = n is a, c, g, or t
  variation 76: note = n is a, c, g, or t
gggccagtggcgcaatggataacgcgtctgactacggatcagaagattccaggttcgactcctggctggctcgnnn

SEQ ID NO: 1249 - DNA, 76 bp, synthetic construct
  misc_feature 1..76: note = Description of sequence: Consensus sequence
  source 1..76: mol_type = other DNA
  variation 74: note = n is a, c, g, or t
  variation 75: note = n is a, c, g, or t
  variation 76: note = n is a, c, g, or t
ggccgcgtggcctaatggataaggcgtctgattccggatcagaagattgagggttcgagtcccttcgtggtcgnnn

SEQ ID NO: 1250 - DNA, 76 bp, synthetic construct
  misc_feature 1..76: note = Description of sequence: Consensus sequence
  source 1..76: mol_type = other DNA
  variation 74: note = n is a, c, g, or t
  variation 75: note = n is a, c, g, or t
  variation 76: note = n is a, c, g, or t
gccccagtggcctaatggataaggcactggcctcctaagccagggattgtgggttcgagtcccacctggggtannn

SEQ ID NO: 1251 - DNA, 76 bp, synthetic construct
  misc_feature 1..76: note = Description of sequence: Consensus sequence
  source 1..76: mol_type = other DNA
  variation 74: note = n is a, c, g, or t
  variation 75: note = n is a, c, g, or t
  variation 76: note = n is a, c, g, or t
gaccgcgtggcctaatggataaggcgtctgacttcggatcagaagattgagggttcgagtccctccgtggtcgnnn

SEQ ID NO: 1252 - DNA, 77 bp, synthetic construct
  misc_feature 1..77: note = Description of sequence: Consensus sequence
  source 1..77: mol_type = other DNA
  variation 21: note = n is a, c, g, or t
  variation 65: note = n is a, c, g, or t
  variation 75: note = n is a, c, g, or t
  variation 76: note = n is a, c, g, or t
  variation 77: note = n is a, c, g, or t
ggctctgtggcgcaatggatnagcgcattggacttctaattcaaaggttgcgggttcgagtcccnccagagtcgnnn

SEQ ID NO: 1253 - DNA, 78 bp, synthetic construct
  misc_feature 1..78: note = Description of sequence: Consensus sequence
  source 1..78: mol_type = other DNA
  variation 43: note = n is a, c, g, or t
  variation 76: note = n is a, c, g, or t
  variation 77: note = n is a, c, g, or t
  variation 78: note = n is a, c, g, or t
gtctctgtggcgcaatcggttagcgcgttcggctgttaaccgnaaaggttggtggttcgagcccacccagggacgnnn

SEQ ID NO: 1254 - DNA, 75 bp, synthetic construct
  misc_feature 1..75: note = Description of sequence: Consensus sequence
  source 1..75: mol_type = other DNA
  variation 73: note = n is a, c, g, or t
  variation 74: note = n is a, c, g, or t
  variation 75: note = n is a, c, g, or t
tcctcgttagtatagtggtgagtatccccgcctgtcacgcgggagaccggggttcgattccccgacggggagnnn

SEQ ID NO: 1255 - DNA, 76 bp, synthetic construct
  misc_feature 1..76: note = Description of sequence: Consensus sequence
  source 1..76: mol_type = other DNA
  variation 16: note = n is a, c, g, or t
  variation 74: note = n is a, c, g, or t
  variation 75: note = n is a, c, g, or t
  variation 76: note = n is a, c, g, or t
gggggtatagctcagngggtagagcatttgactgcagatcaagaggtccccggttcaaatccgggtgccccctnnn

SEQ ID NO: 1256 - DNA, 75 bp, synthetic construct
  misc_feature 1..75: note = Description of sequence: Consensus sequence
  source 1..75: mol_type = other DNA
  variation 20: note = n is a, c, g, or t
  variation 73: note = n is a, c, g, or t
  variation 74: note = n is a, c, g, or t
  variation 75: note = n is a, c, g, or t
ggttccatggtgtaatggtnagcactctggactctgaatccagcgatccgagttcaagtctcggtggaacctnnn

SEQ ID NO: 1257 - DNA, 75 bp, synthetic construct
  misc_feature 1..75: note = Description of sequence: Consensus sequence
  source 1..75: mol_type = other DNA
  variation 73: note = n is a, c, g, or t
  variation 74: note = n is a, c, g, or t
  variation 75: note = n is a, c, g, or t
ggtcccatggtgtaatggttagcactctggactttgaatccagcgatccgagttcaaatctcggtgggacctnnn

SEQ ID NO: 1258 - DNA, 75 bp, synthetic construct
  misc_feature 1..75: note = Description of sequence: Consensus sequence
  source 1..75: mol_type = other DNA
  variation 73: note = n is a, c, g, or t
  variation 74: note = n is a, c, g, or t
  variation 75: note = n is a, c, g, or t
tccctggtggtctagtggttaggattcggcgctctcaccgccgcggcccgggttcgattcccggtcagggaannn

SEQ ID NO: 1259 - DNA, 75 bp, synthetic construct
  misc_feature 1..75: note = Description of sequence: Consensus sequence
  source 1..75: mol_type = other DNA
  variation 42: note = n is a, c, g, or t
  variation 73: note = n is a, c, g, or t
  variation 74: note = n is a, c, g, or t
  variation 75: note = n is a, c, g, or t
tccctggtggtctagtggctaggattcggcgctttcaccgcngcggcccgggttcgattcccggtcagggaannn

SEQ ID NO: 1260 - DNA, 74 bp, synthetic construct
  misc_feature 1..74: note = Description of sequence: Consensus sequence
  source 1..74: mol_type = other DNA
  variation 40: note = n is a, c, g, or t
  variation 72: note = n is a, c, g, or t
  variation 73: note = n is a, c, g, or t
  variation 74: note = n is a, c, g, or t
gcattggtggttcagtggtagaattctcgcctcccacgcnggagacccgggttcgattcccggccaatgcannn

SEQ ID NO: 1261 - DNA, 74 bp, synthetic construct
  misc_feature 1..74: note = Description of sequence: Consensus sequence
  source 1..74: mol_type = other DNA
  variation 72: note = n is a, c, g, or t
  variation 73: note = n is a, c, g, or t
  variation 74: note = n is a, c, g, or t
gcattggtggttcagtggtagaattctcgcctgccacgcgggaggcccgggttcgattcccggccaatgcannn

SEQ ID NO: 1262 - DNA, 75 bp, synthetic construct
  misc_feature 1..75: note = Description of sequence: Consensus sequence
  source 1..75: mol_type = other DNA
  variation 73: note = n is a, c, g, or t
  variation 74: note = n is a, c, g, or t
  variation 75: note = n is a, c, g, or t
gcgttggtggtatagtggtgagcatagctgccttccaagcagttgacccgggttcgattcccggccaacgcannn

SEQ ID NO: 1263 - DNA, 77 bp, synthetic construct
  misc_feature 1..77: note = Description of sequence: Consensus sequence
  source 1..77: mol_type = other DNA
  variation 75: note = n is a, c, g, or t
  variation 76: note = n is a, c, g, or t
  variation 77: note = n is a, c, g, or t
ggccggttagctcagttggttagagcgtggtgctaataacgccaaggtcgcgggttcgatccccgtacgggccannn

SEQ ID NO: 1264 - DNA, 77 bp, synthetic construct
  misc_feature 1..77: note = Description of sequence: Consensus sequence
  source 1..77: mol_type = other DNA
  variation 75: note = n is a, c, g, or t
  variation 76: note = n is a, c, g, or t
  variation 77: note = n is a, c, g, or t
gctccagtggcgcaatcggttagcgcgcggtacttataatgccgaggttgtgagttcgagcctcacctggagcannn

SEQ ID NO: 1265 - DNA, 85 bp, synthetic construct
  misc_feature 1..85: note = Description of sequence: Consensus sequence
  source 1..85: mol_type = other DNA
  variation 83: note = n is a, c, g, or t
  variation 84: note = n is a, c, g, or t
  variation 85: note = n is a, c, g, or t
ggtagcgtggccgagcggtctaaggcgctggattaaggctccagtctcttcgggggcgtgggttcgaatcccaccgctgccannn

SEQ ID NO: 1266 - DNA, 88 bp, synthetic construct
  misc_feature 1..88: note = Description of sequence: Consensus sequence
  source 1..88: mol_type = other DNA
  variation 21: note = n is a, c, g, or t
  variation 52: note = n is a, c, g, or t
  variation 86: note = n is a, c, g, or t
  variation 87: note = n is a, c, g, or t
  variation 88: note = n is a, c, g, or t
gtcaggatggccgagtggtcntaaggcgccagactcaagttctggtctccgnatggaggcgtgggttcgaatcccacttctgacannn

SEQ ID NO: 1267 - DNA, 86 bp, synthetic construct
  misc_feature 1..86: note = Description of sequence: Consensus sequence
  source 1..86: mol_type = other DNA
  variation 84: note = n is a, c, g, or t
  variation 85: note = n is a, c, g, or t
  variation 86: note = n is a, c, g, or t
gtcaggatggccgagcggtctaaggcgctgcgttcaggtcgcagtctcccctggaggcgtgggttcgaatcccactcctgacannn

SEQ ID NO: 1268 - DNA, 86 bp, synthetic construct
  misc_feature 1..86: note = Description of sequence: Consensus sequence
  source 1..86: mol_type = other DNA
  variation 84: note = n is a, c, g, or t
  variation 85: note = n is a, c, g, or t
  variation 86: note = n is a, c, g, or t
accaggatggccgagtggttaaggcgttggacttaagatccaatggacagatgtccgcgtgggttcgaaccccactcctggtannn

SEQ ID NO: 1269 - DNA, 85 bp, synthetic construct
  misc_feature 1..85: note = Description of sequence: Consensus sequence
  source 1..85: mol_type = other DNA
  variation 54: note = n is a, c, g, or t
  variation 83: note = n is a, c, g, or t
  variation 84: note = n is a, c, g, or t
  variation 85: note = n is a, c, g, or t
ggtagcgtggccgagcggtctaaggcgctggatttaggctccagtctcttcggnggcgtgggttcgaatcccaccgctgccannn

SEQ ID NO: 1270 - DNA, 79 bp, synthetic construct
  misc_feature 1..79: note = Description of sequence: Consensus sequence
  source 1..79: mol_type = other DNA
  variation 74: note = n is a, c, g, or t
  variation 75: note = n is a, c, g, or t
  variation 76: note = n is a, c, g, or t
  variation 77: note = n is a, c, g, or t
  variation 78: note = n is a, c, g, or t
  variation 79: note = n is a, c, g, or t
gcccggctagctcagtcggtagagcatgagactcttaatctcagggtcgtgggttcgagccccacgttgggcgnnnnnn

SEQ ID NO: 1271 - DNA, 76 bp, synthetic construct
  misc_feature 1..76: note = Description of sequence: Consensus sequence
  source 1..76: mol_type = other DNA
  variation 74: note = n is a, c, g, or t
  variation 75: note = n is a, c, g, or t
  variation 76: note = n is a, c, g, or t
gcctggatagctcagtcggtagagcatcagacttttaatctgagggtccagggttcaagtccctgttcaggcgnnn

SEQ ID NO: 1272 - DNA, 76 bp, synthetic construct
  misc_feature 1..76: note = Description of sequence: Consensus sequence
  source 1..76: mol_type = other DNA
  variation 17: note = n is a, c, g, or t
  variation 74: note = n is a, c, g, or t
  variation 75: note = n is a, c, g, or t
  variation 76: note = n is a, c, g, or t
gccctcttagcgcagtnggcagcgcgtcagtctcataatctgaaggtcctgagttcgagcctcagagagggcannn

SEQ ID NO: 1273 - DNA, 77 bp, synthetic construct
  misc_feature 1..77: note = Description of sequence: Consensus sequence
  source 1..77: mol_type = other DNA
  variation 41: note = n is a, c, g, or t
  variation 75: note = n is a, c, g, or t
  variation 76: note = n is a, c, g, or t
  variation 77: note = n is a, c, g, or t
gccgaaatagctcagttgggagagcgttagactgaagatcntaaaggtccctggttcaatcccgggtttcggcannn

SEQ ID NO: 1274 - DNA, 75 bp, synthetic construct
  misc_feature 1..75: note = Description of sequence: Consensus sequence
  source 1..75: mol_type = other DNA
  variation 73: note = n is a, c, g, or t
  variation 74: note = n is a, c, g, or t
  variation 75: note = n is a, c, g, or t
ggctcgttggtctaggggtatgattctcgcttaggatgcgagaggtcccgggttcaaatcccggacgagcccnnn

SEQ ID NO: 1275 - DNA, 75 bp, synthetic construct
  misc_feature 1..75: note = Description of sequence: Consensus sequence
  source 1..75: mol_type = other DNA
  variation 73: note = n is a, c, g, or t
  variation 74: note = n is a, c, g, or t
  variation 75: note = n is a, c, g, or t
ggctcgttggtctaggggtatgattctcgcttcgggtgcgagaggtcccgggttcaaatcccggacgagcccnnn

SEQ ID NO: 1276 - DNA, 75 bp, synthetic construct
  misc_feature 1..75: note = Description of sequence: Consensus sequence
  source 1..75: mol_type = other DNA
  variation 73: note = n is a, c, g, or t
  variation 74: note = n is a, c, g, or t
  variation 75: note = n is a, c, g, or t
ggctcgttggtctaggggtatgattctcgctttgggtgcgagaggtcccgggttcaaatcccggacgagcccnnn

SEQ ID NO: 1277 - DNA, 85 bp, synthetic construct
  misc_feature 1..85: note = Description of sequence: Consensus sequence
  source 1..85: mol_type = other DNA
  variation 83: note = n is a, c, g, or t
  variation 84: note = n is a, c, g, or t
  variation 85: note = n is a, c, g, or t
gtagtcgtggccgagtggttaaggcgatggactagaaatccattggggtttccccgcgcaggttcgaatcctgccgactacgnnn

SEQ ID NO: 1278 - DNA, 85 bp, synthetic construct
  misc_feature 1..85: note = Description of sequence: Consensus sequence
  source 1..85: mol_type = other DNA
  variation 83: note = n is a, c, g, or t
  variation 84: note = n is a, c, g, or t
  variation 85: note = n is a, c, g, or t
gctgtgatggccgagtggttaaggcgttggactcgaaatccaatggggtctccccgcgcaggttcgaatcctgctcacagcgnnn

SEQ ID NO: 1279 - DNA, 87 bp, synthetic construct
  misc_feature 1..87: note = Description of sequence: Consensus sequence
  source 1..87: mol_type = other DNA
  variation 8: note = n is a, c, g, or t
  variation 9: note = n is a, c, g, or t
  variation 85: note = n is a, c, g, or t
  variation 86: note = n is a, c, g, or t
  variation 87: note = n is a, c, g, or t
gacgaggnntggccgagtggttaaggcgatggactgctaatccattgtgctctgcacgcgtgggttcgaatcccatcctcgtcgnnn

SEQ ID NO: 1280 - DNA, 85 bp, synthetic construct
  misc_feature 1..85: note = Description of sequence: Consensus sequence
  source 1..85: mol_type = other DNA
  variation 83: note = n is a, c, g, or t
  variation 84: note = n is a, c, g, or t
  variation 85: note = n is a, c, g, or t
gtagtcgtggccgagtggttaaggcgatggacttgaaatccattggggtctccccgcgcaggttcgaatcctgccggctacgnnn

SEQ ID NO: 1281 - DNA, 77 bp, synthetic construct
  misc_feature 1..77: note = Description of sequence: Consensus sequence
  source 1..77: mol_type = other DNA
  variation 75: note = n is a, c, g, or t
  variation 76: note = n is a, c, g, or t
  variation 77: note = n is a, c, g, or t
ggctccgtggcttagctggttaaagcgcctgtctagtaaacaggagatcctgggttcgaatcccagcggggcctnnn

SEQ ID NO: 1282 - DNA, 78 bp, synthetic construct
  misc_feature 1..78: note = Description of sequence: Consensus sequence
  source 1..78: mol_type = other DNA
  variation 4: note = n is a, c, g, or t
  variation 13: note = n is a, c, g, or t
  variation 17: note = n is a, c, g, or t
  variation 20: note = n is a, c, g, or t
  variation 43: note = n is a, c, g, or t
  variation 51: note = n is a, c, g, or t
  variation 67: note = n is a, c, g, or t
  variation 69: note = n is a, c, g, or t
  variation 76: note = n is a, c, g, or t
  variation 77: note = n is a, c, g, or t
  variation 78: note = n is a, c, g, or t
ggcnctgtggctnagtnggntaaagcgccggtctcgtaaaccnggagatcntgggttcgaatcccancngggcctnnn

SEQ ID NO: 1283 - DNA, 77 bp, synthetic construct
  misc_feature 1..77: note = Description of sequence: Consensus sequence
  source 1..77: mol_type = other DNA
  variation 16: note = n is a, c, g, or t
  variation 75: note = n is a, c, g, or t
  variation 76: note = n is a, c, g, or t
  variation 77: note = n is a, c, g, or t
ggctccatagctcagngggttagagcactggtcttgtaaaccaggggtcgcgagttcaaatctcgctggggcctnnn

SEQ ID NO: 1284 - DNA, 75 bp, synthetic construct
  misc_feature 1..75: note = Description of sequence: Consensus sequence
  source 1..75: mol_type = other DNA
  variation 73: note = n is a, c, g, or t
  variation 74: note = n is a, c, g, or t
  variation 75: note = n is a, c, g, or t
gacctcgtggcgcaacggtagcgcgtctgactccagatcagaaggttgcgtgttcaaatcacgtcggggtcannn

SEQ ID NO: 1285 - DNA, 76 bp, synthetic construct
  misc_feature 1..76: note = Description of sequence: Consensus sequence
  source 1..76: mol_type = other DNA
  variation 74: note = n is a, c, g, or t
  variation 75: note = n is a, c, g, or t
  variation 76: note = n is a, c, g, or t
ccttcgatagctcagctggtagagcggaggactgtagatccttaggtcgctggttcgattccggctcgaaggannn

SEQ ID NO: 1286 - DNA, 76 bp, synthetic construct
  misc_feature 1..76: note = Description of sequence: Consensus sequence
  source 1..76: mol_type = other DNA
  variation 74: note = n is a, c, g, or t
  variation 75: note = n is a, c, g, or t
  variation 76: note = n is a, c, g, or t
gtttccgtagtgtagtggttatcacgttcgcctaacacgcgaaaggtccccggttcgaaaccgggcggaaacannn

SEQ ID NO: 1287 - DNA, 76 bp, synthetic construct
  misc_feature 1..76: note = Description of sequence: Consensus sequence
  source 1..76: mol_type = other DNA
  variation 74: note = n is a, c, g, or t
  variation 75: note = n is a, c, g, or t
  variation 76: note = n is a, c, g, or t
gtttccgtagtgtagtggttatcacgttcgcctcacacgcgaaaggtccccggttcgaaaccgggcggaaacannn

SEQ ID NO: 1288 - DNA, 76 bp, synthetic construct
  misc_feature 1..76: note = Description of sequence: Consensus sequence
  source 1..76: mol_type = other DNA
  variation 74: note = n is a, c, g, or t
  variation 75: note = n is a, c, g, or t
  variation 76: note = n is a, c, g, or t
ggttccatagtgtagtggttatcacgtctgctttacacgcagaaggtcctgggttcgagccccagtggaaccannn

SEQ ID NO: 1289 - DNA, 75 bp, synthetic construct
  misc_feature 1..75: note = Description of sequence: Consensus sequence
  source 1..75: mol_type = other DNA
  variation 73: note = n is a, c, g, or t
  variation 74: note = n is a, c, g, or t
  variation 75: note = n is a, c, g, or t
agcagagtggcgcagcggaagcgtgctgggcccataacccagaggtcgatggatcgaaaccatcctctgctannn

(19) United States
(12) Patent Application Publication
(10) Pub. No.: US 2026/0294955 A1
(43) Pub. Date: Oct. 1, 2026

(54) TREMS FOR USE IN CORRECTION OF MISSENSE MUTATIONS

(71) Applicant: Flagship Pioneering Innovations VI, LLC, Cambridge, MA (US)

(72) Inventors: Theonie Anastassiadis, Somerville, MA (US); Siddharth Shukla, Medford, MA (US); Stephen William Eichhorn, Medford, MA (US); Amanda Marie Papakyrikos, Somerville, MA (US)

(21) Appl. No.: 19/474,579

(22) PCT Filed: Apr. 12, 2024
(86) PCT No.: PCT/US2024/024398

§ 371 Date: Oct. 10, 2025

Related U.S. Application Data
(60) Provisional application No. 63/458,888, filed on Apr. 12, 2023.

(51) Int. Cl. A61K 31/7115; A61K 31/7125; C12N 15/10; C12N 15/11

(52) U.S. Cl. CPC A61K 31/7115; A61K 31/7125; C12N 15/102; C12N 15/111; C12N 2310/315; C12N 2310/321; C12N 2310/322

(57) ABSTRACT

The invention relates generally to tRNA-based effector molecules for use in inserting a missense mutation into an open reading frame (ORF) of a gene, e.g., for treatment of a repeat expansion disease.

### CLAIM OF PRIORITY

This application claims priority to U.S. Application No. 63/458,888, filed on Apr. 12, 2023. The entire contents of the foregoing application is incorporated by reference in its entirety.

### BACKGROUND

Transfer RNAs (tRNAs) are complex, naturally occurring RNA molecules that possess a number of functions including initiation and elongation of proteins.

### SUMMARY

The present disclosure features modified tRNA-based effector molecules (TREMs, e.g., a TREM or TREM fragment), as well as related compositions and uses thereof, for the insertion of a missense mutation into an open-reading frame (ORF) in a gene. In an embodiment, the ORF comprises a missense mutation related to a repeat expansion disease (RED). As provided herein, TREMs are complex molecules which can mediate a variety of cellular processes. The TREMs disclosed herein comprise at least one modification (e.g., a non-naturally occurring modification), e.g., on a component nucleotide (e.g., a nucleobase or sugar) or within an internucleotide region, e.g., the TREM backbone. In one aspect, provided herein is a TREM comprising a sequence of Formula (A): [L1]x-[ASt Domain1]-[L2]x-[DH Domain]-[L3]x-[ACH Domain]-[VL Domain]-[TH Domain]-[L4]x-[ASt Domain2]-[L5]x, wherein independently, [L1] and [VL Domain], are optional and x is independently for each instance 0 or 1. In some embodiments, the TREM comprises a sequence of Formula (A-1): [L1]-[ASt Domain1]-[L2]-[DH Domain]-[L3]-[ACH Domain]-[VL Domain]-[TH Domain]-[L4]-[ASt Domain2], wherein independently, [L1] and [VL Domain], are optional. In an embodiment, one of [L1], [ASt Domain1], [L2]-[DH Domain], [L3], [ACH Domain], [VL Domain], [TH Domain], [L4], and [ASt Domain2] comprises a nucleotide comprising a non-naturally occurring modification.

In an embodiment, the TREM: (a) has the ability to: (i) support protein synthesis, (ii) be charged by a synthetase, (iii) be bound by an elongation factor, (iv) introduce an amino acid into a peptide chain, (v) support elongation, or (vi) support initiation; (b) comprises at least X contiguous nucleotides without a non-naturally occurring modification, wherein X is greater than 3, 4, 5, 6, 7, 8, 9, or 10; (c) comprises at least 3, but less than all of the nucleotides of a type (e.g., A, T, C, G or U) comprise the same non-naturally occurring modification; (d) comprises at least X nucleotides of a type (e.g., A, T, C, G or U) that do not comprise a non-naturally occurring modification, wherein X=than 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 80; (e) comprises no more than 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 80 nucleotides of a type (e.g., A, T, C, G or U) that comprise a non-naturally occurring modification; and/or (f) comprises no more than than 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 80 nucleotides of a type (e.g., A, T, C, G or U) that do not comprise a non-naturally occurring modification.

In an embodiment, the TREM comprises feature (a)(i). In an embodiment, the TREM comprises feature (a)(ii). In an embodiment, the TREM comprises feature (a)(iii). In an embodiment, the TREM comprises feature (a)(iv). In an embodiment, the TREM comprises feature (a)(v). In an embodiment, the TREM comprises feature (a)(vi). In an embodiment, the TREM comprises feature (b). In an embodiment, the TREM comprises feature (c). In an embodiment, the TREM comprises feature (d). In an embodiment, the TREM comprises feature (e). In an embodiment, the TREM comprises feature (f). In an embodiment, the TREM comprises all of features (a)-(f) or a combination thereof.

In an embodiment, the TREM Domain comprising the non-naturally occurring modification has a function, e.g., a domain function described herein.

In an aspect, provided herein is a TREM core fragment comprising a sequence of Formula B:

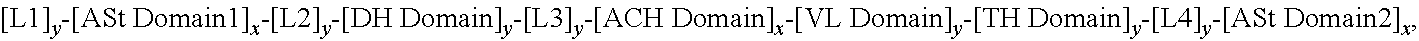

wherein x=1 and y=0 or 1; and one of [ASt Domain1], [ACH Domain], and [ASt Domain2] comprises a nucleotide having a non-naturally occurring modification.

In an embodiment, the TREM has the ability to support protein synthesis. In an embodiment, the TREM has the ability to be able to be charged by a synthetase. In an embodiment, the TREM has the ability to be bound by an elongation factor. In an embodiment, the TREM has the ability to introduce an amino acid into a peptide chain. In an embodiment, the TREM has the ability to support elongation. In an embodiment, the TREM has the ability to support initiation.

In an embodiment, the [ASt Domain 1] and/or [ASt Domain 2] comprising the non-naturally occurring modification has the ability to initiate or elongate a polypeptide chain.

In an embodiment, the [ACH Domain] comprising the non-naturally occurring modification has the ability to mediate pairing with a codon.

In an embodiment, y=1 for any one, two, three, four, five, six, all or a combination of [L1], [L2], [DH Domain], [L3], [VL Domain], [TH Domain], [L4].

In an embodiment, y=0 for any one, two, three, four, five, six, all or a combination of [L1], [L2], [DH Domain], [L3], [VL Domain], [TH Domain], [L4].

In an embodiment, y=1 for linker [L1], and L1 comprises a nucleotide having a non-naturally occurring modification.

In an embodiment, y=1 for linker [L2], and L2 comprises a nucleotide having a non-naturally occurring modification.

In an embodiment, y=1 for [DH Domain (DHD)], and DHD comprises a nucleotide having a non-naturally occurring modification. In an embodiment, the DHD comprising the non-naturally occurring modification has the ability to mediate recognition of aminoacyl-tRNA synthetase.

In an embodiment, y=1 for linker [L3], and L3 comprises a nucleotide having a non-naturally occurring modification.

In an embodiment, y=1 for [VL Domain (VLD)], and VLD comprises a nucleotide having a non-naturally occurring modification.

In an embodiment, y=1 for [TH Domain (THD)], and THD comprises a nucleotide having a non-naturally occurring modification. In an embodiment, the THD comprising the non-naturally occurring modification has the ability to mediate recognition of the ribosome.

In an embodiment, y=1 for linker [L4], and L4 comprises a nucleotide having a non-naturally occurring modification.

In another aspect, the disclosure provides a TREM fragment comprising a portion of a TREM, wherein the TREM comprises a sequence of Formula (A-1):

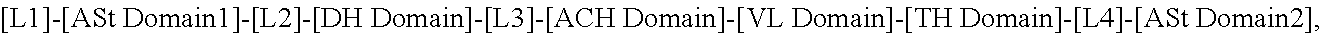

and wherein the TREM fragment comprises a non-naturally occurring modification.

In an embodiment, the TREM fragment comprises one, two, three or all or any combination of the following: (a) a TREM half (e.g., from a cleavage in the ACH Domain, e.g., in the anticodon sequence, e.g., a 5′-half or a 3′ half); (b) a 5′ fragment (e.g., a fragment comprising the 5′ end, e.g., from a cleavage in a DH Domain or the ACH Domain); (c) a 3′ fragment (e.g., a fragment comprising the 3′ end, e.g., from a cleavage in the TH Domain); or (d) an internal fragment (e.g., from a cleavage in any one of the ACH Domain, DH Domain or TH Domain).

In an embodiment, the TREM fragment comprises (a) a TREM half which comprises a nucleotide having a non-naturally occurring modification.

In an embodiment, the TREM fragment comprises (b) a 5′ fragment which comprises a nucleotide having a non-naturally occurring modification.

In an embodiment, the TREM fragment comprises (c) a 3′ fragment which comprises a nucleotide having a non-naturally occurring modification.

In an embodiment, the TREM fragment comprises (d) an internal fragment which comprises a nucleotide having a non-naturally occurring modification.

In an embodiment, of any of the TREMs, TREM core fragments, or TREM fragments disclosed herein, the TREM Domain comprises a plurality of nucleotides each having a non-naturally occurring modification. In an embodiment, the non-naturally occurring modification comprises a nucleobase modification, a sugar (e.g., ribose) modification, or a backbone modification. In an embodiment, the non-naturally occurring modification is a sugar (e.g., ribose) modification. In an embodiment, the non-naturally occurring modification is 2′-ribose modification, e.g., a 2′-OMe, 2′-halo (e.g., 2′-F), 2′-MOE, or 2′-deoxy modification. In an embodiment, the non-naturally occurring modification is a backbone modification, e.g., a phosphorothioate modification.

In an embodiment, of any of the TREMs, TREM core fragments, or TREM fragments disclosed herein, the TREM sequence comprises a CCA sequence on a terminus, e.g., the 3′ terminus. In an embodiment, the TREM sequence does not comprise a CCA sequence on a terminus, e.g., the 3′ terminus.

In an embodiment, of any of the TREMs, TREM core fragments, or TREM fragments disclosed herein, the non-naturally occurring modification is a modification in a base or a backbone of a nucleotide, e.g., a modification chosen from Table 5.

In an embodiment, of any of the TREMs, TREM core fragments, or TREM fragments disclosed herein, the non-naturally occurring modification is a base modification chosen from a modification listed in Table 5.

In an embodiment, of any of the TREMs, TREM core fragments, or TREM fragments disclosed herein, the non-naturally occurring modification is a backbone modification chosen from a modification listed in Table 5.

In an embodiment, of any of the TREMs, TREM core fragments, or TREM fragments disclosed herein, the TREM, TREM core fragment, or TREM fragment is encoded by a sequence provided in Table 1, e.g., any one of SEQ ID NOs 1-451.

In an embodiment, of any of the TREMs, TREM core fragments, or TREM fragments disclosed herein, the TREM, TREM core fragment, or TREM fragment is encoded by a consensus sequence chosen from any one of SEQ ID NOs: 562-621.

In an embodiment, of any of the TREMs, TREM core fragments, or TREM fragments disclosed herein, the TREM, TREM core fragment, or TREM fragment is encoded by a sequence provided in any one of SEQ ID NOs: 625-693. In an embodiment, the TREM, TREM core fragment, or TREM fragment comprises a TREM having at least 70%, 75%, 80%, 85%, 90%, 95%, or 99% sequence identity with a TREM provided by any one of SEQ ID NOs: 625-693. In an embodiment, the TREM, TREM core fragment, or TREM fragment comprises a sequence that differs by no more than 1, 2, 3, 4, 5, 6, 7, 8, 9, or 10 nucleotides from the nucleotide sequence of a TREM provided in any one of SEQ ID NOs: 625-693. In an embodiment, the TREM, TREM core fragment, or TREM fragment comprises 1, 2, 3, 4, 5, 6, 7, 8, 9, or 10 additional non-naturally occurring modifications compared with a TREM, TREM core fragment, or TREM fragment provided in any one of SEQ ID NOs: 625-693 (e.g., 2′-ribose modifications or an internucleotide modification, e.g., 2′OMe, 2′-halo, 2′-MOE, 2′-deoxy, or phosphorothiorate modifications).

In an embodiment, of any of the TREMs, TREM core fragments, or TREM fragments disclosed herein, the TREM, TREM core fragment, or TREM fragment is a TREM provided in any one of TREM NOs: 1-69. In an embodiment, the TREM, TREM core fragment, or TREM fragment comprises 1, 2, 3, 4, 5, 6, 7, 8, 9, or 10 additional non-naturally occurring modifications compared with a TREM provided in any one of TREM NOs. 1-69 (e.g., 2′-ribose modifications or an internucleotide modification, e.g., 2′OMe, 2′-halo, 2′-MOE, 2′-deoxy, or phosphorothiorate modifications).

In another aspect, the disclosure provides a pharmaceutical composition comprising a TREM, a TREM core fragment, or a TREM fragment disclosed herein.

In another aspect, a TREM or a related composition thereof can be used, inter alia, to modulate a functional parameter (e.g., an expression parameter and/or a signaling parameter) of an RNA corresponding to, or a polypeptide encoded by, a nucleic acid sequence comprising an endogenous open reading frame (ORF) having a repeat expansion disease (RED) codon.

In another aspect, provided herein is a method of modulating a functional parameter of an mRNA corresponding to, or polypeptide encoded by, an endogenous open reading frame (ORF) in a subject, which ORF comprises a a repeat expansion disease (RED) codon, contacting the subject with a TREM composition comprising a TREM, a TREM core fragment, or a TREM fragment disclosed herein in an amount and/or for a time sufficient to modulate the functional parameter of the mRNA or polypeptide, wherein the TREM, TREM core fragment or TREM fragment has an anticodon that pairs with the codon having the first sequence, thereby modulating the functional parameter in the subject. In an embodiment, the functional parameter comprises a signaling parameter and/or an expression parameter, e.g., as described herein.

In another aspect, disclosed herein is a method of modulating expression of a protein in a cell, wherein the protein is encoded by a nucleic acid comprising an endogenous open reading frame (ORF), which ORF comprises a repeat expansion disease (RED) codon, comprising contacting the cell with a TREM composition comprising a TREM, a TREM core fragment, or a TREM fragment disclosed herein in an amount and/or for a time sufficient to modulate expression of the encoded protein, wherein the TREM, TREM core fragment or TREM fragment has an anticodon that pairs with the RED codon, thereby modulating expression of the protein in the cell. In an embodiment, the RED codon is CAG.

In another aspect, provided herein is a method of increasing expression of a protein in a subject wherein the protein is encoded by a nucleic acid comprising an endogenous open reading frame (ORF), which ORF comprises a repeat expansion disease (RED) codon, comprising contacting the subject, in an amount and/or for a time sufficient to increase expression of the protein, with a TREM composition that (i) has an anticodon that pairs with the RED codon, (ii) recognizes an aminoacyl-tRNA synthetase specific for Trp, Tyr, Cys, Glu, Lys, Gln, Ser, Leu, Arg, or Gly, (iii) comprises a sequence of Formula A, or (iv) comprises a non-naturally occurring modification. In an embodiment, the RED codon is CAG. In an embodiment, the TREM composition comprises (i). In an embodiment, the TREM composition comprises (ii). In an embodiment, the TREM composition comprises (iii). In an embodiment, the TREM composition comprises (iv). In an embodiment, the TREM composition comprises two of (i)-(iv). In an embodiment, the TREM composition comprises three of (i)-(iv). In an embodiment, the TREM composition comprises each of (i)-(iv).

In another aspect, the disclosure provides a method of treating a subject having an endogenous open reading frame (ORF) which comprises a repeat expansion disease (RED) codon, comprising providing a TREM composition comprising a TREM, a TREM core fragment, or a TREM fragment disclosed herein, wherein the TREM comprises an anticodon that pairs with the RED codon in the ORF; contacting the subject with the composition comprising a TREM, TREM core fragment or TREM fragment in an amount and/or for a time sufficient to treat the subject, thereby treating the subject. In an embodiment, the RED codon is CAG.

In another aspect, the disclosure provides a method of treating a subject having a repeat expansion disease (RED), comprising providing a TREM composition comprising a TREM, a TREM core fragment, or a TREM fragment disclosed herein; contacting the subject with the composition comprising a TREM, TREM core fragment or TREM fragment in an amount and/or for a time sufficient to treat the subject, thereby treating the subject. In an embodiment, the repeat expansion comprises a repeat motif (e.g., a RED codon) provided in Table 9. In an embodiment, the RED is a disease or disorder described herein.

In another aspect, the disclosure provides a method of making a TREM, a TREM core fragment, or a TREM fragment disclosed herein, comprising linking a first nucleotide to a second nucleotide to form the TREM.

In an embodiment, the TREM, TREM core fragment or TREM fragment is non-naturally occurring (e.g., synthetic)

In an embodiment, the TREM, TREM core fragment or TREM fragment is made by cell-free solid phase synthesis.

In another aspect, the disclosure provides a method of modulating a tRNA pool in a cell comprising: providing a TREM, a TREM core fragment, or a TREM fragment disclosed herein, and contacting the cell with the TREM, TREM core fragment or TREM fragment, thereby modulating the tRNA pool in the cell.

In an aspect, the disclosure provides a method of contacting a cell, tissue, or subject with a TREM, a TREM core fragment, or a TREM fragment disclosed herein (e.g., a TREM provided in FIG. 1), comprising: contacting the cell, tissue or subject with the TREM, TREM core fragment or TREM fragment, thereby contacting the cell, tissue, or subject with the TREM, TREM core fragment or TREM fragment.

In another aspect, the disclosure provides a method of delivering a TREM, TREM core fragment or TREM fragment (e.g., a TREM provided in FIG. 1) to a cell, tissue, or subject, comprising: providing a cell, tissue, or subject, and contacting the cell, tissue, or subject, a TREM, a TREM core fragment, or a TREM fragment disclosed herein.

In an aspect, the disclosure provides a method of modulating a tRNA pool in a cell comprising an endogenous open reading frame (ORF), which ORF comprises a codon having a first sequence, comprising:
optionally, acquiring knowledge of the abundance of one or both of (i) and (ii), e.g., acquiring knowledge of the relative amounts of: (i) and (ii) in the cell, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell;
contacting the cell with a TREM, a TREM core fragment, or a TREM fragment disclosed herein (e.g., a TREM provided in FIG. 1), wherein the TREM, TREM core fragment or TREM fragment has an anticodon that pairs with: the codon having the first sequence; or the codon other than the codon having the first sequence, in an amount and/or for a time sufficient to modulate the relative amounts of the first tRNA moiety and the second tRNA moiety in the cell,
thereby modulating the tRNA pool in the cell.

In another aspect, the disclosure provides a method of modulating a tRNA pool in a subject having an ORF, which ORF comprises a codon having a first sequence, comprising:
optionally, acquiring knowledge of the abundance of one or both of (i) and (ii), e.g., acquiring knowledge of the relative amounts of: (i) and (ii) in the subject, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the subject;
contacting the subject with a TREM, a TREM core fragment, or a TREM fragment disclosed herein (e.g., a TREM provided in FIG. 1), wherein the TREM, TREM core fragment or TREM fragment has an anticodon that pairs with: the codon having the first sequence; or the codon other than the codon having the first sequence, in an amount and/or for a time sufficient to modulate the relative amounts of the first tRNA moiety and the second tRNA moiety in the subject,
thereby modulating the tRNA pool in the subject.

In an aspect, the disclosure provides a method of modulating a tRNA pool in a subject having an endogenous ORF comprising a codon comprising a synonymous mutation (a synonymous mutation codon or SMC), comprising:
providing a composition comprising a TREM, a TREM core fragment, or a TREM fragment disclosed herein (e.g., a TREM provided in FIG. 1), wherein the TREM, TREM core fragment or TREM fragment comprises an isoacceptor tRNA moiety comprising an anticodon sequence that pairs with the SMC (the TREM);
contacting the subject with the composition in an amount and/or for a time sufficient to modulate the tRNA pool in the subject,
thereby modulating the tRNA pool in the subject.

In another aspect, the disclosure provides a method of modulating a tRNA pool in a cell comprising an endogenous ORF comprising a codon comprising a SMC, comprising:
providing a composition comprising a TREM, a TREM core fragment, or a TREM fragment disclosed herein (e.g., a TREM provided in FIG. 1), wherein the TREM, TREM core fragment or TREM fragment comprises an isoacceptor tRNA moiety comprising an anticodon sequence that pairs with the SMC (the TREM);
contacting the cell with the composition comprising a TREM in an amount and/or for a time sufficient to modulate the tRNA pool in the cell,
thereby modulating the tRNA pool in the cell.

In an aspect, the disclosure provides a method of modulating expression of a protein in a cell, wherein the protein is encoded by a nucleic acid comprising an ORF, which ORF comprises a codon having a mutation, comprising:
contacting the cell with a composition comprising a TREM, a TREM core fragment, or a TREM fragment disclosed herein (e.g., a TREM provided in FIG. 1) in an amount and/or for a time sufficient to modulate expression of the encoded protein,
wherein the TREM, TREM core fragment or TREM fragment has an anticodon that pairs with the codon having the mutation,
thereby modulating expression of the protein in the cell.

In another aspect, the disclosure provides a method of modulating expression of a protein in a subject, wherein the protein is encoded by a nucleic acid comprising an endogenous ORF, which ORF comprises a codon having a mutation, comprising:
contacting the subject with a composition comprising a TREM, a TREM core fragment, or a TREM fragment disclosed herein (e.g., a TREM provided in FIG. 1), in an amount and/or for a time sufficient to modulate expression of the encoded protein,
wherein the TREM, TREM core fragment or TREM fragment has an anticodon that pairs with the codon having the mutation,
thereby modulating expression of the protein in the subject.

In an embodiment, of any of the methods disclosed herein, the mutation in the ORF is a nonsense mutation, e.g., resulting in a premature stop codon chosen from UAA, UGA or UAG. In an embodiment, the stop codon is UAA. In an embodiment, the stop codon is UGA. In an embodiment, the stop codon is UAG.

In an embodiment, of any of the methods disclosed herein, the TREM comprises an anticodon that pairs with a stop codon.

TREMs of the disclosure include TREMs, TREM core fragments and TREM fragments. TREMs, TREM core fragments or TREM fragments can be modified with non-naturally occurring modifications to, e.g., increase the level and/or activity (e.g., stability) of the TREM. Pharmaceutical TREM compositions, e.g., comprising TREMs having a non-naturally occurring modification, can be administered to cells, tissues, or subjects to modulate these functions, e.g., in vitro or in vivo. Disclosed herein are TREMs, TREM core fragments or TREM fragments comprising non-naturally occurring modifications, TREM compositions, preparations, methods of making TREM compositions and preparations, and methods of using the same.

In an embodiment, the TREM, TREM core fragment, and TREM fragments comprise a non-naturally occurring modification that improves stability or enhances activity of the TREM, TREM core fragment, or TREM fragment.

Additional features of any of the aforesaid TREMs, TREM core fragments, TREM fragments, TREM compositions, preparations, methods of making TREM compositions and preparations, and methods of using TREM compositions and preparations include one or more of the features in the Enumerated Embodiments, Figures, Description, Examples, or Claims.

Those skilled in the art will recognize or be able to ascertain using no more than routine experimentation, many equivalents to the specific embodiments of the invention described herein. Such equivalents are intended to be encompassed by the following Enumerated Embodiments, Drawings, Description, Examples, or Claims.

### BRIEF DESCRIPTION OF DRAWINGS

FIG. 1 is a table listing exemplary TREMs described herein, e.g., TREMs capable of inserting a missense mutation into the ORF of a gene (e.g., a RED codon into the ORF of a gene).

FIG. 2 is a schematic illustrating the proposed function of TREMs capable of inserting a missense mutation into the ORF of a gene.

FIG. 3 is a schematic depicting HTT reporter design. The number of CAG repeats in HTT exon 1 was varied to recapitulate the number of CAG repeats observed in healthy individuals and Huntington's disease patients. Abbreviations: CMV: cytomegalovirus promoter; eGFP: enhanced green fluorescent protein reporter.

FIG. 4 is a line plot depicting the decrease in GFP puncta in HEK293 cells treated with indicated missense tRNA for 48 hours.

FIG. 5 is a graph illustrating the efficacy of exemplary missense TREMs.

FIG. 6 is a graph of HTT protein aggregate measurement using fluorescence microscopy. Reporter plasmids were transfected in Hek293 cells and imaged using phase and GFP channels.

FIG. 7 is a graph depicting the area of the GFP puncta grouped according to their frequency in HEK293 cells.

FIGS. 8A-8D are a set of fluorescence microscopy images depicting aggregates in 63Q and 105Q reporter transfected Hek293 cells compared to empty vector or 21Q reporter transfected cells.

FIG. 9 is a graph showing the effect of missense TREMs on HTT protein aggregation in HEK293 cells.

FIG. 10 is a graph illustrating that missense TREMs block formation of aggregates. ATXN7 10Q or 52Q reporter was co-transfected in HEK293 cells along with missense TREM plasmids and aggregate formation was measured using GFP fluorescence imaging.

FIGS. 11A and 11B are graphs depicting the effect of missense TREMs on ATXN3 reporters. ATXN3 24Q reporter was transfected in HEK293 cells along with missense TREM plasmids and aggregate formation was measured.

FIGS. 12A and B are graphs illustrating the effect of missense TREMs on cell health. HEK293 cells (FIG. 12A) and U87 cells (FIG. 12B) were transfected with missense TREM plasmids for 48 hours.

FIG. 13 is an immunoblot of cell lysates collected from control or missense TREM-transfected HEK293 cells.

FIG. 14 is a graph depicting the effect of autophagy on the removal of HTT 63Q polyQ protein aggregates from missense TREM transfected cells.

FIG. 15 is an immunoblot depicting the effect of autophagy inhibition on polyQ protein accumulation via missense TREMs.

FIG. 16 is an immunoblot of HEK293 cells transfected with exemplary missense TREMs. Cells were transfected with different missense TREM plasmids, cellular lysates were collected, and immunoblotting was performed using anti-LC3B antibody.

### DETAILED DESCRIPTION OF CERTAIN EMBODIMENTS

Repeat expansion diseases (REDs) are a category of human diseases that are caused by an increase in the length of a polynucleotide repeat sequence in the genome above a critical threshold. One class of repeat expansion diseases is characterized by an increase in the length of a critical codon, e.g., a repeat expansion disease codon, in the genome which is then transcribed into an mRNA containing RED codon repeats above a critical threshold. For example, several REDs are characterized by repeated CAG codons in the open reading frame of a key disease protein. The repeated CAG codon results in a poly-glutamine (polyQ) tract in the translated protein product. In polyQ REDs, the polyQ protein aggregates in insoluble assemblies in the cytoplasm and affects cell viability by a gain-of-function mechanism. Non-limiting examples of polyQ repeat expansion diseases include Huntington's Disease (caused by CAG expansion in HTT gene), SCA1 (caused by CAG expansion in ATXN1 gene), SCA2 (caused by CAG expansion in ATXN2 gene), SCA3/MJD (caused by CAG expansion in ATXN3 gene), SCA6 (caused by CAG expansion in CACNA1A gene), SCA7 (caused by CAG expansion in ATXN7 gene), SCA17 (caused by CAG expansion in TBP gene), DRPLA (caused by CAG expansion in ATN1 gene), and SBMA (caused by CAG expansion in AR gene).

Since aggregation of the pathogenic polyQ protein is a uniting feature of polyQ REDs caused by repeat expansion in unrelated genes, methods to reduce the aggregation of polyQ protein in the cytoplasm may have broad therapeutic benefit. In some individuals, genetically-encoded interruption of the CAG repeat length above a pathogenic threshold delays disease onset or reduces its symptoms, suggesting that decreasing the length of the polyQ protein produced from the toxic mRNA may ameliorate or decrease the severity of the disease in patients.

One approach to reducing the length of the polyQ protein translated from the toxic mRNA is to engineer tRNAs that decode the CAG codon with another amino acid, thereby decreasing the number of glutamine residues incorporated in the peptide chain. These tRNAs can be classified as missense tRNAs since they misincorporate an amino acid at a sense codon during mRNA translation.

The present disclosure features tRNA-based effector molecules (TREMs) comprising a non-naturally occurring modification and methods relating thereto. As disclosed herein, TREMs are complex molecules which can mediate a variety of cellular processes. Pharmaceutical TREM compositions, e.g., TREMs comprising a non-naturally occurring modification, can be administered to a cell, a tissue, or to a subject to modulate these functions.

### Definitions

“Acquire” or “acquiring” as the terms are used herein, refer to obtaining possession of a value, e.g., a numerical value, by “directly acquiring” or “indirectly acquiring” the physical entity or value. “Directly acquiring” refers to performing a process (e.g., performing an analytical method) to obtain the value. “Indirectly acquiring” refers to receiving the value from another party or source (e.g., a third party laboratory that directly acquired the or value).

An “isoacceptor,” as that term is used herein, refers to a plurality of tRNA molecule or TREMs wherein each molecule of the plurality comprises a different naturally occurring anticodon sequence and each molecule of the plurality mediates the incorporation of the same amino acid and that amino acid is the amino acid that naturally corresponds to the anticodons of the plurality.

A “modification,” as that term is used herein with reference to a nucleotide, refers to a modification of the chemical structure, e.g., a covalent modification, of the subject nucleotide.

The modification can be naturally occurring or non-naturally occurring. In an embodiment, the modification is non-naturally occurring. In an embodiment, the modification is naturally occurring. In an embodiment, the modification is a synthetic modification. In an embodiment, the modification is a modification provided in Table 5.

A “naturally occurring nucleotide,” as that term is used herein, refers to a nucleotide that does not comprise a non-naturally occurring modification. In an embodiment, it includes a naturally occurring modification.

A “non-naturally occurring modification,” as that term is used herein with reference to a nucleotide, refers to a modification that: (a) a cell, e.g., a human cell, does not make on an endogenous tRNA; or (b) a cell, e.g., a human cell, can make on an endogenous tRNA but wherein such modification is in a location in which it does not occur on a native tRNA, e.g., the modification is in a domain, linker or arm, or on a nucleotide and/or at a position within a domain, linker or arm, which does not have such modification in nature. In either case, the modification is added synthetically, e.g., in a cell free reaction, e.g., in a solid state or liquid phase synthetic reaction. In an embodiment, the non-naturally occurring modification is a modification that is not present (in identity, location or position) if a sequence of the TREM is expressed in a mammalian cell, e.g., a HEK293 cell line. Exemplary non-naturally occurring modifications are found in Table 5.

A “non-naturally modified nucleotide,” as that term is used herein, refers a nucleotide comprising a non-naturally occurring modification on or of a sugar, nucleobase, or phosphate moiety.

A “nucleotide,” as that term is used herein, refers to an entity comprising a sugar, typically a pentameric sugar; a nucleobase; and a phosphate linking group. In an embodiment, a nucleotide comprises a naturally occurring, e.g., naturally occurring in a human cell, nucleotide, e.g., an adenine, thymine, guanine, cytosine, or uracil nucleotide.

A “functional parameter” refers to an expression parameter and/or a signaling parameter. In an embodiment, a functional parameter is an expression parameter. An expression parameter includes an expression parameter of a polypeptide or protein encoded by the endogenous ORF having a first sequence; or an expression parameter of an RNA, e.g., messenger RNA, encoded by the endogenous ORF having a first sequence. In an embodiment, an expression parameter can include:
(a) protein translation;
(b) expression level (e.g., of polypeptide or protein, or mRNA);
(c) post-translational modification of polypeptide or protein;
(d) folding (e.g., of polypeptide or protein, or mRNA),
(e) structure (e.g., of polypeptide or protein, or mRNA),
(f) transduction (e.g., of polypeptide or protein),
(g) compartmentalization (e.g., of polypeptide or protein, or mRNA),
(h) incorporation (e.g., of polypeptide or protein, or mRNA) into a supermolecular structure, e.g., incorporation into a membrane, proteasome, or ribosome,
(i) incorporation into a multimeric polypeptide, e.g., a homo or heterodimer, and/or
(j) stability.

In an embodiment, a functional parameter is a signaling parameter. A signaling parameter can include:
(1) modulation of a signaling pathway, e.g., a cellular signaling pathway which is downstream or upstream of the protein encoded by the endogenous ORF having a first sequence;
(2) cell fate modulation;
(3) ribosome occupancy modulation;
(4) protein translation modulation;
(5) mRNA stability modulation;
(6) protein folding and structure modulation;
(7) protein transduction or compartmentalization modulation; and/or
(8) protein stability modulation.

As used herein, the terms “repeat expansion disease” and “RED” refer to a disease that is associated with an increase in the length of a polynucleotide repeat sequence (e.g., a repeat expansion) in the genome above a critical threshold. REDs may be associated with, e.g., a trinucleotide repeat expansion or a hexanucleotide repeat expansion.

As used herein, the terms “repeat expansion disease codon” and “RED codon” refer to a trinucleotide sequence (e.g., of Table 9) that (a) corresponds to a specific amino acid during protein synthesis (e.g., translation) and (b) is tandemly repeated, e.g., abnormally, in a repeat expansion disease. For example, normal human subjects (e.g., subjects not diagnosed with or experiencing symptoms of Huntington's disease) may have <36 repeats of the RED codon CAG in the HTT gene. In an embodiment, human subjects having Huntington's disease may have at least 36 (e.g., at least 37, 38, 39, 40, 45, 50, 100, or more) repeats of the RED codon CAG in the HTT gene. RED codons are associated with abnormally long amino acid tracts, e.g., poly glutamine (polyQ) tracts, which can trigger protein misfolding and amyloid-like aggregations leading to severe cytotoxicities contributing to, e.g., neurodegeneration.

A “tRNA-based effector molecule” or “TREM,” as that term is used herein, refers to an RNA molecule comprising a structure or property from (a)-(v) below, and which is a recombinant TREM, a synthetic TREM, or a TREM expressed from a heterologous cell. The TREMs described in the present invention are synthetic molecules and are made, e.g., in a cell free reaction, e.g., in a solid state or liquid phase synthetic reaction. TREMs are chemically distinct, e.g., in terms of primary sequence, type or location of modifications from the endogenous tRNA molecules made in cells, e.g., in mammalian cells, e.g., in human cells. A TREM can have a plurality (e.g., 2, 3, 4, 5, 6, 7, 8, 9) of the structures and functions of (a)-(v).

In an embodiment, a TREM is non-native, as evaluated by structure or the way in which it was made.

In an embodiment, a TREM comprises one or more of the following structures or properties:
(a′) an optional linker region of a consensus sequence provided in the “Consensus Sequence” section, e.g., a Linker 1 region;
(a) an amino acid attachment domain that binds an amino acid, e.g., an acceptor stem domain (AStD), wherein an AStD comprises sufficient RNA sequence to mediate, e.g., when present in an otherwise wildtype tRNA, acceptance of an amino acid, e.g., its cognate amino acid or a non-cognate amino acid, and transfer of the amino acid (AA) in the initiation or elongation of a polypeptide chain. Typically, the AStD comprises a 3′-end adenosine (CCA) for acceptor stem charging which is part of synthetase recognition. In an embodiment, the AStD has at least 75, 80, 85, 85, 90, 95, or 100% identity with a naturally occurring AStD, e.g., an AStD encoded by a nucleic acid in Table 1. In an embodiment, the TREM can comprise a fragment or analog of an AStD, e.g., an AStD encoded by a nucleic acid in Table 1, which fragment in embodiments has AStD activity and in other embodiments does not have AStD activity. (One of ordinary skill can determine the relevant corresponding sequence for any of the domains, stems, loops, or other sequence features mentioned herein from a sequence encoded by a nucleic acid in Table 1. E.g., one of ordinary skill can determine the sequence which corresponds to an AStD from a tRNA sequence encoded by a nucleic acid in Table 1.)

In an embodiment, the AStD falls under the corresponding sequence of a consensus sequence provided in the “Consensus Sequence” section, or differs from the consensus sequence by no more than 1, 2, 5, or 10 positions;
In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula I_{ZZZ}, wherein ZZZ indicates any of the twenty amino acids;
In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula II_{ZZZ}, wherein ZZZ indicates any of the twenty amino acids;
In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula III_{ZZZ}, wherein ZZZ indicates any of the twenty amino acids;
(a′-1) a linker comprising residues R₈-R₉ of a consensus sequence provided in the “Consensus Sequence” section, e.g., a Linker 2 region;
(b) a dihydrouridine hairpin domain (DHD), wherein a DHD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a DHD mediates the stabilization of the TREM's tertiary structure. In an embodiment, the DHD has at least 75, 80, 85, 85, 90, 95, or 100% identity with a naturally occurring DHD, e.g., a DHD encoded by a nucleic acid in Table 1. In an embodiment, the TREM can comprise a fragment or analog of a DHD, e.g., a DHD encoded by a nucleic acid in Table 1, which fragment in embodiments has DHD activity and in other embodiments does not have DHD activity.

In an embodiment, the DHD falls under the corresponding sequence of a consensus sequence provided in the “Consensus Sequence” section, or differs from the consensus sequence by no more than 1, 2, 5, or 10 positions;
In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula I_{ZZZ}, wherein ZZZ indicates any of the twenty amino acids;
In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula II_{ZZZ}, wherein ZZZ indicates any of the twenty amino acids;
In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula III_{ZZZ}, wherein ZZZ indicates any of the twenty amino acids;
(b′-1) a linker comprising residue R₂₉ of a consensus sequence provided in the “Consensus Sequence” section, e.g., a Linker 3 region;
(c) an anticodon that binds a respective codon in an mRNA, e.g., an anticodon hairpin domain (ACHD), wherein an ACHD comprises sufficient sequence, e.g., an anticodon triplet, to mediate, e.g., when present in an otherwise wildtype tRNA, pairing (with or without wobble) with a codon; In an embodiment, the ACHD has at least 75, 80, 85, 85, 90, 95, or 100% identity with a naturally occurring ACHD, e.g., an ACHD encoded by a nucleic acid in Table 1. In an embodiment, the TREM can comprise a fragment or analog of an ACHD, e.g., an ACHD encoded by a nucleic acid in Table 1, which fragment in embodiments has ACHD activity and in other embodiments does not have ACHD activity.

In an embodiment, the ACHD falls under the corresponding sequence of a consensus sequence provided in the “Consensus Sequence” section, or differs from the consensus sequence by no more than 1, 2, 5, or 10 positions;
In an embodiment, the ACHD comprises residues -R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula I_{ZZZ}, wherein ZZZ indicates any of the twenty amino acids;
In an embodiment, the ACHD comprises residues -R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula II_{ZZZ}, wherein ZZZ indicates any of the twenty amino acids;
In an embodiment, the ACHD comprises residues -R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula III_{ZZZ}, wherein ZZZ indicates any of the twenty amino acids;
(d) a variable loop domain (VLD), wherein a VLD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a VLD mediates the stabilization of the TREM's tertiary structure. In an embodiment, a VLD modulates, e.g., increases, the specificity of the TREM, e.g., for its cognate amino acid, e.g., the VLD modulates the TREM's cognate adaptor function. In an embodiment, the VLD has at least 75, 80, 85, 85, 90, 95, or 100% identity with a naturally occurring VLD, e.g., a VLD encoded by a nucleic acid in Table 1. In an embodiment, the TREM can comprise a fragment or analog of a VLD, e.g., a VLD encoded by a nucleic acid in Table 1, which fragment in embodiments has VLD activity and in other embodiments does not have VLD activity.

In an embodiment, the VLD falls under the corresponding sequence of a consensus sequence provided in the “Consensus Sequence” section.

In an embodiment, the VLD comprises residue —[R₄₇]ₓ of a consensus sequence provided in the “Consensus Sequence” section, wherein x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271);
(e) a thymine hairpin domain (THD), wherein a THD comprises sufficient RNA sequence, to mediate, e.g., when present in an otherwise wildtype tRNA, recognition of the ribosome, e.g., acts as a recognition site for the ribosome to form a TREM-ribosome complex during translation. In an embodiment, the THD has at least 75, 80, 85, 85, 90, 95, or 100% identity with a naturally occurring THD, e.g., a THD encoded by a nucleic acid in Table 1. In an embodiment, the TREM can comprise a fragment or analog of a THD, e.g., a THD encoded by a nucleic acid in Table 1, which fragment in embodiments has THD activity and in other embodiments does not have THD activity.

In an embodiment, the THD falls under the corresponding sequence of a consensus sequence provided in the “Consensus Sequence” section, or differs from the consensus sequence by no more than 1, 2, 5, or 10 positions;

In an embodiment, the THD comprises residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula I ZZZ, wherein ZZZ indicates any of the twenty amino acids;
In an embodiment, the THD comprises residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula II ZZZ, wherein ZZZ indicates any of the twenty amino acids;
In an embodiment, the THD comprises residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula III 777, wherein ZZZ indicates any of the twenty amino acids;
(e′1) a linker comprising residue R₇₂ of a consensus sequence provided in the “Consensus Sequence” section, e.g., a Linker 4 region;
(f) under physiological conditions, it comprises a stem structure and one or a plurality of loop structures, e.g., 1, 2, or 3 loops. A loop can comprise a domain described herein, e.g., a domain selected from (a)-(e). A loop can comprise one or a plurality of domains. In an embodiment, a stem or loop structure has at least 75, 80, 85, 85, 90, 95, or 100% identity with a naturally occurring stem or loop structure, e.g., a stem or loop structure encoded by a nucleic acid in Table 1. In an embodiment, the TREM can comprise a fragment or analog of a stem or loop structure, e.g., a stem or loop structure encoded by a nucleic acid in Table 1, which fragment in embodiments has activity of a stem or loop structure, and in other embodiments does not have activity of a stem or loop structure;
(g) a tertiary structure, e.g., an L-shaped tertiary structure;
(h) adaptor function, i.e., the TREM mediates acceptance of an amino acid, e.g., its cognate amino acid and transfer of the AA in the initiation or elongation of a polypeptide chain;
(i) cognate adaptor function wherein the TREM mediates acceptance and incorporation of an amino acid (e.g., cognate amino acid) associated in nature with the anti-codon of the TREM to initiate or elongate a polypeptide chain;
(j) non-cognate adaptor function, wherein the TREM mediates acceptance and incorporation of an amino acid (e.g., non-cognate amino acid) other than the amino acid associated in nature with the anti-codon of the TREM in the initiation or elongation of a polypeptide chain;
(k) a regulatory function, e.g., an epigenetic function (e.g., gene silencing function or signaling pathway modulation function), cell fate modulation function, mRNA stability modulation function, protein stability modulation function, protein transduction modulation function, or protein compartmentalization function;
(l) a structure which allows for ribosome binding;
(m) a post-transcriptional modification, e.g., a naturally occurring post-transcriptional modification;
(n) the ability to inhibit a functional property of a tRNA, e.g., any of properties (h)-(k) possessed by a tRNA;
(o) the ability to modulate cell fate;
(p) the ability to modulate ribosome occupancy;
(q) the ability to modulate protein translation;
(r) the ability to modulate mRNA stability;
(s) the ability to modulate protein folding and structure;
(t) the ability to modulate protein transduction or compartmentalization;
(u) the ability to modulate protein stability; or
(v) the ability to modulate a signaling pathway, e.g., a cellular signaling pathway.

In an embodiment, a TREM comprises a full-length tRNA molecule or a fragment thereof.

In an embodiment, a TREM comprises the following properties: (a)-(e).

In an embodiment, a TREM comprises the following properties: (a) and (c).

In an embodiment, a TREM comprises the following properties: (a), (c) and (h).

In an embodiment, a TREM comprises the following properties: (a), (c), (h) and (b).

In an embodiment, a TREM comprises the following properties: (a), (c), (h) and (e).

In an embodiment, a TREM comprises the following properties: (a), (c), (h), (b) and (e).

In an embodiment, a TREM comprises the following properties: (a), (c), (h), (b), (e) and (g).

In an embodiment, a TREM comprises the following properties: (a), (c), (h) and (m).

In an embodiment, a TREM comprises the following properties: (a), (c), (h), (m), and (g).

In an embodiment, a TREM comprises the following properties: (a), (c), (h), (m) and (b).

In an embodiment, a TREM comprises the following properties: (a), (c), (h), (m) and (e).

In an embodiment, a TREM comprises the following properties: (a), (c), (h), (m), (g), (b) and (e).

In an embodiment, a TREM comprises the following properties: (a), (c), (h), (m), (g), (b), (e) and (q).

In an embodiment, a TREM comprises:
(i) an amino acid attachment domain that binds an amino acid (e.g., an AStD, as described in (a) herein; and
(ii) an anticodon that binds a respective codon in an mRNA (e.g., an ACHD, as described in (c) herein).

In an embodiment, the TREM comprises a flexible RNA linker which provides for covalent linkage of (i) to (ii).

In an embodiment, the TREM mediates protein translation.

In an embodiment, a TREM comprises a linker, e.g., an RNA linker, e.g., a flexible RNA linker, which provides for covalent linkage between a first and a second structure or domain. In an embodiment, an RNA linker comprises at least 1, 2, 3, 4, 5, 6, 7, 8, 9, 10, 11, 12, 13, 14 or 15 ribonucleotides. A TREM can comprise one or a plurality of linkers, e.g., in embodiments a TREM comprising (a), (b), (c), (d) and (e) can have a first linker between a first and second domain, and a second linker between a third domain and another domain.

In an embodiment, the TREM comprises a sequence of Formula A: [L1]-[ASt Domain1]-[L2]-[DH Domain]-[L3]-[ACH Domain]-[VL Domain]-[TH Domain]-[L4]-[ASt Domain2].

In an embodiment, a TREM comprises an RNA sequence at least 60, 65, 70, 75, 80, 85, 90, 95, 96, 97, 98 or 99% identical with, or which differs by no more than 1, 2, 3, 4, 5, 10, 15, 20, 25, or 30 ribonucleotides from, an RNA sequence encoded by a DNA sequence listed in Table 1, or a fragment or functional fragment thereof. In an embodiment, a TREM comprises an RNA sequence encoded by a DNA sequence listed in Table 1, or a fragment or functional fragment thereof. In an embodiment, a TREM comprises an RNA sequence encoded by a DNA sequence at least 60, 65, 70, 75, 80, 85, 90, 95, 96, 97, 98 or 99% identical with a DNA sequence listed in Table 1, or a fragment or functional fragment thereof. In an embodiment, a TREM comprises a TREM domain, e.g., a domain described herein, comprising at least 60, 65, 70, 75, 80, 85, 90, 95, 96, 97, 98, or 99% identical with, or which differs by no more than 1, 2, 3, 4, 5, 10, or 15, ribonucleotides from, an RNA encoded by a DNA sequence listed in Table 1, or a fragment or a functional fragment thereof. In an embodiment, a TREM comprises a TREM domain, e.g., a domain described herein, comprising an RNA sequence encoded by DNA sequence listed in Table 1, or a fragment or functional fragment thereof. In an embodiment, a TREM comprises a TREM domain, e.g., a domain described herein, comprising an RNA sequence encoded by DNA sequence at least 60, 65, 70, 75, 80, 85, 90, 95, 96, 97, 98 or 99% identical with a DNA sequence listed in Table 1, or a fragment or functional fragment thereof.

In an embodiment, a TREM is 76-90 nucleotides in length. In embodiments, a TREM or a fragment or functional fragment thereof is between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 20-90 nucleotides, between 20-80 nucleotides, 20-70 nucleotides, between 20-60 nucleotides, between 20-50 nucleotides, between 20-40 nucleotides, between 30-90 nucleotides, between 30-80 nucleotides, between 30-70 nucleotides, between 30-60 nucleotides, or between 30-50 nucleotides.

In an embodiment, a TREM is aminoacylated, e.g., charged, with an amino acid by an aminoacyl tRNA synthetase.

In an embodiment, a TREM is not charged with an amino acid, e.g., an uncharged TREM

(uTREM).

In an embodiment, a TREM comprises less than a full length tRNA. In embodiments, a TREM can correspond to a naturally occurring fragment of a tRNA, or to a non-naturally occurring fragment. Exemplary fragments include: TREM halves (e.g., from a cleavage in the ACHD, e.g., in the anticodon sequence, e.g., 5′-halves or 3′ halves); a 5′ fragment (e.g., a fragment comprising the 5′ end, e.g., from a cleavage in a DHD or the ACHD); a 3′ fragment (e.g., a fragment comprising the 3′ end, e.g., from a cleavage in the THD); or an internal fragment (e.g., from a cleavage in one or more of the ACHD, DHD or THD).

A “TREM core fragment,” as that term is used herein, refers to a portion of the sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1.

A “TREM fragment,” as used herein, refers to a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2].

A “cognate adaptor function TREM,” as that term is used herein, refers to a TREM which mediates initiation or elongation with the AA (the cognate AA) associated in nature with the anti-codon of the TREM.

“Decreased expression,” as that term is used herein, refers to a decrease in comparison to a reference, e.g., in the case where altered control region, or addition of an agent, results in a decreased expression of the subject product, it is decreased relative to an otherwise similar cell without the alteration or addition.

An “exogenous nucleic acid,” as that term is used herein, refers to a nucleic acid sequence that is not present in or differs by at least one nucleotide from the closest sequence in a reference cell, e.g., a cell into which the exogenous nucleic acid is introduced. In an embodiment, an exogenous nucleic acid comprises a nucleic acid that encodes a TREM.

An “exogenous TREM,” as that term is used herein, refers to a TREM that:
(a) differs by at least one nucleotide or one post transcriptional modification from the closest sequence tRNA in a reference cell, e.g., a cell into which the exogenous nucleic acid is introduced;
(b) has been introduced into a cell other than the cell in which it was transcribed;
(c) is present in a cell other than one in which it naturally occurs; or
(d) has an expression profile, e.g., level or distribution, that is non-wildtype, e.g., it is expressed at a higher level than wildtype. In an embodiment, the expression profile can be mediated by a change introduced into a nucleic acid that modulates expression or by addition of an agent that modulates expression of the RNA molecule. In an embodiment, an exogenous TREM comprises 1, 2, 3 or 4 of properties (a)-(d).

A “GMP-grade composition,” as that term is used herein, refers to a composition in compliance with current good manufacturing practice (cGMP) guidelines, or other similar requirements. In an embodiment, a GMP-grade composition can be used as a pharmaceutical product.

As used herein, the terms “increasing” and “decreasing” refer to modulating that results in, respectively, greater or lesser amounts of function, expression, or activity of a particular metric relative to a reference. For example, subsequent to administration to a cell, tissue or subject of a TREM described herein, the amount of a marker of a metric (e.g., protein translation, mRNA stability, protein folding) as described herein may be increased or decreased by at least 5%, 10%, 15%, 20%, 25%, 30%, 35%, 40%, 45%, 50%, 55%, 60%, 65%, 70%, 75%, 80%, 85%, 90%, 95% or 98%, 2×, 3×, 5×, 10× or more relative to the amount of the marker prior to administration or relative to the effect of a negative control agent. The metric may be measured subsequent to administration at a time that the administration has had the recited effect, e.g., at least 12 hours, 24 hours, one week, one month, 3 months, or 6 months, after a treatment has begun.

“Increased expression,” as that term is used herein, refers to an increase in comparison to a reference, e.g., in the case where altered control region, or addition of an agent, results in an increased expression of the subject product, it is increased relative to an otherwise similar cell without the alteration or addition.

A “non-cognate adaptor function TREM,” as that term is used herein, refers to a TREM which mediates initiation or elongation with an AA (a non-cognate AA) other than the AA associated in nature with the anti-codon of the TREM. In an embodiment, a non-cognate adaptor function TREM is also referred to as a mischarged TREM (mTREM).

A “non-naturally occurring sequence,” as that term is used herein, refers to a sequence wherein an Adenine is replaced by a residue other than an analog of Adenine, a Cytosine is replaced by a residue other than an analog of Cytosine, a Guanine is replaced by a residue other than an analog of Guanine, and a Uracil is replaced by a residue other than an analog of Uracil. An analog refers to any possible derivative of the ribonucleotides, A, G, C or U. In an embodiment, a sequence having a derivative of any one of ribonucleotides A, G, C or U is a non-naturally occurring sequence.

A “pharmaceutical TREM composition,” as that term is used herein, refers to a TREM composition that is suitable for pharmaceutical use. Typically, a pharmaceutical TREM composition comprises a pharmaceutical excipient. In an embodiment, the TREM will be the only active ingredient in the pharmaceutical TREM composition. In embodiments the pharmaceutical TREM composition is free, substantially free, or has less than a pharmaceutically acceptable amount, of host cell proteins, DNA, e.g., host cell DNA, endotoxins, and bacteria.

A “post-transcriptional processing,” as that term is used herein, with respect to a subject molecule, e.g., a TREM, RNA or tRNAs, refers to a covalent modification of the subject molecule. In an embodiment, the covalent modification occurs post-transcriptionally. In an embodiment, the covalent modification occurs co-transcriptionally. In an embodiment, the modification is made in vivo, e.g., in a cell used to produce a TREM. In an embodiment, the modification is made ex vivo, e.g., it is made on a TREM isolated or obtained from the cell which produced the TREM. In an embodiment, the post-transcriptional modification is selected from a post-transcriptional modification listed in Table 2.

A “synthetic TREM,” as that term is used herein, refers to a TREM which was synthesized other than in or by a cell having an endogenous nucleic acid encoding the TREM, e.g., a synthetic TREM is synthetized by cell-free solid phase synthesis. A synthetic TREM can have the same, or a different, sequence, or tertiary structure, as a native tRNA.

A “recombinant TREM,” as that term is used herein, refers to a TREM that was expressed in a cell modified by human intervention, having a modification that mediates the production of the TREM, e.g., the cell comprises an exogenous sequence encoding the TREM, or a modification that mediates expression, e.g., transcriptional expression or post-transcriptional modification, of the TREM. A recombinant TREM can have the same, or a different, sequence, set of post-transcriptional modifications, or tertiary structure, as a reference tRNA, e.g., a native tRNA.

As used herein, a “reference TREM” refers to a naturally occurring TREM.

A “tRNA”, as that term is used herein, refers to a naturally occurring transfer ribonucleic acid in its native state.

A “TREM composition,” as that term is used herein, refers to a composition comprising a plurality of TREMs, a plurality of TREM core fragments and/or a plurality of TREM fragments. A TREM composition can comprise one or more species of TREMs, TREM core fragments or TREM fragments. In an embodiment, the composition comprises only a single species of TREM, TREM core fragment or TREM fragment. In an embodiment, the TREM composition comprises a first TREM, TREM core fragment or TREM fragment species; and a second TREM, TREM core fragment or TREM fragment species. In an embodiment, the TREM composition comprises X TREM, TREM core fragment or TREM fragment species, wherein x=2, 3, 4, 5, 6, 7, 8, 9, or 10. In an embodiment, the TREM, TREM core fragment or TREM fragment has at least 70, 75, 80, 85, 90, or 95, or has 100%, identity with a sequence encoded by a nucleic acid in Table 1. A TREM composition can comprise one or more species of TREMs, TREM core fragments or TREM fragments. In an embodiment, the TREM composition is at least 10, 20, 30, 40, 50, 60, 70, 80, 90, 95 or 99% dry weight TREMs (for a liquid composition dry weight refers to the weight after removal of substantially all liquid, e.g., after lyophilization). In an embodiment, the composition is a liquid. In an embodiment, the composition is dry, e.g., a lyophilized material. In an embodiment, the composition is a frozen composition. In an embodiment, the composition is sterile. In an embodiment, the composition comprises at least 0.5 g, 1.0 g, 5.0 g, 10 g, 15 g, 25 g, 50 g, 100 g, 200 g, 400 g, or 500 g (e.g., as determined by dry weight) of TREM.

In an embodiment, at least X % of the TREMs in a TREM composition has a non-naturally occurring modification at a selected position, and X is 80, 90, 95, 96, 97, 98, 99, or 99.5.

In an embodiment, at least X % of the TREMs in a TREM composition has a non-naturally occurring modification at a first position and a non-naturally occurring modification at a second position, and X, independently, is 80, 90, 95, 96, 97, 98, 99, or 99.5. In embodiments, the modification at the first and second position is the same. In embodiments, the modification at the first and second position are different. In embodiments, the nucleotide at the first and second position is the same, e.g., both are adenine. In embodiments, the nucleotide at the first and second position are different, e.g., one is adenine and one is thymine.

In an embodiment, at least X % of the TREMs in a TREM composition has a non-naturally occurring modification at a first position and less than Y % have a non-naturally occurring modification at a second position, wherein X is 80, 90, 95, 96, 97, 98, 99, or 99.5 and Y is 20, 20, 5, 2, 1, 0.1, or 0.01. In embodiments, the nucleotide at the first and second position is the same, e.g., both are adenine. In embodiments the nucleotide at the first and second position are different, e.g., one is adenine and one is thymine.

### TREM, TREM Core Fragment and TREM Fragment

A “tRNA-based effector molecule” or “TREM” refers to an RNA molecule comprising one or more of the properties described herein. A TREM can comprise a non-naturally occurring modification, e.g., as provided in Tables 4, 5, 6 or 7.

In an embodiment, a TREM includes a TREM comprising a sequence of Formula A; a TREM core fragment comprising a sequence of Formula B; or a TREM fragment comprising a portion of a TREM which TREM comprises a sequence of Formula A.

In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2]. In an embodiment, [VL Domain] is optional. In an embodiment, [L1] is optional.

In an embodiment, a TREM core fragment comprises a sequence of Formula B: [L1]_{y}-[ASt Domain1]-[L2]_{y}-[DH Domain]_{y}-[L3]_{y}-[ACH Domain]-[VL Domain]_{y}-[TH Domain]_{y}-[L4]_{y}-[ASt Domain2]ₓ, wherein: x=1 and y=0 or 1. In an embodiment, y=0. In an embodiment, y=1.

In an embodiment, a TREM fragment comprises a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2], and wherein the TREM fragment comprises: one, two, three or all or any combination of the following: a TREM half (e.g., from a cleavage in the ACH Domain, e.g., in the anticodon sequence, e.g., a 5′-half or a 3′ half); a 5′ fragment (e.g., a fragment comprising the 5′ end, e.g., from a cleavage in a DH Domain or the ACH Domain); a 3′ fragment (e.g., a fragment comprising the 3′ end, e.g., from a cleavage in the TH Domain); or an internal fragment (e.g., from a cleavage in any one of the ACH Domain, DH Domain or TH Domain). Exemplary TREM fragments include TREM halves (e.g., from a cleavage in the ACHD, e.g., 5′TREM halves or 3′ TREM halves), a 5′ fragment (e.g., a fragment comprising the 5′ end, e.g., from a cleavage in a DHD or the ACHD), a 3′ fragment (e.g., a fragment comprising the 3′ end of a TREM, e.g., from a cleavage in the THD), or an internal fragment (e.g., from a cleavage in one or more of the ACHD, DHD or THD).

In an embodiment, a TREM, a TREM core fragment or a TREM fragment can be charged with an amino acid (e.g., a cognate amino acid); charged with a non-cognate amino acid (e.g., a mischarged TREM (mTREM)); or not charged with an amino acid (e.g., an uncharged TREM (uTREM)). In an embodiment, a TREM, a TREM core fragment or a TREM fragment can be charged with an amino acid selected from alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.

In some embodiments, a non-extended anticodon is an anticodon of no more than three nucleotides. In an embodiment, a non-extended codon pairs with no more than three codon nucleotides on a nucleic acid being translated.

In an embodiment, the TREM, TREM core fragment or TREM fragment is a cognate TREM. In an embodiment, the TREM, TREM core fragment or TREM fragment is a non-cognate TREM. In an embodiment, the TREM, TREM core fragment or TREM fragment recognizes a codon provided in Table 2 or Table 3.

TABLE 2
List of codons

AAA
AAC
AAG
AAU
ACA
ACC
ACG
ACU
AGA
AGC
AGG
AGU
AUA
AUC
AUG
AUU
CAA
CAC
CAG
CAU
CCA
CCC
CCG
CCU
CGA
CGC
CGG
CGU
CUA
CUC
CUG
CUU
GAA
GAC
GAG
GAU
GCA
GCC
GCG
GCU
GGA
GGC
GGG
GGU
GUA
GUC
GUG
GUU
UAA
UAC
UAG
UAU
UCA
UCC
UCG
UCU
UGA
UGC
UGG
UGU
UUA
UUC
UUG
UUU

TABLE 3
Amino acids and corresponding codons

| Amino Acid | mRNA codons |
|---|---|
| Alanine | GCU, GCC, GCA, GCG |
| Arginine | CGU, CGC, CGA, CGG, AGA, AGG |
| Asparagine | AAU, AAC |
| Aspartate | GAU, GAC |
| Cysteine | UGU, UGC |
| Glutamate | GAA, GAG |
| Glutamine | CAA, CAG |
| Glycine | GGU, GGC, GGA, GGG |
| Histidine | CAU, CAC |
| Isoleucine | AUU, AUC, AUA |
| Leucine | UUA, UUG, CUU, CUC, CUA, CUG |
| Lysine | AAA, AAG |
| Methionine | AUG |
| Phenylalanine | UUU, UUC |
| Proline | CCU, CCC, CCA, CCG |
| Serine | UCU, UCC, UCA, UCG, AGU, AGC |
| Stop | UAA, UAG, UGA |
| Threonine | ACU, ACC, ACA, ACG |
| Tryptophan | UGG |
| Tyrosine | UAU, UAC |
| Valine | GUU, GUC, GUA, GUG |

In an embodiment, a TREM comprises a ribonucleic acid (RNA) sequence encoded by a deoxyribonucleic acid (DNA) sequence disclosed in Table 1, e.g., any one of SEQ ID NOs: 1-451 disclosed in Table 1. In an embodiment, a TREM comprises an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM comprises an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, a TREM, a TREM core fragment, or TREM fragment comprises at least 5, 10, 15, 20, 25, or 30 consecutive nucleotides of an RNA sequence encoded by a DNA sequence disclosed in Table 1, e.g., at least 5, 10, 15, 20, 25, or 30 consecutive nucleotides of an RNA sequence encoded by any one of SEQ ID NOs: 1-451 disclosed in Table 1. In an embodiment, a TREM, a TREM core fragment, or TREM fragment comprises at least 5, 10, 15, 20, 25, or 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM, a TREM core fragment, or TREM fragment comprises at least 5, 10, 15, 20, 25, or 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence provided in Table 1, e.g., any one of SEQ ID NOs: 1-451 disclosed in Table 1. In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence at least 80%, 85%, 90%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence at least 80%, 85%, 90%, 95%, 96%, 97%, 98%, or 99% identical to a DNA sequence provided in Table 1, e.g., any one of SEQ ID NOs: 1-451 disclosed in Table 1.

In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence disclosed in Table 1, e.g., any one of SEQ ID NOs: 1-451 disclosed in Table 1. In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence which is at least 80%, 85%, 90%, 95%, 96%, 97%, 98%, 99% or 100% identical to an RNA sequence encoded by a DNA sequence provided in Table 1, e.g., any one of SEQ ID NOs: 1-451 disclosed in Table 1. In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence with at least 80%, 82%, 85%, 87%, 88%, 90%, 92%, 95%, 96%, 97%, 98%, 99% or 100% identity to a DNA sequence provided in Table 1, e.g., any one of SEQ ID NOs: 1-451 disclosed in Table 1.

In an embodiment, a TREM core fragment or a TREM fragment comprises a sequence of a length of between 10-90 ribonucleotides (rnt), between 10-80 rnt, between 10-70 rnt, between 10-60 rnt, between 10-50 rnt, between 10-40 rnt, between 10-30 rnt, between 10-20 rnt, between 20-90 rnt, between 20-80 rnt, 20-70 rnt, between 20-60 rnt, between 20-50 rnt, between 20-40 rnt, between 30-90 rnt, between 30-80 rnt, between 30-70 rnt, between 30-60 rnt, or between 30-50 rnt.

TABLE 1
List of tRNA Sequences

| SEQ ID | | |
|---|---|---|
| NO | tRNA name | tRNA sequence |
| 1 | Ala_AGC_chr6: | GGGGGTATAGCTCAGTGGTAGAGCGCGTGCT |
| | 28763741-28763812 (−) | TAGCATGCACGAGGTCCTGGGTTCGATCCCC |
| | | AGTACCTCCA |
| 2 | Ala_AGC_chr6: | GGGGAATTAGCTCAAGTGGTAGAGCGCTTGC |
| | 26687485-26687557 (+) | TTAGCACGCAAGAGGTAGTGGGATCGATGCC |
| | | CACATTCTCCA |
| 3 | Ala_AGC_chr6: | GGGGAATTAGCTCAAATGGTAGAGCGCTCGC |
| | 26572092-26572164 (−) | TTAGCATGCGAGAGGTAGCGGGATCGATGCC |
| | | CGCATTCTCCA |
| 4 | Ala_AGC_chr6: | GGGGAATTAGCTCAAGTGGTAGAGCGCTTGC |
| | 26682715-26682787 (+) | TTAGCATGCAAGAGGTAGTGGGATCGATGCC |
| | | CACATTCTCCA |
| 5 | Ala_AGC_chr6: | GGGGAATTAGCTCAAGCGGTAGAGCGCTTGC |
| | 26705606-26705678 (+) | TTAGCATGCAAGAGGTAGTGGGATCGATGCC |
| | | CACATTCTCCA |
| 6 | Ala_AGC_chr6: | GGGGAATTAGCTCAAGTGGTAGAGCGCTTGC |
| | 26673590-26673662 (+) | TTAGCATGCAAGAGGTAGTGGGATCAATGCC |
| | | CACATTCTCCA |
| 7 | Ala_AGC_chr14: | GGGGAATTAGCTCAAGTGGTAGAGCGCTCGC |
| | 89445442-89445514 (+) | TTAGCATGCGAGAGGTAGTGGGATCGATGCC |
| | | CGCATTCTCCA |
| 8 | Ala_AGC_chr6: | GGGGAATTAGCCCAAGTGGTAGAGCGCTTGC |
| | 58196623-58196695 (−) | TTAGCATGCAAGAGGTAGTGGGATCGATGCC |
| | | CACATTCTCCA |
| 9 | Ala_AGC_chr6: | GGGGGTGTAGCTCAGTGGTAGAGCGCGTGCT |
| | 28806221-28806292 (−) | TAGCATGCACGAGGCCCCGGGTTCAATCCCC |
| | | GGCACCTCCA |
| 10 | Ala_AGC_chr6: | GGGGGTGTAGCTCAGTGGTAGAGCGCGTGCT |
| | 28574933-28575004 (+) | TAGCATGTACGAGGTCCCGGGTTCAATCCCC |
| | | GGCACCTCCA |
| 11 | Ala_AGC_chr6: | GGGGATGTAGCTCAGTGGTAGAGCGCATGCT |
| | 28626014-28626085 (−) | TAGCATGCATGAGGTCCCGGGTTCGATCCCC |
| | | AGCATCTCCA |
| 12 | Ala_AGC_chr6: | GGGGGTGTAGCTCAGTGGTAGAGCGCGTGCT |
| | 28678366-28678437 (+) | TAGCATGCACGAGGCCCTGGGTTCAATCCCC |
| | | AGCACCTCCA |
| 13 | Ala_AGC_chr6: | GGGGGTATAGCTCAGCGGTAGAGCGCGTGCT |
| | 28779849-28779920 (−) | TAGCATGCACGAGGTCCTGGGTTCAATCCCC |
| | | AATACCTCCA |
| 14 | Ala_AGC_chr6: | GGGGGTGTAGCTCAGTGGTAGAGCGCGTGCT |
| | 28687481-28687552 (+) | TAGCATGCACGAGGCCCCGGGTTCAATCCCT |
| | | GGCACCTCCA |
| 15 | Ala_AGC_chr2: | GGGGGATTAGCTCAAATGGTAGAGCGCTCGC |
| | 27274082-27274154 (+) | TTAGCATGCGAGAGGTAGCGGGATCGATGCC |
| | | CGCATCCTCCA |
| 16 | Ala_AGC_chr6: | GGGGAATTAGCTCAGGCGGTAGAGCGCTCGC |
| | 26730737-26730809 (+) | TTAGCATGCGAGAGGTAGCGGGATCGACGCC |
| | | CGCATTCTCCA |
| 17 | Ala_CGC_chr6: | GGGGATGTAGCTCAGTGGTAGAGCGCATGCT |
| | 26553731-26553802 (+) | TCGCATGTATGAGGTCCCGGGTTCGATCCCC |
| | | GGCATCTCCA |
| 18 | Ala_CGC_chr6: | GGGGATGTAGCTCAGTGGTAGAGCGCATGCT |
| | 28641613-28641684 (−) | TCGCATGTATGAGGCCCCGGGTTCGATCCCC |
| | | GGCATCTCCA |
| 19 | Ala_CGC_chr2: | GGGGATGTAGCTCAGTGGTAGAGCGCGCGCT |
| | 157257281-157257352 (+) | TCGCATGTGTGAGGTCCCGGGTTCAATCCCC |
| | | GGCATCTCCA |
| 20 | Ala_CGC_chr6: | GGGGGTGTAGCTCAGTGGTAGAGCGCGTGCT |
| | 28697092-28697163 (+) | TCGCATGTACGAGGCCCCGGGTTCGACCCCC |
| | | GGCTCCTCCA |
| 21 | Ala_TGC_chr6: | GGGGGTGTAGCTCAGTGGTAGAGCGCATGCT |
| | 28757547-28757618 (−) | TTGCATGTATGAGGTCCCGGGTTCGATCCCC |
| | | GGCACCTCCA |
| 22 | Ala_TGC_chr6: | GGGGATGTAGCTCAGTGGTAGAGCGCATGCT |
| | 28611222-28611293 (+) | TTGCATGTATGAGGTCCCGGGTTCGATCCCC |
| | | GGCATCTCCA |
| 23 | Ala_TGC_chr5: | GGGGATGTAGCTCAGTGGTAGAGCGCATGCT |
| | 180633868-180633939 (+) | TTGCATGTATGAGGCCCCGGGTTCGATCCCC |
| | | GGCATCTCCA |
| 24 | Ala_TGC_chr12: | GGGGATGTAGCTCAGTGGTAGAGCGCATGCT |
| | 125424512-125424583 (+) | TTGCACGTATGAGGCCCCGGGTTCAATCCCC |
| | | GGCATCTCCA |
| 25 | Ala_TGC_chr6: | GGGGGTGTAGCTCAGTGGTAGAGCGCATGCT |
| | 28785012-28785083 (−) | TTGCATGTATGAGGCCTCGGGTTCGATCCCC |
| | | GACACCTCCA |
| 26 | Ala_TGC_chr6: | GGGGGTGTAGCTCAGTGGTAGAGCACATGCT |
| | 28726141-28726212 (−) | TTGCATGTGTGAGGCCCCGGGTTCGATCCCC |
| | | GGCACCTCCA |
| 27 | Ala_TGC_chr6: | GGGGGTGTAGCTCAGTGGTAGAGCGCATGCT |
| | 28770577-28770647 (−) | TTGCATGTATGAGGCCTCGGTTCGATCCCCG |
| | | ACACCTCCA |
| 28 | Arg_ACG_chr6: | GGGCCAGTGGCGCAATGGATAACGCGTCTGA |
| | 26328368-26328440 (+) | CTACGGATCAGAAGATTCCAGGTTCGACTCC |
| | | TGGCTGGCTCG |
| 29 | Arg_ACG_chr3: | GGGCCAGTGGCGCAATGGATAACGCGTCTGA |
| | 45730491-45730563 (−) | CTACGGATCAGAAGATTCTAGGTTCGACTCC |
| | | TGGCTGGCTCG |
| 30 | Arg_CCG_chr6: | GGCCGCGTGGCCTAATGGATAAGGCGTCTGA |
| | 28710729-28710801 (−) | TTCCGGATCAGAAGATTGAGGGTTCGAGTCC |
| | | CTTCGTGGTCG |
| 31 | Arg_CCG_chr17: | GACCCAGTGGCCTAATGGATAAGGCATCAGC |
| | 66016013-66016085 (−) | CTCCGGAGCTGGGGATTGTGGGTTCGAGTCC |
| | | CATCTGGGTCG |
| 32 | Arg_CCT_chr17: | GCCCCAGTGGCCTAATGGATAAGGCACTGGC |
| | 73030001-73030073 (+) | CTCCTAAGCCAGGGATTGTGGGTTCGAGTCC |
| | | CACCTGGGGTA |
| 33 | Arg_CCT_chr17: | GCCCCAGTGGCCTAATGGATAAGGCACTGGC |
| | 73030526-73030598 (−) | CTCCTAAGCCAGGGATTGTGGGTTCGAGTCC |
| | | CACCTGGGGTG |
| 34 | Arg_CCT_chr16: | GCCCCGGTGGCCTAATGGATAAGGCATTGGC |
| | 3202901-3202973 (+) | CTCCTAAGCCAGGGATTGTGGGTTCGAGTCC |
| | | CACCCGGGGTA |
| 35 | Arg_CCT_chr7: | GCCCCAGTGGCCTAATGGATAAGGCATTGGC |
| | 139025446-139025518 (+) | CTCCTAAGCCAGGGATTGTGGGTTCGAGTCC |
| | | CATCTGGGGTG |
| 36 | Arg_CCT_chr16: | GCCCCAGTGGCCTGATGGATAAGGTACTGGC |
| | 3243918-3243990 (+) | CTCCTAAGCCAGGGATTGTGGGTTCGAGTTC |
| | | CACCTGGGGTA |
| 37 | Arg_TCG_chr15: | GGCCGCGTGGCCTAATGGATAAGGCGTCTGA |
| | 89878304-89878376 (+) | CTTCGGATCAGAAGATTGCAGGTTCGAGTCC |
| | | TGCCGCGGTCG |
| 38 | Arg_TCG_chr6: | GACCACGTGGCCTAATGGATAAGGCGTCTGA |
| | 26323046-26323118 (+) | CTTCGGATCAGAAGATTGAGGGTTCGAATCC |
| | | CTCCGTGGTTA |
| 39 | Arg_TCG_chr17: | GACCGCGTGGCCTAATGGATAAGGCGTCTGA |
| | 73031208-73031280 (+) | CTTCGGATCAGAAGATTGAGGGTTCGAGTCC |
| | | CTTCGTGGTCG |
| 40 | Arg_TCG_chr6: | GACCACGTGGCCTAATGGATAAGGCGTCTGA |
| | 26299905-26299977 (+) | CTTCGGATCAGAAGATTGAGGGTTCGAATCC |
| | | CTTCGTGGTTA |
| 41 | Arg_TCG_chr6: | GACCACGTGGCCTAATGGATAAGGCGTCTGA |
| | 28510891-28510963 (−) | CTTCGGATCAGAAGATTGAGGGTTCGAATCC |
| | | CTTCGTGGTTG |
| 42 | Arg_TCG_chr9: | GGCCGTGTGGCCTAATGGATAAGGCGTCTGA |
| | 112960803-112960875 (+) | CTTCGGATCAAAAGATTGCAGGTTTGAGTTC |
| | | TGCCACGGTCG |
| 43 | Arg_TCT_chr1: | GGCTCCGTGGCGCAATGGATAGCGCATTGGA |
| | 94313129-94313213 (+) | CTTCTAGAGGCTGAAGGCATTCAAAGGTTCC |
| | | GGGTTCGAGTCCCGGCGGAGTCG |
| 44 | Arg_TCT_chr17: | GGCTCTGTGGCGCAATGGATAGCGCATTGGA |
| | 8024243-8024330 (+) | CTTCTAGTGACGAATAGAGCAATTCAAAGGT |
| | | TGTGGGTTCGAATCCCACCAGAGTCG |
| 45 | Arg_TCT_chr9: | GGCTCTGTGGCGCAATGGATAGCGCATTGGA |
| | 131102355-131102445 (−) | CTTCTAGCTGAGCCTAGTGTGGTCATTCAAA |
| | | GGTTGTGGGTTCGAGTCCCACCAGAGTCG |
| 46 | Arg_TCT_chr11: | GGCTCTGTGGCGCAATGGATAGCGCATTGGA |
| | 59318767-59318852 (+) | CTTCTAGATAGTTAGAGAAATTCAAAGGTTG |
| | | TGGGTTCGAGTCCCACCAGAGTCG |
| 47 | Arg_TCT_chr1: | GTCTCTGTGGCGCAATGGACGAGCGCGCTGG |
| | 159111401-159111474 (−) | ACTTCTAATCCAGAGGTTCCGGGTTCGAGTC |
| | | CCGGCAGAGATG |
| 48 | Arg_TCT_chr6: | GGCTCTGTGGCGCAATGGATAGCGCATTGGA |
| | 27529963-27530049 (+) | CTTCTAGCCTAAATCAAGAGATTCAAAGGTT |
| | | GCGGGTTCGAGTCCCTCCAGAGTCG |
| 49 | Asn_GTT_chr1: | GTCTCTGTGGCGCAATCGGTTAGCGCGTTCG |
| | 161510031-161510104 (+) | GCTGTTAACCGAAAGGTTGGTGGTTCGATCC |
| | | CACCCAGGGACG |
| 50 | Asn_GTT_chr1: | GTCTCTGTGGCGCAATCGGCTAGCGCGTTTG |
| | 143879832-143879905 (−) | GCTGTTAACTAAAAGGTTGGCGGTTCGAACC |
| | | CACCCAGAGGCG |
| 51 | Asn_GTT_chr1: | GTCTCTGTGGTGCAATCGGTTAGCGCGTTCCG |
| | 144301611-144301684 (+) | CTGTTAACCGAAAGCTTGGTGGTTCGAGCCC |
| | | ACCCAGGGATG |
| 52 | Asn_GTT_chr1: | GTCTCTGTGGCGCAATCGGCTAGCGCGTTTG |
| | 149326272-149326345 (−) | GCTGTTAACTAAAAAGTTGGTGGTTCGAACA |
| | | CACCCAGAGGCG |
| 53 | Asn_GTT_chr1: | GTCTCTGTGGCGCAATCGGTTAGCGCGTTCG |
| | 148248115-148248188 (+) | GCTGTTAACCGAAAGGTTGGTGGTTCGAGCC |
| | | CACCCAGGGACG |
| 54 | Asn_GTT_chr1: | GTCTCTGTGGCGCAATCGGTTAGCGCATTCG |
| | 148598314-148598387 (−) | GCTGTTAACCGAAAGGTTGGTGGTTCGAGCC |
| | | CACCCAGGGACG |
| 55 | Asn_GTT_chr1: | GTCTCTGTGGCGCAATCGGTTAGCGCGTTCG |
| | 17216172-17216245 (+) | GCTGTTAACCGAAAGATTGGTGGTTCGAGCC |
| | | CACCCAGGGACG |
| 56 | Asn_GTT_chr1: | GTCTCTGTGGCGCAATCGGTTAGCGCGTTCG |
| | 16847080-16847153 (−) | GCTGTTAACTGAAAGGTTGGTGGTTCGAGCC |
| | | CACCCAGGGACG |
| 57 | Asn_GTT_chr1: | GTCTCTGTGGCGCAATGGGTTAGCGCGTTCG |
| | 149230570-149230643 (−) | GCTGTTAACCGAAAGGTTGGTGGTTCGAGCC |
| | | CATCCAGGGACG |
| 58 | Asn_GTT_chr1: | GTCTCTGTGGCGTAGTCGGTTAGCGCGTTCG |
| | 148000805-148000878 (+) | GCTGTTAACCGAAAAGTTGGTGGTTCGAGCC |
| | | CACCCAGGAACG |
| 59 | Asn_GTT_chr1: | GTCTCTGTGGCGCAATCGGCTAGCGCGTTTG |
| | 149711798-149711871 (−) | GCTGTTAACTAAAAGGTTGGTGGTTCGAACC |
| | | CACCCAGAGGCG |
| 60 | Asn_GTT_chr1: | GTCTCTGTGGCGCAATCGGTTAGCGCGTTCG |
| | 145979034-145979107 (−) | GCTGTTAACTGAAAGGTTAGTGGTTCGAGCC |
| | | CACCCGGGGACG |
| 61 | Asp_GTC_chr12: | TCCTCGTTAGTATAGTGGTTAGTATCCCCGCC |
| | 98897281-98897352 (+) | TGTCACGCGGGAGACCGGGGTTCAATTCCCC |
| | | GACGGGGAG |
| 62 | Asp_GTC_chr1: | TCCTCGTTAGTATAGTGGTGAGTATCCCCGCC |
| | 161410615-161410686 (−) | TGTCACGCGGGAGACCGGGGTTCGATTCCCC |
| | | GACGGGGAG |
| 63 | Asp_GTC_chr6: | TCCTCGTTAGTATAGTGGTGAGTGTCCCCGTC |
| | 27551236-27551307 (−) | TGTCACGCGGGAGACCGGGGTTCGATTCCCC |
| | | GACGGGGAG |
| 64 | Cys_GCA_chr7: | GGGGGCATAGCTCAGTGGTAGAGCATTTGAC |
| | 149007281-149007352 (+) | TGCAGATCAAGAGGTCCCTGGTTCAAATCCA |
| | | GGTGCCCCCT |
| 65 | Cys_GCA_chr7: | GGGGGTATAGCTCAGGGGTAGAGCATTTGAC |
| | 149074601-149074672 (−) | TGCAGATCAAGAGGTCCCTGGTTCAAATCCA |
| | | GGTGCCCCCC |
| 66 | Cys_GCA_chr7: | GGGGGTATAGCTTAGCGGTAGAGCATTTGAC |
| | 149112229-149112300 (−) | TGCAGATCAAGAGGTCCCCGGTTCAAATCCG |
| | | GGTGCCCCCT |
| 67 | Cys_GCA_chr7: | GGGGGTATAGCTTAGGGGTAGAGCATTTGAC |
| | 149344046-149344117 (−) | TGCAGATCAAAAGGTCCCTGGTTCAAATCCA |
| | | GGTGCCCCTT |
| 68 | Cys_GCA_chr7: | GGGGGTATAGCTCAGGGGTAGAGCATTTGAC |
| | 149052766-149052837 (−) | TGCAGATCAAGAGGTCCCCAGTTCAAATCTG |
| | | GGTGCCCCCT |
| 69 | Cys_GCA_chr17: | GGGGGTATAGCTCAGGGGTAGAGCATTTGAC |
| | 37017937-37018008 (−) | TGCAGATCAAGAAGTCCCCGGTTCAAATCCG |
| | | GGTGCCCCCT |
| 70 | Cys_GCA_chr7: | GGGGGTATAGCTCAGGGGTAGAGCATTTGAC |
| | 149281816-149281887 (+) | TGCAGATCAAGAGGTCTCTGGTTCAAATCCA |
| | | GGTGCCCCCT |
| 71 | Cys_GCA_chr7: | GGGGGTATAGCTCAGGGGTAGAGCACTTGAC |
| | 149243631-149243702 (+) | TGCAGATCAAGAAGTCCTTGGTTCAAATCCA |
| | | GGTGCCCCCT |
| 72 | Cys_GCA_chr7: | GGGGATATAGCTCAGGGGTAGAGCATTTGAC |
| | 149388272-149388343 (−) | TGCAGATCAAGAGGTCCCCGGTTCAAATCCG |
| | | GGTGCCCCCC |
| 73 | Cys_GCA_chr7: | GGGGGTATAGTTCAGGGGTAGAGCATTTGAC |
| | 149072850-149072921 (−) | TGCAGATCAAGAGGTCCCTGGTTCAAATCCA |
| | | GGTGCCCCCT |
| 74 | Cys_GCA_chr7: | GGGGGTATAGCTCAGGGGTAGAGCATTTGAC |
| | 149310156-149310227 (−) | TGCAAATCAAGAGGTCCCTGATTCAAATCCA |
| | | GGTGCCCCCT |
| 75 | Cys_GCA_chr4: | GGGGGTATAGCTCAGTGGTAGAGCATTTGAC |
| | 124430005-124430076 (−) | TGCAGATCAAGAGGTCCCCGGTTCAAATCCG |
| | | GGTGCCCCCT |
| 76 | Cys_GCA_chr7: | GGGCGTATAGCTCAGGGGTAGAGCATTTGAC |
| | 149295046-149295117 (+) | TGCAGATCAAGAGGTCCCCAGTTCAAATCTG |
| | | GGTGCCCCCT |
| 77 | Cys_GCA_chr7: | GGGGGTATAGCTCACAGGTAGAGCATTTGAC |
| | 149361915-149361986 (+) | TGCAGATCAAGAGGTCCCCGGTTCAAATCTG |
| | | GGTGCCCCCT |
| 78 | Cys_GCA_chr7: | GGGCGTATAGCTCAGGGGTAGAGCATTTGAC |
| | 149253802-149253871 (+) | TGCAGATCAAGAGGTCCCCAGTTCAAATCTG |
| | | GGTGCCCA |
| 79 | Cys_GCA_chr7: | GGGGGTATAGCTCACAGGTAGAGCATTTGAC |
| | 149292305-149292376 (−) | TGCAGATCAAGAGGTCCCCGGTTCAAATCCG |
| | | GTTACTCCCT |
| 80 | Cys_GCA_chr7: | GGGGGTATAGCTCAGGGGTAGAGCACTTGAC |
| | 149286164-149286235 (−) | TGCAGATCAAGAGGTCCCTGGTTCAAATCCA |
| | | GGTGCCCCCT |
| 81 | Cys_GCA_chr17: | GGGGGTATAGCTCAGTGGTAGAGCATTTGAC |
| | 37025545-37025616 (−) | TGCAGATCAAGAGGTCCCTGGTTCAAATCCG |
| | | GGTGCCCCCT |
| 82 | Cys_GCA_chr15: | GGGGGTATAGCTCAGTGGGTAGAGCATTTGA |
| | 80036997-80037069 (+) | CTGCAGATCAAGAGGTCCCCGGTTCAAATCC |
| | | GGGTGCCCCCT |
| 83 | Cys_GCA_chr3: | GGGGGTGTAGCTCAGTGGTAGAGCATTTGAC |
| | 131947944-131948015 (−) | TGCAGATCAAGAGGTCCCTGGTTCAAATCCA |
| | | GGTGCCCCCT |
| 84 | Cys_GCA_chr1: | GGGGGTATAGCTCAGGTGGTAGAGCATTTGA |
| | 93981834-93981906 (−) | CTGCAGATCAAGAGGTCCCCGGTTCAAATCC |
| | | GGGTGCCCCCT |
| 85 | Cys_GCA_chr14: | GGGGGTATAGCTCAGGGGTAGAGCATTTGAC |
| | 73429679-73429750 (+) | TGCAGATCAAGAGGTCCCCGGTTCAAATCCG |
| | | GGTGCCCCCT |
| 86 | Cys_GCA_chr3: | GGGGGTATAGCTCAGGGGTAGAGCATTTGAC |
| | 131950642-131950713 (−) | TGCAGATCAAGAGGTCCCTGGTTCAAATCCA |
| | | GGTGCCCCCT |
| 87 | Gln_CTG_chr6: | GGTTCCATGGTGTAATGGTTAGCACTCTGGA |
| | 18836402-18836473 (+) | CTCTGAATCCAGCGATCCGAGTTCAAATCTC |
| | | GGTGGAACCT |
| 88 | Gln_CTG_chr6: | GGTTCCATGGTGTAATGGTTAGCACTCTGGA |
| | 27515531-27515602 (−) | CTCTGAATCCAGCGATCCGAGTTCAAGTCTC |
| | | GGTGGAACCT |
| 89 | Gln_CTG_chr1: | GGTTCCATGGTGTAATGGTGAGCACTCTGGA |
| | 145963304-145963375 (+) | CTCTGAATCCAGCGATCCGAGTTCGAGTCTC |
| | | GGTGGAACCT |
| 90 | Gln_CTG_chr1: | GGTTCCATGGTGTAATGGTAAGCACTCTGGA |
| | 147737382-147737453 (−) | CTCTGAATCCAGCGATCCGAGTTCGAGTCTC |
| | | GGTGGAACCT |
| 91 | Gln_CTG_chr6: | GGTTCCATGGTGTAATGGTTAGCACTCTGGA |
| | 27263212-27263283 (+) | CTCTGAATCCGGTAATCCGAGTTCAAATCTC |
| | | GGTGGAACCT |
| 92 | Gln_CTG_chr6: | GGCCCCATGGTGTAATGGTCAGCACTCTGGA |
| | 27759135-27759206 (−) | CTCTGAATCCAGCGATCCGAGTTCAAATCTC |
| | | GGTGGGACCC |
| 93 | Gln_CTG_chr1: | GGTTCCATGGTGTAATGGTAAGCACTCTGGA |
| | 147800937-147801008 (+) | CTCTGAATCCAGCCATCTGAGTTCGAGTCTCT |
| | | GTGGAACCT |
| 94 | Gln_TTG_chr17: | GGTCCCATGGTGTAATGGTTAGCACTCTGGA |
| | 47269890-47269961 (+) | CTTTGAATCCAGCGATCCGAGTTCAAATCTC |
| | | GGTGGGACCT |
| 95 | Gln_TTG_chr6: | GGTCCCATGGTGTAATGGTTAGCACTCTGGA |
| | 28557156-28557227 (+) | CTTTGAATCCAGCAATCCGAGTTCGAATCTC |
| | | GGTGGGACCT |
| 96 | Gln_TTG_chr6: | GGCCCCATGGTGTAATGGTTAGCACTCTGGA |
| | 26311424-26311495 (−) | CTTTGAATCCAGCGATCCGAGTTCAAATCTC |
| | | GGTGGGACCT |
| 97 | Gln_TTG_chr6: | GGTCCCATGGTGTAATGGTTAGCACTCTGGG |
| | 145503859-145503930 (+) | CTTTGAATCCAGCAATCCGAGTTCGAATCTTG |
| | | GTGGGACCT |
| 98 | Glu_CTC_chr1: | TCCCTGGTGGTCTAGTGGTTAGGATTCGGCG |
| | 145399233-145399304 (−) | CTCTCACCGCCGCGGCCCGGGTTCGATTCCC |
| | | GGTCAGGGAA |
| 99 | Glu_CTC_chr1: | TCCCTGGTGGTCTAGTGGTTAGGATTCGGCG |
| | 249168447-249168518 (+) | CTCTCACCGCCGCGGCCCGGGTTCGATTCCC |
| | | GGTCAGGAAA |
| 100 | Glu_TTC_chr2: | TCCCATATGGTCTAGCGGTTAGGATTCCTGGT |
| | 131094701-131094772 (−) | TTTCACCCAGGTGGCCCGGGTTCGACTCCCG |
| | | GTATGGGAA |
| 101 | Glu_TTC_chr13: | TCCCACATGGTCTAGCGGTTAGGATTCCTGGT |
| | 45492062-45492133 (−) | TTTCACCCAGGCGGCCCGGGTTCGACTCCCG |
| | | GTGTGGGAA |
| 102 | Glu_TTC_chr1: | TCCCTGGTGGTCTAGTGGCTAGGATTCGGCG |
| | 17199078-17199149 (+) | CTTTCACCGCCGCGGCCCGGGTTCGATTCCCG |
| | | GCCAGGGAA |
| 103 | Glu_TTC_chr1: | TCCCTGGTGGTCTAGTGGCTAGGATTCGGCG |
| | 71686174-16861845 (−) | CTTTCACCGCCGCGGCCCGGGTTCGATTCCCG |
| | | GTCAGGGAA |
| 104 | Gly_CCC_chr1: | GCATTGGTGGTTCAGTGGTAGAATTCTCGCCT |
| | 16872434-16872504 (−) | CCCACGCGGGAGACCCGGGTTCAATTCCCGG |
| | | CCAATGCA |
| 105 | Gly_CCC_chr2: | GCGCCGCTGGTGTAGTGGTATCATGCAAGAT |
| | 70476123-70476193 (−) | TCCCATTCTTGCGACCCGGGTTCGATTCCCGG |
| | | GCGGCGCA |
| 106 | Gly_CCC_chr17: | GCATTGGTGGTTCAATGGTAGAATTCTCGCCT |
| | 19764175-19764245 (+) | CCCACGCAGGAGACCCAGGTTCGATTCCTGG |
| | | CCAATGCA |
| 107 | Gly_GCC_chr1: | GCATGGGTGGTTCAGTGGTAGAATTCTCGCC |
| | 161413094-161413164 (+) | TGCCACGCGGGAGGCCCGGGTTCGATTCCCG |
| | | GCCCATGCA |
| 108 | Gly_GCC_chr1: | GCATTGGTGGTTCAGTGGTAGAATTCTCGCCT |
| | 161493637-161493707 (−) | GCCACGCGGGAGGCCCGGGTTCGATTCCCGG |
| | | CCAATGCA |
| 109 | Gly_GCC_chr16: | GCATTGGTGGTTCAGTGGTAGAATTCTCGCCT |
| | 70812114-70812184 (−) | GCCACGCGGGAGGCCCGGGTTTGATTCCCGG |
| | | CCAGTGCA |
| 110 | Gly_GCC_chr1: | GCATAGGTGGTTCAGTGGTAGAATTCTTGCC |
| | 161450356-161450426 (+) | TGCCACGCAGGAGGCCCAGGTTTGATTCCTG |
| | | GCCCATGCA |
| 111 | Gly_GCC_chr16: | GCATTGGTGGTTCAGTGGTAGAATTCTCGCCT |
| | 70822597-70822667 (+) | GCCATGCGGGCGGCCGGGCTTCGATTCCTGG |
| | | CCAATGCA |
| 112 | Gly_TCC_chr19: | GCGTTGGTGGTATAGTGGTTAGCATAGCTGC |
| | 4724082-4724153 (+) | CTTCCAAGCAGTTGACCCGGGTTCGATTCCC |
| | | GGCCAACGCA |
| 113 | Gly_TCC_chr1: | GCGTTGGTGGTATAGTGGTGAGCATAGCTGC |
| | 145397864-145397935 (−) | CTTCCAAGCAGTTGACCCGGGTTCGATTCCC |
| | | GGCCAACGCA |
| 114 | Gly_TCC_chr17: | GCGTTGGTGGTATAGTGGTAAGCATAGCTGC |
| | 8124866-8124937 (+) | CTTCCAAGCAGTTGACCCGGGTTCGATTCCC |
| | | GGCCAACGCA |
| 115 | Gly_TCC_chr1: | GCGTTGGTGGTATAGTGGTGAGCATAGTTGC |
| | 161409961-161410032 (−) | CTTCCAAGCAGTTGACCCGGGCTCGATTCCC |
| | | GCCCAACGCA |
| 116 | His_GTG_chr1: | GCCGTGATCGTATAGTGGTTAGTACTCTGCGT |
| | 145396881-145396952 (−) | TGTGGCCGCAGCAACCTCGGTTCGAATCCGA |
| | | GTCACGGCA |
| 117 | His_GTG_chr1: | GCCATGATCGTATAGTGGTTAGTACTCTGCG |
| | 149155828-149155899 (−) | CTGTGGCCGCAGCAACCTCGGTTCGAATCCG |
| | | AGTCACGGCA |
| 118 | Ile_AAT_chr6: | GGCCGGTTAGCTCAGTTGGTTAGAGCGTGGC |
| | 58149254-58149327 (+) | GCTAATAACGCCAAGGTCGCGGGTTCGATCC |
| | | CCGTACGGGCCA |
| 119 | Ile_AAT_chr6: | GGCCGGTTAGCTCAGTTGGTTAGAGCGTGGT |
| | 27655967-27656040 (+) | GCTAATAACGCCAAGGTCGCGGGTTCGATCC |
| | | CCGTACTGGCCA |
| 120 | Ile_AAT_chr6: | GGCTGGTTAGCTCAGTTGGTTAGAGCGTGGT |
| | 27242990-27243063 (−) | GCTAATAACGCCAAGGTCGCGGGTTCGATCC |
| | | CCGTACTGGCCA |
| 121 | Ile_AAT_chr17: | GGCCGGTTAGCTCAGTTGGTTAGAGCGTGGT |
| | 8130309-8130382 (−) | GCTAATAACGCCAAGGTCGCGGGTTCGAACC |
| | | CCGTACGGGCCA |
| 122 | Ile_AAT_chr6: | GGCCGGTTAGCTCAGTTGGTTAGAGCGTGGT |
| | 26554350-26554423 (+) | GCTAATAACGCCAAGGTCGCGGGTTCGATCC |
| | | CCGTACGGGCCA |
| 123 | Ile_AAT_chr6: | GGCCGGTTAGCTCAGTTGGTTAGAGCGTGGT |
| | 26745255-26745328 (−) | GCTAATAACGCTAAGGTCGCGGGTTCGATCC |
| | | CCGTACTGGCCA |
| 124 | Ile_AAT_chr6: | GGCCGGTTAGCTCAGTTGGTCAGAGCGTGGT |
| | 26721221-26721294 (−) | GCTAATAACGCCAAGGTCGCGGGTTCGATCC |
| | | CCGTACGGGCCA |
| 125 | Ile_AAT_chr6: | GGCCGGTTAGCTCAGTCGGCTAGAGCGTGGT |
| | 27636362-27636435 (+) | GCTAATAACGCCAAGGTCGCGGGTTCGATCC |
| | | CCGTACGGGCCA |
| 126 | Ile_AAT_chr6: | GGCTGGTTAGTTCAGTTGGTTAGAGCGTGGT |
| | 27241739-27241812 (+) | GCTAATAACGCCAAGGTCGTGGGTTCGATCC |
| | | CCATATCGGCCA |
| 127 | Ile_GAT_chrX: | GGCCGGTTAGCTCAGTTGGTAAGAGCGTGGT |
| | 3756418-3756491 (−) | GCTGATAACACCAAGGTCGCGGGCTCGACTC |
| | | CCGCACCGGCCA |
| 128 | Ile_TAT_chr19: | GCTCCAGTGGCGCAATCGGTTAGCGCGCGGT |
| | 39902808-39902900 (−) | ACTTATATGACAGTGCGAGCGGAGCAATGCC |
| | | GAGGTTGTGAGTTCGATCCTCACCTGGAGCA |
| 129 | Ile_TAT_chr2: | GCTCCAGTGGCGCAATCGGTTAGCGCGCGGT |
| | 43037676-43037768 (+) | ACTTATACAGCAGTACATGCAGAGCAATGCC |
| | | GAGGTTGTGAGTTCGAGCCTCACCTGGAGCA |
| 130 | Ile_TAT_chr6: | GCTCCAGTGGCGCAATCGGTTAGCGCGCGGT |
| | 26988125-26988218 (+) | ACTTATATGGCAGTATGTGTGCGAGTGATGC |
| | | CGAGGTTGTGAGTTCGAGCCTCACCTGGAGC |
| | | A |
| 131 | Ile_TAT_chr6: | GCTCCAGTGGCGCAATCGGTTAGCGCGCGGT |
| | 27599200-27599293 (+) | ACTTATACAACAGTATATGTGCGGGTGATGC |
| | | CGAGGTTGTGAGTTCGAGCCTCACCTGGAGC |
| | | A |
| 132 | Ile_TAT_chr6: | GCTCCAGTGGCGCAATCGGTTAGCGCGCGGT |
| | 28505367-28505460 (+) | ACTTATAAGACAGTGCACCTGTGAGCAATGC |
| | | CGAGGTTGTGAGTTCAAGCCTCACCTGGAGC |
| | | A |
| 133 | Leu_AAG_chr5: | GGTAGCGTGGCCGAGCGGTCTAAGGCGCTGG |
| | 180524474-180524555 (−) | ATTAAGGCTCCAGTCTCTTCGGAGGCGTGGG |
| | | TTCGAATCCCACCGCTGCCA |
| 134 | Leu_AAG_chr5: | GGTAGCGTGGCCGAGCGGTCTAAGGCGCTGG |
| | 180614701-180614782 (+) | ATTAAGGCTCCAGTCTCTTCGGGGGCGTGGG |
| | | TTCGAATCCCACCGCTGCCA |
| 135 | Leu_AAG_chr6: | GGTAGCGTGGCCGAGCGGTCTAAGGCGCTGG |
| | 28956779-28956860 (+) | ATTAAGGCTCCAGTCTCTTCGGGGGCGTGGG |
| | | TTCAAATCCCACCGCTGCCA |
| 136 | Leu_AAG_chr6: | GGTAGCGTGGCCGAGTGGTCTAAGACGCTGG |
| | 28446400-28446481 (−) | ATTAAGGCTCCAGTCTCTTCGGGGGCGTGGG |
| | | TTTGAATCCCACCGCTGCCA |
| 137 | Leu_CAA_chr6: | GTCAGGATGGCCGAGTGGTCTAAGGCGCCAG |
| | 28864000-28864105 (−) | ACTCAAGCTAAGCTTCCTCCGCGGTGGGGAT |
| | | TCTGGTCTCCAATGGAGGCGTGGGTTCGAAT |
| | | CCCACTTCTGACA |
| 138 | Leu_CAA_chr6: | GTCAGGATGGCCGAGTGGTCTAAGGCGCCAG |
| | 28908830-28908934 (+) | ACTCAAGCTTGGCTTCCTCGTGTTGAGGATTC |
| | | TGGTCTCCAATGGAGGCGTGGGTTCGAATCC |
| | | CACTTCTGACA |
| 139 | Leu_CAA_chr6: | GTCAGGATGGCCGAGTGGTCTAAGGCGCCAG |
| | 27573417-27573524 (−) | ACTCAAGCTTACTGCTTCCTGTGTTCGGGTCT |
| | | TCTGGTCTCCGTATGGAGGCGTGGGTTCGAA |
| | | TCCCACTTCTGACA |
| 140 | Leu_CAA_chr6: | GTCAGGATGGCCGAGTGGTCTAAGGCGCCAG |
| | 27570348-27570454 (−) | ACTCAAGTTGCTACTTCCCAGGTTTGGGGCTT |
| | | CTGGTCTCCGCATGGAGGCGTGGGTTCGAAT |
| | | CCCACTTCTGACA |
| 141 | Leu_CAA_chr1: | GTCAGGATGGCCGAGTGGTCTAAGGCGCCAG |
| | 249168054-249168159 (+) | ACTCAAGGTAAGCACCTTGCCTGCGGGCTTT |
| | | CTGGTCTCCGGATGGAGGCGTGGGTTCGAAT |
| | | CCCACTTCTGACA |
| 142 | Leu_CAA_chr11: | GCCTCCTTAGTGCAGTAGGTAGCGCATCAGT |
| | 9296790-9296863 (+) | CTCAAAATCTGAATGGTCCTGAGTTCAAGCC |
| | | TCAGAGGGGGCA |
| 143 | Leu_CAA_chr1: | GTCAGGATGGCCGAGCAGTCTTAAGGCGCTG |
| | 161581736-161581819 (−) | CGTTCAAATCGCACCCTCCGCTGGAGGCGTG |
| | | GGTTCGAATCCCACTTTTGACA |
| 144 | Leu_CAG_chr1: | GTCAGGATGGCCGAGCGGTCTAAGGCGCTGC |
| | 161411323-161411405 (+) | GTTCAGGTCGCAGTCTCCCCTGGAGGCGTGG |
| | | GTTCGAATCCCACTCCTGACA |
| 145 | Leu_CAG_chr16: | GTCAGGATGGCCGAGCGGTCTAAGGCGCTGC |
| | 57333863-57333945 (+) | GTTCAGGTCGCAGTCTCCCCTGGAGGCGTGG |
| | | GTTCGAATCCCACTTCTGACA |
| 146 | Leu_TAA_chr6: | ACCAGGATGGCCGAGTGGTTAAGGCGTTGGA |
| | 144537684-144537766 (+) | CTTAAGATCCAATGGACATATGTCCGCGTGG |
| | | GTTCGAACCCCACTCCTGGTA |
| 147 | Leu_TAA_chr6: | ACCGGGATGGCCGAGTGGTTAAGGCGTTGGA |
| | 27688898-27688980 (−) | CTTAAGATCCAATGGGCTGGTGCCCGCGTGG |
| | | GTTCGAACCCCACTCTCGGTA |
| 148 | Leu_TAA_chr11: | ACCAGAATGGCCGAGTGGTTAAGGCGTTGGA |
| | 59319228-59319310 (+) | CTTAAGATCCAATGGATTCATATCCGCGTGG |
| | | GTTCGAACCCCACTTCTGGTA |
| 149 | Leu_TAA_chr6: | ACCGGGATGGCTGAGTGGTTAAGGCGTTGGA |
| | 27198334-27198416 (−) | CTTAAGATCCAATGGACAGGTGTCCGCGTGG |
| | | GTTCGAGCCCCACTCCCGGTA |
| 150 | Leu_TAG_chr17: | GGTAGCGTGGCCGAGCGGTCTAAGGCGCTGG |
| | 8023632-8023713 (−) | ATTTAGGCTCCAGTCTCTTCGGAGGCGTGGG |
| | | TTCGAATCCCACCGCTGCCA |
| 151 | Leu_TAG_chr14: | GGTAGTGTGGCCGAGCGGTCTAAGGCGCTGG |
| | 21093529-21093610 (+) | ATTTAGGCTCCAGTCTCTTCGGGGGCGTGGG |
| | | TTCGAATCCCACCACTGCCA |
| 152 | Leu_TAG_chr16: | GGTAGCGTGGCCGAGTGGTCTAAGGCGCTGG |
| | 22207032-22207113 (−) | ATTTAGGCTCCAGTCATTTCGATGGCGTGGGT |
| | | TCGAATCCCACCGCTGCCA |
| 153 | Lys_CTT_chr14: | GCCCGGCTAGCTCAGTCGGTAGAGCATGGGA |
| | 58706613-58706685 (−) | CTCTTAATCCCAGGGTCGTGGGTTCGAGCCC |
| | | CACGTTGGGCG |
| 154 | Lys_CTT_chr19: | GCCCAGCTAGCTCAGTCGGTAGAGCATAAGA |
| | 36066750-36066822 (+) | CTCTTAATCTCAGGGTTGTGGATTCGTGCCCC |
| | | ATGCTGGGTG |
| 155 | Lys_CTT_chr19: | GCAGCTAGCTCAGTCGGTAGAGCATGAGACT |
| | 52425393-52425466 (−) | CTTAATCTCAGGGTCATGGGTTCGTGCCCCAT |
| | | GTTGGGTGCCA |
| 156 | Lys_CTT_chr1: | GCCCGGCTAGCTCAGTCGGTAGAGCATGAGA |
| | 145395522-145395594 (−) | CTCTTAATCTCAGGGTCGTGGGTTCGAGCCCC |
| | | ACGTTGGGCG |
| 157 | Lys_CTT_chr16: | GCCCGGCTAGCTCAGTCGGTAGAGCATGAGA |
| | 3207406-3207478 (−) | CCCTTAATCTCAGGGTCGTGGGTTCGAGCCC |
| | | CACGTTGGGCG |
| 158 | Lys_CTT_chr16: | GCCCGGCTAGCTCAGTCGGTAGAGCATGGGA |
| | 3241501-3241573 (+) | CTCTTAATCTCAGGGTCGTGGGTTCGAGCCCC |
| | | ACGTTGGGCG |
| 159 | Lys_CTT_chr16: | GCCCGGCTAGCTCAGTCGATAGAGCATGAGA |
| | 3230555-3230627 (−) | CTCTTAATCTCAGGGTCGTGGGTTCGAGCCG |
| | | CACGTTGGGCG |
| 160 | Lys_CTT_chr1: | GCCCAGCTAGCTCAGTCGGTAGAGCATGAGA |
| | 55423542-55423614 (−) | CTCTTAATCTCAGGGTCATGGGTTTGAGCCCC |
| | | ACGTTTGGTG |
| 161 | Lys_CTT_chr16: | GCCTGGCTAGCTCAGTCGGCAAAGCATGAGA |
| | 3214939-3215011 (+) | CTCTTAATCTCAGGGTCGTGGGCTCGAGCTCC |
| | | ATGTTGGGCG |
| 162 | Lys_CTT_chr5: | GCCCGACTACCTCAGTCGGTGGAGCATGGGA |
| | 26198539-26198611 (−) | CTCTTCATCCCAGGGTTGTGGGTTCGAGCCCC |
| | | ACATTGGGCA |
| 163 | Lys_TTT_chr16: | GCCTGGATAGCTCAGTTGGTAGAGCATCAGA |
| | 73512216-73512288 (−) | CTTTTAATCTGAGGGTCCAGGGTTCAAGTCCC |
| | | TGTTCAGGCA |
| 164 | Lys_TTT_chr12: | ACCCAGATAGCTCAGTCAGTAGAGCATCAGA |
| | 27843306-27843378 (+) | CTTTTAATCTGAGGGTCCAAGGTTCATGTCCC |
| | | TTTTTGGGTG |
| 165 | Lys_TTT_chr11: | GCCTGGATAGCTCAGTTGGTAGAGCATCAGA |
| | 122430655-122430727 (+) | CTTTTAATCTGAGGGTCCAGGGTTCAAGTCCC |
| | | TGTTCAGGCG |
| 166 | Lys_TTT_chr1: | GCCCGGATAGCTCAGTCGGTAGAGCATCAGA |
| | 204475655-204475727 (+) | CTTTTAATCTGAGGGTCCAGGGTTCAAGTCCC |
| | | TGTTCGGGCG |
| 167 | Lys_TTT_chr6: | GCCTGGATAGCTCAGTCGGTAGAGCATCAGA |
| | 27559593-27559665 (−) | CTTTTAATCTGAGGGTCCAGGGTTCAAGTCCC |
| | | TGTTCAGGCG |
| 168 | Lys_TTT_chr11: | GCCCGGATAGCTCAGTCGGTAGAGCATCAGA |
| | 59323902-59323974 (+) | CTTTTAATCTGAGGGTCCGGGGTTCAAGTCCC |
| | | TGTTCGGGCG |
| 169 | Lys_TTT_chr6: | GCCTGGGTAGCTCAGTCGGTAGAGCATCAGA |
| | 27302769-27302841 (−) | CTTTTAATCTGAGGGTCCAGGGTTCAAGTCCC |
| | | TGTCCAGGCG |
| 170 | Lys_TTT_chr6: | GCCTGGATAGCTCAGTTGGTAGAACATCAGA |
| | 28715521-28715593 (+) | CTTTTAATCTGACGGTGCAGGGTTCAAGTCCC |
| | | TGTTCAGGCG |
| 171 | Met_CAT_chr8: | GCCTCGTTAGCGCAGTAGGTAGCGCGTCAGT |
| | 124169470-124169542 (−) | CTCATAATCTGAAGGTCGTGAGTTCGATCCTC |
| | | ACACGGGGCA |
| 172 | Met_CAT_chr16: | GCCCTCTTAGCGCAGTGGGCAGCGCGTCAGT |
| | 71460396-71460468 (+) | CTCATAATCTGAAGGTCCTGAGTTCGAGCCT |
| | | CAGAGAGGGCA |
| 173 | Met_CAT_chr6: | GCCTCCTTAGCGCAGTAGGCAGCGCGTCAGT |
| | 28912352-28912424 (+) | CTCATAATCTGAAGGTCCTGAGTTCGAACCT |
| | | CAGAGGGGGCA |
| 174 | Met_CAT_chr6: | GCCCTCTTAGCGCAGCGGGCAGCGCGTCAGT |
| | 26735574-26735646 (−) | CTCATAATCTGAAGGTCCTGAGTTCGAGCCT |
| | | CAGAGAGGGCA |
| 175 | Met_CAT_chr6: | GCCCTCTTAGCGCAGCTGGCAGCGCGTCAGT |
| | 26701712-26701784 (+) | CTCATAATCTGAAGGTCCTGAGTTCAAGCCT |
| | | CAGAGAGGGCA |
| 176 | Met_CAT_chr16: | GCCTCGTTAGCGCAGTAGGCAGCGCGTCAGT |
| | 87417628-87417700 (−) | CTCATAATCTGAAGGTCGTGAGTTCGAGCCT |
| | | CACACGGGGCA |
| 177 | Met_CAT_chr6: | GCCCTCTTAGTGCAGCTGGCAGCGCGTCAGT |
| | 58168492-58168564 (−) | TTCATAATCTGAAAGTCCTGAGTTCAAGCCTC |
| | | AGAGAGGGCA |
| 178 | Phe_GAA_chr6: | GCCGAAATAGCTCAGTTGGGAGAGCGTTAGA |
| | 28758499-28758571 (−) | CTGAAGATCTAAAGGTCCCTGGTTCGATCCC |
| | | GGGTTTCGGCA |
| 179 | Phe_GAA_chr11: | GCCGAAATAGCTCAGTTGGGAGAGCGTTAGA |
| | 59333853-59333925 (−) | CTGAAGATCTAAAGGTCCCTGGTTCAATCCC |
| | | GGGTTTCGGCA |
| 180 | Phe_GAA_chr6: | GCCGAGATAGCTCAGTTGGGAGAGCGTTAGA |
| | 28775610-28775682 (−) | CTGAAGATCTAAAGGTCCCTGGTTCAATCCC |
| | | GGGTTTCGGCA |
| 181 | Phe_GAA_chr6: | GCCGAAATAGCTCAGTTGGGAGAGCGTTAGA |
| | 28791093-28791166 (−) | CCGAAGATCTTAAAGGTCCCTGGTTCAATCC |
| | | CGGGTTTCGGCA |
| 182 | Phe_GAA_chr6: | GCTGAAATAGCTCAGTTGGGAGAGCGTTAGA |
| | 28731374-28731447 (−) | CTGAAGATCTTAAAGTTCCCTGGTTCAACCCT |
| | | GGGTTTCAGCC |
| 183 | Pro_AGG_chr16: | GGCTCGTTGGTCTAGGGGTATGATTCTCGCTT |
| | 3241989-3242060 (+) | AGGATGCGAGAGGTCCCGGGTTCAAATCCCG |
| | | GACGAGCCC |
| 184 | Pro_AGG_chr1: | GGCTCGTTGGTCTAGGGGTATGATTCTCGCTT |
| | 167684725-167684796 (−) | AGGGTGCGAGAGGTCCCGGGTTCAAATCCCG |
| | | GACGAGCCC |
| 185 | Pro_CGG_chr1: | GGCTCGTTGGTCTAGGGGTATGATTCTCGCTT |
| | 167683962-167684033 (+) | CGGGTGCGAGAGGTCCCGGGTTCAAATCCCG |
| | | GACGAGCCC |
| 186 | Pro_CGG_chr6: | GGCTCGTTGGTCTAGGGGTATGATTCTCGCTT |
| | 27059521-27059592 (+) | CGGGTGTGAGAGGTCCCGGGTTCAAATCCCG |
| | | GACGAGCCC |
| 187 | Pro_TGG_chr14: | GGCTCGTTGGTCTAGTGGTATGATTCTCGCTT |
| | 21101165-21101236 (+) | TGGGTGCGAGAGGTCCCGGGTTCAAATCCCG |
| | | GACGAGCCC |
| 188 | Pro_TGG_chr11: | GGCTCGTTGGTCTAGGGGTATGATTCTCGGTT |
| | 75946869-75946940 (−) | TGGGTCCGAGAGGTCCCGGGTTCAAATCCCG |
| | | GACGAGCCC |
| 189 | Pro_TGG_chr5: | GGCTCGTTGGTCTAGGGGTATGATTCTCGCTT |
| | 180615854-180615925 (−) | TGGGTGCGAGAGGTCCCGGGTTCAAATCCCG |
| | | GACGAGCCC |
| 190 | SeC_TCA_chr19: | GCCCGGATGATCCTCAGTGGTCTGGGGTGCA |
| | 45981859-45981945 (−) | GGCTTCAAACCTGTAGCTGTCTAGCGACAGA |
| | | GTGGTTCAATTCCACCTTTCGGGCG |
| 191 | SeC_TCA_chr22: | GCTCGGATGATCCTCAGTGGTCTGGGGTGCA |
| | 44546537-44546620 (+) | GGCTTCAAACCTGTAGCTGTCTAGTGACAGA |
| | | GTGGTTCAATTCCACCTTTGTA |
| 192 | Ser_AGA_chr6: | GTAGTCGTGGCCGAGTGGTTAAGGCGATGGA |
| | 27509554-27509635 (−) | CTAGAAATCCATTGGGGTTTCCCCGCGCAGG |
| | | TTCGAATCCTGCCGACTACG |
| 193 | Ser_AGA_chr6: | GTAGTCGTGGCCGAGTGGTTAAGGCGATGGA |
| | 26327817-26327898 (+) | CTAGAAATCCATTGGGGTCTCCCCGCGCAGG |
| | | TTCGAATCCTGCCGACTACG |
| 194 | Ser_AGA_chr6: | GTAGTCGTGGCCGAGTGGTTAAGGCGATGGA |
| | 27499987-27500068 (+) | CTAGAAATCCATTGGGGTTTCCCCACGCAGG |
| | | TTCGAATCCTGCCGACTACG |
| 195 | Ser_AGA_chr6: | GTAGTCGTGGCCGAGTGGTTAAGGTGATGGA |
| | 27521192-27521273 (−) | CTAGAAACCCATTGGGGTCTCCCCGCGCAGG |
| | | TTCGAATCCTGCCGACTACG |
| 196 | Ser_CGA_chr17: | GCTGTGATGGCCGAGTGGTTAAGGCGTTGGA |
| | 8042199-8042280 (−) | CTCGAAATCCAATGGGGTCTCCCCGCGCAGG |
| | | TTCGAATCCTGCTCACAGCG |
| 197 | Ser_CGA_chr6: | GCTGTGATGGCCGAGTGGTTAAGGCGTTGGA |
| | 27177628-27177709 (+) | CTCGAAATCCAATGGGGTCTCCCCGCGCAGG |
| | | TTCAAATCCTGCTCACAGCG |
| 198 | Ser_CGA_chr6: | GCTGTGATGGCCGAGTGGTTAAGGTGTTGGA |
| | 27640229-27640310 (−) | CTCGAAATCCAATGGGGGTTCCCCGCGCAGG |
| | | TTCAAATCCTGCTCACAGCG |
| 199 | Ser_CGA_chr12: | GTCACGGTGGCCGAGTGGTTAAGGCGTTGGA |
| | 56584148-56584229 (+) | CTCGAAATCCAATGGGGTTTCCCCGCACAGG |
| | | TTCGAATCCTGTTCGTGACG |
| 200 | Ser_GCT_chr6: | GACGAGGTGGCCGAGTGGTTAAGGCGATGG |
| | 27065085-27065166 (+) | ACTGCTAATCCATTGTGCTCTGCACGCGTGG |
| | | GTTCGAATCCCACCCTCGTCG |
| 201 | Ser_GCT_chr6: | GACGAGGTGGCCGAGTGGTTAAGGCGATGG |
| | 27265775-27265856 (+) | ACTGCTAATCCATTGTGCTCTGCACGCGTGG |
| | | GTTCGAATCCCACCTTCGTCG |
| 202 | Ser_GCT_chr11: | GACGAGGTGGCCGAGTGGTTAAGGCGATGG |
| | 66115591-66115672 (+) | ACTGCTAATCCATTGTGCTTTGCACGCGTGGG |
| | | TTCGAATCCCATCCTCGTCG |
| 203 | Ser_GCT_chr6: | GACGAGGTGGCCGAGTGGTTAAGGCGATGG |
| | 28565117-28565198 (−) | ACTGCTAATCCATTGTGCTCTGCACGCGTGG |
| | | GTTCGAATCCCATCCTCGTCG |
| 204 | Ser_GCT_chr6: | GACGAGGTGGCCGAGTGGTTAAGGCGATGG |
| | 28180815-28180896 (+) | ACTGCTAATCCATTGTGCTCTGCACACGTGG |
| | | GTTCGAATCCCATCCTCGTCG |
| 205 | Ser_GCT_chr6: | GGAGAGGCCTGGCCGAGTGGTTAAGGCGATG |
| | 26305718-26305801 (−) | GACTGCTAATCCATTGTGCTCTGCACGCGTG |
| | | GGTTCGAATCCCATCCTCGTCG |
| 206 | Ser_TGA_chr10: | GCAGCGATGGCCGAGTGGTTAAGGCGTTGGA |
| | 69524261-69524342 (+) | CTTGAAATCCAATGGGGTCTCCCCGCGCAGG |
| | | TTCGAACCCTGCTCGCTGCG |
| 207 | Ser_TGA_chr6: | GTAGTCGTGGCCGAGTGGTTAAGGCGATGGA |
| | 42751368-27513549 (+) | CTTGAAATCCATTGGGGTTTCCCCGCGCAGG |
| | | TTCGAATCCTGCCGACTACG |
| 208 | Ser_TGA_chr6: | GTAGTCGTGGCCGAGTGGTTAAGGCGATGGA |
| | 26312824-26312905 (−) | CTTGAAATCCATTGGGGTCTCCCCGCGCAGG |
| | | TTCGAATCCTGCCGACTACG |
| 209 | Ser_TGA_chr6: | GTAGTCGTGGCCGAGTGGTTAAGGCGATGGA |
| | 27473607-27473688 (−) | CTTGAAATCCATTGGGGTTTCCCCGCGCAGG |
| | | TTCGAATCCTGTCGGCTACG |
| 210 | Thr_AGT_chr17: | GGCGCCGTGGCTTAGTTGGTTAAAGCGCCTG |
| | 8090478-8090551 (+) | TCTAGTAAACAGGAGATCCTGGGTTCGAATC |
| | | CCAGCGGTGCCT |
| 211 | Thr_AGT_chr6: | GGCTCCGTGGCTTAGCTGGTTAAAGCGCCTG |
| | 26533145-26533218 (−) | TCTAGTAAACAGGAGATCCTGGGTTCGAATC |
| | | CCAGCGGGGCCT |
| 212 | Thr_AGT_chr6: | GGCTCCGTAGCTTAGTTGGTTAAAGCGCCTG |
| | 28693795-28693868 (+) | TCTAGTAAACAGGAGATCCTGGGTTCGACTC |
| | | CCAGCGGGGCCT |
| 213 | Thr_AGT_chr6: | GGCTTCGTGGCTTAGCTGGTTAAAGCGCCTG |
| | 27694473-27694546 (+) | TCTAGTAAACAGGAGATCCTGGGTTCGAATC |
| | | CCAGCGAGGCCT |
| 214 | Thr_AGT_chr17: | GGCGCCGTGGCTTAGCTGGTTAAAGCGCCTG |
| | 8042770-8042843 (−) | TCTAGTAAACAGGAGATCCTGGGTTCGAATC |
| | | CCAGCGGTGCCT |
| 215 | Thr_AGT_chr6: | GGCCCTGTGGCTTAGCTGGTCAAAGCGCCTG |
| | 27130050-27130123 (+) | TCTAGTAAACAGGAGATCCTGGGTTCGAATC |
| | | CCAGCGGGGCCT |
| 216 | Thr_CGT_chr6: | GGCTCTATGGCTTAGTTGGTTAAAGCGCCTGT |
| | 28456770-28456843 (−) | CTCGTAAACAGGAGATCCTGGGTTCGACTCC |
| | | CAGTGGGGCCT |
| 217 | Thr_CGT_chr16: | GGCGCGGTGGCCAAGTGGTAAGGCGTCGGTC |
| | 14379750-14379821 (+) | TCGTAAACCGAAGATCACGGGTTCGAACCCC |
| | | GTCCGTGCCT |
| 218 | Thr_CGT_chr6: | GGCTCTGTGGCTTAGTTGGCTAAAGCGCCTG |
| | 28615984-28616057 (−) | TCTCGTAAACAGGAGATCCTGGGTTCGAATC |
| | | CCAGCGGGGCCT |
| 219 | Thr_CGT_chr17: | GGCGCGGTGGCCAAGTGGTAAGGCGTCGGTC |
| | 29877093-29877164 (+) | TCGTAAACCGAAGATCGCGGGTTCGAACCCC |
| | | GTCCGTGCCT |
| 220 | Thr_CGT_chr6: | GGCCCTGTAGCTCAGCGGTTGGAGCGCTGGT |
| | 27586135-27586208 (+) | CTCGTAAACCTAGGGGTCGTGAGTTCAAATC |
| | | TCACCAGGGCCT |
| 221 | Thr_TGT_chr6: | GGCTCTATGGCTTAGTTGGTTAAAGCGCCTGT |
| | 28442329-28442402 (−) | CTTGTAAACAGGAGATCCTGGGTTCGAATCC |
| | | CAGTAGAGCCT |
| 222 | Thr_TGT_chr1: | GGCTCCATAGCTCAGTGGTTAGAGCACTGGT |
| | 222638347-222638419 (+) | CTTGTAAACCAGGGGTCGCGAGTTCGATCCT |
| | | CGCTGGGGCCT |
| 223 | Thr_TGT_chr14: | GGCTCCATAGCTCAGGGGTTAGAGCGCTGGT |
| | 21081949-21082021 (−) | CTTGTAAACCAGGGGTCGCGAGTTCAATTCT |
| | | CGCTGGGGCCT |
| 224 | Thr_TGT_chr14: | GGCTCCATAGCTCAGGGGTTAGAGCACTGGT |
| | 21099319-21099391 (−) | CTTGTAAACCAGGGGTCGCGAGTTCAAATCT |
| | | CGCTGGGGCCT |
| 225 | Thr_TGT_chr14: | GGCCCTATAGCTCAGGGGTTAGAGCACTGGT |
| | 21149849-21149921 (+) | CTTGTAAACCAGGGGTCGCGAGTTCAAATCT |
| | | CGCTGGGGCCT |
| 226 | Thr_TGT_chr5: | GGCTCCATAGCTCAGGGGTTAGAGCACTGGT |
| | 180618687-180618758 (−) | CTTGTAAACCAGGGTCGCGAGTTCAAATCTC |
| | | GCTGGGGCCT |
| 227 | Trp_CCA_chr17: | GGCCTCGTGGCGCAACGGTAGCGCGTCTGAC |
| | 8124187-8124258 (−) | TCCAGATCAGAAGGTTGCGTGTTCAAATCAC |
| | | GTCGGGGTCA |
| 228 | Trp_CCA_chr17: | GACCTCGTGGCGCAATGGTAGCGCGTCTGAC |
| | 19411494-19411565 (+) | TCCAGATCAGAAGGTTGCGTGTTCAAGTCAC |
| | | GTCGGGGTCA |
| 229 | Trp_CCA_chr6: | GACCTCGTGGCGCAACGGTAGCGCGTCTGAC |
| | 26319330-26319401 (−) | TCCAGATCAGAAGGTTGCGTGTTCAAATCAC |
| | | GTCGGGGTCA |
| 230 | Trp_CCA_chr12: | GACCTCGTGGCGCAACGGTAGCGCGTCTGAC |
| | 98898030-98898101 (+) | TCCAGATCAGAAGGCTGCGTGTTCGAATCAC |
| | | GTCGGGGTCA |
| 231 | Trp_CCA_chr7: | GACCTCGTGGCGCAACGGCAGCGCGTCTGAC |
| | 99067307-99067378 (+) | TCCAGATCAGAAGGTTGCGTGTTCAAATCAC |
| | | GTCGGGGTCA |
| 232 | Tyr_ATA_chr2: | CCTTCAATAGTTCAGCTGGTAGAGCAGAGGA |
| | 219110549-219110641 (+) | CTATAGCTACTTCCTCAGTAGGAGACGTCCTT |
| | | AGGTTGCTGGTTCGATTCCAGCTTGAAGGA |
| 233 | Tyr_GTA_chr6: | CCTTCGATAGCTCAGTTGGTAGAGCGGAGGA |
| | 26569086-26569176 (+) | CTGTAGTTGGCTGTGTCCTTAGACATCCTTAG |
| | | GTCGCTGGTTCGAATCCGGCTCGAAGGA |
| 234 | Tyr_GTA_chr2: | CCTTCGATAGCTCAGTTGGTAGAGCGGAGGA |
| | 27273650-27273738 (+) | CTGTAGTGGATAGGGCGTGGCAATCCTTAGG |
| | | TCGCTGGTTCGATTCCGGCTCGAAGGA |
| 235 | Tyr_GTA_chr6: | CCTTCGATAGCTCAGTTGGTAGAGCGGAGGA |
| | 26577332-26577420 (+) | CTGTAGGCTCATTAAGCAAGGTATCCTTAGG |
| | | TCGCTGGTTCGAATCCGGCTCGGAGGA |
| 236 | Tyr_GTA_chr14: | CCTTCGATAGCTCAGCTGGTAGAGCGGAGGA |
| | 21125623-21125716 (−) | CTGTAGATTGTATAGACATTTGCGGACATCCT |
| | | TAGGTCGCTGGTTCGATTCCAGCTCGAAGGA |
| 237 | Tyr_GTA_chr8: | CCTTCGATAGCTCAGCTGGTAGAGCGGAGGA |
| | 67025602-67025694 (+) | CTGTAGCTACTTCCTCAGCAGGAGACATCCTT |
| | | AGGTCGCTGGTTCGATTCCGGCTCGAAGGA |
| 238 | Tyr_GTA_chr8: | CCTTCGATAGCTCAGCTGGTAGAGCGGAGGA |
| | 67026223-67026311 (+) | CTGTAGGCGCGCGCCCGTGGCCATCCTTAGG |
| | | TCGCTGGTTCGATTCCGGCTCGAAGGA |
| 239 | Tyr_GTA_chr14: | CCTTCGATAGCTCAGCTGGTAGAGCGGAGGA |
| | 21121258-21121351 (−) | CTGTAGCCTGTAGAAACATTTGTGGACATCC |
| | | TTAGGTCGCTGGTTCGATTCCGGCTCGAAGG |
| | | A |
| 240 | Tyr_GTA_chr14: | CCTTCGATAGCTCAGCTGGTAGAGCGGAGGA |
| | 21131351-21131444 (−) | CTGTAGATTGTACAGACATTTGCGGACATCC |
| | | TTAGGTCGCTGGTTCGATTCCGGCTCGAAGG |
| | | A |
| 241 | Tyr_GTA_chr14: | CCTTCGATAGCTCAGCTGGTAGAGCGGAGGA |
| | 21151432-21151520 (+) | CTGTAGTACTTAATGTGTGGTCATCCTTAGGT |
| | | CGCTGGTTCGATTCCGGCTCGAAGGA |
| 242 | Tyr_GTA_chr6: | CCTTCGATAGCTCAGCTGGTAGAGCGGAGGA |
| | 26595102-26595190 (+) | CTGTAGGGGTTTGAATGTGGTCATCCTTAGGT |
| | | CGCTGGTTCGAATCCGGCTCGGAGGA |
| 243 | Tyr_GTA_chr14: | CCTTCGATAGCTCAGCTGGTAGAGCGGAGGA |
| | 21128117-21128210 (−) | CTGTAGACTGCGGAAACGTTTGTGGACATCC |
| | | TTAGGTCGCTGGTTCAATTCCGGCTCGAAGG |
| | | A |
| 244 | Tyr_GTA_chr6: | CTTTCGATAGCTCAGTTGGTAGAGCGGAGGA |
| | 26575798-26575887 (+) | CTGTAGGTTCATTAAACTAAGGCATCCTTAG |
| | | GTCGCTGGTTCGAATCCGGCTCGAAGGA |
| 245 | Tyr_GTA_chr8: | TCTTCAATAGCTCAGCTGGTAGAGCGGAGGA |
| | 66609532-66609619 (−) | CTGTAGGTGCACGCCCGTGGCCATTCTTAGG |
| | | TGCTGGTTTGATTCCGACTTGGAGAG |
| 246 | Val_AAC_chr3: | GTTTCCGTAGTGTAGTGGTTATCACGTTCGCC |
| | 169490018-169490090 (+) | TAACACGCGAAAGGTCCCCGGTTCGAAACCG |
| | | GGCGGAAACA |
| 247 | Val_AAC_chr5: | GTTTCCGTAGTGTAGTGGTCATCACGTTCGCC |
| | 180615416-180615488 (−) | TAACACGCGAAAGGTCCCCGGTTCGAAACCG |
| | | GGCGGAAACA |
| 248 | Val_AAC_chr6: | GTTTCCGTAGTGTAGTGGTTATCACGTTCGCC |
| | 27618707-27618779 (−) | TAACACGCGAAAGGTCCCTGGATCAAAACCA |
| | | GGCGGAAACA |
| 249 | Val_AAC_chr6: | GTTTCCGTAGTGTAGTGGTTATCACGTTCGCC |
| | 27648885-27648957 (−) | TAACACGCGAAAGGTCCGCGGTTCGAAACCG |
| | | GGCGGAAACA |
| 250 | Val_AAC_chr6: | GTTTCCGTAGTGTAGTGGTTATCACGTTTGCC |
| | 27203288-27203360 (+) | TAACACGCGAAAGGTCCCCGGTTCGAAACCG |
| | | GGCAGAAACA |
| 251 | Val_AAC_chr6: | GGGGGTGTAGCTCAGTGGTAGAGCGTATGCT |
| | 28703206-28703277 (−) | TAACATTCATGAGGCTCTGGGTTCGATCCCC |
| | | AGCACTTCCA |
| 252 | Val_CAC_chr1: | GTTTCCGTAGTGTAGTGGTTATCACGTTCGCC |
| | 161369490-161369562 (−) | TCACACGCGAAAGGTCCCCGGTTCGAAACCG |
| | | GGCGGAAACA |
| 253 | Val_CAC_chr6: | GCTTCTGTAGTGTAGTGGTTATCACGTTCGCC |
| | 27248049-27248121 (−) | TCACACGCGAAAGGTCCCCGGTTCGAAACCG |
| | | GGCAGAAGCA |
| 254 | Val_CAC_chr19: | GTTTCCGTAGTGTAGCGGTTATCACATTCGCC |
| | 4724647-4724719 (−) | TCACACGCGAAAGGTCCCCGGTTCGATCCCG |
| | | GGCGGAAACA |
| 255 | Val_CAC_chr1: | GTTTCCGTAGTGTAGTGGTTATCACGTTCGCC |
| | 814929555-149298627 (−) | TCACACGCGAAAGGTCCCCGGTTCGAAACTG |
| | | GGCGGAAACA |
| 256 | Val_CAC_chr1: | GTTTCCGTAGTGTAGTGGTTATCACGTTCGCC |
| | 414968088-149684161 (−) | TCACACGCGTAAAGGTCCCCGGTTCGAAACC |
| | | GGGCGGAAACA |
| 257 | Val_CAC_chr6: | GTTTCCGTAGTGGAGTGGTTATCACGTTCGCC |
| | 27173867-27173939 (−) | TCACACGCGAAAGGTCCCCGGTTTGAAACCA |
| | | GGCGGAAACA |
| 258 | Val_TAC_chr11: | GGTTCCATAGTGTAGTGGTTATCACGTCTGCT |
| | 59318102-59318174 (−) | TTACACGCAGAAGGTCCTGGGTTCGAGCCCC |
| | | AGTGGAACCA |
| 259 | Val_TAC_chr11: | GGTTCCATAGTGTAGCGGTTATCACGTCTGCT |
| | 59318460-59318532 (−) | TTACACGCAGAAGGTCCTGGGTTCGAGCCCC |
| | | AGTGGAACCA |
| 260 | Val_TAC_chr10: | GGTTCCATAGTGTAGTGGTTATCACATCTGCT |
| | 5895674-5895746 (−) | TTACACGCAGAAGGTCCTGGGTTCAAGCCCC |
| | | AGTGGAACCA |
| 261 | Val_TAC_chr6: | GTTTCCGTGGTGTAGTGGTTATCACATTCGCC |
| | 27258405-27258477 (+) | TTACACGCGAAAGGTCCTCGGGTCGAAACCG |
| | | AGCGGAAACA |
| 262 | iMet_CAT_chr1: | AGCAGAGTGGCGCAGCGGAAGCGTGCTGGG |
| | 153643726-153643797 (+) | CCCATAACCCAGAGGTCGATGGATCGAAACC |
| | | ATCCTCTGCTA |
| 263 | iMet_CAT_chr6: | AGCAGAGTGGCGCAGCGGAAGCGTGCTGGG |
| | 27745664-27745735 (+) | CCCATAACCCAGAGGTCGATGGATCTAAACC |
| | | ATCCTCTGCTA |
| 264 | Glu_TTC_chr1: | TCCCTGGTGGTCTAGTGGCTAGGATTCGGCG |
| | 16861773-16861845 (−) | CTTTCACCGCCGCGGCCCGGGTTCGATTCCCG |
| | | GTCAGGGAAT |
| 265 | Gly_CCC_chr1: | GCGTTGGTGGTTTAGTGGTAGAATTCTCGCCT |
| | 17004765-17004836 (−) | CCCATGCGGGAGACCCGGGTTCAATTCCCGG |
| | | CCACTGCAC |
| 266 | Gly_CCC_chr1: | GGCCTTGGTGGTGCAGTGGTAGAATTCTCGC |
| | 17053779-17053850 (+) | CTCCCACGTGGGAGACCCGGGTTCAATTCCC |
| | | GGCCAATGCA |
| 267 | Glu_TTC_chr1: | GTCCCTGGTGGTCTAGTGGCTAGGATTCGGC |
| | 17199077-17199149 (+) | GCTTTCACCGCCGCGGCCCGGGTTCGATTCCC |
| | | GGCCAGGGAA |
| 268 | Asn_GTT_chr1: | TGTCTCTGTGGCGCAATCGGTTAGCGCGTTCG |
| | 17216171-17216245 (+) | GCTGTTAACCGAAAGATTGGTGGTTCGAGCC |
| | | CACCCAGGGACG |
| 269 | Arg_TCT_chr1: | TGGCTCCGTGGCGCAATGGATAGCGCATTGG |
| | 94313128-94313213 (+) | ACTTCTAGAGGCTGAAGGCATTCAAAGGTTC |
| | | CGGGTTCGAGTCCCGGCGGAGTCG |
| 270 | Lys_CTT_chr1: | GCCCGGCTAGCTCAGTCGGTAGAGCATGAGA |
| | 514539521-145395594 (−) | CTCTTAATCTCAGGGTCGTGGGTTCGAGCCCC |
| | | ACGTTGGGCGC |
| 271 | His_GTG_chr1: | GCCGTGATCGTATAGTGGTTAGTACTCTGCGT |
| | 145396880-145396952 (−) | TGTGGCCGCAGCAACCTCGGTTCGAATCCGA |
| | | GTCACGGCAG |
| 272 | Gly_TCC_chr1: | GCGTTGGTGGTATAGTGGTGAGCATAGCTGC |
| | 145397863-145397935 (−) | CTTCCAAGCAGTTGACCCGGGTTCGATTCCC |
| | | GGCCAACGCAG |
| 273 | Glu_CTC_chr1: | TCCCTGGTGGTCTAGTGGTTAGGATTCGGCG |
| | 145399232-145399304 (−) | CTCTCACCGCCGCGGCCCGGGTTCGATTCCC |
| | | GGTCAGGGAAA |
| 274 | Gln_CTG_chr1: | AGGTTCCATGGTGTAATGGTGAGCACTCTGG |
| | 145963303-145963375 (+) | ACTCTGAATCCAGCGATCCGAGTTCGAGTCT |
| | | CGGTGGAACCT |
| 275 | Asn_GTT_chr1: | TGTCTCTGTGGCGTAGTCGGTTAGCGCGTTCG |
| | 148000804-148000878 (+) | GCTGTTAACCGAAAAGTTGGTGGTTCGAGCC |
| | | CACCCAGGAACG |
| 276 | Asn_GTT_chr1: | TGTCTCTGTGGCGCAATCGGTTAGCGCGTTCG |
| | 148248114-148248188 (+) | GCTGTTAACCGAAAGGTTGGTGGTTCGAGCC |
| | | CACCCAGGGACG |
| 277 | Asn_GTT_chr1: | GTCTCTGTGGCGCAATCGGTTAGCGCATTCG |
| | 148598313-148598387 (−) | GCTGTTAACCGAAAGGTTGGTGGTTCGAGCC |
| | | CACCCAGGGACGC |
| 278 | Asn_GTT_chr1: | GTCTCTGTGGCGCAATGGGTTAGCGCGTTCG |
| | 149230569-149230643 (−) | GCTGTTAACCGAAAGGTTGGTGGTTCGAGCC |
| | | CATCCAGGGACGC |
| 279 | Val_CAC_chr1: | GCACTGGTGGTTCAGTGGTAGAATTCTCGCC |
| | 149294665-149294736 (−) | TCACACGCGGGACACCCGGGTTCAATTCCCG |
| | | GTCAAGGCAA |
| 280 | Val_CAC_chr1: | GTTTCCGTAGTGTAGTGGTTATCACGTTCGCC |
| | 149298554-149298627 (−) | TCACACGCGAAAGGTCCCCGGTTCGAAACTG |
| | | GGCGGAAACAG |
| 281 | Gly_CCC_chr1: | GCACTGGTGGTTCAGTGGTAGAATTCTCGCC |
| | 149680209-149680280 (−) | TCCCACGCGGGAGACCCGGGTTTAATTCCCG |
| | | GTCAAGATAA |
| 282 | Val_CAC_chr1: | GTTTCCGTAGTGTAGTGGTTATCACGTTCGCC |
| | 149684087-149684161 (−) | TCACACGCGTAAAGGTCCCCGGTTCGAAACC |
| | | GGGCGGAAACAT |
| 283 | Met_CAT_chr1: | TAGCAGAGTGGCGCAGCGGAAGCGTGCTGG |
| | 153643725-153643797 (+) | GCCCATAACCCAGAGGTCGATGGATCGAAAC |
| | | CATCCTCTGCTA |
| 284 | Val_CAC_chr1: | GTTTCCGTAGTGTAGTGGTTATCACGTTCGCC |
| | 161369489-161369562 (−) | TCACACGCGAAAGGTCCCCGGTTCGAAACCG |
| | | GGCGGAAACAA |
| 285 | Asp_GTC_chr1: | TCCTCGTTAGTATAGTGGTGAGTATCCCCGCC |
| | 161410614-161410686 (−) | TGTCACGCGGGAGACCGGGGTTCGATTCCCC |
| | | GACGGGGAGG |
| 286 | Gly_GCC_chr1: | TGCATGGGTGGTTCAGTGGTAGAATTCTCGC |
| | 161413093-161413164 (+) | CTGCCACGCGGGAGGCCCGGGTTCGATTCCC |
| | | GGCCCATGCA |
| 287 | Glu_CTC_chr1: | TCCCTGGTGGTCTAGTGGTTAGGATTCGGCG |
| | 161417017-161417089 (−) | CTCTCACCGCCGCGGCCCGGGTTCGATTCCC |
| | | GGTCAGGGAAG |
| 288 | Asp_GTC_chr1: | ATCCTTGTTACTATAGTGGTGAGTATCTCTGC |
| | 161492934-161493006 (+) | CTGTCATGCGTGAGAGAGGGGGTCGATTCCC |
| | | CGACGGGGAG |
| 289 | Gly_GCC_chr1: | GCATTGGTGGTTCAGTGGTAGAATTCTCGCCT |
| | 161493636-161493707 (−) | GCCACGCGGGAGGCCCGGGTTCGATTCCCGG |
| | | CCAATGCAC |
| 290 | Leu_CAG_chr1: | GTCAGGATGGCCGAGCGGTCTAAGGCGCTGC |
| | 161500131-161500214 (−) | GTTCAGGTCGCAGTCTCCCCTGGAGGCGTGG |
| | | GTTCGAATCCCACTCCTGACAA |
| 291 | Gly_TCC_chr1: | CGCGTTGGTGGTATAGTGGTGAGCATAGCTG |
| | 161500902-161500974 (+) | CCTTCCAAGCAGTTGACCCGGGTTCGATTCCC |
| | | GGCCAACGCA |
| 292 | Asn_GTT_chr1: | CGTCTCTGTGGCGCAATCGGTTAGCGCGTTC |
| | 161510030-161510104 (+) | GGCTGTTAACCGAAAGGTTGGTGGTTCGATC |
| | | CCACCCAGGGACG |
| 293 | Glu_TTC_chr1: | CGCGTTGGTGGTGTAGTGGTGAGCACAGCTG |
| | 161582507-161582579 (+) | CCTTTCAAGCAGTTAACGCGGGTTCGATTCCC |
| | | GGGTAACGAA |
| 294 | Pro_CGG_chr1: | CGGCTCGTTGGTCTAGGGGTATGATTCTCGCT |
| | 167683961-167684033 (+) | TCGGGTGCGAGAGGTCCCGGGTTCAAATCCC |
| | | GGACGAGCCC |
| 295 | Pro_AGG_chr1: | GGCTCGTTGGTCTAGGGGTATGATTCTCGCTT |
| | 167684724-167684796 (−) | AGGGTGCGAGAGGTCCCGGGTTCAAATCCCG |
| | | GACGAGCCCT |
| 296 | Lys_TTT_chr1: | CGCCCGGATAGCTCAGTCGGTAGAGCATCAG |
| | 204475654-204475727 (+) | ACTTTTAATCTGAGGGTCCAGGGTTCAAGTC |
| | | CCTGTTCGGGCG |
| 297 | Lys_TTT_chr1: | GCCCGGATAGCTCAGTCGGTAGAGCATCAGA |
| | 204476157-204476230 (−) | CTTTTAATCTGAGGGTCCAGGGTTCAAGTCCC |
| | | TGTTCGGGCGT |
| 298 | Leu_CAA_chr1: | TGTCAGGATGGCCGAGTGGTCTAAGGCGCCA |
| | 249168053-249168159 (+) | GACTCAAGGTAAGCACCTTGCCTGCGGGCTT |
| | | TCTGGTCTCCGGATGGAGGCGTGGGTTCGAA |
| | | TCCCACTTCTGACA |
| 299 | Glu_CTC_chr1: | TTCCCTGGTGGTCTAGTGGTTAGGATTCGGCG |
| | 249168446-249168518 (+) | CTCTCACCGCCGCGGCCCGGGTTCGATTCCC |
| | | GGTCAGGAAA |
| 300 | Tyr_GTA_chr2: | GCCTTCGATAGCTCAGTTGGTAGAGCGGAGG |
| | 27273649-27273738 (+) | ACTGTAGTGGATAGGGCGTGGCAATCCTTAG |
| | | GTCGCTGGTTCGATTCCGGCTCGAAGGA |
| 301 | Ala_AGC_chr2: | CGGGGGATTAGCTCAAATGGTAGAGCGCTCG |
| | 27274081-27274154 (+) | CTTAGCATGCGAGAGGTAGCGGGATCGATGC |
| | | CCGCATCCTCCA |
| 302 | Ile_TAT_chr2: | AGCTCCAGTGGCGCAATCGGTTAGCGCGCGG |
| | 43037675-43037768 (+) | TACTTATACAGCAGTACATGCAGAGCAATGC |
| | | CGAGGTTGTGAGTTCGAGCCTCACCTGGAGC |
| | | A |
| 303 | Gly_CCC_chr2: | GCGCCGCTGGTGTAGTGGTATCATGCAAGAT |
| | 70476122-70476193 (−) | TCCCATTCTTGCGACCCGGGTTCGATTCCCGG |
| | | GCGGCGCAT |
| 304 | Glu_TTC_chr2: | TCCCATATGGTCTAGCGGTTAGGATTCCTGGT |
| | 131094700-131094772 (−) | TTTCACCCAGGTGGCCCGGGTTCGACTCCCG |
| | | GTATGGGAAC |
| 305 | Ala_CGC_chr2: | GGGGGATGTAGCTCAGTGGTAGAGCGCGCGC |
| | 157257280-157257352 (+) | TTCGCATGTGTGAGGTCCCGGGTTCAATCCCC |
| | | GGCATCTCCA |
| 306 | Gly_GCC_chr2: | GCATTGGTGGTTCAGTGGTAGAATTCTCGCCT |
| | 157257658-157257729 (−) | GCCACGCGGGAGGCCCGGGTTCGATTCCCGG |
| | | CCAATGCAA |
| 307 | Arg_ACG_chr3: | GGGCCAGTGGCGCAATGGATAACGCGTCTGA |
| | 45730490-45730563 (−) | CTACGGATCAGAAGATTCTAGGTTCGACTCC |
| | | TGGCTGGCTCGC |
| 308 | Val_AAC_chr3: | GGTTTCCGTAGTGTAGTGGTTATCACGTTCGC |
| | 169490017-169490090 (+) | CTAACACGCGAAAGGTCCCCGGTTCGAAACC |
| | | GGGCGGAAACA |
| 309 | Val_AAC_chr5: | AGTTTCCGTAGTGTAGTGGTTATCACGTTCGC |
| | 180596609-180596682 (+) | CTAACACGCGAAAGGTCCCCGGTTCGAAACC |
| | | GGGCGGAAACA |
| 310 | Leu_AAG_chr5: | AGGTAGCGTGGCCGAGCGGTCTAAGGCGCTG |
| | 180614700-180614782 (+) | GATTAAGGCTCCAGTCTCTTCGGGGGCGTGG |
| | | GTTCGAATCCCACCGCTGCCA |
| 311 | Val_AAC_chr5: | GTTTCCGTAGTGTAGTGGTCATCACGTTCGCC |
| | 180615415-180615488 (−) | TAACACGCGAAAGGTCCCCGGTTCGAAACCG |
| | | GGCGGAAACAT |
| 312 | Pro_TGG_chr5: | GGCTCGTTGGTCTAGGGGTATGATTCTCGCTT |
| | 180615853-180615925 (−) | TGGGTGCGAGAGGTCCCGGGTTCAAATCCCG |
| | | GACGAGCCCA |
| 313 | Thr_TGT_chr5: | GGCTCCATAGCTCAGGGGTTAGAGCACTGGT |
| | 180618686-180618758 (−) | CTTGTAAACCAGGGTCGCGAGTTCAAATCTC |
| | | GCTGGGGCCTG |
| 314 | Ala_TGC_chr5: | TGGGGATGTAGCTCAGTGGTAGAGCGCATGC |
| | 180633867-180633939 (+) | TTTGCATGTATGAGGCCCCGGGTTCGATCCCC |
| | | GGCATCTCCA |
| 315 | Lys_CTT_chr5: | CGCCCGGCTAGCTCAGTCGGTAGAGCATGAG |
| | 180634754-180634827 (+) | ACTCTTAATCTCAGGGTCGTGGGTTCGAGCC |
| | | CCACGTTGGGCG |
| 316 | Val_AAC_chr5: | GTTTCCGTAGTGTAGTGGTTATCACGTTCGCC |
| | 180645269-180645342 (−) | TAACACGCGAAAGGTCCCCGGTTCGAAACCG |
| | | GGCGGAAACAA |
| 317 | Lys_CTT_chr5: | GCCCGGCTAGCTCAGTCGGTAGAGCATGAGA |
| | 180648978-180649051 (−) | CTCTTAATCTCAGGGTCGTGGGTTCGAGCCCC |
| | | ACGTTGGGCGT |
| 318 | Val_CAC_chr5: | GTTTCCGTAGTGTAGTGGTTATCACGTTCGCC |
| | 180649394-180649467 (−) | TCACACGCGAAAGGTCCCCGGTTCGAAACCG |
| | | GGCGGAAACAC |
| 319 | Met_CAT_chr6: | CAGCAGAGTGGCGCAGCGGAAGCGTGCTGG |
| | 26286753-26286825 (+) | GCCCATAACCCAGAGGTCGATGGATCGAAAC |
| | | CATCCTCTGCTA |
| 320 | Ser_GCT_chr6: | GGAGAGGCCTGGCCGAGTGGTTAAGGCGATG |
| | 26305717-26305801 (−) | GACTGCTAATCCATTGTGCTCTGCACGCGTG |
| | | GGTTCGAATCCCATCCTCGTCGC |
| 321 | Gln_TTG_chr6: | GGCCCCATGGTGTAATGGTTAGCACTCTGGA |
| | 26311423-26311495 (−) | CTTTGAATCCAGCGATCCGAGTTCAAATCTC |
| | | GGTGGGACCTG |
| 322 | Gln_TTG_chr6: | GGCCCCATGGTGTAATGGTTAGCACTCTGGA |
| | 26311974-26312046 (−) | CTTTGAATCCAGCGATCCGAGTTCAAATCTC |
| | | GGTGGGACCTA |
| 323 | Ser_TGA_chr6: | GTAGTCGTGGCCGAGTGGTTAAGGCGATGGA |
| | 26312823-26312905 (−) | CTTGAAATCCATTGGGGTCTCCCCGCGCAGG |
| | | TTCGAATCCTGCCGACTACGG |
| 324 | Met_CAT_chr6: | AGCAGAGTGGCGCAGCGGAAGCGTGCTGGG |
| | 26313351-26313423 (−) | CCCATAACCCAGAGGTCGATGGATCGAAACC |
| | | ATCCTCTGCTAT |
| 325 | Arg_TCG_chr6: | GGACCACGTGGCCTAATGGATAAGGCGTCTG |
| | 26323045-26323118 (+) | ACTTCGGATCAGAAGATTGAGGGTTCGAATC |
| | | CCTCCGTGGTTA |
| 326 | Ser_AGA_chr6: | TGTAGTCGTGGCCGAGTGGTTAAGGCGATGG |
| | 26327816-26327898 (+) | ACTAGAAATCCATTGGGGTCTCCCCGCGCAG |
| | | GTTCGAATCCTGCCGACTACG |
| 327 | Met_CAT_chr6: | AGCAGAGTGGCGCAGCGGAAGCGTGCTGGG |
| | 26330528-26330600 (−) | CCCATAACCCAGAGGTCGATGGATCGAAACC |
| | | ATCCTCTGCTAG |
| 328 | Leu_CAG_chr6: | CGTCAGGATGGCCGAGCGGTCTAAGGCGCTG |
| | 26521435-26521518 (+) | CGTTCAGGTCGCAGTCTCCCCTGGAGGCGTG |
| | | GGTTCGAATCCCACTCCTGACA |
| 329 | Thr_AGT_chr6: | GGCTCCGTGGCTTAGCTGGTTAAAGCGCCTG |
| | 26533144-26533218 (−) | TCTAGTAAACAGGAGATCCTGGGTTCGAATC |
| | | CCAGCGGGGCCTG |
| 330 | Arg_ACG_chr6: | AGGGCCAGTGGCGCAATGGATAACGCGTCTG |
| | 26537725-26537798 (+) | ACTACGGATCAGAAGATTCCAGGTTCGACTC |
| | | CTGGCTGGCTCG |
| 331 | Val_CAC_chr6: | GGTTTCCGTAGTGTAGTGGTTATCACGTTCGC |
| | 26538281-26538354 (+) | CTCACACGCGAAAGGTCCCCGGTTCGAAACC |
| | | GGGCGGAAACA |
| 332 | Ala_CGC_chr6: | AGGGGATGTAGCTCAGTGGTAGAGCGCATGC |
| | 26553730-26553802 (+) | TTCGCATGTATGAGGTCCCGGGTTCGATCCCC |
| | | GGCATCTCCA |
| 333 | Ile_AAT_chr6: | TGGCCGGTTAGCTCAGTTGGTTAGAGCGTGG |
| | 26554349-26554423 (+) | TGCTAATAACGCCAAGGTCGCGGGTTCGATC |
| | | CCCGTACGGGCCA |
| 334 | Pro_AGG_chr6: | CGGCTCGTTGGTCTAGGGGTATGATTCTCGCT |
| | 26555497-26555569 (+) | TAGGGTGCGAGAGGTCCCGGGTTCAAATCCC |
| | | GGACGAGCCC |
| 335 | Lys_CTT_chr6: | AGCCCGGCTAGCTCAGTCGGTAGAGCATGAG |
| | 26556773-26556846 (+) | ACTCTTAATCTCAGGGTCGTGGGTTCGAGCC |
| | | CCACGTTGGGCG |
| 336 | Tyr_GTA_chr6: | TCCTTCGATAGCTCAGTTGGTAGAGCGGAGG |
| | 26569085-26569176 (+) | ACTGTAGTTGGCTGTGTCCTTAGACATCCTTA |
| | | GGTCGCTGGTTCGAATCCGGCTCGAAGGA |
| 337 | Ala_AGC_chr6: | GGGGAATTAGCTCAAATGGTAGAGCGCTCGC |
| | 26572091-26572164 (−) | TTAGCATGCGAGAGGTAGCGGGATCGATGCC |
| | | CGCATTCTCCAG |
| 338 | Met_CAT_chr6: | CGCCCTCTTAGCGCAGCGGGCAGCGCGTCAG |
| | 26766443-26766516 (+) | TCTCATAATCTGAAGGTCCTGAGTTCGAGCCT |
| | | CAGAGAGGGCA |
| 339 | Ile_TAT_chr6: | TGCTCCAGTGGCGCAATCGGTTAGCGCGCGG |
| | 26988124-26988218 (+) | TACTTATATGGCAGTATGTGTGCGAGTGATG |
| | | CCGAGGTTGTGAGTTCGAGCCTCACCTGGAG |
| | | CA |
| 340 | His_GTG_chr6: | TGCCGTGATCGTATAGTGGTTAGTACTCTGCG |
| | 27125905-27125977 (+) | TTGTGGCCGCAGCAACCTCGGTTCGAATCCG |
| | | AGTCACGGCA |
| 341 | Ile_AAT_chr6: | GGCCGGTTAGCTCAGTTGGTTAGAGCGTGGT |
| | 27144993-27145067 (−) | GCTAATAACGCCAAGGTCGCGGGTTCGATCC |
| | | CCGTACGGGCCAC |
| 342 | Val_AAC_chr6: | AGTTTCCGTAGTGTAGTGGTTATCACGTTTGC |
| | 27203287-27203360 (+) | CTAACACGCGAAAGGTCCCCGGTTCGAAACC |
| | | GGGCAGAAACA |
| 343 | Val_CAC_chr6: | GCTTCTGTAGTGTAGTGGTTATCACGTTCGCC |
| | 27248048-27248121 (−) | TCACACGCGAAAGGTCCCCGGTTCGAAACCG |
| | | GGCAGAAGCAA |
| 344 | Asp_GTC_chr6: | TTCCTCGTTAGTATAGTGGTGAGTATCCCCGC |
| | 27447452-27447524 (+) | CTGTCACGCGGGAGACCGGGGTTCGATTCCC |
| | | CGACGGGGAG |
| 345 | Ser_TGA_chr6: | GTAGTCGTGGCCGAGTGGTTAAGGCGATGGA |
| | 27473606-27473688 (−) | CTTGAAATCCATTGGGGTTTCCCCGCGCAGG |
| | | TTCGAATCCTGTCGGCTACGG |
| 346 | Gln_CTG_chr6: | AGGTTCCATGGTGTAATGGTTAGCACTCTGG |
| | 27487307-27487379 (+) | ACTCTGAATCCAGCGATCCGAGTTCAAATCT |
| | | CGGTGGAACCT |
| 347 | Asp_GTC_chr6: | TCCTCGTTAGTATAGTGGTGAGTGTCCCCGTC |
| | 27551235-27551307 (−) | TGTCACGCGGGAGACCGGGGTTCGATTCCCC |
| | | GACGGGGAGA |
| 348 | Val_AAC_chr6: | GTTTCCGTAGTGTAGTGGTTATCACGTTCGCC |
| | 27618706-27618779 (−) | TAACACGCGAAAGGTCCCTGGATCAAAACCA |
| | | GGCGGAAACAA |
| 349 | Ile_AAT_chr6: | CGGCCGGTTAGCTCAGTTGGTTAGAGCGTGG |
| | 27655966-27656040 (+) | TGCTAATAACGCCAAGGTCGCGGGTTCGATC |
| | | CCCGTACTGGCCA |
| 350 | Gln_CTG_chr6: | GGCCCCATGGTGTAATGGTCAGCACTCTGGA |
| | 27759134-27759206 (−) | CTCTGAATCCAGCGATCCGAGTTCAAATCTC |
| | | GGTGGGACCCA |
| 351 | Gln_TTG_chr6: | GGCCCCATGGTGTAATGGTTAGCACTCTGGA |
| | 27763639-27763711 (−) | CTTTGAATCCAGCGATCCGAGTTCAAATCTC |
| | | GGTGGGACCTT |
| 352 | Ala_AGC_chr6: | TGGGGGTGTAGCTCAGTGGTAGAGCGCGTGC |
| | 28574932-28575004 (+) | TTAGCATGTACGAGGTCCCGGGTTCAATCCC |
| | | CGGCACCTCCA |
| 353 | Ala_AGC_chr6: | GGGGATGTAGCTCAGTGGTAGAGCGCATGCT |
| | 28626013-28626085 (−) | TAGCATGCATGAGGTCCCGGGTTCGATCCCC |
| | | AGCATCTCCAG |
| 354 | Ala_CGC_chr6: | AGGGGGTGTAGCTCAGTGGTAGAGCGCGTGC |
| | 28697091-28697163 (+) | TTCGCATGTACGAGGCCCCGGGTTCGACCCC |
| | | CGGCTCCTCCA |
| 355 | Ala_AGC_chr6: | GGGGGTGTAGCTCAGTGGTAGAGCGCGTGCT |
| | 28806220-28806292 (−) | TAGCATGCACGAGGCCCCGGGTTCAATCCCC |
| | | GGCACCTCCAT |
| 356 | Ala_AGC_chr6: | GGGGGTGTAGCTCAGTGGTAGAGCGCGTGCT |
| | 28831461-28831533 (−) | TAGCATGCACGAGGCCCCGGGTTCAATCCCC |
| | | GGCACCTCCAG |
| 357 | Leu_CAA_chr6: | GTCAGGATGGCCGAGTGGTCTAAGGCGCCAG |
| | 28863999-28864105 (−) | ACTCAAGCTAAGCTTCCTCCGCGGTGGGGAT |
| | | TCTGGTCTCCAATGGAGGCGTGGGTTCGAAT |
| | | CCCACTTCTGACAC |
| 358 | Leu_CAA_chr6: | TGTCAGGATGGCCGAGTGGTCTAAGGCGCCA |
| | 28908829-28908934 (+) | GACTCAAGCTTGGCTTCCTCGTGTTGAGGATT |
| | | CTGGTCTCCAATGGAGGCGTGGGTTCGAATC |
| | | CCACTTCTGACA |
| 359 | Gln_CTG_chr6: | GGTTCCATGGTGTAATGGTTAGCACTCTGGA |
| | 28909377-28909449 (−) | CTCTGAATCCAGCGATCCGAGTTCAAATCTC |
| | | GGTGGAACCTT |
| 360 | Leu_AAG_chr6: | GGTAGCGTGGCCGAGCGGTCTAAGGCGCTGG |
| | 28911398-28911480 (−) | ATTAAGGCTCCAGTCTCTTCGGGGGCGTGGG |
| | | TTCGAATCCCACCGCTGCCAG |
| 361 | Met_CAT_chr6: | TGCCTCCTTAGCGCAGTAGGCAGCGCGTCAG |
| | 28912351-28912424 (+) | TCTCATAATCTGAAGGTCCTGAGTTCGAACCT |
| | | CAGAGGGGGCA |
| 362 | Lys_TTT_chr6: | AGCCCGGATAGCTCAGTCGGTAGAGCATCAG |
| | 28918805-28918878 (+) | ACTTTTAATCTGAGGGTCCAGGGTTCAAGTC |
| | | CCTGTTCGGGCG |
| 363 | Met_CAT_chr6: | GCCTCCTTAGCGCAGTAGGCAGCGCGTCAGT |
| | 28921041-28921114 (−) | CTCATAATCTGAAGGTCCTGAGTTCGAACCT |
| | | CAGAGGGGGCAG |
| 364 | Glu_CTC_chr6: | TTCCCTGGTGGTCTAGTGGTTAGGATTCGGCG |
| | 28949975-28950047 (+) | CTCTCACCGCCGCGGCCCGGGTTCGATTCCC |
| | | GGTCAGGGAA |
| 365 | Leu_TAA_chr6: | CACCAGGATGGCCGAGTGGTTAAGGCGTTGG |
| | 144537683-144537766 (+) | ACTTAAGATCCAATGGACATATGTCCGCGTG |
| | | GGTTCGAACCCCACTCCTGGTA |
| 366 | Pro_AGG_chr7: | TGGCTCGTTGGTCTAGGGGTATGATTCTCGCT |
| | 128423503-128423575 (+) | TAGGGTGCGAGAGGTCCCGGGTTCAAATCCC |
| | | GGACGAGCCC |
| 367 | Arg_CCT_chr7: | AGCCCCAGTGGCCTAATGGATAAGGCATTGG |
| | 139025445-139025518 (+) | CCTCCTAAGCCAGGGATTGTGGGTTCGAGTC |
| | | CCATCTGGGGTG |
| 368 | Cys_GCA_chr7: | GGGGATATAGCTCAGGGGTAGAGCATTTGAC |
| | 149388271-149388343 (−) | TGCAGATCAAGAGGTCCCCGGTTCAAATCCG |
| | | GGTGCCCCCCC |
| 369 | Tyr_GTA_chr8: | CCCTTCGATAGCTCAGCTGGTAGAGCGGAGG |
| | 67025601-67025694 (+) | ACTGTAGCTACTTCCTCAGCAGGAGACATCC |
| | | TTAGGTCGCTGGTTCGATTCCGGCTCGAAGG |
| | | A |
| 370 | Tyr_GTA_chr8: | CCCTTCGATAGCTCAGCTGGTAGAGCGGAGG |
| | 67026222-67026311 (+) | ACTGTAGGCGCGCGCCCGTGGCCATCCTTAG |
| | | GTCGCTGGTTCGATTCCGGCTCGAAGGA |
| 371 | Ala_AGC_chr8: | TGGGGGATTAGCTCAAATGGTAGAGCGCTCG |
| | 67026423-67026496 (+) | CTTAGCATGCGAGAGGTAGCGGGATCGATGC |
| | | CCGCATCCTCCA |
| 372 | Ser_AGA_chr8: | GTAGTCGTGGCCGAGTGGTTAAGGCGATGGA |
| | 96281884-96281966 (−) | CTAGAAATCCATTGGGGTCTCCCCGCGCAGG |
| | | TTCGAATCCTGCCGACTACGG |
| 373 | Met_CAT_chr8: | GCCTCGTTAGCGCAGTAGGTAGCGCGTCAGT |
| | 124169469-124169542 (−) | CTCATAATCTGAAGGTCGTGAGTTCGATCCTC |
| | | ACACGGGGCAC |
| 374 | Arg_TCT_chr9: | GGCTCTGTGGCGCAATGGATAGCGCATTGGA |
| | 131102354-131102445 (−) | CTTCTAGCTGAGCCTAGTGTGGTCATTCAAA |
| | | GGTTGTGGGTTCGAGTCCCACCAGAGTCGA |
| 375 | Asn_GTT_chr10: | GTCTCTGTGGCGCAATCGGTTAGCGCGTTCG |
| | 22518437-22518511 (−) | GCTGTTAACCGAAAGGTTGGTGGTTCGAGCC |
| | | CACCCAGGGACGC |
| 376 | Ser_TGA_chr10: | GGCAGCGATGGCCGAGTGGTTAAGGCGTTGG |
| | 69524260-69524342 (+) | ACTTGAAATCCAATGGGGTCTCCCCGCGCAG |
| | | GTTCGAACCCTGCTCGCTGCG |
| 377 | Val_TAC_chr11: | GGTTCCATAGTGTAGTGGTTATCACGTCTGCT |
| | 59318101-59318174 (−) | TTACACGCAGAAGGTCCTGGGTTCGAGCCCC |
| | | AGTGGAACCAT |
| 378 | Val_TAC_chr11: | GGTTCCATAGTGTAGCGGTTATCACGTCTGCT |
| | 59318459-59318532 (−) | TTACACGCAGAAGGTCCTGGGTTCGAGCCCC |
| | | AGTGGAACCAC |
| 379 | Arg_TCT_chr11: | TGGCTCTGTGGCGCAATGGATAGCGCATTGG |
| | 59318766-59318852 (+) | ACTTCTAGATAGTTAGAGAAATTCAAAGGTT |
| | | GTGGGTTCGAGTCCCACCAGAGTCG |
| 380 | Leu_TAA_chr11: | TACCAGAATGGCCGAGTGGTTAAGGCGTTGG |
| | 59319227-59319310 (+) | ACTTAAGATCCAATGGATTCATATCCGCGTG |
| | | GGTTCGAACCCCACTTCTGGTA |
| 381 | Lys_TTT_chr11: | GGCCCGGATAGCTCAGTCGGTAGAGCATCAG |
| | 59323901-59323974 (+) | ACTTTTAATCTGAGGGTCCGGGGTTCAAGTC |
| | | CCTGTTCGGGCG |
| 382 | Phe_GAA_chr11: | GCCGAAATAGCTCAGTTGGGAGAGCGTTAGA |
| | 59324969-59325042 (−) | CTGAAGATCTAAAGGTCCCTGGTTCGATCCC |
| | | GGGTTTCGGCAG |
| 383 | Lys_TTT_chr11: | GCCCGGATAGCTCAGTCGGTAGAGCATCAGA |
| | 59327807-59327880 (−) | CTTTTAATCTGAGGGTCCAGGGTTCAAGTCCC |
| | | TGTTCGGGCGG |
| 384 | Phe_GAA_chr11: | GCCGAAATAGCTCAGTTGGGAGAGCGTTAGA |
| | 59333852-59333925 (−) | CTGAAGATCTAAAGGTCCCTGGTTCAATCCC |
| | | GGGTTTCGGCAG |
| 385 | Ser_GCT_chr11: | GGACGAGGTGGCCGAGTGGTTAAGGCGATG |
| | 66115590-66115672 (+) | GACTGCTAATCCATTGTGCTTTGCACGCGTGG |
| | | GTTCGAATCCCATCCTCGTCG |
| 386 | Pro_TGG_chr11: | GGCTCGTTGGTCTAGGGGTATGATTCTCGGTT |
| | 75946868-75946940 (−) | TGGGTCCGAGAGGTCCCGGGTTCAAATCCCG |
| | | GACGAGCCCC |
| 387 | Ser_CGA_chr12: | AGTCACGGTGGCCGAGTGGTTAAGGCGTTGG |
| | 56584147-56584229 (+) | ACTCGAAATCCAATGGGGTTTCCCCGCACAG |
| | | GTTCGAATCCTGTTCGTGACG |
| 388 | Asp_GTC_chr12: | CTCCTCGTTAGTATAGTGGTTAGTATCCCCGC |
| | 98897280-98897352 (+) | CTGTCACGCGGGAGACCGGGGTTCAATTCCC |
| | | CGACGGGGAG |
| 389 | Trp_CCA_chr12: | GGACCTCGTGGCGCAACGGTAGCGCGTCTGA |
| | 98898029-98898101 (+) | CTCCAGATCAGAAGGCTGCGTGTTCGAATCA |
| | | CGTCGGGGTCA |
| 390 | Ala_TGC_chr12: | GGGGATGTAGCTCAGTGGTAGAGCGCATGCT |
| | 125406300-125406372 (−) | TTGCATGTATGAGGCCCCGGGTTCGATCCCC |
| | | GGCATCTCCAT |
| 391 | Phe_GAA_chr12: | GCCGAAATAGCTCAGTTGGGAGAGCGTTAGA |
| | 125412388-125412461 (−) | CTGAAGATCTAAAGGTCCCTGGTTCGATCCC |
| | | GGGTTTCGGCAC |
| 392 | Ala_TGC_chr12: | AGGGGATGTAGCTCAGTGGTAGAGCGCATGC |
| | 125424511-125424583 (+) | TTTGCACGTATGAGGCCCCGGGTTCAATCCC |
| | | CGGCATCTCCA |
| 393 | Asn_GTT_chr13: | GTCTCTGTGGCGCAATCGGTTAGCGCGTTCG |
| | 31248100-31248174 (−) | GCTGTTAACCGAAAGGTTGGTGGTTCGAGCC |
| | | CACCCAGGGACGG |
| 394 | Glu_TTC_chr13: | TCCCACATGGTCTAGCGGTTAGGATTCCTGGT |
| | 45492061-45492133 (−) | TTTCACCCAGGCGGCCCGGGTTCGACTCCCG |
| | | GTGTGGGAAC |
| 395 | Thr_TGT_chr14: | GGCTCCATAGCTCAGGGGTTAGAGCGCTGGT |
| | 21081948-21082021 (−) | CTTGTAAACCAGGGGTCGCGAGTTCAATTCT |
| | | CGCTGGGGCCTG |
| 396 | Leu_TAG_chr14: | TGGTAGTGTGGCCGAGCGGTCTAAGGCGCTG |
| | 21093528-21093610 (+) | GATTTAGGCTCCAGTCTCTTCGGGGGCGTGG |
| | | GTTCGAATCCCACCACTGCCA |
| 397 | Thr_TGT_chr14: | GGCTCCATAGCTCAGGGGTTAGAGCACTGGT |
| | 21099318-21099391 (−) | CTTGTAAACCAGGGGTCGCGAGTTCAAATCT |
| | | CGCTGGGGCCTC |
| 398 | Pro_TGG_chr14: | TGGCTCGTTGGTCTAGTGGTATGATTCTCGCT |
| | 21101164-21101236 (+) | TTGGGTGCGAGAGGTCCCGGGTTCAAATCCC |
| | | GGACGAGCCC |
| 399 | Tyr_GTA_chr14: | CCTTCGATAGCTCAGCTGGTAGAGCGGAGGA |
| | 21131350-21131444 (−) | CTGTAGATTGTACAGACATTTGCGGACATCC |
| | | TTAGGTCGCTGGTTCGATTCCGGCTCGAAGG |
| | | AA |
| 400 | Thr_TGT_chr14: | AGGCCCTATAGCTCAGGGGTTAGAGCACTGG |
| | 21149848-21149921 (+) | TCTTGTAAACCAGGGGTCGCGAGTTCAAATC |
| | | TCGCTGGGGCCT |
| 401 | Tyr_GTA_chr14: | TCCTTCGATAGCTCAGCTGGTAGAGCGGAGG |
| | 21151431-21151520 (+) | ACTGTAGTACTTAATGTGTGGTCATCCTTAGG |
| | | TCGCTGGTTCGATTCCGGCTCGAAGGA |
| 402 | Pro_TGG_chr14: | TGGCTCGTTGGTCTAGGGGTATGATTCTCGCT |
| | 21152174-21152246 (+) | TTGGGTGCGAGAGGTCCCGGGTTCAAATCCC |
| | | GGACGAGCCC |
| 403 | Lys_CTT_chr14: | GCCCGGCTAGCTCAGTCGGTAGAGCATGGGA |
| | 58706612-58706685 (−) | CTCTTAATCCCAGGGTCGTGGGTTCGAGCCC |
| | | CACGTTGGGCGC |
| 404 | Ile_AAT_chr14: | CGGCCGGTTAGCTCAGTTGGTTAGAGCGTGG |
| | 102783428-102783502 (+) | TGCTAATAACGCCAAGGTCGCGGGTTCGATC |
| | | CCCGTACGGGCCA |
| 405 | Glu_TTC_chr15: | TCCCACATGGTCTAGCGGTTAGGATTCCTGGT |
| | 26327380-26327452 (−) | TTTCACCCAGGCGGCCCGGGTTCGACTCCCG |
| | | GTGTGGGAAT |
| 406 | Ser_GCT_chr15: | GACGAGGTGGCCGAGTGGTTAAGGCGATGG |
| | 40886022-40886104 (−) | ACTGCTAATCCATTGTGCTCTGCACGCGTGG |
| | | GTTCGAATCCCATCCTCGTCGA |
| 407 | His_GTG_chr15: | GCCGTGATCGTATAGTGGTTAGTACTCTGCGT |
| | 04549803-45490875 (−) | TGTGGCCGCAGCAACCTCGGTTCGAATCCGA |
| | | GTCACGGCAT |
| 408 | His_GTG_chr15: | CGCCGTGATCGTATAGTGGTTAGTACTCTGC |
| | 45493348-45493420 (+) | GTTGTGGCCGCAGCAACCTCGGTTCGAATCC |
| | | GAGTCACGGCA |
| 409 | Gln_CTG_chr15: | GGTTCCATGGTGTAATGGTTAGCACTCTGGA |
| | 16616399-66161471 (−) | CTCTGAATCCAGCGATCCGAGTTCAAATCTC |
| | | GGTGGAACCTG |
| 410 | Lys_CTT_chr15: | TGCCCGGCTAGCTCAGTCGGTAGAGCATGGG |
| | 27915903-79152976 (+) | ACTCTTAATCCCAGGGTCGTGGGTTCGAGCC |
| | | CCACGTTGGGCG |
| 411 | Arg_TCG_chr15: | GGGCCGCGTGGCCTAATGGATAAGGCGTCTG |
| | 89878303-89878376 (+) | ACTTCGGATCAGAAGATTGCAGGTTCGAGTC |
| | | CTGCCGCGGTCG |
| 412 | Gly_CCC_chr16: | GCGCCGCTGGTGTAGTGGTATCATGCAAGAT |
| | 686735-686806 (−) | TCCCATTCTTGCGACCCGGGTTCGATTCCCGG |
| | | GCGGCGCAC |
| 413 | Arg_CCG_chr16: | GGGCCGCGTGGCCTAATGGATAAGGCGTCTG |
| | 3200674-3200747 (+) | ATTCCGGATCAGAAGATTGAGGGTTCGAGTC |
| | | CCTTCGTGGTCG |
| 414 | Arg_CCT_chr16: | CGCCCCGGTGGCCTAATGGATAAGGCATTGG |
| | 3202900-3202973 (+) | CCTCCTAAGCCAGGGATTGTGGGTTCGAGTC |
| | | CCACCCGGGGTA |
| 415 | Lys_CTT_chr16: | GCCCGGCTAGCTCAGTCGGTAGAGCATGAGA |
| | 3207405-3207478 (−) | CCCTTAATCTCAGGGTCGTGGGTTCGAGCCC |
| | | CACGTTGGGCGT |
| 416 | Thr_CGT_chr16: | AGGCGCGGTGGCCAAGTGGTAAGGCGTCGGT |
| | 14379749-14379821 (+) | CTCGTAAACCGAAGATCACGGGTTCGAACCC |
| | | CGTCCGTGCCT |
| 417 | Leu_TAG_chr16: | GGTAGCGTGGCCGAGTGGTCTAAGGCGCTGG |
| | 22207031-22207113 (−) | ATTTAGGCTCCAGTCATTTCGATGGCGTGGGT |
| | | TCGAATCCCACCGCTGCCAC |
| 418 | Leu_AAG_chr16: | GGGTAGCGTGGCCGAGCGGTCTAAGGCGCTG |
| | 22308460-22308542 (+) | GATTAAGGCTCCAGTCTCTTCGGGGGCGTGG |
| | | GTTCGAATCCCACCGCTGCCA |
| 419 | Leu_CAG_chr16: | AGTCAGGATGGCCGAGCGGTCTAAGGCGCTG |
| | 57333862-57333945 (+) | CGTTCAGGTCGCAGTCTCCCCTGGAGGCGTG |
| | | GGTTCGAATCCCACTTCTGACA |
| 420 | Leu_CAG_chr16: | GTCAGGATGGCCGAGCGGTCTAAGGCGCTGC |
| | 57334391-57334474 (−) | GTTCAGGTCGCAGTCTCCCCTGGAGGCGTGG |
| | | GTTCGAATCCCACTTCTGACAG |
| 421 | Met_CAT_chr16: | GCCTCGTTAGCGCAGTAGGCAGCGCGTCAGT |
| | 87417627-87417700 (−) | CTCATAATCTGAAGGTCGTGAGTTCGAGCCT |
| | | CACACGGGGCAG |
| 422 | Leu_TAG_chr17: | GGTAGCGTGGCCGAGCGGTCTAAGGCGCTGG |
| | 8023631-8023713 (−) | ATTTAGGCTCCAGTCTCTTCGGAGGCGTGGG |
| | | TTCGAATCCCACCGCTGCCAG |
| 423 | Arg_TCT_chr17: | TGGCTCTGTGGCGCAATGGATAGCGCATTGG |
| | 8024242-8024330 (+) | ACTTCTAGTGACGAATAGAGCAATTCAAAGG |
| | | TTGTGGGTTCGAATCCCACCAGAGTCG |
| 424 | Gly_GCC_chr17: | CGCATTGGTGGTTCAGTGGTAGAATTCTCGC |
| | 8029063-8029134 (+) | CTGCCACGCGGGAGGCCCGGGTTCGATTCCC |
| | | GGCCAATGCA |
| 425 | Ser_CGA_chr17: | GCTGTGATGGCCGAGTGGTTAAGGCGTTGGA |
| | 8042198-8042280 (−) | CTCGAAATCCAATGGGGTCTCCCCGCGCAGG |
| | | TTCGAATCCTGCTCACAGCGT |
| 426 | Thr_AGT_chr17: | GGCGCCGTGGCTTAGCTGGTTAAAGCGCCTG |
| | 8042769-8042843 (−) | TCTAGTAAACAGGAGATCCTGGGTTCGAATC |
| | | CCAGCGGTGCCTG |
| 427 | Trp_CCA_chr17: | CGACCTCGTGGCGCAACGGTAGCGCGTCTGA |
| | 8089675-8089747 (+) | CTCCAGATCAGAAGGTTGCGTGTTCAAATCA |
| | | CGTCGGGGTCA |
| 428 | Ser_GCT_chr17: | AGACGAGGTGGCCGAGTGGTTAAGGCGATG |
| | 8090183-8090265 (+) | GACTGCTAATCCATTGTGCTCTGCACGCGTG |
| | | GGTTCGAATCCCATCCTCGTCG |
| 429 | Thr_AGT_chr17: | CGGCGCCGTGGCTTAGTTGGTTAAAGCGCCT |
| | 8090477-8090551 (+) | GTCTAGTAAACAGGAGATCCTGGGTTCGAAT |
| | | CCCAGCGGTGCCT |
| 430 | Trp_CCA_chr17: | GGCCTCGTGGCGCAACGGTAGCGCGTCTGAC |
| | 8124186-8124258 (−) | TCCAGATCAGAAGGTTGCGTGTTCAAATCAC |
| | | GTCGGGGTCAA |
| 431 | Gly_TCC_chr17: | AGCGTTGGTGGTATAGTGGTAAGCATAGCTG |
| | 8124865-8124937 (+) | CCTTCCAAGCAGTTGACCCGGGTTCGATTCCC |
| | | GGCCAACGCA |
| 432 | Asp_GTC_chr17: | TCCTCGTTAGTATAGTGGTGAGTATCCCCGCC |
| | 8125555-8125627 (−) | TGTCACGCGGGAGACCGGGGTTCGATTCCCC |
| | | GACGGGGAGA |
| 433 | Pro_CGG_chr17: | GGCTCGTTGGTCTAGGGGTATGATTCTCGCTT |
| | 8126150-8126222 (−) | CGGGTGCGAGAGGTCCCGGGTTCAAATCCCG |
| | | GACGAGCCCT |
| 434 | Thr_AGT_chr17: | GGCGCCGTGGCTTAGTTGGTTAAAGCGCCTG |
| | 8129552-8129626 (−) | TCTAGTAAACAGGAGATCCTGGGTTCGAATC |
| | | CCAGCGGTGCCTT |
| 435 | Ser_AGA_chr17: | GTAGTCGTGGCCGAGTGGTTAAGGCGATGGA |
| | 8129927-8130009 (−) | CTAGAAATCCATTGGGGTCTCCCCGCGCAGG |
| | | TTCGAATCCTGCCGACTACGT |
| 436 | Trp_CCA_chr17: | TGACCTCGTGGCGCAATGGTAGCGCGTCTGA |
| | 19411493-19411565 (+) | CTCCAGATCAGAAGGTTGCGTGTTCAAGTCA |
| | | CGTCGGGGTCA |
| 437 | Thr_CGT_chr17: | AGGCGCGGTGGCCAAGTGGTAAGGCGTCGGT |
| | 29877092-29877164 (+) | CTCGTAAACCGAAGATCGCGGGTTCGAACCC |
| | | CGTCCGTGCCT |
| 438 | Cys_GCA_chr17: | AGGGGGTATAGCTCAGTGGTAGAGCATTTGA |
| | 37023897-37023969 (+) | CTGCAGATCAAGAGGTCCCCGGTTCAAATCC |
| | | GGGTGCCCCCT |
| 439 | Cys_GCA_chr17: | GGGGGTATAGCTCAGTGGTAGAGCATTTGAC |
| | 37025544-37025616 (−) | TGCAGATCAAGAGGTCCCTGGTTCAAATCCG |
| | | GGTGCCCCCTC |
| 440 | Cys_GCA_chr17: | GGGGGTATAGCTCAGTGGTAGAGCATTTGAC |
| | 37309986-37310058 (−) | TGCAGATCAAGAGGTCCCCGGTTCAAATCCG |
| | | GGTGCCCCCTC |
| 441 | Gln_TTG_chr17: | AGGTCCCATGGTGTAATGGTTAGCACTCTGG |
| | 47269889-47269961 (+) | ACTTTGAATCCAGCGATCCGAGTTCAAATCT |
| | | CGGTGGGACCT |
| 442 | Arg_CCG_chr17: | GACCCAGTGGCCTAATGGATAAGGCATCAGC |
| | 66016012-66016085 (−) | CTCCGGAGCTGGGGATTGTGGGTTCGAGTCC |
| | | CATCTGGGTCGC |
| 443 | Arg_CCT_chr17: | AGCCCCAGTGGCCTAATGGATAAGGCACTGG |
| | 73030000-73030073 (+) | CCTCCTAAGCCAGGGATTGTGGGTTCGAGTC |
| | | CCACCTGGGGTA |
| 444 | Arg_CCT_chr17: | GCCCCAGTGGCCTAATGGATAAGGCACTGGC |
| | 07303525-73030598 (−) | CTCCTAAGCCAGGGATTGTGGGTTCGAGTCC |
| | | CACCTGGGGTGT |
| 445 | Arg_TCG_chr17: | AGACCGCGTGGCCTAATGGATAAGGCGTCTG |
| | 17303207-73031280 (+) | ACTTCGGATCAGAAGATTGAGGGTTCGAGTC |
| | | CCTTCGTGGTCG |
| 446 | Asn_GTT_chr19: | CGTCTCTGTGGCGCAATCGGTTAGCGCGTTC |
| | 1383561-1383635 (+) | GGCTGTTAACCGAAAGGTTGGTGGTTCGAGC |
| | | CCACCCAGGGACG |
| 447 | Gly_TCC_chr19: | GGCGTTGGTGGTATAGTGGTTAGCATAGCTG |
| | 4724081-4724153 (+) | CCTTCCAAGCAGTTGACCCGGGTTCGATTCCC |
| | | GGCCAACGCA |
| 448 | Val_CAC_chr19: | GTTTCCGTAGTGTAGCGGTTATCACATTCGCC |
| | 4724646-4724719 (−) | TCACACGCGAAAGGTCCCCGGTTCGATCCCG |
| | | GGCGGAAACAG |
| 449 | Thr_AGT_chr19: | TGGCGCCGTGGCTTAGTTGGTTAAAGCGCCT |
| | 33667962-33668036 (+) | GTCTAGTAAACAGGAGATCCTGGGTTCGAAT |
| | | CCCAGCGGTGCCT |
| 450 | Ile_TAT_chr19: | GCTCCAGTGGCGCAATCGGTTAGCGCGCGGT |
| | 39902807-39902900 (−) | ACTTATATGACAGTGCGAGCGGAGCAATGCC |
| | | GAGGTTGTGAGTTCGATCCTCACCTGGAGCA |
| | | C |
| 451 | Gly_GCC_chr21: | GCATGGGTGGTTCAGTGGTAGAATTCTCGCC |
| | 18827106-18827177 (−) | TGCCACGCGGGAGGCCCGGGTTCGATTCCCG |
| | | GCCCATGCAG |

### Non-Naturally Occurring Modification

A TREM, a TREM core fragment or a TREM fragment described herein comprises a non-naturally occurring modification, e.g., a modification described in Table 5. A non-naturally occurring modification can be made according to methods known in the art. Exemplary methods of making non-naturally occurring modifications are provided in Examples 4 and 5.

In an embodiment, a non-naturally occurring modification is a modification that a cell, e.g., a human cell, does not make on an endogenous tRNA.

In an embodiment, a non-naturally occurring modification is a modification that a cell, e.g., a human cell, can make on an endogenous tRNA, but wherein such modification is in a location in which it does not occur on a native tRNA. In an embodiment, the non-naturally occurring modification is in a domain, linker or arm which does not have such modification in nature. In an embodiment, the non-naturally occurring modification is at a position within a domain, linker or arm, which does not have such modification in nature. In an embodiment, the non-naturally occurring modification is on a nucleotide which does not have such modification in nature. In an embodiment, the non-naturally occurring modification is on a nucleotide at a position within a domain, linker or arm, which does not have such modification in nature.

In an embodiment, a TREM, a TREM core fragment or a TREM fragment described herein comprises a modification provided in Table 5, or a combination thereof. The modifications provided in Table 5 are non-naturally occurring or occur naturally in RNAs, and are used herein on a synthetic TREM, a TREM core fragment or a TREM fragment at a position that does not occur in nature.

TABLE 5
Exemplary modifications
Chemical Modification

| | |
|---|---|
| (S)-constrained ethyl (cEt) | 5-(methoxycarbonyl-methyl)uracil |
| (±)1-(2-Hydroxypropyl)pseudouridine | 5-(methyl) 2(thio)uracil |
| (2R)-1-(2-Hydroxypropyl)pseudouridine | 5-(methyl) 2,4 (dithio)uracil |
| (2S)-1-(2-Hydroxypropyl)pseudouridine | 5-(methyl) 4 (thio)uracil |
| (3-(3-amino-3-carboxypropyl)uridine | 5-(methyl)-2-(thio)pseudouracil |
| (E)-5-(2-Bromo-vinyl)ara-uridine | 5-(methyl)-2-(thio)uracil |
| (E)-5-(2-Bromo-vinyl)cytidine | 5-(methyl)-2,4 (dithio)pseudouracil |
| (E)-5-(2-Bromo-vinyl)uridine | 5-(methyl)-2,4-(dithio)uracil |
| (E)-vinylphosphonate | 5-(methyl)-4 (thio)pseudouracil |
| (R) 5′-C-methyl | 5-(methyl)isocarbostyrilyl |
| (R) 5′-C-methyl with phosphate | 5-(methyl)pseudouracil |
| (S) 5′-C-methyl | 5-(methylaminomethyl)-2 (thio)uracil |
| (S) 5′-C-methyl with phosphate | 5-(methylaminomethyl)-2,4(dithio)uracil |
| (Z)-5-(2-Bromo-vinyl)ara-uridine | 5-(methylaminomethyl)-4-(thio)uracil |
| (Z)-5-(2-Bromo-vinyl)uridine | 5-(propynyl)uracil |
| 1(4-Nitro-phenyl)pseudouridine | 5-(propynyl)cytosine |
| 1-(aminocarbonylethylenyl)-2(thio)- | 5-(trifluoromethyl)cytosine |
| pseudouracil | |
| 1-(aminocarbonylethylenyl)-2,4- | 5-(trifluoromethyl)uracil |
| (dithio)pseudouracil | |
| 1-(2,2,2-Trifluoroethyl)-pseudouridine | 5,2′-O-dimethylcytidine |
| 1-(2,2,3,3,3-Pentafluoropropyl)pseudouridine | 5,2′-O-dimethyluridine |
| 1-(2,2-Diethoxyethyl)pseudouridine | 5,6-dihydro-uridine |
| 1-(2,4,6-Trimethylbenzyl)pseudouridine | 5-Aminoallyl-cytosine |
| 1-(2,4,6-Trimethyl-benzyl)pseudo-uridine | 5-aminoallyl-uridine |
| 1-(2,4,6-Trimethyl-phenyl)pseudo-uridine | 5-aminomethy 1-2-thiouridine |
| 1-(2-Amino-2-carboxyethyl)pseudo-uridine | 5-aza-2-thio-zebularine |
| 1-(2-Amino-ethyl)pseudouridine | 5-aza-cytidine |
| 1-(2-Hydroxyethyl)pseudouridine | 5-aza-uridine |
| 1-(2-Methoxyethyl)pseudouridine | 5-aza-zebularine |
| 1-(3,4-Bis- | 5-bromo-cytidine |
| trifluoromethoxvbenzyl)pseudouridine | |
| 1-(3,4-Dimethoxybenzyl)pseudouridine | 5-bromo-uridine |
| 1-(3-Amino-3-carboxypropyl)pseudo-uridine | 5-carbamoylmethyl-2′-O-methyluridine |
| 1-(3-Amino-propyl)pseudouridine | 5-carbamoylmethyluridine |
| 1-(3-Cyclopropyl-prop-2-ynyl)pseudouridine | 5-carboxyhydroxymethyluridine |
| TP | |
| 1-(4-Amino-4-carboxybutyl)pseudouridine | 5-carboxyhydroxymethyluridine methyl ester |
| 1-(4-Amino-benzyl)pseudouridine | 5-carboxymethylaminomethyl-2-thiouridine |
| 1-(4-Amino-butyl)pseudouridine | 5-carboxymethylaminomethyl-2′-O- |
| | methyluridine |
| 1-(4-Amino-phenyl)pseudouridine | 5-carboxymethylaminomethyl-2-thiouridine |
| 1-(4-Azidobenzyl)pseudouridine | 5-carboxymethylaminomethyluridine |
| 1-(4-Bromobenzyl)pseudouridine | 5-carboxymethyluridine |
| 1-(4-Chlorobenzyl)pseudouridine | 5-Cyanocytidine |
| 1-(4-Fluorobenzyl)pseudouridine | 5-Cyanouridine |
| 1-(4-Iodobenzyl)pseudouridine | 5-Dimethylaminouridine |
| 1-(4-Methanesulfonylbenzyl)pseudouridine | 5-Ethynylara-cytidine |
| 1-(4-Methoxybenzyl)pseudouridine | 5-Ethynylcytidine |
| 1-(4-Methoxy-phenyl)pseudouridine | 5-formyl-2′-O-methylcytidine |
| 1-(4-Methylbenzyl)pseudouridine | 5-formylcytidine |
| 1-(4-Nitrobenzyl)pseudouridine | 5′-Homo-adenosine |
| 1-(4-Thiomethoxybenzyl)pseudouridine | 5′-Homo-cytidine |
| 1-(4-Trifluoromethoxybenzyl)pseudouridine | 5′-Homo-guanosine |
| 1-(4-Trifluoromethylbenzyl)pseudouridine | 5′-Homo-uridine |
| 1-(5-Amino-pentyl)pseudouridine | 5-hydroxymethylcytidine |
| 1-(6-Amino-hexyl)pseudouridine | 5-hydroxyuridine |
| 1-(aminoalkylamino-carbonylethylenyl)- | 5-iodo-2′-fluoro-deoxyuridine |
| 2(thio)-pseudouracil | |
| 1-(aminoalkylaminocarbonylethylenyl)-2,4- | 5-iodo-cytidine |
| (dithio)pseudouracil | |
| 1-(aminoalkylaminocarbonylethylenyl)- | 5-iodo-uridine |
| pseudouracil | |
| 1-(aminoalkylaminocarbonylethylenyl)-4- | 5-methoxycarbonylmethy 1-2-thiouridine |
| (thio)pseudouracil | |
| 1-(aminocarbonylethylenyl)-4- | 5-methoxycarbonylmethyl-2′-O- |
| (thio)pseudouracil | methyluridine |
| 1-(aminocarbonylethylenyl)-pseudouracil | 5-methoxycarbonylmethyluridine |
| 1-(aza)-2-(thio)-3-(aza)-phenoxazin-1-yl | 5-Methoxycytidine |
| 1,2′ -O-dimethyladenosine | 5-methoxyuridine |
| 1,2′-O-dimethylguanosine | 5-methyl-2-thiouridine |
| 1,2′-O-dimethylinosine | 5-methylaminomethyl-2-selenouridine |
| 1,3-(diaza)-2-(oxo)-phenthiazin-1-yl | 5-methylaminomethyl-2-thiouridine |
| 1,3-(diaza)-2-(oxo)-phenoxazin-1-yl | 5-methylaminomethyluridine |
| 1,3,5-(triaza)-2,6-(dioxa)-naphthalene | 5-methylcytidine |
| 1,6-Dimethyl-pseudouridine | 5-Methyldihydrouridine |
| 1-{3-[2-(2-Aminoethoxy)-ethoxy]- | 5-methyluridine |
| propionvl}pseudouridine | |
| 1-Acetylpseudouridine | 5-methyl-zebularine |
| 1-Allylpseudouridine | 5-nitroindole |
| 1-Aminomethyl-pseudo-uridine | 5-Oxyacetic acid- Uridine |
| 1-Benzoylpseudouridine | 5-Oxyacetic acid-methyl ester-Uridin Nl- |
| | methyl-pseudouridine |
| 1-Benzyloxymethylpseudouridine | 5-Phenylethynyluridine |
| 1-Benzyl-pseudo-uridine | 5′-phosphorothioate |
| 1-Biotinylpseudouridine | 5-propynyl cytosine |
| 1-Butyl-pseudo-uridine | 5-propynyl uracil |
| 1-carboxymethyl-pseudouridine | 5-taurinomethyl-2-thiouridine |
| 1-Cyanomethylpseudouridine | 5-taurinomethyluridine |
| 1-Cyclobutylmethyl-pseudo-uridine | 5-Trideuteromethyl-6-deuterouridine |
| 1-Cyclobutyl-pseudo-uridine | 5-Trifluoromethyl-Cytidine |
| 1-Cycloheptylmethyl-pseudo-uridine | 5-Trifluoromethyl-Uridine |
| 1-Cycloheptyl-pseudo-uridine | 5-uracil |
| 1-Cyclohexylmethyl-pseudo-uridine | 5-Vinylarauridine |
| 1-Cyclohexyl-pseudo-uridine | 6 (azo)uracil |
| 1-Cyclooctylmethyl-pseudo-uridine | 6-(2,2,2-Trifluoroethyl)-pseudo-uridine |
| 1-Cyclooctyl-pseudo-uridine | 6-(4-Morpholino)-pseudo-uridine |
| 1-Cyclopentylmethyl-pseudo-uridine | 6-(4-Thiomorpholino)-pseudo-uridine |
| 1-Cyclopentyl-pseudo-uridine | 6-(alkyl)guanine |
| 1-Cyclopropylmethyl-pseudo-uridine | 6-(alkyl)adenine |
| 1-Cyclopropyl-pseudo-uridine | 6-(aza)pyrimidine |
| 1-deazaadenosine | 6-(azo)cytosine |
| 1-Ethyl-pseudo-uridine | 6-(azo)thymine |
| 1-Hexyl-pseudo-uridine | 6-(azo)uracil |
| 1-Homoallylpseudouridine | 6-(methyl)-7-(aza)indolyl |
| 1-Hydroxymethylpseudouridine | 6-(methyl)adenine |
| 1-iso-propyl-pseudo-uridine | 6-(methyl)guanine |
| 1-Me-2-thio-pseudo-uridine | 6-(Substituted-Phenyl)-pseudo-uridine |
| 1-Me-4-thio-pseudo-uridine | 6-Amino-pseudo-uridine |
| 1-Me-alpha-thio-pseudo-uridine | 6-aza-cytidine |
| 1-Me-guanosine | 6-aza-uridine |
| 1-Methanesulfonylmethylpseudouridine | 6-Azido-pseudo-uridine |
| 1-Methoxymethylpseudouridine | 6-Bromo-pseudo-uridine |
| 1-Methyl-6-amino-pseudo-uridine | 6-Butyl-pseudo-uridine |
| 1-Methyl-6-bromo-pseudo-uridine | 6-Chloro-pseudo-uridine |
| 1-Methyl-6-cyano-pseudo-uridine | 6-chloro-purine |
| 1-Methyl-6-hydroxyamino-pseudo-uridine | 6-Cyano-pseudo-uridine |
| 1-Methyl-6-trifluoromethoxy-pseudo-uridine | 6-Dimethylamino-pseudo-uridine |
| 1-methyladenosine | 6-Ethoxy-pseudo-uridine |
| 1-methylguanosine | 6-Ethylcarboxylate-pseudo-uridine |
| 1-methylinosine | 6-Ethyl-pseudo-uridine |
| 1-methylpseduouridine | 6-Fluoro-pseudo-uridine |
| 1-methyl-pseudoisocytidine | 6-Formyl-pseudo-uridine |
| 1-methyl-pseudouridine | 6-Hydroxyamino-pseudo-uridine |
| 1-Methyl-pseudo-UTP | 6-Hydroxy-pseudo-uridine |
| 1-Morpholinomethylpseudouridine | 6-Iodo-pseudo-uridine |
| 1-Pentyl-pseudo-uridine | 6-iso-Propyl-pseudo-uridine |
| 1-Phenyl-pseudo-uridine | 6-methoxy-guanosine |
| 1-Pivaloylpseudouridine | 6-Methoxy-pseudo-uridine |
| 1-Propargylpseudouridine | 6-Methylamino-pseudo-uridine |
| 1-Propyl-pseudo-uridine | 6-methyl-guanosine |
| 1-propynyl-pseudouridine | 6-Methyl-pseudo-uridine |
| 1-propynyl-uridine | 6-Phenyl-pseudo-uridine |
| 1-p-tolyl-pseudo-uridine | 6-phenyl-pyrrolo-pyrimidin-2-on-3-yl |
| 1-substituted 2-(thio)-pseudouracil | 6-Propyl-pseudo-uridine |
| 1-substituted 2,4-(dithio)pseudouracil | 6-tert-Butyl-pseudo- uridine |
| 1-substituted 4-(thio)pseudouracil | 6-thio-7-deaza-8-aza-guanosine |
| 1-substituted pseudouracil | 6-thio-7-deaza-guanosine |
| 1-taurinomethyl-pseudouridine | 6-thio-7-methyl-guanosine |
| 1-tert-Butyl-pseudo-uridine | 6-thio-guanosine |
| 1-Thiomethoxymethylpseudouridine | 6-Trifluoromethoxy-pseudo-uridine |
| 1-Thiomorpholinomethylpseudouridine | 6-Trifluoromethyl-pseudo-uridine |
| 1-Trifluoroacetylpseudouridine | 7-(alkyl)guanine |
| 1-Trifluoromethylpseudouridine | 7-(aminoalkylhydroxy)-1-(aza)-2-(thio)-3- |
| | (aza)-phenthiazin-1-yl |
| 1-Vinylpseudouridine | 7-(aminoalkylhydroxy)-1-(aza)-2-(thio)-3- |
| | (aza)-phenoxazin-1-yl |
| 2-(amino)purine | 7-(aminoalkylhydroxy)-1,3-(diaza)-2-(oxo)- |
| | phenthiazin-1-yl |
| 2-(thio)pseudouracil | 7-(aminoalkylhydroxy)-1,3-(diaza)-2-(oxo)- |
| | phenoxazin-1-yl |
| 2′-alpha-Ethynylcytidine | 7-(aza)indolyl |
| 2′-alpha-Ethynylguanosine | 7-(deaza)adenine |
| 2′-alpha-Ethynyluridine | 7-(deaza)guanine |
| 2′-alpha-Trifluoromethyladenosine | 7-(guanidiniumalkylhydroxy)-1-(aza)-2- |
| | (thio)-3-(aza)-phenoxazinl-yl |
| 2′-alpha-Trifluoromethylguanosine | 7-(guanidiniumalkylhydroxy)-1-(aza)-2- |
| | (thio)-3-(aza)-phenthiazin-1-yl |
| 2′-alpha-Trifluoromethyluridine | 7-(guanidiniumalkylhydroxy)-1-(aza)-2- |
| | (thio)-3-(aza)-phenoxazin-1-yl |
| 2′-Amino-2′-deoxycytosine | 7-(guanidiniumalkyl-hydroxy)-1,3-(diaza)-2- |
| | (oxo)-phenthiazin-1-yl |
| 2′ -amino-2′-deoxyribose | 7-(guanidiniumalkylhydroxy)-1,3-(diaza)-2- |
| | (oxo)- phenoxazin-1-yl |
| 2′-alpha-Trifluoromethylcytidine | 7-(methyl)guanine |
| 2′-Azido-2′-deoxycytosine | 7-(propynyl)isocarbostyrily1 |
| 2′-azido-2′-deoxyribose | 7-(propynyl)isocarbostyrilyl |
| 2′-Azido-deoxyuridine | 7-propynyl(aza)indolyl |
| 2′-beta-Ethynyladenosine | 7-aminomethyl-7-deazaguanosine |
| 2′-beta-Ethynylguanosine | 7-cyano-7-deazaguanosine |
| 2′-beta-Ethynyluridine | 7-deaza-2- aminopurine |
| 2′-beta-Trifluoromethyluridine | 7-deaza-2,6-diaminopurine |
| 2′-beta-Ethynylcytidine | 7-deaza-2-amino-purine |
| 2′-bromo-deoxyuridine | 7-deaza-8-aza-2,6-diaminopurine |
| 2′-deoxyuridine | 7-deaza-8-aza-2-aminopurine |
| 2′-Deoxy-2′,2′-difluoroadenosine | 7-deaza-8-aza-adenine |
| 2′-Deoxy-2′,2′-difluorocytidine | 7-deaza-8-aza-adenosine |
| 2′-Deoxy-2′,2′-difluoroguanosine | 7-deaza-8-aza-guanosine |
| 2′-Deoxy-2′,2′-difluorouridine | 7-deaza-adenosine |
| 2′-Deoxy-2′-alpha-aminocytidine | 7-deaza-guanosine |
| 2′-Deoxy-2′-alpha-aminouridine TP | 7-deaza-inosinyl |
| 2′-Deoxy-2′-alpha-azidocytidine | 7-methyl-8-oxo-guanosine |
| 2′-Deoxy-2′-alpha-azidouridine TP | 7-methyladenine |
| 2′-Deoxy-2′-alpha-mercaptoadenosine | 7-methylguanosine |
| 2′-Deoxy-2′-alpha-mercaptocytidine | 7-methylinosine |
| 2′-Deoxy-2′-alpha-mercaptoguanosine | 7-substituted 1-(aza)-2-(thio)-3-(aza)- |
| | phenoxazin-1-yl |
| 2′-Deoxy-2′-alpha-thiomethoxyadenosine | 7-substituted 1,3-(diaza)-2-(oxo)-phenoxazin- |
| | 1-yl |
| 2′-Deoxy-2′-alpha-thiomethoxycytidine | 8-(alkenyl)adenine |
| 2′-Deoxy-2′-alpha-thiomethoxyguanosine | 8-(alkenyl)guanine |
| 2′-Deoxy-2′-alpha-thiomethoxyuridine | 8-(alkyl)adenine |
| 2′-Deoxy-2′-alpha-mercaptouridine | 8-(alkyl)guanine |
| 2′-Deoxy-2′-beta-aminoadenosine | 8-(alkynyl)adenine |
| 2′-Deoxy-2′-beta-aminoguanosine | 8-(alkynyl)guanine |
| 2′-Deoxy-2′-beta-aminouridine | 8-(amino)adenine |
| 2′-Deoxy-2′-beta-azidoadenosine | 8-(amino)guanine |
| 2′-Deoxy-2′-beta-azidocytidine | 8-(halo)adenine |
| 2′-Deoxy-2′-beta-azidoguanosine | 8-(halo)guanine |
| 2′-Deoxy-2′-beta-azidouridine | 8-(hydroxyl)adenine |
| 2′-Deoxy-2′-beta-aminocytidine | 8-(hydroxyl)guanine |
| 2′-Deoxy-2′-beta-bromoadenosine | 8-(thioalkyl)adenine |
| 2′-Deoxy-2′-beta-bromocytidine | 8-(thioalkyl)guanine |
| 2′-Deoxy-2′-beta-bromoguanosine | 8-(thiol)adenine |
| 2′-Deoxy-2′-beta-bromouridine | 8-(thiol)guanine |
| 2′-Deoxy-2′-beta-chloroadenosine | 8-Aza-adenosine |
| 2′-Deoxy-2′-beta-chlorocytidine | 8-azido-adenosine |
| 2′-Deoxy-2′-beta-chloroguanosine | 8-bromo-adenosine |
| 2′-Deoxy-2′-beta-chlorouridine | 8-bromo-guanosine |
| 2′-Deoxy-2′-beta-fluoroadenosine | 8-oxo-guanosine |
| 2′-Deoxy-2′-beta-fluorocytidine | 8-Trifluoromethyladenosine |
| 2′-Deoxy-2′-beta-fluoroguanosine | 9-(methyl)-imidizopyridinyl |
| 2′-Deoxy-2′-beta-fluorouridine | 9-Deazaadenosine |
| 2′-Deoxy-2′-beta-iodoadenosine | 9-Deazaguanosine |
| 2′-Deoxy-2′-beta-iodocytidine | alkene containing backbones |
| 2′-Deoxy-2′-beta-iodoguanosine | alkyl phosphonates |
| 2′-Deoxy-2′-beta-iodouridine | allyamino-thymidine |
| 2′-Deoxy-2′-beta-mercaptoadenosine | allyamino-uracil |
| 2′-Deoxy-2′-beta-mercaptocytidine | alpha-thio-cytidine |
| 2′-Deoxy-2′-beta-mercaptoguanosine | alpha-thio-guanosine |
| 2′-Deoxy-2′-beta-mercaptouridine | alpha-thio-pseudo-uridine |
| 2′-Deoxy-2′-beta-thiomethoxyadenosine | alpha-thio-uridine |
| 2′-Deoxy-2′-beta-thiomethoxycytidine TP | altriol |
| 2′-Deoxy-2′-beta-thiomethoxyuridine | aminoalkylphosphoramidates |
| 2′-deoxyuridine | aminoalkylphosphotriesters |
| 2′-F-5-Methyl-2′-deoxyuridine | aminoindolyl |
| 2′-Fluoro | anthracenyl |
| 2′-fluoro-modified bases | archaeosine |
| 2′-fluorouridine | aza cytosine |
| 2′-methyl, 2′-amino, 2′-azido, 2′-fluoro- | aza thymidine |
| adenine | |
| 2′-methyl, 2′-amino, 2′-azido, 2′-fluroo- | aza uracil |
| cytidine | |
| 2′-OH-ara-adenosine | aza adenine |
| 2′-OH-ara-cytidine | azaguanine |
| 2′-OH-ara-guanosine | bis-ortho-(aminoalkylhydroxy)-6-phenyl- |
| | pyrrolo-nvrimidin-2-on-3-yl |
| 2′-OH-ara-uridine | bis-ortho-substituted-6-phenyl-pyrrolo- |
| | pyrimidin-2-on-3-yl |
| 2′-OMe-2-Aminoadenosine | boranophosphates |
| 2′-OMe-5-Me-uridine | -CH2-O-N(CH3)-CH2- |
| 2′-OMe-pseudouridine | -CH2-N(CH3)-N(CH3)-CH2- |
| 2′-O-Methyl-5-(1-propynyl)cytidine | -CH2-NH-CH2- |
| 2′-O-Methyl-5-(1-propynyl)uridine | chiral phosphonates |
| 2′-O-methyladenosine | chiral phosphorothioates |
| 2′-O-methylation | Constrained nucleic acid (CNA) |
| 2′-O-methylcytidine | deaza cytosine |
| 2′-O-methylguanosine | deaza guanine |
| 2′-O-methylinosine | deaza thymidine |
| 2′-O-methyl-ribose | deaza uracil |
| 2′-O-methyluridine | deazaadenine |
| 2′-O-ribosyladenosine (phosphate) | deoxy-thymidine |
| 2-(alkyl)guanine | difluorotolyl |
| 2-(alkyl)adenine | dihydropseudouridine |
| 2-(amino)adenine | dihydrouridine |
| 2-(aminoalkyl)adenine | DNA |
| 2-(aminopropyl)adenine | epoxyqueuosine |
| 2-(halo)adenine | Fluoro hexitol nucleic acid (FHNA) |
| 2-(methylthio) N6 (isopentenyl)adenine | formacetyl and thioformacetyl backbones |
| 2-(propyl)adenine | Formycin A |
| 2-(propyl)guanine | Formycin B |
| 2-(thio)cytosine | galactosyl-queuosine |
| 2-(thio)uracil | GNA (glycol nucleic acid) |
| 2,2′-anhydro-cytidine | hydroxywybutosine |
| 2,2′-anhydro-uridine | hypoxanthine |
| 2,4-(dithio)pseudouracil | imidizopyridinyl |
| 2,4,5-(trimethyl)phenyl | inosinyl |
| 2,6-(diamino)purine | isocarbostyrilyl |
| 2,6-diaminopurine | isoguanosine |
| 2′-alpha-ethynyladenosine | isopentenyladenosine |
| 2′-Amino-2′-deoxy-guanosine | isowyosme |
| 2′-Amino-2′-deoxy-uridine | 1 -Alkyl-6-homoallyl-pseudo-uridine |
| 2-amino-6-Chloro-purine | 1 -Methyl-6-(2,2,2-Trifluoroethyl)pseudo- |
| | uridine |
| 2-aminoadenine | 1 -Methyl-6-(4-thiomorpholino)-pseudo- |
| | uridine |
| 2-Aminoadenosine | 1 -Methyl-6-azido-pseudo-uridine |
| 2-aminopurine | 1 -Methyl-6-chloro-pseudo-uridine |
| 2-Amino-riboside | 1 -Methyl-6-dimethylamino-pseudo-uridine |
| 2-aza-inosinyl | 1 -Methyl-6-ethoxy-pseudo-uridine |
| 2′-azido-2′-deoxyadenosine | 1 -Methyl-6-ethylcarboxylate-pseudo-uridine |
| 2′-Azido-2′-deoxy-guanosine | 1 -Methyl-6-fluoro-pseudo-uridine |
| 2′-Azido-2′-deoxy-uridine | 1 -Methyl-6-hydroxy-pseudo-uridine |
| 2-Azidoadenosine | 1 -Methyl-6-iodo-pseudo-uridine |
| 2′-beta-Trifluoromethyladenosine | 1 -Methyl-6-methylamino-pseudo-uridine |
| 2′-beta-Trifluoromethylguanosine | 1-[3-(2-{2-[2-(2-Aminoethoxy)-ethoxy]- |
| | ethoxy}-ethoxy)-propionyl]pseudouridine |
| 2-Bromoadenosine | 1-Alkyl-6-(1-propynyl)-pseudo-uridine |
| 2′-beta-Trifluoromethylcytidine | 1-Alkyl-6-(2-propynyl)-pseudo-uridine |
| 2-Chloroadenosine | 1-Alkyl-6-allyl-pseudo-uridine |
| 2′-Deoxy-2′-alpha-aminoadenosine | 1-Alkyl-6-ethynyl-pseudo-uridine |
| 2′-Deoxy-2′-alpha-aminoguanosine | 1-Alkyl-6-vinyl-pseudo-uridine |
| 2′-Deoxy-2′-alpha-azidoadenosine | 1-Biotinyl-PEG2-pseudouridine |
| 2′-Deoxy-2′-alpha-azidoguanosine | 1-methyl-1-deaza-pseudoisocytidine |
| 2′-Deoxy-2′-beta-thiomethoxyguanosine | 1-methyl-1-deaza-pseudouridine |
| 2′-Fluor-N4-Bz-cytidine | 1-Methyl-3-(3-amino-3- |
| | carboxyproovl)pseudo-Uridine |
| 2′-fluoro-2′-deoxyribose | 1-Methyl-3-(3-amino-3-carboxypropyl) |
| | pseudouridine |
| 2-Fluoroadenosine | 1-methyl-3-(3-amino-5- |
| | carboxypropyl)pseudouridine |
| 2′-Fluoro-N2-isobutyl-guanosine | 1-Methyl-6-(4-morpholino)-pseudo-uridine |
| 2′-Fluoro-N4-Acetyl-cytidine | 1-Methyl-6-(substituted phenyl)pseudo- |
| | uridine |
| 2′-Fluoro-N6-Bz-deoxyadenosine | 1-Methyl-6-butyl-pseudo-uridine |
| 2-Iodoadenosine | 1-Methyl-6-ethyl-pseudo-uridine |
| 2-Mercaptoadenosine | 1-Methyl-6-formyl-pseudo-uridine |
| 2-methoxy-4-thio-pseudouridine | 1-Methyl-6-iso-propyl-pseudo-uridine |
| 2-methoxy-4-thio-uridine | 1-Methyl-6-methoxy-pseudo-uridine |
| 2-methoxy-5-methyl-cytidine | 1-Methyl-6-phenyl-pseudo-uridine |
| 2-methoxy-adenine | 1-Methyl-6-propyl-pseudo-uridine |
| 2-methoxy-cytidine | 1-Methyl-6-tert-butyl-pseudo-uridine |
| 2-methoxyuridine | 1-methyl-6-thio-guanosine |
| 2′-methyl, 2′-amino, 2′-azido, 2′-fluro- | 1-Methyl-6-trifluoromethyl-pseudo-uridine |
| guanosine | |
| 2′-methyl, 2′-amino, 2′-azido, 2′fluro-uridine | Locked nucleic acid (LNA) |
| 2-methyladenosine | 1-taurinomethyl-1-methyl-uridine |
| 2-methylpseudouridine | 1-taurinomethyl-4-thio-uridine |
| 2-methylthioadenine | lysidine |
| 2-methylthio-N6 isopentenyladenosine | mannosyl-queuosine |
| 2-methylthio-N6-(cis- | Methyl phosphonate |
| hydroxyisopentenyl)adenosine | |
| 2-methylthio-N6-hydroxynorvalyl | methylene (methylimino) |
| carbamoyladenosine | |
| 2-methylthio-N6-isopentenyladenosine | methylene formacetyl and thioformacetyl |
| | backbones |
| 2-methylthio-N6-methyladenosine | methyleneimino and methylenehydrazino |
| | backbones |
| 2-methylthio-N6-threonyl | methylphosphonates |
| carbamoyladenosine | |
| 2′-O-methoxyethyl (MOE) | methylwyosine |
| 2′-O-methoxyethylribose (MOE) | morpholino linkages |
| 2′-O-methyl | mosme |
| 2′-O-methyladenosine | N (methyl)guanine |
| 2′-O-methylcytidine | -N(CH3)-CH2-CH2- |
| 2′-O-methylguanosine | N-(methyl)guanine |
| 2′-O-methylinosine | N2,7,2′-O-trimethylguanosine |
| 2′O-methyl-N2-isobutyl-guanosine | N2,2′-O-dimethylguanosine |
| 2′-O-Methyl-N4-Acetyl-cytidine | N2,7-dimethylguanosine |
| 2′-O-methyl-N4-Bz-cytidine | N2,N2,2′-O-trimethylguanosine |
| 2′-O-methyl-N6-Bz-deoxyadenosine | N2,N2,7-trimethylguanosine |
| 2′-O-methylpseudouridine | N2,N2-dimethyl-6-thio-guanosine |
| 2′-O-methyluridine | N2,N2-dimethylguanosine |
| 2′-O-ribosyladenosine (phosphate) | N2-isobutyl-guanosine |
| 2′-O-ribosylguanosine (phosphate) | N2-methyl-6-thio-guanosine |
| 2-oxo-7-aminopyridopyrimidin-3-yl | N2-methylguanosine |
| 2-oxo-pyridopyrimidine-3-yl | N2-substituted purines |
| 2-pyridinone | N3 (methyl)uracil |
| 2-thio-1-methyl-1-deaza-pseudouridine | N4 (acetyl)cytosine |
| 2-thio-1-methyl-pseudouridine | N4,2′-O-dimethylcytidine |
| 2-thio-2′-O-methyluridine | N4,N4-Dimethyl-2′-OMe-Cytidine |
| 2-thio-5-aza-uridine | N4-acetyl-2′-O-methylcytidine |
| 2-thio-5-methyl-cytidine | N4-acetylcytidine |
| 2-thiocytidine | N4-Amino-cytidine |
| 2-thio-dihydropseudouridine | N4-Benzoyl-cytidine |
| 2-thio-dihydrouridine | N4-methylcytidine |
| 2-thio-pseudouridine | N6-(19-Amino-pentaoxanonadecyl)adenosine |
| 2-thiouridine | N6-(cis-hydroxyisopentenyl)adenosine |
| 2-thio-zebularine | N6-(isopentyl)adenine |
| 2-Trifluoromethyladenosine | N6-(methyl)adenine |
| 3-(deaza)-5-(aza)cytosine | N6, N6 (dimethyl)adenine |
| 3-(methyl)cytosine | N6,2′-O-dimethyladenosine |
| 3-nitropyrrole | N6,N6,2′-O-trimethyladenosine |
| 3-(3-amino-3-carboxypropyl)uracil | N6,N6-dimethyladenosine |
| 3-(3-amino-3-carboxypropyl)uridine | N6-acetyladenosine |
| 3-(alkyl)cytosine | N6-cis-hydroxy-isopentenyl-adenosine |
| 3-(methyl)-7-(propynyl)isocarbostyrily1 | N6-glycinylcarbamoyladenosine |
| 3-(methyl)cytidine | N6-hydroxynorvalylcarbamoyladenosine |
| 3-(methyl)isocarbostyrilyl | N6-isopentenyladenosine |
| 3,2′-0-dimethyluridine | N6-methyl-2-amino-purine |
| 3′-alkylene phosphonates | N6-methyladenosine |
| 3-alkyl-pseudouridine | N6-methyl-N6-threonylcarbamoyladenosine |
| 3′-aminophosphoramidate | N6-substituted purines |
| 3-deaza-3-bromoadenosine | N6-threonylcarbamoyladenosine |
| 3-deaza-3-chloroadenosine | N-alkylated derivative |
| 3-deaza-3-fluoroadenosine | napthalenyl |
| 3-deaza-3-iodoadenosine | nitrobenzimidazolyl |
| 3-deazaadenosine | nitroimidazolyl |
| 3′-ethynylcytidine | nitroindazolyl |
| 3-methylcytidine | nitropyrazolyl |
| 3-methyl-pseudouridine | N1-methyl-adenosine |
| 3-methyluridine | N1-methyl-guanosine |
| 4′-azidoadenosine | nubularine |
| 4′-azidouridine | O6-substituted purines |
| 4′-ethynyladenosine | O-alkylated derivative |
| 4′-ethynylcytidine | oligonucleosides with heteroatom |
| | internucleoside linkage |
| 4′-ethynylguanosine | ortho-(aminoalkylhydroxy)-6-phenyl-pyrrolo- |
| | pyrimidin-2- on-3-yl |
| 4′-ethynyluridine | ortho-substituted-6-phenyl-pyrrolo-pyrimidin- |
| | 2-on-3-yl |
| 4-(fluoro)-6-(methyl)benzimidazole | Oxoformycin TP |
| 4-(methyl)benzimidazole | para-(aminoalkylhydroxy)-6-phenyl-pyrrolo- |
| | pyrimidin-2-on-3-yl |
| 4-(methyl)indolyl | para-substituted-6-phenyl-pyrrolo-pyrimidin- |
| | 2-on-3-yl |
| 4-(thio)pseudouracil | pentacenyl |
| 4-(thio)uracil | peroxywybutosine |
| 4,2′-O-dimethylcytidine | phenanthracenyl |
| 4,6-(dimethyl)indolyl | phenyl |
| 4′-azidocytidine | phosphinates |
| 4′-azidoguanosine | phosphonoacetates |
| 4′-carbocyclic adenosine | phosphoramidates |
| 4′-carbocyclic cytidine | Phosphorodiamidate Morpholino Oligomer |
| | (PMO) |
| 4′-carbocyclic guanosine | phosphorodithioates |
| 4′-carbocyclic uridine | Phosphorothioate |
| 4-demethylwyosine | phosphorothioate internucleoside linkages |
| 4-methoxy-1-methyl-pseudoisocytidine | phosphorothioates |
| 4-methoxy-2-thio-pseudouridine | phosphotriesters |
| 4-methoxy-pseudoisocytidine | PNA |
| 4-methoxy-pseudouridine | propynyL-7-(aza)indolyl |
| 4-methylcytidine | pseudoisocytidine |
| 4-thio-1-methyl-1-deaza-pseudoisocytidine | Pseudo-iso-cytidine |
| 4-thio-1-methyl-pseudoisocytidine | pseudouracil |
| 4-thio-1-methyl-pseudouridine | pseudouridine |
| 4-thio-pseudoisocytidine | Pseudouridine 1-(4-methylbenzenesulfonic |
| | acid) |
| 4-thio-pseudouridine | Pseudouridine 1-(4-methylbenzoic acid) TP |
| 4-thiouracil | Pseudouridine 1-methylphosphonic acid |
| 4-thiouridine | Pseudouridine 1-[3-(2-ethoxy)]propionic acid |
| 5 (halo)cytosine | Pseudouridine 1-[3-{2-(2-[2-(2-ethoxy)- |
| | ethoxy]-ethoxy)-ethoxy}]propionic acid |
| 5 (methyl) 4 (thio)uracil | Pseudouridine 1-[3-{2-(2-[2-{2(2-ethoxy)- |
| | ethoxy}-ethoxy]-ethoxy)-ethoxy}]propionic |
| | acid |
| 5 (methyl)cytosine | Pseudouridine 1-[3-{2-(2-[2-ethoxy]-ethoxy)- |
| | ethoxv}]propionic acid |
| 5 (methylaminomethyl)-2 (thio)uracil | Pseudouridine 1-[3-{2-(2-ethoxy)-ethoxv}] |
| | propionic acid |
| 5 (methylaminomethyl)-2,4 (dithio)uracil | Pseudouridine TP 1-methylphosphonic acid |
| | diethyl ester |
| 5 (methylaminomethyl)-4 (thio)uracil | Pseudo-uridine-1-2-ethanoic acid |
| 5 (propynyl)cytosine | Pseudo-uridine-N1-5-pentanoic acid |
| 5 (propynyl)uracil | Pseudo-uridine-N1-3-propionic acid |
| 5 (trifluoromethyl)cytosine | Pseudo-uridine-N1-4-butanoic acid |
| 5 (trifluoromethyl)uracil | Pseudo-uridine-N1-6-hexanoic acid |
| 5 nitroindole | Pseudo-uridine-N1-methyl-p-benzoic acid |
| 5 substituted pyrimidines | Pseudo-uridine-N1-p-benzoic acid |
| 5-(1,3-diazole-1-alkyl)uracil | Pseudo-uridine-N1-7-heptanoic acid |
| 5-(1-Propynyl)ara-cytidine | pyrenyl |
| 5-(1-Propynyl)ara-uridine | pyridin-4-one ribonucleoside |
| 5-(2-aminopropyl)uracil | pyridopyrimidin-3-yl |
| 5-(2-carbomethoxyvinyl)uridine | pyridopyrimidin-3-yl, 2-oxo-7-amino- |
| | pyridopyrimidin-3-yl |
| 5-(2-Chloro-phenyl)-2-thiocytidine | pyrrolo-cytidine |
| 5-(2-Furanyl)uridine | pyrrolo-pseudoisocytidine |
| 5-(4-Amino-phenyl)-2-thiocytidine | pyrrolo-pyrimidin-2-on-3-yl |
| 5-(alkyl)-2-(thio)pseudouracil | pyrrolopyrimidinyl |
| 5-(alkyl)-4 (thio)pseudouracil | pyrrolopyrizinyl |
| 5-(alkyl)-2,4 (dithio)pseudouracil | Pyrrolosine |
| 5-(alkyl)cytosine | siloxane backbones |
| 5-(alkyl)pseudouracil | stilbenzyl |
| 5-(alkyl)uracil | substituted 1,2,4-triazoles |
| 5-(alkynyl)cytosine | sulfamate backbones |
| 5-(alkynyl)uracil | sulfide sulfoxide and sulfone backbones |
| 5-(allylamino)uracil | sulfonate and sulfonamide backbones |
| 5-(aminoalkyl)uracil | tetracenyl |
| 5-(carboxyhydroxymethyl)uridine | thio-adenosine |
| 5-(carboxyhydroxymethyl)uridine methyl | thionoalkylphosphonates |
| ester | |
| 5-(cyanoalkyl)uracil | thionoalkylphosphotriesters |
| 5-(dialkylaminoalkyl)uracil | thionophosphoramidates |
| 5-(dimethylaminoalkyl)uracil | Tricyclo-DNA (tcDNA) |
| 5-(guanidiniumalkyl)uracil | tubercidine |
| 5-(halo)cytosine | undermodified hydroxywybutosine |
| 5-(halo)uracil | uridine 5-oxyacetic acid |
| 5-(iso-Pentenylaminomethyl)-2-thiouridine | uridine 5-oxyacetic acid methyl ester |
| 5-(iso-Pentenylaminomethyl)-2′-O- | wybutosine |
| methyluridine | |
| 5-(iso-Pentenylaminomethyl)uridine | wyosme |
| 5-(1,3-diazole-1-alkyl)uracil | xanthine |
| 5-(methoxy)uracil | Xanthosine |
| 5-(methoxycarbonylmethyl)-2-(thio)uracil | zebularine |

### TREM, TREM Core Fragment and TREM Fragment Fusions

In an embodiment, a TREM, a TREM core fragment or a TREM fragment disclosed herein comprises an additional moiety, e.g., a fusion moiety. In an embodiment, the fusion moiety can be used for purification, to alter folding of the TREM, TREM core fragment or TREM fragment, or as a targeting moiety. In an embodiment, the fusion moiety can comprise a tag, a linker, can be cleavable or can include a binding site for an enzyme. In an embodiment, the fusion moiety can be disposed at the N terminal of the TREM or at the C terminal of the TREM, TREM core fragment or TREM fragment. In an embodiment, the fusion moiety can be encoded by the same or different nucleic acid molecule that encodes the TREM, TREM core fragment or TREM fragment.

### TREM Consensus Sequence

In an embodiment, a TREM disclosed herein comprises a consensus sequence provided herein.

In an embodiment, a TREM disclosed herein comprises a consensus sequence of Formula I_{ZZZ}, wherein _{ZZZ} indicates any of the twenty amino acids and Formula I corresponds to all species.

In an embodiment, a TREM disclosed herein comprises a consensus sequence of Formula II_{ZZZ}, wherein _{ZZZ} indicates any of the twenty amino acids and Formula II corresponds to mammals.

In an embodiment, a TREM disclosed herein comprises a consensus sequence of Formula III_{ZZZ}, wherein _{ZZZ} indicates any of the twenty amino acids and Formula III corresponds to humans.

In an embodiment, _{ZZZ} indicates any of the twenty amino acids: alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.

In an embodiment, a TREM disclosed herein comprises a property selected from the following:
a) under physiological conditions residue R₀ forms a linker region, e.g., a Linker 1 region;
b) under physiological conditions residues R₁-R₂-R₃-R₄-R₅-R₆-R₇ and residues R₆₅-R₆₆-R₆₇-R₆₈-R₆₉-R₇₀-R₇₁ form a stem region, e.g., an AStD stem region;
c) under physiological conditions residues R₈-R₉ forms a linker region, e.g., a Linker 2 region;
d) under physiological conditions residues -R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ form a stem-loop region, e.g., a D arm Region;
e) under physiological conditions residue -R₂₉ forms a linker region, e.g., a Linker 3 Region;
f) under physiological conditions residues -R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ form a stem-loop region, e.g., an AC arm region;
g) under physiological conditions residue —[R₄₇]ₓ comprises a variable region, e.g., as described herein;
h) under physiological conditions residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ form a stem-loop region, e.g., a T arm Region; or
i) under physiological conditions residue R₇₂ forms a linker region, e.g., a Linker 4 region.

### Alanine TREM Consensus Sequence

In an embodiment, a TREM disclosed herein comprises the sequence of Formula I_{ALA} (SEQ ID NO: 562),

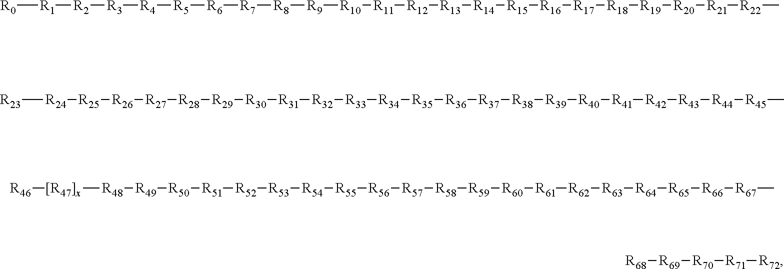

wherein R is a ribonucleotide residue and the consensus for Ala is: R₀=absent; R₁₄, R₅₇=are independently A or absent; R₂₆=A, C, G or absent; R₅, R₆, R₁₅, R₁₆, R₂₁, R₃₀, R₃₁, R₃₂, R₃₄, R₃₇, R₄₁, R₄₂, R₄₃, R₄₄, R₄₅, R₄₈, R₄₉, R₅₀, R₅₈, R₅₉, R₆₃, R₆₄, R₆₆, R₆₇=are independently N or absent; R₁₁, R₃₅, R₆₅=are independently A, C, U or absent; R₁, R₉, R₂₀, R₃₈, R₄₀, R₅₁, R₅₂, R₅₆=are independently A, G or absent; R₇, R₂₂, R₂₅, R₂₇, R₂₉, R₄₆, R₅₃, R₇₂=are independently A, G, U or absent; R₂₄, R₆₉=are independently A, U or absent; R₇₀, R₇₁=are independently C or absent; R₃, R₄=are independently C, G or absent; R₁₂, R₃₃, R₃₆, R₆₂, R₆₈=are independently C, G, U or absent; R₁₃, R₁₇, R₂₈, R₃₉, R₅₅, R₆₀, R₆₁=are independently C, U or absent; R₁₀, R₁₉, R₂₃=are independently G or absent; R₂=G, U or absent; R₈, R₁₈, R₅₄=are independently U or absent; [R₄₇]ₓ=N or absent; wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271),
provided that the TREM has one or both of the following properties: no more than 15% of the residues are N; or no more than 20 residues are absent.

In an embodiment, a TREM disclosed herein comprises the sequence of Formula II_{ALA} (SEQ ID NO: 563),

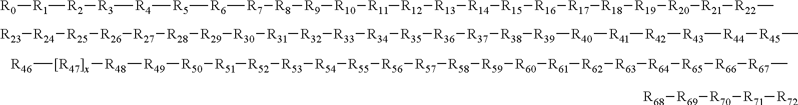

wherein R is a ribonucleotide residue and the consensus for Ala is:
R₀, R₁₈=are absent;
R₁₄, R₂₄, R₅₇=are independently A or absent;
R₁₅, R₂₆, R₆₄=are independently A, C, G or absent;
R₁₆, R₃₁, R₅₀, R₅₉=are independently N or absent;
R₁₁, R₃₂, R₃₇, R₄₁, R₄₃, R₄₅, R₄₉, R₆₅, R₆₆=are independently A, C, U or absent;
R₁, R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃, R₇₂=are independently A, G, U or absent;
R₆, R₃₅, R₆₉=are independently A, U or absent;
R₅₅, R₆₀, R₇₀, R₇₁=are independently C or absent;
R₃=C, G or absent;
R₁₂, R₃₆, R₄₈=are independently C, G, U or absent;
R₁₃, R₁₇, R₂₈, R₃₀, R₃₄, R₃₉, R₅₈, R₆₁, R₆₂, R₆₇, R₆₈=are independently C, U or absent;
R₄, R₁₀, R₁₉, R₂₀, R₂₃, R₅₂=are independently G or absent;
R₂, R₈, R₃₃=are independently G, U or absent;
R₂₁, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ALA} (SEQ ID NO: 564),

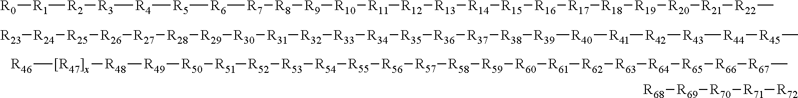

wherein R is a ribonucleotide residue and the consensus for Ala is:
R₀, R₁₈=are absent;
R₁₄, R₂₄, R₅₇, R₇₂=are independently A or absent;
R₁₅, R₂₆, R₆₄=are independently A, C, G or absent;
R₁₆, R₃₁, R₅₀=are independently N or absent;
R₁₁, R₃₂, R₃₇, R₄₁, R₄₃, R₄₅, R₄₉, R₆₅, R₆₆=are independently A, C, U or absent;
R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃=are independently A, G, U or absent;
R₆, R₃₅=are independently A, U or absent;
R₅₅, R₆₀, R₆₁, R₇₀, R₇₁=are independently C or absent;
R₁₂, R₄₈, R₅₉=are independently C, G, U or absent;
R₁₃, R₁₇, R₂₈, R₃₀, R₃₄, R₃₉, R₅₈, R₆₂, R₆₇, R₆₈=are independently C, U or absent;
R₁, R₂, R₃, R₄, R₁₀, R₁₉, R₂₀, R₂₃, R₅₂=are independently G or absent;
R₃₃, R₃₆=are independently G, U or absent;
R₈, R₂₁, R₅₄, R₆₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Arginine TREM Consensus Sequence

In an embodiment, a TREM disclosed herein comprises the sequence of Formula I_{ARG} (SEQ ID NO: 565),

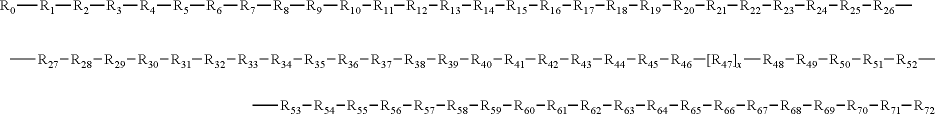

wherein R is a ribonucleotide residue and the consensus for Arg is:
R₅₇=A or absent;
R₉, R₂₇=are independently A, C, G or absent;
R₁, R₂, R₃, R₄, R₅, R₆, R₇, R₁₁, R₁₂, R₁₆, R₂₁, R₂₂, R₂₃, R₂₅, R₂₆, R₂₉, R₃₀, R₃₁, R₃₂, R₃₃, R₃₄, R₃₇, R₄₂, R₄₄, R₄₅,
R₄₆, R₄₈, R₄₉, R₅₀, R₅₁, R₅₈, R₆₂, R₆₃, R₆₄, R₆₅, R₆₆, R₆₇, R₆₈, R₆₉, R₇₀, R₇₁=are independently N or absent;
R₁₃, R₁₇, R₄₁=are independently A, C, U or absent;
R₁₉, R₂₀, R₂₄, R₄₀, R₅₆=are independently A, G or absent;
R₁₄, R₁₅, R₇₂=are independently A, G, U or absent;
R₁₈=A, U or absent;
R₃₈=C or absent;
R₃₅, R₄₃, R₆₁=are independently C, G, U or absent;
R₂₈, R₅₅, R₅₉, R₆₀=are independently C, U or absent;
R₀, R₁₀, R₅₂=are independently G or absent;
R₈, R₃₉=are independently G, U or absent;
R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, provided that the TREM has one or both of the following properties: no more than 15% of the residues are N; or no more than 20 residues are absent.

In an embodiment, a TREM disclosed herein comprises the sequence of Formula II_{ARG} (SEQ ID NO: 566),

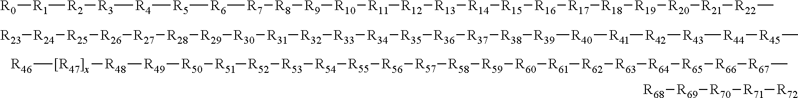

wherein R is a ribonucleotide residue and the consensus for Arg is:
R₁₈=absent;
R₂₄, R₅₇=are independently A or absent;
R₄₁=A, C or absent;
R₃, R₇, R₃₄, R₅₀=are independently A, C, G or absent;
R₂, R₅, R₆, R₁₂, R₂₆, R₃₂, R₃₇, R₄₄, R₅₈, R₆₆, R₆₇, R₆₈, R₇₀=are independently N or absent;
R₄₉, R₇₁=are independently A, C, U or absent;
R₁, R₁₅, R₁₉, R₂₅, R₂₇, R₄₀, R₄₅, R₄₆, R₅₆, R₇₂=are independently A, G or absent;
R₁₄, R₂₉, R₆₃=are independently A, G, U or absent;
R₁₆, R₂₁=are independently A, U or absent;
R₃₈, R₆₁=are independently C or absent;
R₃₃, R₄₈=are independently C, G or absent;
R₄, R₉, R₁₁, R₄₃, R₆₂, R₆₄, R₆₉=are independently C, G, U or absent;
R₁₃, R₂₂, R₂₈, R₃₀, R₃₁, R₃₅, R₅₅, R₆₀, R₆₅=are independently C, U or absent;
R₉, R₁₀, R₂₀, R₂₃, R₅₁, R₅₂=are independently G or absent;
R₈, R₃₉, R₄₂=are independently G, U or absent;
R₁₇, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ARG} (SEQ ID NO: 567),

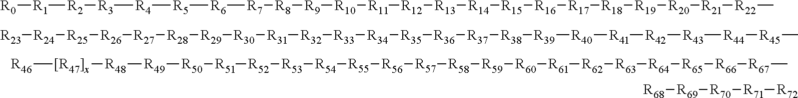

wherein R is a ribonucleotide residue and the consensus for Arg is:
R₁₈=is absent;
R₁₅, R₂₁, R₂₄, R₄₁, R₅₇=are independently A or absent;
R₃₄, R₄₄=are independently A, C or absent;
R₃, R₅, R₅₈=are independently A, C, G or absent;
R₂, R₆, R₆₆, R₇₀=are independently N or absent;
R₃₇, R₄₉=are independently A, C, U or absent;
R₁, R₂₅, R₂₉, R₄₀, R₄₅, R₄₆, R₅₀=are independently A, G or absent;
R₁₄, R₆₃, R₆₈=are independently A, G, U or absent;
R₁₆=A, U or absent;
R₃₈, R₆₁=are independently C or absent;
R₇, R₁₁, R₁₂, R₂₆, R₄₈=are independently C, G or absent;
R₆₄, R₆₇, R₆₉=are independently C, G, U or absent;
R₄, R₁₃, R₂₂, R₂₈, R₃₀, R₃₁, R₃₅, R₄₃, R₅₅, R₆₀, R₆₂, R₆₅, R₇₁=are independently C, U or absent;
R₉, R₁₀, R₁₉, R₂₀, R₂₃, R₂₇, R₃₃, R₅₁, R₅₂, R₅₆, R₇₂=are independently G or absent;
R₈, R₉, R₃₂, R₃₉, R₄₂=are independently G, U or absent;
R₁₇, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Asparagine TREM Consensus Sequence

In an embodiment, a TREM disclosed herein comprises the sequence of Formula I_{ASN} (SEQ ID NO: 568),

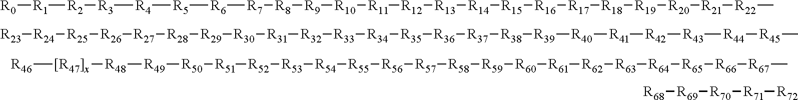

wherein R is a ribonucleotide residue and the consensus for Asn is:
R₉, R₁₈=are absent;
R₄₁=A or absent;
R₁₄, R₄₈, R₅₆=are independently A, C, G or absent;
R₂, R₄, R₅, R₆, R₁₂, R₁₇, R₂₆, R₂₉, R₃₀, R₃₁, R₄₄, R₄₅, R₄₆, R₄₉, R₅₀, R₅₈, R₆₂, R₆₃, R₆₅, R₆₆, R₆₇, R₆₈, R₇₀, R₇₁=are independently N or absent;
R₁₁, R₁₃, R₂₂, R₄₂, R₅₅, R₅₉=are independently A, C, U or absent;
R₉, R₁₅, R₂₄, R₂₇, R₃₄, R₃₇, R₅₁, R₇₂=are independently A, G or absent;
R₁, R₇, R₂₅, R₆₉=are independently A, G, U or absent;
R₄₀, R₅₇=are independently A, U or absent;
R₆₀=C or absent;
R₃₃=C, G or absent;
R₂₁, R₃₂, R₄₃, R₆₄=are independently C, G, U or absent;
R₃, R₁₆, R₂₈, R₃₅, R₃₆, R₆₁=are independently C, U or absent;
R₁₀, R₁₉, R₂₀, R₅₂=are independently G or absent;
R₅₄=G, U or absent;
R₈, R₂₃, R₃₈, R₃₉, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ASN} (SEQ ID NO: 569),

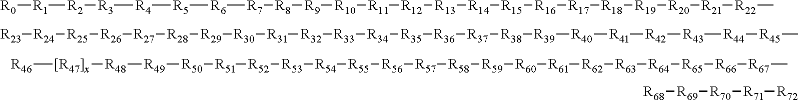

wherein R is a ribonucleotide residue and the consensus for Asn is:
R₉, R₁₈=are absent
R₂₄, R₄₁, R₄₆, R₆₂=are independently A or absent;
R₅₉=A, C or absent;
R₁₄, R₅₆, R₆₆=are independently A, C, G or absent;
R₁₇, R₂₉=are independently N or absent;
R₁₁, R₂₆, R₄₂, R₅₅=are independently A, C, U or absent;
R₁, R₉, R₁₂, R₁₅, R₂₅, R₃₄, R₃₇, R₄₈, R₅₁, R₆₇, R₆₈, R₆₉, R₇₀, R₇₂=are independently A, G or absent;
R₄₄, R₄₅, R₅₈=are independently A, G, U or absent;
R₄₀, R₅₇=are independently A, U or absent;
R₅, R₂₈, R₆₀=are independently C or absent;
R₃₃, R₆₅=are independently C, G or absent;
R₂₁, R₄₃, R₇₁=are independently C, G, U or absent;
R₃, R₆, R₁₃, R₂₂, R₃₂, R₃₅, R₃₆, R₆₁, R₆₃, R₆₄=are independently C, U or absent;
R₇, R₁₀, R₁₉, R₂₀, R₂₇, R₄₉, R₅₂=are independently G or absent;
R₅₄=G, U or absent;
R₂, R₄, R₈, R₁₆, R₂₃, R₃₀, R₃₁, R₃₈, R₃₉, R₅₀, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ASN} (SEQ ID NO: 570),

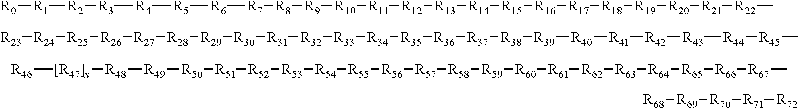

wherein R is a ribonucleotide residue and the consensus for Asn is:
R₉, R₁₈=are absent
R₂₄, R₄₀, R₄₁, R₄₆, R₆₂=are independently A or absent;
R₅₉=A, C or absent;
R₁₄, R₅₆, R₆₆=are independently A, C, G or absent;
R₁₁, R₂₆, R₄₂, R₅₅=are independently A, C, U or absent;
R₁, R₉, R₁₂, R₁₅, R₃₄, R₃₇, R₄₈, R₅₁, R₆₇, R₆₈, R₆₉, R₇₀=are independently A, G or absent;
R₄₄, R₄₅, R₅₈=are independently A, G, U or absent;
R₅₇=A, U or absent;
R₅, R₂₈, R₆₀=are independently C or absent;
R₃₃, R₆₅=are independently C, G or absent;
R₁₇, R₂₁, R₂₉=are independently C, G, U or absent;
R₃, R₆, R₁₃, R₂₂, R₃₂, R₃₅, R₃₆, R₄₃, R₆₁, R₆₃, R₆₄, R₇₁=are independently C, U or absent;
R₇, R₁₀, R₁₉, R₂₀, R₂₅, R₂₇, R₄₉, R₅₂, R₇₂=are independently G or absent;
R₅₄=G, U or absent;
R₂, R₄, R₈, R₁₆, R₂₃, R₃₀, R₃₁, R₃₈, R₃₉, R₅₀, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Aspartate TREM Consensus Sequence

In an embodiment, a TREM disclosed herein comprises the sequence of Formula I_{ASP} (SEQ ID NO: 571),

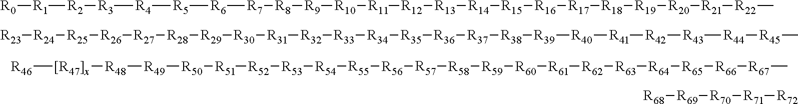

wherein R is a ribonucleotide residue and the consensus for Asp is:
R₀=absent
R₂₄, R₇₁=are independently A, C or absent;
R₃₃, R₄₆=are independently A, C, G or absent;
R₂, R₃, R₄, R₅, R₆, R₁₂, R₁₆, R₂₂, R₂₆, R₂₉, R₃₁, R₃₂, R₄₄, R₄₈, R₄₉, R₅₈, R₆₃, R₆₄, R₆₆, R₆₇, R₆₈, R₆₉=are
independently N or absent;
R₁₃, R₂₁, R₃₄, R₄₁, R₅₇, R₆₅=are independently A, C, U or absent;
R₉, R₁₀, R₁₄, R₁₅, R₂₀, R₂₇, R₃₇, R₄₀, R₅₁, R₅₆, R₇₂=are independently A, G or absent;
R₇, R₂₅, R₄₂=are independently A, G, U or absent;
R₃₉=C or absent;
R₅₀, R₆₂=are independently C, G or absent;
R₃₀, R₄₃, R₄₅, R₅₅, R₇₀=are independently C, G, U or absent;
R₈, R₁₁, R₁₇, R₁₈, R₂₈, R₃₅, R₅₃, R₅₉, R₆₀, R₆₁=are independently C, U or absent;
R₁₉, R₅₂=are independently G or absent;
R₁=G, U or absent;
R₂₃, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ASP} (SEQ ID NO: 572),

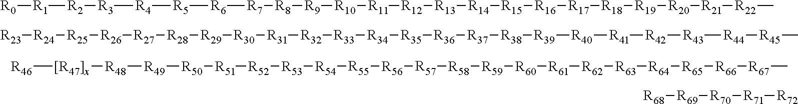

wherein R is a ribonucleotide residue and the consensus for Asp is:
R₉, R₁₇, R₁₈, R₂₃=are independently absent;
R₉, R₄₀=are independently A or absent;
R₂₄, R₇₁=are independently A, C or absent;
R₆₇, R₆₈=are independently A, C, G or absent;
R₂, R₆, R₆₆=are independently N or absent;
R₅₇, R₆₃=are independently A, C, U or absent;
R₁₀, R₁₄, R₂₇, R₃₃, R₃₇, R₄₄, R₄₆, R₅₁, R₅₆, R₆₄, R₇₂=are independently A, G or absent;
R₇, R₁₂, R₂₆, R₆₅=are independently A, U or absent;
R₃₉, R₆₁, R₆₂=are independently C or absent;
R₃, R₃₁, R₄₅, R₇₀=are independently C, G or absent;
R₄, R₅, R₂₉, R₄₃, R₅₅=are independently C, G, U or absent;
R₈, R₁₁, R₁₃, R₃₀, R₃₂, R₃₄, R₃₅, R₄₁, R₄₈, R₅₃, R₅₉, R₆₀=are independently C, U or absent;
R₁₅, R₁₉, R₂₀, R₂₅, R₄₂, R₅₀, R₅₂=are independently G or absent;
R₁, R₂₂, R₄₉, R₅₈, R₆₉=are independently G, U or absent;
R₁₆, R₂₁, R₂₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ASP} (SEQ ID NO: 573),

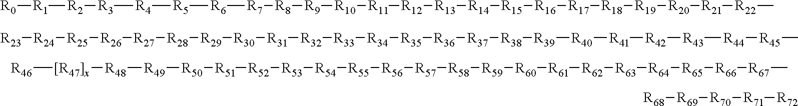

wherein R is a ribonucleotide residue and the consensus for Asp is:
R₉, R₁₇, R₁₈, R₂₃=are absent
R₉, R₁₂, R₄₀, R₆₅, R₇₁=are independently A or absent;
R₂, R₂₄, R₅₇=are independently A, C or absent;
R₆, R₁₄, R₂₇, R₄₆, R₅₁, R₅₆, R₆₄, R₆₇, R₆₈=are independently A, G or absent;
R₃, R₃₁, R₃₅, R₃₉, R₆₁, R₆₂=are independently C or absent;
R₆₆=C, G or absent;
R₅, R₈, R₂₉, R₃₀, R₃₂, R₃₄, R₄₁, R₄₃, R₄₈, R₅₅, R₅₉, R₆₀, R₆₃=are independently C, U or absent;
R₁₀, R₁₅, R₁₉, R₂₀, R₂₅, R₃₃, R₃₇, R₄₂, R₄₄, R₄₅, R₄₉, R₅₀, R₅₂, R₆₉, R₇₀, R₇₂=are independently G or
absent;
R₂₂, R₅₈=are independently G, U or absent;
R₁, R₄, R₇, R₁₁, R₁₃, R₁₆, R₂₁, R₂₆, R₂₈, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Cysteine TREM Consensus Sequence

In an embodiment, a TREM disclosed herein comprises the sequence of Formula I_{CYS} (SEQ ID NO: 574),

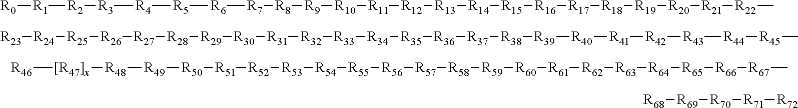

wherein R is a ribonucleotide residue and the consensus for Cys is:
R₀=absent
R₁₄, R₃₉, R₅₇=are independently A or absent;
R₄₁=A, C or absent;
R₁₀, R₁₅, R₂₇, R₃₃, R₆₂=are independently A, C, G or absent;
R₃, R₄, R₅, R₆, R₁₂, R₁₃, R₁₆, R₂₄, R₂₆, R₂₉, R₃₀, R₃₁, R₃₂, R₃₄, R₄₂, R₄₄, R₄₅, R₄₆, R₄₈, R₄₉, R₅₈, R₆₃, R₆₄, R₆₆,
R₆₇, R₆₈, R₆₉, R₀=are independently N or absent;
R₆₅=A, C, U or absent;
R₉, R₂₅, R₃₇, R₄₀, R₅₂, R₅₆=are independently A, G or absent;
R₇, R₂₀, R₅₁=are independently A, G, U or absent;
R₁₈, R₃₈, R₅₅=are independently C or absent;
R₂=C, G or absent;
R₂₁, R₂₈, R₄₃, R₅₀=are independently C, G, U or absent;
R₁₁, R₂₂, R₂₃, R₃₅, R₃₆, R₅₉, R₆₀, R₆₁, R₇₁, R₇₂=are independently C, U or absent;
R₁, R₁₉=are independently G or absent;
R₁₇=G, U or absent;
R₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, provided that the TREM has one or both of the following properties: no more than 15% of the residues are N; or no more than 20 residues are absent.

In an embodiment, a TREM disclosed herein comprises the sequence of Formula II_{CYS} (SEQ ID NO: 575),

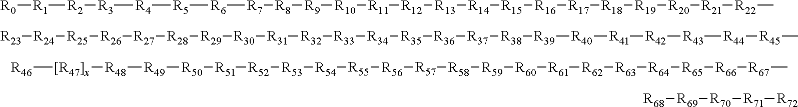

wherein R is a ribonucleotide residue and the consensus for Cys is:
R₉, R₁₈, R₂₃=are absent; R₁₄, R₂₄, R₂₆, R₂₉, R₃₉, R₄₁, R₄₅, R₅₇=are independently A or absent; R₄₄=A, C or absent; R₂₇, R₆₂=are independently A, C, G or absent;
R₁₆=A, C, G, U or absent;
R₃₀, R₇₀=are independently A, C, U or absent; R₅, R₇, R₉, R₂₅, R₃₄, R₃₇, R₄₀, R₄₆, R₅₂, R₅₆, R₅₈, R₆₆=are independently A, G or absent; R₂₀, R₅₁=are independently A, G, U or absent;
R₃₅, R₃₈, R₄₃, R₅₅, R₆₉=are independently C or absent; R₂, R₄, R₁₅=are independently C, G or absent;
R₁₃=C, G, U or absent;
R₆, R₁₁, R₂₈, R₃₆, R₄₈, R₄₉, R₅₀, R₆₀, R₆₁, R₆₇, R₆₈, R₇₁, R₇₂=are independently C, U or absent; R₁, R₃, R₁₀, R₁₉, R₃₃, R₆₃=are independently G or absent; R₈, R₁₇, R₂₁, R₆₄=are independently G, U or absent;
R₁₂, R₂₂, R₃₁, R₃₂, R₄₂, R₅₃, R₅₄, R₆₅=are independently U or absent;
R₅₉=U, or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{CYS} (SEQ ID NO: 576),

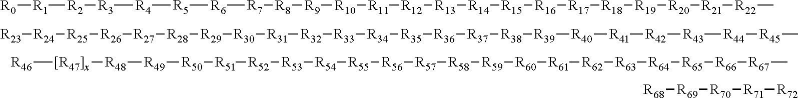

wherein R is a ribonucleotide residue and the consensus for Cys is:
R₉, R₁₈, R₂₃=are absent
R₁₄, R₂₄, R₂₆, R₂₉, R₃₄, R₃₉, R₄₁, R₄₅, R₅₇, R₅₈=are independently A or absent;
R₄₄, R₇₀=are independently A, C or absent; R₆₂=A, C, G or absent;
R₁₆=N or absent;
R₅, R₇, R₉, R₂₀, R₄₀, R₄₆, R₅₁, R₅₂, R₅₆, R₆₆=are independently A, G or absent;
R₂₈, R₃₅, R₃₈, R₄₃, R₅₅, R₆₇, R₆₉=are independently C or absent; R₄, R₁₅=are independently C, G or absent; R₆, R₁₁, R₁₃, R₃₀, R₄₈, R₄₉, R₅₀, R₆₀, R₆₁, R₆₈, R₇₁, R₇₂=are independently C, U or absent; R₁, R₂, R₃, R₁₀, R₁₉, R₂₅, R₂₇, R₃₃, R₃₇, R₆₃=are independently G or absent; R₈, R₂₁, R₆₄=are independently G, U or absent;
R₁₂, R₁₇, R₂₂, R₃₁, R₃₂, R₃₆, R₄₂, R₅₃, R₅₄, R₅₉, R₆₅=are independently U or absent; [R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamine TREM Consensus Sequence

In an embodiment, a TREM disclosed herein comprises the sequence of Formula I_{GLN} (SEQ ID NO: 577),

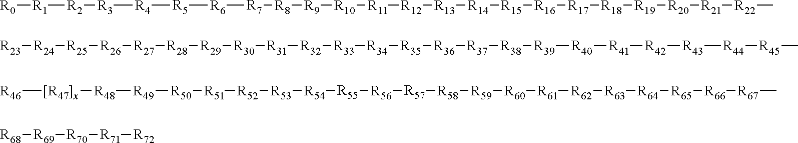

wherein R is a ribonucleotide residue and the consensus for Gln is:
R₉, R₁₈=are absent;
R₁₄, R₂₄, R₅₇=are independently A or absent;
R₉, R₂₆, R₂₇, R₃₃, R₅₆=are independently A, C, G or absent;
R₂, R₄, R₅, R₆, R₁₂, R₁₃, R₁₆, R₂₁, R₂₂, R₂₅, R₂₉, R₃₀, R₃₁, R₃₂, R₃₄, R₄₁, R₄₂, R₄₄, R₄₅, R₄₆, R₄₈, R₄₉, R₅₀, R₅₈, R₆₂, R₆₃, R₆₆, R₆₇, R₆₈, R₆₉, R₇₀=are independently N or absent;
R₁₇, R₂₃, R₄₃, R₆₅, R₇₁=are independently A, C, U or absent;
R₁₅, R₄₀, R₅₁, R₅₂=are independently A, G or absent;
R₁, R₇, R₇₂=are independently A, G, U or absent;
R₃, R₁₁, R₃₇, R₆₀, R₆₄=are independently C, G, U or absent;
R₂₈, R₃₅, R₅₅, R₅₉, R₆₁=are independently C, U or absent;
R₁₀, R₁₉, R₂₀=are independently G or absent;
R₃₉=G, U or absent;
R₈, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{GLN} (SEQ ID NO: 578),

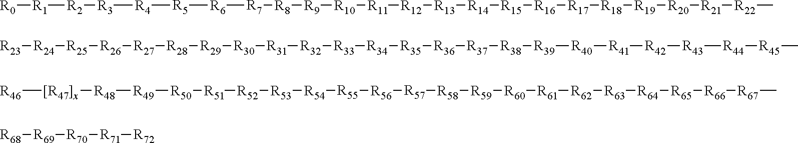

wherein R is a ribonucleotide residue and the consensus for Gln is:
R₉, R₁₈, R₂₃=are absent
R₁₄, R₂₄, R₅₇=are independently A or absent;
R₁₇, R₇₁=are independently A, C or absent;
R₂₅, R₂₆, R₃₃, R₄₄, R₄₆, R₅₆, R₆₉=are independently A, C, G or absent;
R₄, R₅, R₁₂, R₂₂, R₂₉, R₃₀, R₄₈, R₄₉, R₆₃, R₆₇, R₆₈=are independently N or absent;
R₃₁, R₄₃, R₆₂, R₆₅, R₇₀=are independently A, C, U or absent;
R₁₅, R₂₇, R₃₄, R₄₀, R₄₁, R₅₁, R₅₂=are independently A, G or absent;
R₂, R₇, R₂₁, R₄₅, R₅₀, R₅₈, R₆₆, R₇₂=are independently A, G, U or absent;
R₃, R₁₃, R₃₂, R₃₇, R₄₂, R₆₀, R₆₄=are independently C, G, U or absent;
R₆, R₁₁, R₂₈, R₃₅, R₅₅, R₅₉, R₆₁=are independently C, U or absent;
R₉, R₁₀, R₁₉, R₂₀=are independently G or absent;
R₁, R₁₆, R₃₉=are independently G, U or absent;
R₈, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{GLN} (SEQ ID NO: 579),

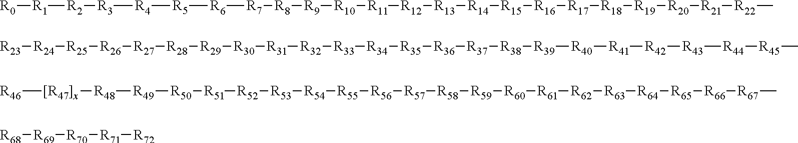

wherein R is a ribonucleotide residue and the consensus for Gln is:
R₉, R₁₈, R₂₃=are absent
R₁₄, R₂₄, R₄₁, R₅₇=are independently A or absent;
R₁₇, R₇₁=are independently A, C or absent;
R₅, R₂₅, R₂₆, R₄₆, R₅₆, R₆₉=are independently A, C, G or absent;
R₄, R₂₂, R₂₉, R₃₀, R₄₈, R₄₉, R₆₃, R₆₈=are independently N or absent;
R₄₃, R₆₂, R₆₅, R₇₀=are independently A, C, U or absent;
R₁₅, R₂₇, R₃₃, R₃₄, R₄₀, R₅₁, R₅₂=are independently A, G or absent;
R₂, R₇, R₁₂, R₄₅, R₅₀, R₅₈, R₆₆=are independently A, G, U or absent;
R₃₁=A, U or absent;
R₃₂, R₄₄, R₆₀=are independently C, G or absent;
R₃, R₁₃, R₃₇, R₄₂, R₆₄, R₆₇=are independently C, G, U or absent;
R₆, R₁₁, R₂₈, R₃₅, R₅₅, R 59, R₆₁=are independently C, U or absent;
R₉, R₁₀, R₁₉, R₂₀=are independently G or absent;
R₁, R₂₁, R₃₉, R₇₂=are independently G, U or absent;
R₈, R₁₆, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamate TREM Consensus Sequence

In an embodiment, a TREM disclosed herein comprises the sequence of Formula I_{GLU} (SEQ ID NO: 580),

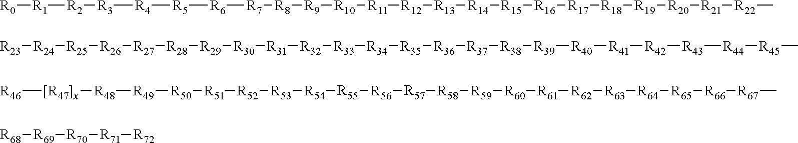

wherein R is a ribonucleotide residue and the consensus for Glu is:
R₀=absent;
R₃₄, R₄₃, R₆₈, R₆₉=are independently A, C, G or absent;
R₁, R₂, R₅, R₆, R₉, R₁₂, R₁₆, R₂₀, R₂₁, R₂₆, R₂₇, R₂₉, R₃₀, R₃₁, R₃₂, R₃₃, R₄₁, R₄₄, R₄₅, R₄₆, R₄₈, R₅₀, R₅₁, R₅₈, R₆₃, R₆₄, R₆₅, R₆₆, R₇₀, R₇₁=are independently N or absent;
R₁₃, R₁₇, R₂₃, R₆₁=are independently A, C, U or absent;
R₁₀, R₁₄, R₂₄, R₄₀, R₅₂, R₅₆=are independently A, G or absent;
R₇, R₁₅, R₂₅, R₆₇, R₇₂=are independently A, G, U or absent;
R₁₁, R₅₇=are independently A, U or absent;
R₃₉=C, G or absent;
R₃, R₄, R₂₂, R₄₂, R₄₉, R₅₅, R₆₂=are independently C, G, U or absent;
R₁₈, R₂₈, R₃₅, R₃₇, R₅₃, R₅₉, R₆₀=are independently C, U or absent;
R₁₉=G or absent;
R₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{GLU} (SEQ ID NO: 581),

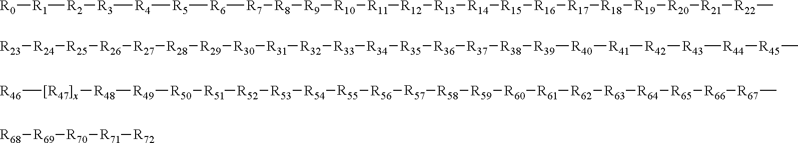

wherein R is a ribonucleotide residue and the consensus for Glu is:
R₉, R₁₈, R₂₃=are absent
R₁₇, R₄₀=are independently A or absent;
R₂₆, R₂₇, R₃₄, R₄₃, R₆₈, R₆₉, R₇₁=are independently A, C, G or absent;
R₁, R₂, R₅, R₁₂, R₂₁, R₃₁, R₃₃, R₄₁, R₄₅, R₄₈, R₅₁, R₅₈, R₆₆, R₇₀=are independently N or absent;
R₄₄, R₆₁=are independently A, C, U or absent;
R₉, R₁₄, R₂₄, R₂₅, R₅₂, R₅₆, R₆₃=are independently A, G or absent;
R₇, R₁₅, R₄₆, R₅₀, R₆₇, R₇₂=are independently A, G, U or absent;
R₂₉, R₅₇=are independently A, U or absent;
R₆₀=C or absent;
R₃₉=C, G or absent;
R₃, R₆, R₂₀, R₃₀, R₃₂, R₄₂, R₅₅, R₆₂, R₆₅=are independently C, G, U or absent;
R₄, R₈, R₁₆, R₂₈, R₃₅, R₃₇, R₄₉, R₅₃, R₅₉=are independently C, U or absent;
R₁₀, R₁₉=are independently G or absent;
R₂₂, R₆₄=are independently G, U or absent;
R₁₁, R₁₃, R₃₆, R₃₈, R₅₄=are independently U or absent;
[R₄₇]ₓ=Nor absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{GLU} (SEQ ID NO: 582),

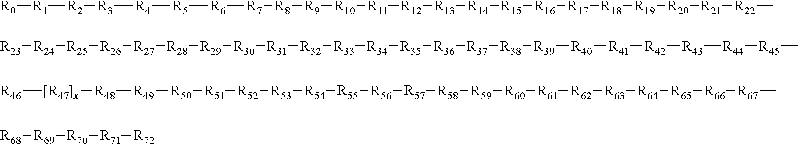

wherein R is a ribonucleotide residue and the consensus for Glu is:
R₉, R₁₇, R₁₈, R₂₃=are absent
R₁₄, R₂₇, R₄₀, R₇₁=are independently A or absent;
R₄₄=A, C or absent;
R₄₃=A, C, G or absent;
R₁, R₃₁, R₃₃, R₄₅, R₅₁, R₆₆=are independently N or absent;
R₂₁, R₄₁=are independently A, C, U or absent;
R₇, R₂₄, R₂₅, R₅₀, R₅₂, R₅₆, R₆₃, R₆₈, R₇₀=are independently A, G or absent;
R₅, R₄₆=are independently A, G, U or absent;
R₂₉, R₅₇, R₆₇, R₇₂=are independently A, U or absent;
R₂, R₃₉, R₆₀=are independently C or absent;
R₃, R₁₂, R₂₀, R₂₆, R₃₄, R₆₉=are independently C, G or absent;
R₆, R₃₀, R₄₂, R₄₈, R₆₅=are independently C, G, U or absent;
R₄, R₁₆, R₂₈, R₃₅, R₃₇, R₄₉, R₅₃, R₅₅, R₅₈, R₆₁, R₆₂=are independently C, U or absent;
R₉, R₁₀, R₁₉, R₆₄=are independently G or absent;
R₁₅, R₂₂, R₃₂=are independently G, U or absent;
R₈, R₁₁, R₁₃, R₃₆, R₃₈, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glycine TREM Consensus Sequence

In an embodiment, a TREM disclosed herein comprises the sequence of Formula I_{GLY} (SEQ ID NO: 583),

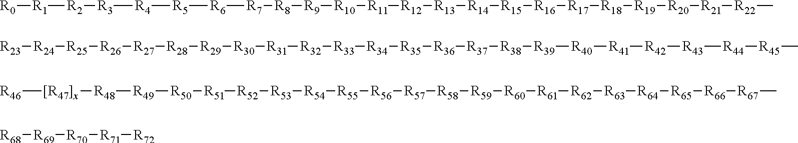

wherein R is a ribonucleotide residue and the consensus for Gly is:
R₀=absent;
R₂₄=A or absent;
R₃, R₉, R₄₀, R₅₀, R₅₁=are independently A, C, G or absent;
R₄, R₅, R₆, R₇, R₁₂, R₁₆, R₂₁, R₂₂, R₂₆, R₂₉, R₃₀, R₃₁, R₃₂, R₃₃, R₃₄, R₄₁, R₄₂, R₄₃, R₄₄, R₄₅, R₄₆, R₄₈, R₄₉, R₅₈, R
63, R₆₄, R₆₅, R₆₆, R₆₇, R₆₈=are independently N or absent;
R₅₉=A, C, U or absent;
R₁, R₁₀, R₁₄, R₁₅, R₂₇, R₅₆=are independently A, G or absent;
R₂₀, R₂₅=are independently A, G, U or absent;
R₅₇, R₇₂=are independently A, U or absent;
R₃₈, R₃₉, R₆₀=are independently C or absent;
R₅₂=C, G or absent;
R₂, R₁₉, R₃₇, R₅₄, R₅₅, R₆₁, R₆₂, R₆₉, R₀=are independently C, G, U or absent;
R₁₁, R₁₃, R₁₇, R₂₈, R₃₅, R₃₆, R₇₁=are independently C, U or absent;
R₈, R₁₈, R₂₃, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{GLY} (SEQ ID NO: 584),

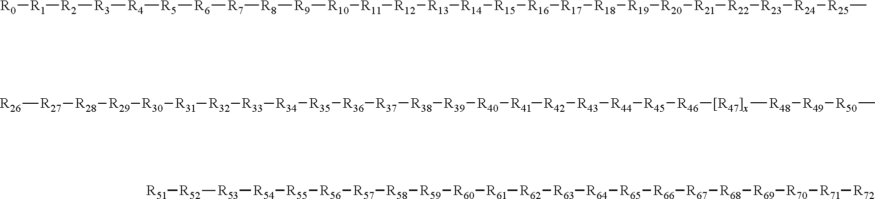

wherein R is a ribonucleotide residue and the consensus for Gly is:
R₉, R₁₈, R₂₃=are absent
R₂₄, R₂₇, R₄₀, R₇₂=are independently A or absent;
R₂₆=A, C or absent;
R₃, R₇, R₆₈=are independently A, C, G or absent;
R₅, R₃₀, R₄₁, R₄₂, R₄₄, R₄₉, R₆₇=are independently A, C, G, U or absent;
R₃₁, R₃₂, R₃₄=are independently A, C, U or absent;
R₉, R₁₀, R₁₄, R₁₅, R₃₃, R₅₀, R₅₆=are independently A, G or absent;
R₁₂, R₁₆, R₂₂, R₂₅, R₂₉, R₄₆=are independently A, G, U or absent;
R₅₇=A, U or absent;
R₁₇, R₃₈, R₃₉, R₆₀, R₆₁, R₇₁=are independently C or absent;
R₆, R₅₂, R₆₄, R₆₆=are independently C, G or absent;
R₂, R₄, R₃₇, R₄₈, R₅₅, R₆₅=are independently C, G, U or absent;
R₁₃, R₃₅, R₄₃, R₆₂, R₆₉=are independently C, U or absent;
R₁, R₁₉, R₂₀, R₅₁, R₇₀=are independently G or absent;
R₂₁, R₄₅, R₆₃=are independently G, U or absent;
R₈, R₁₁, R₂₈, R₃₆, R₅₃, R₅₄, R₅₈, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, provided that the TREM has one or both of the following properties: no more than 15% of the residues are N; or no more than 20 residues are absent.

In an embodiment, a TREM disclosed herein comprises the sequence of Formula III_{GLY} (SEQ ID NO: 585),

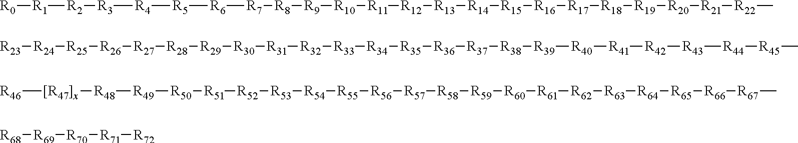

wherein R is a ribonucleotide residue and the consensus for Gly is:
R₉, R₁₈, R₂₃=are absent
R₂₄, R₂₇, R₄₀, R₇₂=are independently A or absent;
R₂₆=A, C or absent;
R₃, R₇, R₄₉, R₆₈=are independently A, C, G or absent;
R₅, R₃₀, R₄₁, R₄₄, R₆₇=are independently N or absent;
R₃₁, R₃₂, R₃₄=are independently A, C, U or absent;
R₉, R₁₀, R₁₄, R₁₅, R₃₃, R₅₀, R₅₆=are independently A, G or absent;
R₁₂, R₂₅, R₂₉, R₄₂, R₄₆=are independently A, G, U or absent;
R₁₆, R₅₇=are independently A, U or absent;
R₁₇, R₃₈, R₃₉, R₆₀, R₆₁, R₇₁=are independently C or absent;
R₆, R₅₂, R₆₄, R₆₆=are independently C, G or absent;
R₃₇, R₄₈, R₆₅=are independently C, G, U or absent;
R₂, R₄, R₁₃, R₃₅, R₄₃, R₅₅, R₆₂, R₆₉=are independently C, U or absent;
R₁, R₁₉, R₂₀, R₅₁, R₇₀=are independently G or absent;
R₂₁, R₂₂, R₄₅, R₆₃=are independently G, U or absent;
R₈, R₁₁, R₂₈, R₃₆, R₅₃, R₅₄, R₅₈, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Histidine TREM Consensus Sequence

In an embodiment, a TREM disclosed herein comprises the sequence of Formula I_{HIS} (SEQ ID NO: 586),

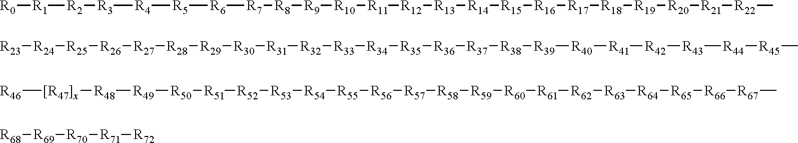

wherein R is a ribonucleotide residue and the consensus for His is:
R₂₃=absent;
R₁₄, R₂₄, R₅₇=are independently A or absent;
R₇₂=A, C or absent;
R₉, R₂₇, R₄₃, R₄₈, R₆₉=are independently A, C, G or absent;
R₃, R₄, R₅, R₆, R₁₂, R₂₅, R₂₆, R₂₉, R₃₀, R₃₁, R₃₄, R₄₂, R₄₅, R₄₆, R₄₉, R₅₀, R₅₈, R₆₂, R₆₃, R₆₆, R₆₇, R₆₈=are
independently N or absent;
R₁₃, R₂₁, R₄₁, R₄₄, R₆₅=are independently A, C, U or absent;
R₄₀, R₅₁, R₅₆, R₇₀=are independently A, G or absent;
R₇, R₃₂=are independently A, G, U or absent;
R₅₅, R₆₀=are independently C or absent;
R₁₁, R₁₆, R₃₃, R₆₄=are independently C, G, U or absent;
R₂, R₁₇, R₂₂, R₂₈, R₃₅, R₅₃, R₅₉, R₆₁, R₇₁=are independently C, U or absent;
R₁, R₁₀, R₁₅, R₁₉, R₂₀, R₃₇, R₃₉, R₅₂=are independently G or absent;
R₀=G, U or absent;
R₈, R₁₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{HIS} (SEQ ID NO: 587),

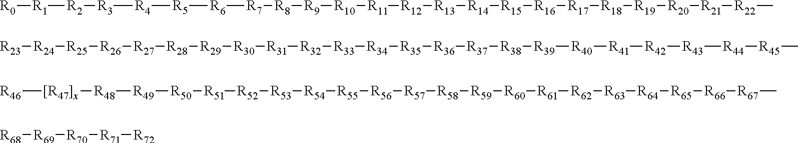

wherein R is a ribonucleotide residue and the consensus for His is:
R₉, R₁₇, R₁₈, R₂₃=are absent;
R₇, R₁₂, R₁₄, R₂₄, R₂₇, R₄₅, R₅₇, R₅₈, R₆₃, R₆₇, R₇₂=are independently A or absent;
R₃=A, C, U or absent;
R₄, R₄₃, R₅₆, R₇₀=are independently A, G or absent;
R₄₉=A, U or absent;
R₂, R₂₈, R₃₀, R₄₁, R₄₂, R₄₄, R₄₈, R₅₅, R₆₀, R₆₆, R₇₁=are independently C or absent;
R₂₅=C, G or absent;
R₉=C, G, U or absent;
R₈, R₁₃, R₂₆, R₃₃, R₃₅, R₅₀, R₅₃, R₆₁, R₆₈=are independently C, U or absent;
R₁, R₆, R₁₀, R₁₅, R₁₉, R₂₀, R₃₂, R₃₄, R₃₇, R₃₉, R₄₀, R₄₆, R₅₁, R₅₂, R₆₂, R₆₄, R₆₉=are independently G or absent;
R₁₆=G, U or absent;
R₅, R₁₁, R₂₁, R₂₂, R₂₉, R₃₁, R₃₆, R₃₈, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{HIS} (SEQ ID NO: 588),

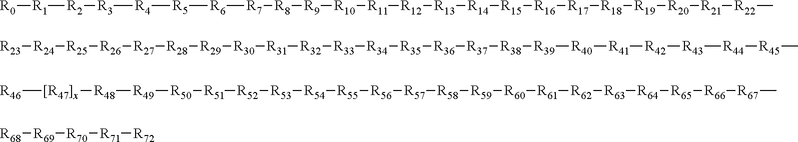

wherein R is a ribonucleotide residue and the consensus for His is:
R₀, R₁₇, R₁₈, R₂₃=are absent
R₇, R₁₂, R₁₄, R₂₄, R₂₇, R₄₅, R₅₇, R₅₈, R₆₃, R₆₇, R₇₂=are independently A or absent;
R₃=A, C or absent;
R₄, R₄₃, R₅₆, R₇₀=are independently A, G or absent;
R₄₉=A, U or absent;
R₂, R₂₈, R₃₀, R₄₁, R₄₂, R₄₄, R₄₈, R₅₅, R₆₀, R₆₆, R₇₁=are independently C or absent;
R₈, R₉, R₂₆, R₃₃, R₃₅, R₅₀, R₆₁, R₆₈=are independently C, U or absent;
R₁, R₆, R₁₀, R₁₅, R₁₉, R₂₀, R₂₅, R₃₂, R₃₄, R₃₇, R₃₉, R₄₀, R₄₆, R₅₁, R₅₂, R₆₂, R₆₄, R₆₉=are independently G or absent;
R₅, R₁₁, R₁₃, R₁₆, R₂₁, R₂₂, R₂₉, R₃₁, R₃₆, R₃₈, R₅₃, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Isoleucine TREM Consensus Sequence

In an embodiment, a TREM disclosed herein comprises the sequence of Formula I_{ILE} (SEQ ID NO: 589),

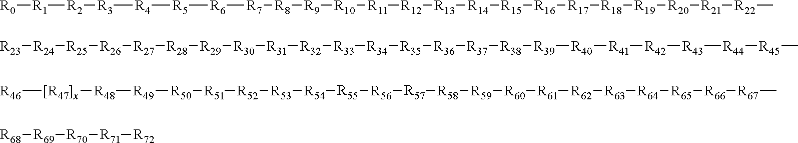

wherein R is a ribonucleotide residue and the consensus for Ile is:
R₂₃=absent;
R₃₈, R₄₁, R₅₇, R₇₂=are independently A or absent;
R₁, R₂₆=are independently A, C, G or absent;
R₉, R₃, R₄, R₆, R₁₆, R₃₁, R₃₂, R₃₄, R₃₇, R₄₂, R₄₃, R₄₄, R₄₅, R₄₆, R₄₈, R₄₉, R₅₀, R₅₈, R₅₉, R₆₂, R₆₃, R₆₄, R₆₆, R₆₇, R₆₈, R₆₉=are independently N or absent;
R₂₂, R₆₁, R₆₅=are independently A, C, U or absent;
R₉, R₁₄, R₁₅, R₂₄, R₂₇, R₄₀=are independently A, G or absent;
R₇, R₂₅, R₂₉, R₅₁, R₅₆=are independently A, G, U or absent;
R₁₈, R₅₄=are independently A, U or absent;
R₆₀=C or absent;
R₂, R₅₂, R₇₀=are independently C, G or absent;
R₅, R₁₂, R₂₁, R₃₀, R₃₃, R₇₁=are independently C, G, U or absent;
R₁₁, R₁₃, R₁₇, R₂₈, R₃₅, R₅₃, R₅₅=are independently C, U or absent;
R₁₀, R₁₉, R₂₀=are independently G or absent;
R₈, R₃₆, R₃₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ILE} (SEQ ID NO: 590),

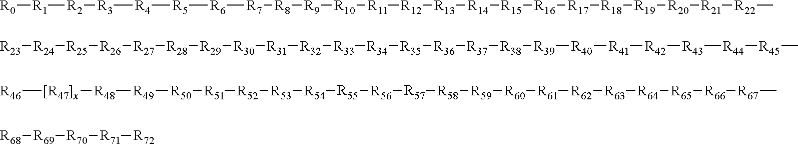

wherein R is a ribonucleotide residue and the consensus for Ile is:
R₉, R₁₈, R₂₃=are absent
R₂₄, R₃₈, R₄₀, R₄₁, R₅₇, R₇₂=are independently A or absent;
R₂₆, R₆₅=are independently A, C or absent;
R₅₈, R₅₉, R₆₇=are independently N or absent;
R₂₂=A, C, U or absent;
R₆, R₉, R₁₄, R₁₅, R₂₉, R₃₄, R₄₃, R₄₆, R₄₈, R₅₀, R₅₁, R₆₃, R₆₉=are independently A, G or absent;
R₃₇, R₅₆=are independently A, G, U or absent;
R₅₄=A, U or absent;
R₂₈, R₃₅, R₆₀, R₆₂, R₇₁=are independently C or absent;
R₂, R₅₂, R₇₀=are independently C, G or absent;
R₅=C, G, U or absent;
R₃, R₄, R₁₁, R₁₃, R₁₇, R₂₁, R₃₀, R₄₂, R₄₄, R₄₅, R₄₉, R₅₃, R₅₅, R₆₁, R₆₄, R₆₆=are independently C, U or absent;
R₁, R₁₀, R₁₉, R₂₀, R₂₅, R₂₇, R₃₁, R₆₈=are independently G or absent;
R₇, R₁₂, R₃₂=are independently G, U or absent;
R₈, R₁₆, R₃₃, R₃₆, R₃₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ILE} (SEQ ID NO: 591),

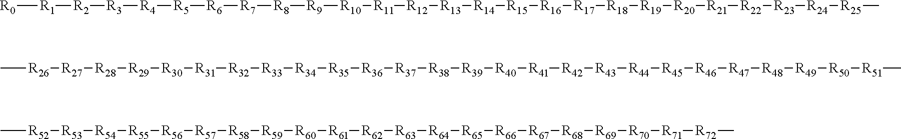

wherein R is a ribonucleotide residue and the consensus for Ile is:
R₉, R₁₈, R₂₃=are absent
R₁₄, R₂₄, R₃₈, R₄₀, R₄₁, R₅₇, R₇₂=are independently A or absent;
R₂₆, R₆₅=are independently A, C or absent;
R₂₂, R₅₉=are independently A, C, U or absent;
R₆, R₉, R₁₅, R₃₄, R₄₃, R₄₆, R₅₁, R₅₆, R₆₃, R₆₉=are independently A, G or absent;
R₃₇=A, G, U or absent;
R₁₃, R₂₈, R₃₅, R₄₄, R₅₅, R₆₀, R₆₂, R₇₁=are independently C or absent;
R₂, R₅, R₀=are independently C, G or absent;
R₅₈, R₆₇=are independently C, G, U or absent;
R₃, R₄, R₁₁, R₁₇, R₂₁, R₃₀, R₄₂, R₄₅, R₄₉, R₅₃, R₆₁, R₆₄, R₆₆=are independently C, U or absent;
R₁, R₁₀, R₁₉, R₂₀, R₂₅, R₂₇, R₂₉, R₃₁, R₃₂, R₄₈, R₅₀, R₅₂, R₆₈=are independently G or absent;
R₇, R₁₂=are independently G, U or absent;
R₈, R₁₆, R₃₃, R₃₆, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Methionine TREM Consensus Sequence

In an embodiment, a TREM disclosed herein comprises the sequence of Formula I_{MET} (SEQ ID NO: 592),

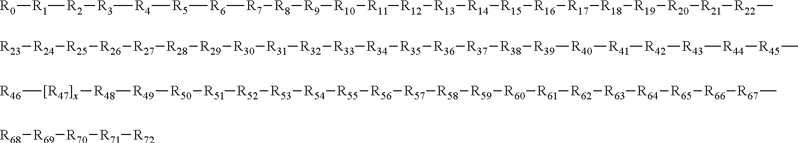

wherein R is a ribonucleotide residue and the consensus for Met is:
R₉, R₂₃=are absent;
R₁₄, R₃₈, R₄₀, R₅₇=are independently A or absent;
R₆₀=A, C or absent;
R₃₃, R₄₈, R₇₀=are independently A, C, G or absent;
R₁, R₃, R₄, R₅, R₆, R₁₁, R₁₂, R₁₆, R₁₇, R₂₁, R₂₂, R₂₆, R₂₇, R₂₉, R₃₀, R₃₁, R₃₂, R₄₂, R₄₄, R₄₅, R₄₆, R₄₉, R₅₀, R₅₈, R₆₂, R₆₃, R₆₆, R₆₇, R₆₈, R₆₉, R₇₁=are independently N or absent;
R₁₈, R₃₅, R₄₁, R₅₉, R₆₅=are independently A, C, U or absent;
R₉, R₁₅, R₅₁=are independently A, G or absent;
R₇, R₂₄, R₂₅, R₃₄, R₅₃, R₅₆=are independently A, G, U or absent;
R₇₂=A, U or absent;
R₃₇=C or absent;
R₁₀, R₅₅=are independently C, G or absent;
R₂, R₁₃, R₂₈, R₄₃, R₆₄=are independently C, G, U or absent;
R₃₆, R₆₁=are independently C, U or absent;
R₁₉, R₂₀, R₅₂=are independently G or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{MET} (SEQ ID NO: 593),

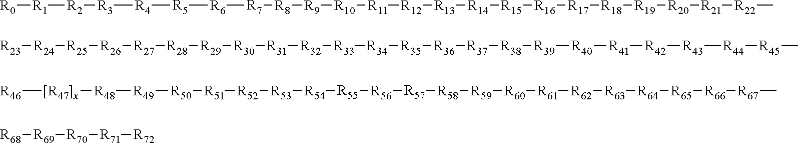

wherein R is a ribonucleotide residue and the consensus for Met is:
R₉, R₁₈, R₂₂, R₂₃=are absent
R₁₄, R₂₄, R₃₈, R₄₀, R₄₁, R₅₇, R₇₂=are independently A or absent;
R₅₉, R₆₀, R₆₂, R₆₅=are independently A, C or absent;
R₆, R₄₅, R₆₇=are independently A, C, G or absent;
R₄=N or absent;
R₂₁, R₄₂=are independently A, C, U or absent;
R₁, R₉, R₂₇, R₂₉, R₃₂, R₄₆, R₅₁=are independently A, G or absent;
R₁₇, R₄₉, R₅₃, R₅₆, R₅₈=are independently A, G, U or absent;
R₆₃=A, U or absent;
R₃, R₁₃, R₃₇=are independently C or absent;
R₄₈, R₅₅, R₆₄, R₇₀=are independently C, G or absent;
R₂, R₅, R₆₆, R₆₈=are independently C, G, U or absent;
R₁₁, R₁₆, R₂₆, R₂₈, R₃₀, R₃₁, R₃₅, R₃₆, R₄₃, R₄₄, R₆₁, R₇₁=are independently C, U or absent;
R₁₀, R₁₂, R₁₅, R₁₉, R₂₀, R₂₅, R₃₃, R₅₂, R₆₉=are independently G or absent;
R₇, R₃₄, R₅₀=are independently G, U or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{MET} (SEQ ID NO: 594),

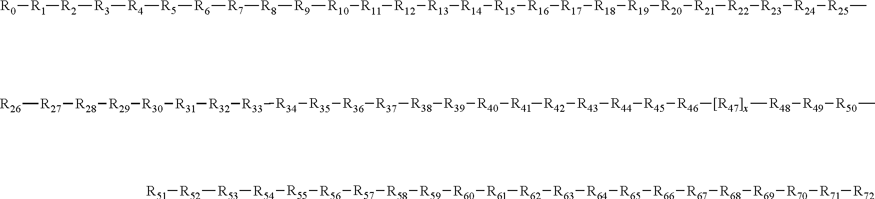

wherein R is a ribonucleotide residue and the consensus for Met is:
R₉, R₁₈, R₂₂, R₂₃=are absent
R₁₄, R₂₄, R₃₈, R₄₀, R₄₁, R₅₇, R₇₂=are independently A or absent;
R₅₉, R₆₂, R₆₅=are independently A, C or absent;
R₆, R₆₇=are independently A, C, G or absent;
R₄, R₂₁=are independently A, C, U or absent;
R₁, R₉, R₂₇, R₂₉, R₃₂, R₄₅, R₄₆, R₅₁=are independently A, G or absent;
R₁₇, R₅₆, R₅₈=are independently A, G, U or absent;
R₄₉, R₅₃, R₆₃=are independently A, U or absent;
R₃, R₁₃, R₂₆, R₃₇, R₄₃, R₆₀=are independently C or absent;
R₂, R₄₈, R₅₅, R₆₄, R₇₀=are independently C, G or absent;
R₅, R₆₆=are independently C, G, U or absent;
R₁₁, R₁₆, R₂₈, R₃₀, R₃₁, R₃₅, R₃₆, R₄₂, R₄₄, R₆₁, R₇₁=are independently C, U or absent;
R₁₀, R₁₂, R₁₅, R₁₉, R₂₀, R₂₅, R₃₃, R₅₂, R₆₉=are independently G or absent;
R₇, R₃₄, R₅₀, R₆₈=are independently G, U or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, provided that the TREM has one or both of the following properties: no more than 15% of the residues are N; or no more than 20 residues are absent.

### Leucine TREM Consensus Sequence

In an embodiment, a TREM disclosed herein comprises the sequence of Formula I_{LEU} (SEQ ID NO: 595),

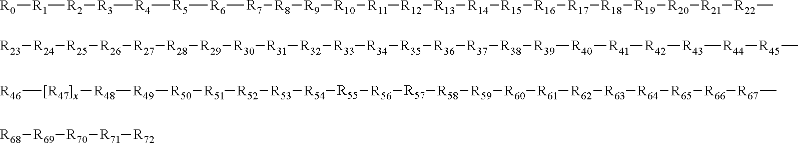

wherein R is a ribonucleotide residue and the consensus for Leu is:
R₀=absent;
R₃₈, R₅₇=are independently A or absent;
R₆₀=A, C or absent;
R₁, R₁₃, R₂₇, R₄₈, R₅₁, R₅₆=are independently A, C, G or absent;
R₂, R₃, R₄, R₅, R₆, R₇, R₉, R₁₀, R₁₁, R₁₂, R₁₆, R₂₃, R₂₆, R₂₈, R₂₉, R₃₀, R₃₁, R₃₂, R₃₃, R₃₄, R₃₇, R₄₁, R₄₂, R₄₃, R₄₄,
R₄₅, R₄₆, R₄₉, R₅₀, R₅₈, R₆₂, R₆₃, R₆₅, R₆₆, R₆₇, R₆₈, R₆₉, R₇₀=are independently N or absent;
R₁₇, R₁₈, R₂₁, R₂₂, R₂₅, R₃₅, R₅₅=are independently A, C, U or absent;
R₁₄, R₁₅, R₃₉, R₇₂=are independently A, G or absent;
R₂₄, R₄₀=are independently A, G, U or absent;
R₅₂, R₆₁, R₆₄, R₇₁=are independently C, G, U or absent;
R₃₆, R₅₃, R₅₉=are independently C, U or absent;
R₁₉=G or absent;
R₂₀=G, U or absent;
R₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{LEU} (SEQ ID NO: 596),

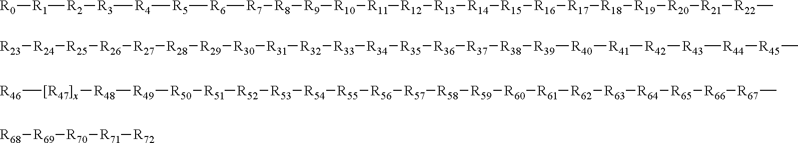

wherein R is a ribonucleotide residue and the consensus for Leu is:
R₀=absent
R₃₈, R₅₇, R₇₂=are independently A or absent;
R₆₀=A, C or absent;
R₄, R₅, R₄₈, R₅₀, R₅₆, R₆₉=are independently A, C, G or absent;
R₆, R₃₃, R₄₁, R₄₃, R₄₆, R₄₉, R₅₈, R₆₃, R₆₆, R₇₀=are independently N or absent;
R₁₁, R₁₂, R₁₇, R₂₁, R₂₂, R₂₈, R₃₁, R₃₇, R₄₄, R₅₅=are independently A, C, U or absent;
R₁, R₉, R₁₄, R₁₅, R₂₄, R₂₇, R₃₄, R₃₉=are independently A, G or absent;
R₇, R₂₉, R₃₂, R₄₀, R₄₅=are independently A, G, U or absent;
R₂₅=A, U or absent;
R₁₃=C, G or absent;
R₂, R₃, R₁₆, R₂₆, R₃₀, R₅₂, R₆₂, R₆₄, R₆₅, R₆₇, R₆₈=are independently C, G, U or absent;
R₁₈, R₃₅, R₄₂, R₅₃, R₅₉, R₆₁, R₇₁=are independently C, U or absent;
R₁₉, R₅₁=are independently G or absent;
R₁₀, R₂₀=are independently G, U or absent;
R₈, R₂₃, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{LEU} (SEQ ID NO: 597),

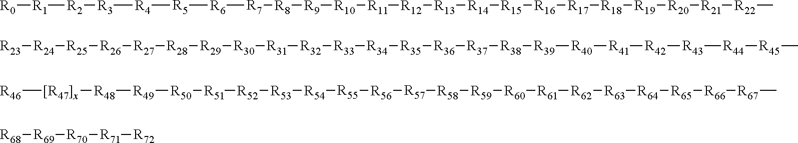

wherein R is a ribonucleotide residue and the consensus for Leu is:
R₀=absent
R₃₈, R₅₇, R₇₂=are independently A or absent;
R₆₀=A, C or absent;
R₄, R₅, R₄₈, R₅₀, R₅₆, R₅₈, R₆₉=are independently A, C, G or absent;
R₆, R₃₃, R₄₃, R₄₆, R₄₉, R₆₃, R₆₆, R₇₀=are independently N or absent;
R₁₁, R₁₂, R₁₇, R₂₁, R₂₂, R₂₈, R₃₁, R₃₇, R₄₁, R₄₄, R₅₅=are independently A, C, U or absent;
R₁, R₉, R₁₄, R₁₅, R₂₄, R₂₇, R₃₄, R₃₉=are independently A, G or absent;
R₇, R₂₉, R₃₂, R₄₀, R₄₅=are independently A, G, U or absent;
R₂₅=A, U or absent;
R₁₃=C, G or absent;
R₂, R₃, R₁₆, R₃₀, R₅₂, R₆₂, R₆₄, R₆₇, R₆₈=are independently C, G, U or absent;
R₁₈, R₃₅, R₄₂, R₅₃, R₅₉, R₆₁, R₆₅, R₇₁=are independently C, U or absent;
R₁₉, R₅₁=are independently G or absent;
R₁₀, R₂₀, R₂₆=are independently G, U or absent;
R₈, R₂₃, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Lysine TREM Consensus Sequence

In an embodiment, a TREM disclosed herein comprises the sequence of Formula I_{LYS} (SEQ ID NO: 598),

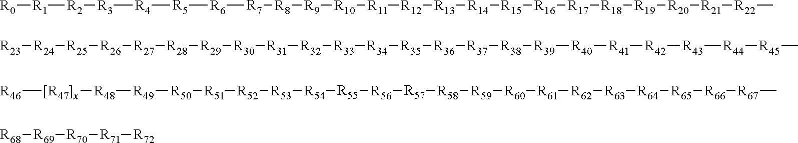

wherein R is a ribonucleotide residue and the consensus for Lys is:
R₀=absent
R₁₄=A or absent;
R₄₀, R₄₁=are independently A, C or absent;
R₃₄, R₄₃, R₅₁=are independently A, C, G or absent;
R₁, R₂, R₃, R₄, R₅, R₆, R₇, R₁₁, R₁₂, R₁₆, R₂₁, R₂₆, R₃₀, R₃₁, R₃₂, R₄₄, R₄₅, R₄₆, R₄₈, R₄₉, R₅₀, R₅₈, R₆₂, R₆₃, R₆₅,
R₆₆, R₆₇, R₆₈, R₆₉, R₇₀=are independently N or absent;
R₁₃, R₁₇, R₅₉, R₇₁=are independently A, C, U or absent;
R₉, R₁₅, R₁₉, R₂₀, R₂₅, R₂₇, R₅₂, R₅₆=are independently A, G or absent;
R₂₄, R₂₉, R₇₂=are independently A, G, U or absent;
R₁₈, R₅₇=are independently A, U or absent;
R₁₀, R₃₃=are independently C, G or absent;
R₄₂, R₆₁, R₆₄=are independently C, G, U or absent;
R₂₈, R₃₅, R₃₆, R₃₇, R₅₃, R₅₅, R₆₀=are independently C, U or absent;
R₈, R₂₂, R₂₃, R₃₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{LYS} (SEQ ID NO: 599),

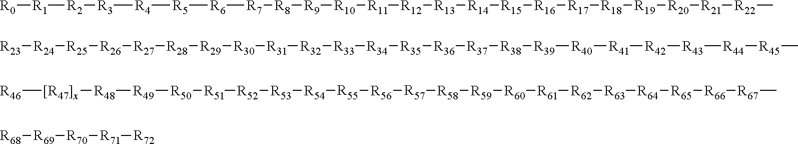

wherein R is a ribonucleotide residue and the consensus for Lys is:
R₉, R₁₈, R₂₃=are absent
R₁₄=A or absent;
R₄₀, R₄₁, R₄₃=are independently A, C or absent;
R₃, R₇=are independently A, C, G or absent;
R₁, R₆, R₁₁, R₃₁, R₄₅, R₄₈, R₄₉, R₆₃, R₆₅, R₆₆, R₆₈=are independently N or absent;
R₂, R₁₂, R₁₃, R₁₇, R₄₄, R₆₇, R₇₁=are independently A, C, U or absent;
R₉, R₁₅, R₁₉, R₂₀, R₂₅, R₂₇, R₃₄, R₅₀, R₅₂, R₅₆, R₇₀, R₇₂=are independently A, G or absent;
R₅, R₂₄, R₂₆, R₂₉, R₃₂, R₄₆, R₆₉=are independently A, G, U or absent;
R₅₇=A, U or absent;
R₁₀, R₆₁=are independently C, G or absent;
R₄, R₁₆, R₂₁, R₃₀, R₅₈, R₆₄=are independently C, G, U or absent;
R₂₈, R₃₅, R₃₆, R₃₇, R₄₂, R 53, R $5, R₅₉, R₆₀, R₆₂=are independently C, U or absent;
R₃₃, R₅₁=are independently G or absent;
R₈-G, U or absent;
R₂₂, R₃₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{LYS} (SEQ ID NO: 600),

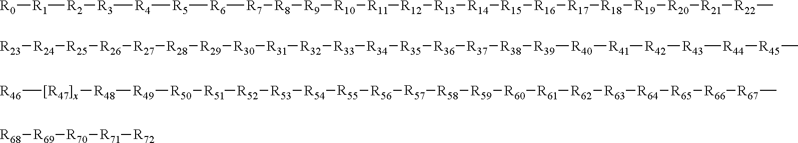

wherein R is a ribonucleotide residue and the consensus for Lys is:
R₉, R₁₈, R₂₃=absent
R₉, R₁₄, R₃₄, R₄₁=are independently A or absent;
R₄₀=A, C or absent;
R₁, R₃, R₇, R₃₁=are independently A, C, G or absent;
R₄₈, R₆₅, R₆₈=are independently N or absent;
R₂, R₁₃, R₁₇, R₄₄, R₆₃, R₆₆=are independently A, C, U or absent;
R₅, R₁₅, R₁₉, R₂₀, R₂₅, R₂₇, R₂₉, R₅₀, R₅₂, R₅₆, R₇₀, R₇₂=are independently A, G or absent;
R₆, R₂₄, R₃₂, R₄₉=are independently A, G, U or absent;
R₁₂, R₂₆, R₄₆, R₅₇=are independently A, U or absent;
R₁₁, R₂₈, R₃₅, R₄₃=are independently C or absent;
R₁₀, R₄₅, R₆₁=are independently C, G or absent;
R₄, R₂₁, R₆₄=are independently C, G, U or absent;
R₃₇, R₅₃, R₅₅, R₅₉, R₆₀, R₆₂, R₆₇, R₇₁=are independently C, U or absent;
R₃₃, R₅₁=are independently G or absent;
R₈, R₃₀, R₅₈, R₆₉=are independently G, U or absent;
R₁₆, R₂₂, R₃₆, R₃₈, R₃₉, R₄₂, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Phenylalanine TREM Consensus Sequence

In an embodiment, a TREM disclosed herein comprises the sequence of Formula I_{PHE} (SEQ ID NO: 601),

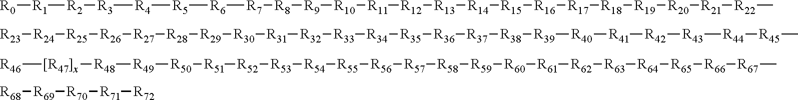

wherein R is a ribonucleotide residue and the consensus for Phe is:
R₉, R₂₃=are absent
R₉, R₁₄, R₃₈, R 39, R₅₇, R₇₂=are independently A or absent;
R₇₁=A, C or absent;
R₄₁, R₇₀=are independently A, C, G or absent;
R₄, R₅, R₆, R₃₀, R₃₁, R₃₂, R₃₄, R₄₂, R₄₄, R₄₅, R₄₆, R₄₈, R₄₉, R₅₈, R₆₂, R₆₃, R₆₆, R₆₇, R₆₈, R₆₉=are
independently N or absent;
R₁₆, R₆₁, R₆₅=are independently A, C, U or absent;
R₁₅, R₂₆, R₂₇, R₂₉, R₄₀, R₅₆=are independently A, G or absent;
R₇, R₅₁=are independently A, G, U or absent;
R₂₂, R₂₄=are independently A, U or absent;
R₅₅, R₆₀=are independently C or absent;
R₂, R₃, R₂₁, R₃₃, R₄₃, R₅₀, R₆₄=are independently C, G, U or absent;
R₁₁, R₁₂, R₁₃, R₁₇, R₂₈, R₃₅, R₃₆, R₅₉=are independently C, U or absent;
R₁₀, R₁₉, R₂₀, R₂₅, R₃₇, R₅₂=are independently G or absent;
R₁=G, U or absent;
R₈, R₁₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{PHE} (SEQ ID NO: 602),

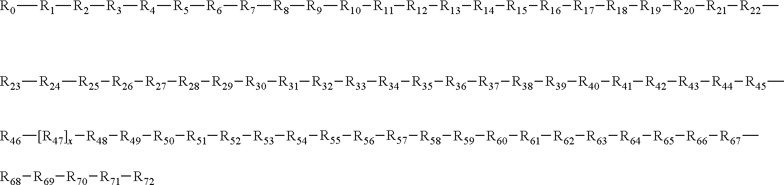

wherein R is a ribonucleotide residue and the consensus for Phe is:
R₉, R₁₈, R₂₃=absent
R₁₄, R₂₄, R₃₈, R₃₉, R₅₇, R₇₂=are independently A or absent;
R₄₆, R₇₁=are independently A, C or absent;
R₄, R₇₀=are independently A, C, G or absent;
R₄₅=A, C, U or absent;
R₆, R₇, R₁₅, R₂₆, R₂₇, R₃₂, R₃₄, R₄₀, R₄₁, R₅₆, R₆₉=are independently A, G or absent;
R₂₉=A, G, U or absent;
R₅, R₉, R₆₇=are independently A, U or absent;
R₃₅, R₄₉, R₅₅, R₆₀=are independently C or absent;
R₂₁, R₄₃, R₆₂=are independently C, G or absent;
R₂, R₃₃, R₆₈=are independently C, G, U or absent;
R₃, R₁₁, R₁₂, R₁₃, R₂₈, R₃₀, R₃₆, R₄₂, R₄₄, R₄₈, R₅₈, R₅₉, R₆₁, R₆₆=are independently C, U or absent;
R₁₀, R₁₉, R₂₀, R₂₅, R₃₇, R₅₁, R₅₂, R₆₃, R₆₄=are independently G or absent;
R₁, R₃₁, R₅₀=are independently G, U or absent;
R₈, R₁₆, R₁₇, R₂₂, R₅₃, R₅₄, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, provided that the TREM has one or both of the following properties: no more than 15% of the residues are N; or no more than 20 residues are absent.

In an embodiment, a TREM disclosed herein comprises the sequence of Formula III_{PHE} (SEQ ID NO: 603),

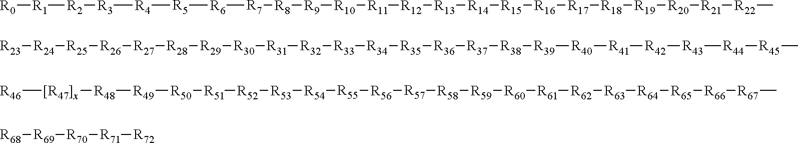

wherein R is a ribonucleotide residue and the consensus for Phe is:
R₀, R₁₈, R₂₂, R₂₃=absent
R₅, R₇, R₁₄, R₂₄, R₂₆, R₃₂, R₃₄, R₃₈, R₃₉, R₄₁, R₅₇, R₇₂=are independently A or absent;
R₄₆=A, C or absent;
R₇₀=A, C, G or absent;
R₄, R₆, R₁₅, R₅₆, R₆₉=are independently A, G or absent;
R₉, R₄₅=are independently A, U or absent;
R₂, R₁₁, R₁₃, R₃₅, R₄₃, R₄₉, R₅₅, R₆₀, R₆₈, R₇₁=are independently C or absent;
R₃₃=C, G or absent;
R₃, R₂₈, R₃₆, R₄₈, R₅₈, R₅₉, R₆₁=are independently C, U or absent;
R₁, R₁₀, R₁₉, R₂₀, R₂₁, R₂₅, R₂₇, R₂₉, R₃₇, R₄₀, R₅₁, R₅₂, R₆₂, R₆₃, R₆₄=are independently G or absent;
R₈, R₁₂, R₁₆, R₁₇, R₃₀, R₃₁, R₄₂, R₄₄, R₅₀, R₅₃, R₅₄, R₆₅, R₆₆, R₆₇=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, provided that the TREM has one or both of the following properties: no more than 15% of the residues are N; or no more than 20 residues are absent.

### Proline TREM Consensus Sequence

In an embodiment, a TREM disclosed herein comprises the sequence of Formula I_{PRO} (SEQ ID NO: 604),

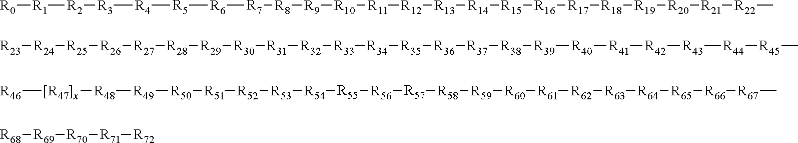

wherein R is a ribonucleotide residue and the consensus for Pro is:
R₀=absent
R₁₄, R₅₇=are independently A or absent;
R₇₀, R₇₂=are independently A, C or absent;
R₉, R₂₆, R₂₇=are independently A, C, G or absent;
R₄, R₅, R₆, R₁₆, R₂₁, R₂₉, R₃₀, R₃₁, R₃₂, R₃₃, R₃₄, R₃₇, R₄₁, R₄₂, R₄₃, R₄₄, R₄₅, R₄₆, R₄₈, R₄₉, R₅₀, R₅₈, R₆₁, R₆₂,
R₆₃, R₆₄, R₆₆, R₆₇, R₆₈=are independently N or absent;
R₃₅, R₆₅=are independently A, C, U or absent;
R₂₄, R₄₀, R₅₆=are independently A, G or absent;
R₇, R₂₅, R₅₁=are independently A, G, U or absent;
R₅₅, R₆₀=are independently C or absent;
R₁, R₃, R₇₁=are independently C, G or absent;
R₁₁, R₁₂, R₂₀, R₆₉=are independently C, G, U or absent;
R₁₃, R₁₇, R₁₈, R₂₂, R₂₃, R₂₈, R₅₉=are independently C, U or absent;
R₁₀, R₁₅, R₁₉, R₃₈, R₃₉, R₅₂=are independently G or absent;
R₂=are independently G, U or absent;
R₈, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{PRO} (SEQ ID NO: 605),

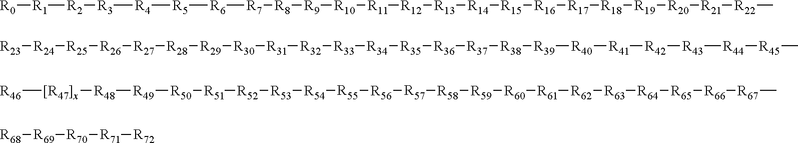

wherein R is a ribonucleotide residue and the consensus for Pro is:
R₉, R₁₇, R₁₈, R₂₂, R₂₃=absent;
R₁₄, R₄₅, R₅₆, R₅₇, R₅₈, R₆₅, R₆₈=are independently A or absent;
R₆₁=A, C, G or absent;
R₄₃=N or absent;
R₃₇=A, C, U or absent;
R₂₄, R₂₇, R₃₃, R₄₀, R₄₄, R₆₃=are independently A, G or absent;
R₃, R₁₂, R₃₀, R₃₂, R₄₈, R₅₅, R₆₀, R₇₀, R₇₁, R₇₂=are independently C or absent;
R₅, R₃₄, R₄₂, R₆₆=are independently C, G or absent;
R₂₀=C, G, U or absent;
R₃₅, R₄₁, R₄₉, R₆₂=are independently C, U or absent;
R₁, R₂, R₆, R₉, R₁₀, R₁₅, R₁₉, R₂₆, R₃₈, R₃₉, R₄₆, R₅₀, R₅₁, R₅₂, R₆₄, R₆₇, R₆₉=are independently G or
absent;
R₁₁, R₁₆=are independently G, U or absent;
R₄, R₇, R₈, R₁₃, R₂₁, R₂₅, R₂₈, R₂₉, R₃₁, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{PRO} (SEQ ID NO: 606),

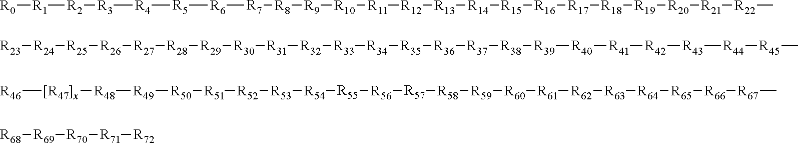

wherein R is a ribonucleotide residue and the consensus for Pro is:
R₉, R₁₇, R₁₈, R₂₂, R₂₃=absent
R₁₄, R₄₅, R₅₆, R₅₇, R₅₈, R₆₅, R₆₈=are independently A or absent;
R₃₇=A, C, U or absent;
R₂₄, R₂₇, R₄₀=are independently A, G or absent;
R₃, R₅, R₁₂, R₃₀, R₃₂, R₄₈, R₄₉, R₅₅, R₆₀, R₆₁, R₆₂, R₆₆, R₇₀, R₇₁, R₇₂=are independently C or absent;
R₃₄, R₄₂=are independently C, G or absent;
R₄₃=C, G, U or absent;
R₄₁=C, U or absent;
R₁, R₂, R₆, R₉, R₁₀, R₁₅, R₁₉, R₂₀, R₂₆, R₃₃, R₃₈, R₃₉, R₄₄, R₄₆, R₅₀, R₅₁, R₅₂, R₆₃, R₆₄, R₆₇, R₆₉=are
independently G or absent;
R₁₆=G, U or absent;
R₄, R₇, R₈, R₁₁, R₁₃, R₂₁, R₂₅, R₂₈, R₂₉, R₃₁, R₃₅, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Serine TREM Consensus Sequence

In an embodiment, a TREM disclosed herein comprises the sequence of Formula I_{SER} (SEQ ID NO: 607),

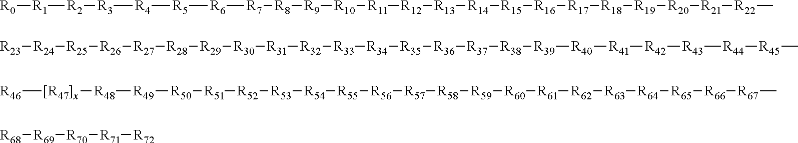

wherein R is a ribonucleotide residue and the consensus for Ser is:
R₀=absent;
R₁₄, R₂₄, R₅₇=are independently A or absent;
R₄₁=A, C or absent;
R₂, R₃, R₄, R₅, R₆, R₇, R₉, R₁₀, R₁₁, R₁₂, R₁₃, R₁₆, R₂₁, R₂₅, R₂₆, R₂₇, R₂₈, R₃₀, R₃₁, R₃₂, R₃₃, R₃₄, R₃₇, R₄₂, R₄₃,
R₄₄, R₄₅, R₄₆, R₄₈, R₄₉, R₅₀, R₆₂, R₆₃, R₆₄, R₆₅, R₆₆, R₆₇, R₆₈, R₆₉, R₇₀=are independently N or absent;
R₁₈=A, C, U or absent;
R₁₅, R₄₀, R₅₁, R₅₆=are independently A, G or absent;
R₁, R₂₉, R₅₈, R₇₂=are independently A, G, U or absent;
R₃₉=A, U or absent;
R₆₀=C or absent;
R₃₈=C, G or absent;
R₁₇, R₂₂, R₂₃, R₇₁=are independently C, G, U or absent;
R₈, R₃₅, R₃₆, R₅₅, R₅₉, R₆₁=are independently C, U or absent;
R₁₉, R₂₀=are independently G or absent;
R₅₂=G, U or absent;
R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{SER} (SEQ ID NO: 608),

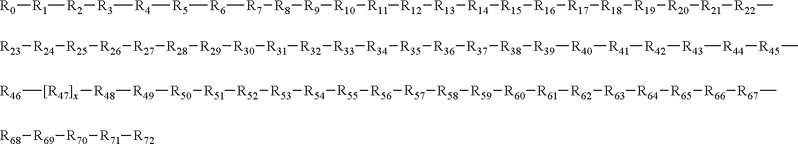

wherein R is a ribonucleotide residue and the consensus for Ser is:
R₉, R₂₃=absent
R₁₄, R₂₄, R₄₁, R₅₇=are independently A or absent;
R₄₄=A, C or absent;
R₂₅, R₄₅, R₄₈=are independently A, C, G or absent;
R₂, R₃, R₄, R₅, R₃₇, R₅₀, R₆₂, R₆₆, R₆₇, R₆₉, R₇₀=are independently N or absent;
R₁₂, R₂₈, R₆₅=are independently A, C, U or absent;
R₉, R₁₅, R₂₉, R₃₄, R₄₀, R₅₆, R₆₃=are independently A, G or absent;
R₇, R₂₆, R₃₀, R₃₃, R₄₆, R₅₈, R₇₂=are independently A, G, U or absent;
R₃₉=A, U or absent;
R₁₁, R₃₅, R₆₀, R₆₁=are independently C or absent;
R₁₃, R₃₈=are independently C, G or absent;
R₆, R₁₇, R₃₁, R₄₃, R₆₄, R₆₈=are independently C, G, U or absent;
R₃₆, R₄₂, R₄₉, R₅₅, R₅₉, R₇₁=are independently C, U or absent;
R₁₀, R₁₉, R₂₀, R₂₇, R₅₁=are independently G or absent;
R₁, R₁₆, R₃₂, R₅₂=are independently G, U or absent;
R₈, R₁₈, R₂₁, R₂₂, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{SER} (SEQ ID NO: 609),

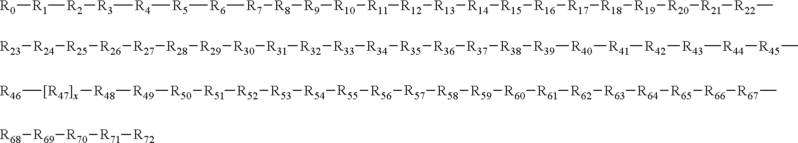

wherein R is a ribonucleotide residue and the consensus for Ser is:
R₉, R₂₃=absent
R₁₄, R₂₄, R₄₁, R₅₇, R₅₈=are independently A or absent;
R₄₄=A, C or absent;
R₂₅, R₄₈=are independently A, C, G or absent;
R₂, R₃, R₅, R₃₇, R₆₆, R₆₇, R₆₉, R₇₀=are independently N or absent;
R₁₂, R₂₈, R₆₂=are independently A, C, U or absent;
R₇, R₉, R₁₅, R₂₉, R₃₃, R₃₄, R₄₀, R₄₅, R₅₆, R₆₃=are independently A, G or absent;
R₄, R₂₆, R₄₆, R 50=are independently A, G, U or absent;
R₃₀, R₃₉=are independently A, U or absent;
R₁₁, R₁₇, R₃₅, R₉, R₆₁=are independently C or absent;
R₁₃, R₃₈=are independently C, G or absent;
R₆, R₆₄=are independently C, G, U or absent;
R₃₁, R₄₂, R₄₃, R₄₉, R₅₅, R₅₉, R₆₅, R₆₈, R₇₁=are independently C, U or absent;
R₁₀, R₁₉, R₂₀, R₂₇, R₅₁, R₅₂=are independently G or absent;
R₁, R₁₆, R₃₂, R₇₂=are independently G, U or absent;
R₈, R₁₈, R₂₁, R₂₂, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Threonine TREM Consensus Sequence

In an embodiment, a TREM disclosed herein comprises the sequence of Formula I_{THR} (SEQ ID NO: 610),

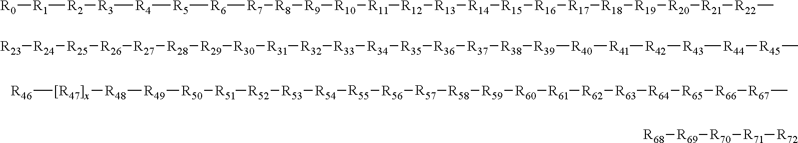

wherein R is a ribonucleotide residue and the consensus for Thr is:
R₉, R₂₃=absent
R₁₄, R₄₁, R₅₇=are independently A or absent;
R₅₆, R₇₀=are independently A, C, G or absent;
R₄, R₅, R₆, R₇, R₁₂, R₁₆, R₂₆, R₃₀, R₃₁, R₃₂, R₃₄, R₃₇, R₄₂, R₄₄, R₄₅, R₄₆, R₄₈, R₄₉, R₅₀, R₅₈, R₆₂, R₆₃, R₆₄, R₆₅, R₆₆, R₆₇, R₆₈, R₇₂=are independently N or absent;
R₁₃, R₁₇, R₂₁, R₃₅, R₆₁=are independently A, C, U or absent;
R₁, R₉, R₂₄, R₂₇, R₂₉, R₆₉=are independently A, G or absent;
R₁₅, R₂₅, R₅₁=are independently A, G, U or absent;
R₄₀, R₅₃=are independently A, U or absent;
R₃₃, R₄₃=are independently C, G or absent;
R₂, R₃, R₅₉=are independently C, G, U or absent;
R₁₁, R₁₈, R₂₂, R₂₈, R₃₆, R₅₄, R₅₅, R₆₀, R₇₁=are independently C, U or absent;
R₁₀, R₂₀, R₃₈, R₅₂=are independently G or absent;
R₁₉=G, U or absent;
R₈, R₃₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{THR} (SEQ ID NO: 611),

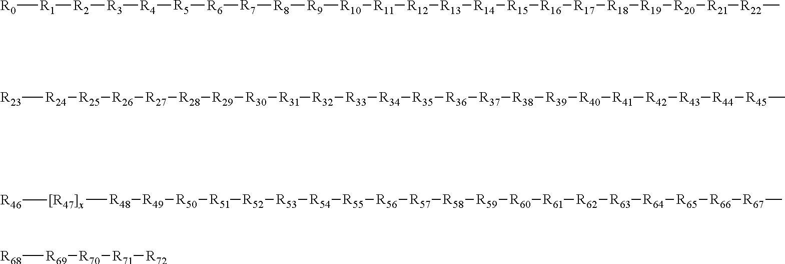

wherein R is a ribonucleotide residue and the consensus for Thr is:
R₉, R₁₈, R₂₃=absent
R₁₄, R₄₁, R₅₇=are independently A or absent;
R₉, R₄₂, R₄₄, R₄₈, R₅₆, R₇₀=are independently A, C, G or absent;
R₄, R₆, R₁₂, R₂₆, R₄₉, R₅₈, R₆₃, R₆₄, R₆₆, R₆₈=are independently N or absent;
R₁₃, R₂₁, R₃₁, R₃₇, R₆₂=are independently A, C, U or absent;
R₁, R₁₅, R₂₄, R₂₇, R₂₉, R₄₆, R₅₁, R₆₉=are independently A, G or absent;
R₇, R₂₅, R₄₅, R₅₀, R₆₇=are independently A, G, U or absent;
R₄₀, R₅₃=are independently A, U or absent;
R₃₅=C or absent;
R₃₃, R₄₃=are independently C, G or absent;
R₂, R₃, R₅, R₁₆, R₃₂, R₃₄, R₅₉, R₆₅, R₇₂=are independently C, G, U or absent;
R₁₁, R₁₇, R₂₂, R₂₈, R₃₀, R₃₆, R₅₅, R₆₀, R₆₁, R₇₁=are independently C, U or absent;
R₁₀, R₁₉, R₂₀, R₃₈, R₅₂=are independently G or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{THR} (SEQ ID NO: 612),

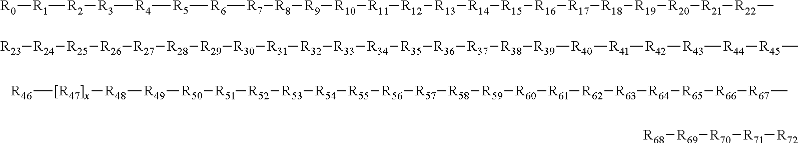

wherein R is a ribonucleotide residue and the consensus for Thr is:
R₉, R₁₈, R₂₃=absent
R₁₄, R₄₀, R₄₁, R₅₇=are independently A or absent;
R₄₄=A, C or absent;
R₉, R₄₂, R₄₈, R₅₆=are independently A, C, G or absent;
R₄, R₆, R₁₂, R₂₆, R₅₈, R₆₄, R₆₆, R₆₈=are independently N or absent;
R₁₃, R₂₁, R₃₁, R₃₇, R₄₉, R₆₂=are independently A, C, U or absent;
R₁, R₁₅, R₂₄, R₂₇, R₂₉, R₄₆, R₅₁, R₆₉=are independently A, G or absent;
R₇, R₂₅, R₄₅, R₅₀, R₆₃, R₆₇=are independently A, G, U or absent;
R₅₃=A, U or absent;
R₃₅=C or absent;
R₂, R₃₃, R₄₃, R₇₀=are independently C, G or absent;
R₅, R₁₆, R₃₄, R₅₉, R₆₅=are independently C, G, U or absent;
R₃, R₁₁, R₂₂, R₂₈, R₃₀, R₃₆, R₅₅, R₆₀, R₆₁, R₇₁=are independently C, U or absent;
R₁₀, R₁₉, R₂₀, R₃₈, R₅₂=are independently G or absent;
R₃₂=G, U or absent;
R₈, R₁₇, R₃₉, R₅₄, R₇₂=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tryptophan TREM Consensus Sequence

In an embodiment, a TREM disclosed herein comprises the sequence of Formula I_{TRP} (SEQ ID NO: 613),

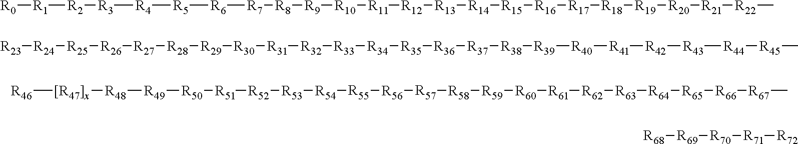

wherein R is a ribonucleotide residue and the consensus for Trp is:
R₀=absent;
R₂₄, R₃₉, R₄₁, R₅₇=are independently A or absent;
R₂, R₃, R₂₆, R₂₇, R₄₀, R₄₈=are independently A, C, G or absent;
R₄, R₅, R₆, R₂₉, R₃₀, R₃₁, R₃₂, R₃₄, R₄₂, R₄₄, R₄₅, R₄₆, R₄₉, R₅₁, R₅₈, R₆₃, R₆₆, R₆₇, R₆₈=are independently N or absent;
R₁₃, R₁₄, R₁₆, R₁₈, R₂₁, R₆₁, R₆₅, R₇₁=are independently A, C, U or absent;
R₁, R₉, R₁₀, R₁₅, R₃₃, R₅₀, R₅₆=are independently A, G or absent;
R₇, R₂₅, R₇₂=are independently A, G, U or absent;
R₃₇, R₃₈, R₅₅, R₆₀=are independently C or absent;
R₁₂, R₃₅, R₄₃, R₆₄, R₆₉, R₇₀=are independently C, G, U or absent;
R₁₁, R₁₇, R₂₂, R₂₈, R₅₉, R₆₂=are independently C, U or absent;
R₁₉, R₂₀, R₅₂=are independently G or absent;
R₈, R₂₃, R₃₆, R₅₃, R₅₄=are independently U or absent; [R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{TRP} (SEQ ID NO: 614),

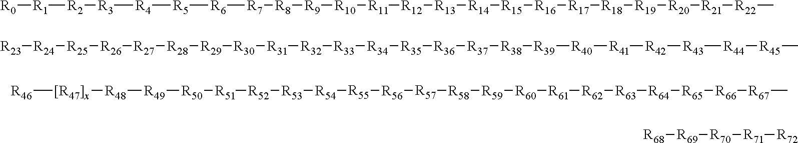

wherein R is a ribonucleotide residue and the consensus for Trp is:
R₉, R₁₈, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A, C or absent;
R₆, R₄₄=are independently A, C, G or absent;
R₂₁=A, C, U or absent;
R₂, R₇, R₁₅, R₂₅, R₃₃, R₃₄, R₄₅, R₅₆, R₆₃=are independently A, G or absent;
R₅₈=A, G, U or absent;
R₄₆=A, U or absent;
R₃₇, R₃₈, R₅₅, R₆₀, R₆₂=are independently C or absent;
R₁₂, R₂₆, R₂₇, R₃₅, R₄₀, R₄₈, R₆₇=are independently C, G or absent;
R₃₂, R₄₃, R₆₈=are independently C, G, U or absent;
R₁₁, R₁₆, R₂₈, R₃₁, R₄₉, R₅₉, R₆₅, R₇₀=are independently C, U or absent;
R₁, R₉, R₁₀, R₁₉, R₂₀, R₅₀, R₅₂, R₆₉=are independently G or absent;
R₅, R₈, R₂₉, R₃₀, R₄₂, R₅₁, R₆₄, R₆₆=are independently G, U or absent;
R₁₇, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{TRP} (SEQ ID NO: 615),

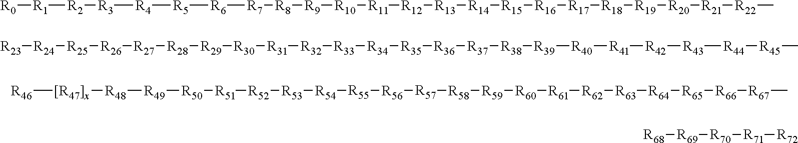

wherein R is a ribonucleotide residue and the consensus for Trp is:
R₉, R₁₈, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A, C or absent;
R₆, R₄₄=are independently A, C, G or absent;
R₂₁=A, C, U or absent;
R₂, R₇, R₁₅, R₂₅, R₃₃, R₃₄, R₄₅, R₅₆, R₆₃=are independently A, G or absent;
R₅₈=A, G, U or absent;
R₄₆=A, U or absent;
R₃₇, R₃₈, R₅₅, R₆₀, R₆₂=are independently C or absent;
R₁₂, R₂₆, R₂₇, R₃₅, R₄₀, R₄₈, R₆₇=are independently C, G or absent;
R₃₂, R₄₃, R₆₈=are independently C, G, U or absent;
R₁₁, R₁₆, R₂₈, R₃₁, R₄₉, R₅₉, R₆₅, R₇₀=are independently C, U or absent;
R₁, R₉, R₁₀, R₁₉, R₂₀, R 50, R 52, R₆₉=are independently G or absent;
R₅, R₈, R₂₉, R₃₀, R₄₂, R₅₁, R₆₄, R₆₆=are independently G, U or absent;
R₁₇, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tyrosine TREM Consensus Sequence

In an embodiment, a TREM disclosed herein comprises the sequence of Formula I_{TYR} (SEQ ID NO: 616),

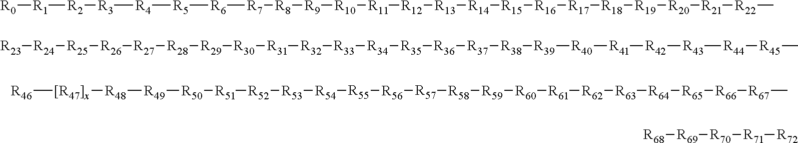

wherein R is a ribonucleotide residue and the consensus for Tyr is:
R₀=absent
R₁₄, R₃₉, R₅₇=are independently A or absent;
R₄₁, R₄₈, R₅₁, R₇₁=are independently A, C, G or absent;
R₃, R₄, R₅, R₆, R₉, R₁₀, R₁₂, R₁₃, R₁₆, R₂₅, R₂₆, R₃₀, R₃₁, R₃₂, R₄₂, R₄₄, R₄₅, R₄₆, R₄₉, R₅₀, R₅₈, R₆₂, R₆₃, R₆₆, R₆₇, R₆₈, R₆₉, R₇₀=are independently N or absent;
R₂₂, R₆₅=are independently A, C, U or absent;
R₁₅, R₂₄, R₂₇, R₃₃, R₃₇, R₄₀, R₅₆=are independently A, G or absent;
R₇, R₂₉, R₃₄, R₇₂=are independently A, G, U or absent;
R₂₃, R₅₃=are independently A, U or absent;
R₃₅, R₆₀=are independently C or absent;
R₂₀=C, G or absent;
R₁, R₂, R₂₈, R₆₁, R₆₄=are independently C, G, U or absent;
R₁₁, R₁₇, R₂₁, R₄₃, R₅₅=are independently C, U or absent;
R₁₉, R₅₂=are independently G or absent;
R₈, R₁₈, R₃₆, R₃₈, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{TYR} (SEQ ID NO: 617),

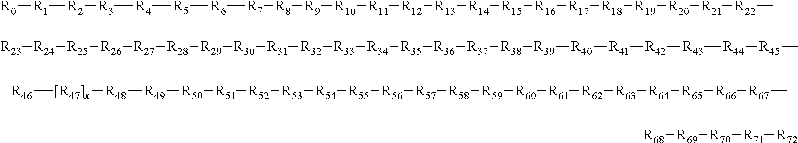

wherein R is a ribonucleotide residue and the consensus for Tyr is:
R₉, R₁₈, R₂₃=absent
R₇, R₉, R₁₄, R₂₄, R₂₆, R₃₄, R₃₉, R₅₇=are independently A or absent;
R₄₄, R₆₉=are independently A, C or absent;
R₇₁=A, C, G or absent;
R₆₈=N or absent;
R₅₈=A, C, U or absent;
R₃₃, R₃₇, R₄₁, R₅₆, R₆₂, R₆₃=are independently A, G or absent;
R₆, R₂₉, R₇₂=are independently A, G, U or absent;
R₃₁, R₄₅, R₅₃=are independently A, U or absent;
R₁₃, R₃₅, R₄₉, R₆₀=are independently C or absent;
R₂₀, R₄₈, R₆₄, R₆₇, R₇₀=are independently C, G or absent;
R₁, R₂, R₅, R₁₆, R₆₆=are independently C, G, U or absent;
R₁₁, R₂₁, R₂₈, R₄₃, R₅₅, R₆₁=are independently C, U or absent;
R₁₀, R₁₅, R₁₉, R₂₅, R₂₇, R₄₀, R₅₁, R₅₂=are independently G or absent;
R₃, R₄, R₃₀, R₃₂, R₄₂, R₄₆=are independently G, U or absent;
R₈, R₁₂, R₁₇, R₂₂, R₃₆, R₃₈, R₅₀, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{TYR} (SEQ ID NO: 618),

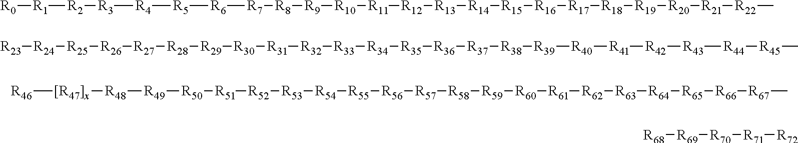

wherein R is a ribonucleotide residue and the consensus for Tyr is:
R₉, R₁₈, R₂₃=absent
R₇, R₉, R₁₄, R₂₄, R₂₆, R₃₄, R₃₉, R₅₇, R₇₂=are independently A or absent;
R₄₄, R₆₉=are independently A, C or absent;
R₇₁=A, C, G or absent;
R₃₇, R₄₁, R₅₆, R₆₂, R₆₃=are independently A, G or absent;
R₆, R₂₉, R₆₈=are independently A, G, U or absent;
R₃₁, R₄₅, R₅₈=are independently A, U or absent;
R₁₃, R₂₈, R₃₅, R₄₉, R₆₀, R₆₁=are independently C or absent;
R₅, R₄₈, R₆₄, R₆₇, R₇₀=are independently C, G or absent;
R₁, R₂=are independently C, G, U or absent;
R₁₁, R₁₆, R₂₁, R₄₃, R₅₅, R₆₆=are independently C, U or absent;
R₁₀, R₁₅, R₁₉, R₂₀, R₂₅, R₂₇, R₃₃, R₄₀, R₅₁, R₅₂=are independently G or absent;
R₃, R₄, R₃₀, R₃₂, R₄₂, R₄₆=are independently G, U or absent;
R₈, R₁₂, R₁₇, R₂₂, R₃₆, R₃₈, R₅₀, R₅₃, R₅₄, R₅₉, R₆₅=are independently U or absent; [R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Valine TREM Consensus Sequence

In an embodiment, a TREM disclosed herein comprises the sequence of Formula I_{VAL} (SEQ ID NO: 619),

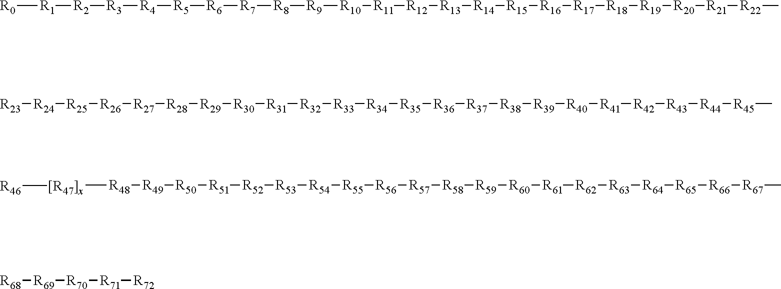

wherein R is a ribonucleotide residue and the consensus for Val is:
R₉, R₂₃=absent;
R₂₄, R₃₈, R₅₇=are independently A or absent;
R₉, R₇₂=are independently A, C, G or absent;
R₂, R₄, R₅, R₆, R₇, R₁₂, R₁₅, R₁₆, R₂₁, R₂₅, R₂₆, R₂₉, R₃₁, R₃₂, R₃₃, R₃₄, R₃₇, R₄₁, R₄₂, R₄₃, R₄₄, R₄₅, R₄₆, R₄₈, R_{4 9}, R₅₀, R₅₈, R₆₁, R₆₂, R₆₃, R₆₄, R₆₅, R₆₆, R₆₇, R₆₈, R₆₉, R₇₀=are independently N or absent;
R₁₇, R₃₅, R₅₉=are independently A, C, U or absent;
R₁₀, R₁₄, R₂₇, R₄₀, R₅₂, R₅₆=are independently A, G or absent;
R₁, R₃, R₅₁, R₅₃=are independently A, G, U or absent;
R₃₉=C or absent;
R₁₃, R₃₀, R₅₅=are independently C, G, U or absent;
R₁₁, R₂₂, R₂₈, R₆₀, R₇₁=are independently C, U or absent;
R₁₉=G or absent;
R₂₀=G, U or absent;
R₈, R₁₈, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{VAL} (SEQ ID NO: 620),

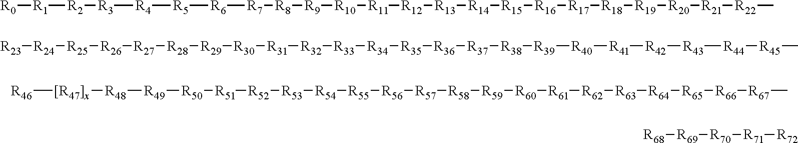

wherein R is a ribonucleotide residue and the consensus for Val is:
R₉, R₁₈, R₂₃=absent;
R₂₄, R₃₈, R₅₇=are independently A or absent;
R₆₄, R₇₀, R₇₂=are independently A, C, G or absent;
R₁₅, R₁₆, R₂₆, R₂₉, R₃₁, R₃₂, R₄₃, R₄₄, R₄₅, R₄₉, R₅₀, R₅₈, R₆₂, R₆₅=are independently N or absent;
R₆, R₁₇, R₃₄, R₃₇, R₄₁, R₅₉=are independently A, C, U or absent;
R₉, R₁₀, R₁₄, R₂₇, R₄₀, R₄₆, R₅₁, R₅₂, R₅₆=are independently A, G or absent;
R₇, R₁₂, R₂₅, R₃₃, R₅₃, R₆₃, R₆₆, R₆₈=are independently A, G, U or absent;
R₆₉=A, U or absent;
R₃₉=C or absent;
R₅, R₆₇=are independently C, G or absent;
R₂, R₄, R₁₃, R₄₈, R₅₅, R₆₁=are independently C, G, U or absent;
R₁₁, R₂₂, R₂₈, R₃₀, R₃₅, R₆₀, R₇₁=are independently C, U or absent;
R₁₉=G or absent;
R₁, R₃, R₂₀, R₄₂=are independently G, U or absent;
R₈, R₂₁, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, provided that the TREM has one or both of the following properties: no more than 15% of the residues are N; or no more than 20 residues are absent.

In an embodiment, a TREM disclosed herein comprises the sequence of Formula III_{VAL} (SEQ ID NO: 621),

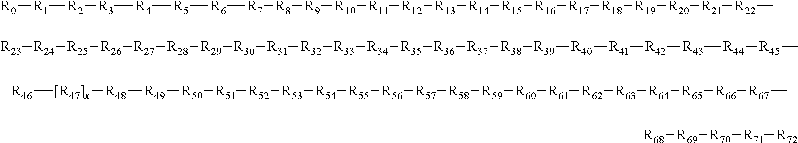

wherein R is a ribonucleotide residue and the consensus for Val is:
R₉, R₁₈, R₂₃=absent
R₂₄, R₃₈, R₄₀, R₅₇, R₇₂=are independently A or absent;
R₂₉, R₆₄, R₇₀=are independently A, C, G or absent;
R₄₉, R₅₀, R₆₂=are independently N or absent;
R₁₆, R₂₆, R₃₁, R₃₂, R₃₇, R₄₁, R₄₃, R₅₉, R₆₅=are independently A, C, U or absent;
R₉, R₁₄, R₂₇, R₄₆, R₅₂, R₅₆, R₆₆=are independently A, G or absent;
R₇, R₁₂, R₂₅, R₃₃, R₄₄, R₄₅, R₅₃, R₅₈, R₆₃, R₆₈=are independently A, G, U or absent;
R₆₉=A, U or absent;
R₃₉=C or absent;
R₅, R₆₇=are independently C, G or absent;
R₂, R₄, R₁₃, R₁₅, R₄₈, R₅₅=are independently C, G, U or absent;
R₆, R₁₁, R₂₂, R₂₈, R₃₀, R₃₄, R₃₅, R₆₀, R₆₁, R₇₁=are independently C, U or absent;
R₁₀, R₁₉, R₅₁=are independently G or absent;
R₁, R₃, R₂₀, R₄₂=are independently G, U or absent;
R₈, R₁₇, R₂₁, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Variable Region Consensus Sequence

In an embodiment, a TREM disclosed herein comprises a variable region at position R₄₇. In an embodiment, the variable region is 1-271 ribonucleotides in length (e.g. 1-250, 1-225, 1-200, 1-175, 1-150, 1-125, 1-100, 1-75, 1-50, 1-40, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 10-271, 20-271, 30-271, 40-271, 50-271, 60-271, 70-271, 80-271, 100-271, 125-271, 150-271, 175-271, 200-271, 225-271, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 125, 150, 175, 200, 225, 250, or 271 ribonucleotides). In an embodiment, the variable region comprises any one, all or a combination of Adenine, Cytosine, Guanine or Uracil.

In an embodiment, the variable region comprises a ribonucleic acid (RNA) Sequence encoded by a deoxyribonucleic acid (DNA) sequence disclosed in Table 4, e.g., any one of SEQ ID NOs: 452-561 disclosed in Table 4.

TABLE 4
Exemplary variable region sequences.

| | SEQ ID NO | SEQUENCE |
|---|---|---|
| 1 | 452 | AAAATATAAATATATTTC |
| 2 | 453 | AAGCT |
| 3 | 454 | AAGTT |
| 4 | 455 | AATTCTTCGGAATGT |
| 5 | 456 | AGA |
| 6 | 457 | AGTCC |
| 7 | 458 | CAACC |
| 8 | 459 | CAATC |
| 9 | 460 | CAGC |
| 10 | 461 | CAGGCGGGTTCTGCCCGCGC |
| 11 | 462 | CATACCTGCAAGGGTATC |
| 12 | 463 | CGACCGCAAGGTTGT |
| 13 | 464 | CGACCTTGCGGTCAT |
| 14 | 465 | CGATGCTAATCACATCGT |
| 15 | 466 | CGATGGTGACATCAT |
| 16 | 467 | CGATGGTTTACATCGT |
| 17 | 468 | CGCCGTAAGGTGT |
| 18 | 469 | CGCCTTAGGTGT |
| 19 | 470 | CGCCTTTCGACGCGT |
| 20 | 471 | CGCTTCACGGCGT |
| 21 | 472 | CGGCAGCAATGCTGT |
| 22 | 473 | CGGCTCCGCCTTC |
| 23 | 474 | CGGGTATCACAGGGTC |
| 24 | 475 | CGGTGCGCAAGCGCTGT |
| 25 | 476 | CGTACGGGTGACCGTACC |
| 26 | 477 | CGTCAAAGACTTC |
| 27 | 478 | CGTCGTAAGACTT |
| 28 | 479 | CGTTGAATAAACGT |
| 29 | 480 | CTGTC |
| 30 | 481 | GGCC |
| 31 | 482 | GGGGATT |
| 32 | 483 | GGTC |
| 33 | 484 | GGTTT |
| 34 | 485 | GTAG |
| 35 | 486 | TAACTAGATACTTTCAGAT |
| 36 | 487 | TACTCGTATGGGTGC |
| 37 | 488 | TACTTTGCGGTGT |
| 38 | 489 | TAGGCGAGTAACATCGTGC |
| 39 | 490 | TAGGCGTGAATAGCGCCTC |
| 40 | 491 | TAGGTCGCGAGAGCGGCGC |
| 41 | 492 | TAGGTCGCGTAAGCGGCGC |
| 42 | 493 | TAGGTGGTTATCCACGC |
| 43 | 494 | TAGTC |
| 44 | 495 | TAGTT |
| 45 | 496 | TATACGTGAAAGCGTATC |
| 46 | 497 | TATAGGGTCAAAAACTCTATC |
| 47 | 498 | TATGCAGAAATACCTGCATC |
| 48 | 499 | TCCCCATACGGGGGC |
| 49 | 500 | TCCCGAAGGGGTTC |
| 50 | 501 | TCTACGTATGTGGGC |
| 51 | 502 | TCTCATAGGAGTTC |
| 52 | 503 | TCTCCTCTGGAGGC |
| 53 | 504 | TCTTAGCAATAAGGT |
| 54 | 505 | TCTTGTAGGAGTTC |
| 55 | 506 | TGAACGTAAGTTCGC |
| 56 | 507 | TGAACTGCGAGGTTCC |
| 57 | 508 | TGAC |
| 58 | 509 | TGACCGAAAGGTCGT |
| 59 | 510 | TGACCGCAAGGTCGT |
| 60 | 511 | TGAGCTCTGCTCTC |
| 61 | 512 | TGAGGCCTCACGGCCTAC |
| 62 | 513 | TGAGGGCAACTTCGT |
| 63 | 514 | TGAGGGTCATACCTCC |
| 64 | 515 | TGAGGGTGCAAATCCTCC |
| 65 | 516 | TGCCGAAAGGCGT |
| 66 | 517 | TGCCGTAAGGCGT |
| 67 | 518 | TGCGGTCTCCGCGC |
| 68 | 519 | TGCTAGAGCAT |
| 69 | 520 | TGCTCGTATAGAGCTC |
| 70 | 521 | TGGACAATTGTCTGC |
| 71 | 522 | TGGACAGATGTCCGT |
| 72 | 523 | TGGACAGGTGTCCGC |
| 73 | 524 | TGGACGGTTGTCCGC |
| 74 | 525 | TGGACTTGTGGTC |
| 75 | 526 | TGGAGATTCTCTCCGC |
| 76 | 527 | TGGCATAGGCCTGC |
| 77 | 528 | TGGCTTATGTCTAC |
| 78 | 529 | TGGGAGTTAATCCCGT |
| 79 | 530 | TGGGATCTTCCCGC |
| 80 | 531 | TGGGCAGAAATGTCTC |
| 81 | 532 | TGGGCGTTCGCCCGC |
| 82 | 533 | TGGGCTTCGCCCGC |
| 83 | 534 | TGGGGGATAACCCCGT |
| 84 | 535 | TGGGGGTTTCCCCGT |
| 85 | 536 | TGGT |
| 86 | 537 | TGGTGGCAACACCGT |
| 87 | 538 | TGGTTTATAGCCGT |
| 88 | 539 | TGTACGGTAATACCGTACC |
| 89 | 540 | TGTCCGCAAGGACGT |
| 90 | 541 | TGTCCTAACGGACGT |
| 91 | 542 | TGTCCTATTAACGGACGT |
| 92 | 543 | TGTCCTTCACGGGCGT |
| 93 | 544 | TGTCTTAGGACGT |
| 94 | 545 | TGTGCGTTAACGCGTACC |
| 95 | 546 | TGTGTCGCAAGGCACC |
| 96 | 547 | TGTTCGTAAGGACTT |
| 97 | 548 | TTCACAGAAATGTGTC |
| 98 | 549 | TTCCCTCGTGGAGT |
| 99 | 550 | TTCCCTCTGGGAGC |
| 100 | 551 | TTCCCTTGTGGATC |
| 101 | 552 | TTCCTTCGGGAGC |
| 102 | 553 | TTCTAGCAATAGAGT |
| 103 | 554 | TTCTCCACTGGGGAGC |
| 104 | 555 | TTCTCGAGAGGGAGC |
| 105 | 556 | TTCTCGTATGAGAGC |
| 106 | 557 | TTTAAGGTTTTCCCTTAAC |
| 107 | 558 | TTTCATTGTGGAGT |
| 108 | 559 | TTTCGAAGGAATCC |
| 109 | 560 | TTTCTTCGGAAGC |
| 110 | 561 | TTTGGGGCAACTCAAC |

### Corresponding Nucleotide Positions

To determine if a selected nucleotide position in a candidate sequence corresponds to a selected position in a reference sequence (e.g., SEQ ID NO: 622, SEQ ID NO: 623, SEQ ID NO: 624), one or more of the following Evaluations is performed.

### Evaluation A:

1. The candidate sequence is aligned with each of the consensus sequences in Tables 9 and 10. The consensus sequence(s) having the most positions aligned (and which has at least 60% of the positions of the candidate sequence aligned) is selected.

The alignment is performed as is follows. The candidate sequence and an isodecoder consensus sequence from Tables 10A-10B are aligned based on a global pairwise alignment calculated with the Needleman-Wunsch algorithm when run with match scores from Table 11, a mismatch penalty of −1, a gap opening penalty of −1, and a gap extension penalty of −0.5, and no penalty for end gaps. The alignment with the highest overall alignment score is then used to determine the percent similarity between the candidate and the consensus sequence by counting the number of matched positions in the alignment, dividing it by the larger of the number of non-N bases in the candidate sequence or the consensus sequence, and multiplying the result by 100. In cases where multiple alignments (of the candidate and a single consensus sequence) tie for the same score, the percent similarity is the largest percent similarity calculated from the tied alignments. This process is repeated for the candidate sequence with each of the remaining isodecoder consensus sequences in Tables 10A-10B, and the alignment resulting in the greatest percent similarity is selected. If this alignment has a percent similarity equal to or greater than 60%, it is considered a valid alignment and used to relate positions in the candidate sequence to those in the consensus sequence, otherwise the candidate sequence is considered to have not aligned to any of the isodecoder consensus sequences. If there is a tie at this point, all tied consensus sequences are taken forward to step 2 in the analysis.

2. Using the selected consensus sequence(s) from step 1, one determines the consensus sequence position number that aligns with the selected position (e.g., a modified position) in the candidate sequence. One then assigns the position number of the aligned position in the consensus sequence to the selected position in the candidate sequence, in other words, the selected position in the candidate sequence is numbered according to the numbering of the consensus sequence. If there were tied consensus sequences from step one, and they give different position numbers in this step 2, then all such position numbers are taken forward to step 5.

3. The reference sequence is aligned with the consensus sequence chosen in step 1. The alignment is performed as described in step 1.

4. From the alignment in step 3, one determines the consensus sequence position number that aligns with the selected position (e.g., a modified position) in the reference sequence. One then assigns the position number of the aligned position in the consensus sequence to the selected position in the reference sequence, in other words, the selected position in the reference sequence is numbered according to the numbering of the consensus sequence. If there is a tie at this point, all tied consensus sequences are taken forward to step 5 in the analysis.

5. If a value for a position number determined for the reference sequence in step 2 is the same as the value for the position number determined for the candidate sequence in step 4, the positions are defined as corresponding.

### Evaluation B:

The reference sequence (e.g., a TREM sequence described herein) and the candidate sequence are aligned with one another. The alignment is performed as follows.

The reference sequence and the candidate sequence are aligned based on a global pairwise alignment calculated with the Needleman-Wunsch algorithm when run with match scores from Table 11, a mismatch penalty of −1, a gap opening penalty of −1, and a gap extension penalty of −0.5, and no penalty for end gaps. The alignment with the highest overall alignment score is then used to determine the percent similarity between the candidate and reference sequence by counting the number of matched based in the alignment, dividing it by the larger of the number of non-N bases in the candidate or reference sequence, and multiplying the result by 100. In cases where multiple alignments tie for the same score, the percent similarity is the largest percent similarity calculated from the tied alignments. If this alignment has a percent similarity equal to or greater than 60%, it is considered a valid alignment and used to relate positions in the candidate sequence to those in the reference sequence, otherwise the candidate sequence is considered to have not aligned to the reference sequence.

If the selected nucleotide position in the reference sequence (e.g., a modified position) is paired with a selected nucleotide position (e.g., a modified position) in the candidate sequence, the positions are defined as corresponding.

### Evaluation C:

The candidate sequence is assigned a nucleotide position number according to the comprehensive tRNA numbering system (CtNS), also referred to as the tRNAviz method (e.g., as described in Lin et al., Nucleic Acids Research, 47: W1, pages W542-W547, 2 Jul. 2019), which serves as a global numbering system for tRNA molecules. The alignment is performed as follows.

1. The candidate sequence is assigned a nucleotide position according to the tRNAviz method. For a novel sequence not present in the tRNAviz database, the numbering for the closest sequence in the database is obtained. For example, if a TREM differs at any given nucleotide position from a sequence in the database, the numbering for the tRNA having the wildtype sequence at said given nucleotide position is used.

2. The reference sequence is assigned a nucleotide position according to the method described in 1.

3. If a value for a position number determined for the reference sequence in step 1 is the same as the value for the position number determined for the candidate sequence in step 2, the positions are defined as corresponding.

If the selected position in the reference sequence and the candidate sequence are found to be corresponding in at least one of Evaluations A, B, and C, the positions correspond. For example, if two positions are found to be corresponding under Evaluation A, but do not correspond under Evaluation B or Evaluation C, the positions are defined as corresponding. Similarly, if two positions are found to be corresponding under Evaluation B, but do not correspond under Evaluation A or Evaluation C, the positions are defined as corresponding. In addition, if two positions are found to be corresponding under Evaluation C, but do not correspond under Evaluation A or Evaluation B, the positions are defined as corresponding.

The numbering given above is used for ease of presentation and does not imply a required sequence. If more than one Evaluation is performed, they can be performed in any order.

TABLE 6
Consensus sequence computationally
generated for each isodecoder by aligning
members of the isodecoder family

| SEQ ID | Amino | | |
|---|---|---|---|
| NO. | Acid | Anticodon | Consensus sequence |
| 1200 | Ala | AGC | GGGGAATTAGCTCAAGTGGTAGAGCGCTTG |
| | | | CTTAGCATGCAAGAGGTAGTGGGATCGATG |
| | | | CCCACATTCTCCA |
| 1201 | Ala | CGC | GGGGATGTAGCTCAGTGGTAGAGCGCATGC |
| | | | TTCGCATGTATGAGGTCCCGGGTTCGATCCC |
| | | | CGGCATCTCCA |
| 1202 | Ala | TGC | GGGGGTGTAGCTCAGTGGTAGAGCGCATGC |
| | | | TTTGCATGTATGAGGCCCCGGGTTCGATCCC |
| | | | CGGCACCTCCA |
| 1203 | Arg | ACG | GGGCCAGTGGCGCAATGGATAACGCGTCTG |
| | | | ACTACGGATCAGAAGATTCCAGGTTCGACTC |
| | | | CTGGCTGGCTCG |
| 1204 | Arg | CCG | GGCCGCGTGGCCTAATGGATAAGGCGTCTG |
| | | | ATTCCGGATCAGAAGATTGAGGGTTCGAGTC |
| | | | CCTTCGTGGTCG |
| 1205 | Arg | CCT | GCCCCAGTGGCCTAATGGATAAGGCACTGG |
| | | | CCTCCTAAGCCAGGGATTGTGGGTTCGAGTC |
| | | | CCACCTGGGGTA |
| 1206 | Arg | TCG | GACCGCGTGGCCTAATGGATAAGGCGTCTG |
| | | | ACTTCGGATCAGAAGATTGAGGGTTCGAGTC |
| | | | CCTCCGTGGTCG |
| 1207 | Arg | TCT | GGCTCTGTGGCGCAATGGATNAGCGCATTG |
| | | | GACTTCTAATTCAAAGGTTGCGGGTTCGAGT |
| | | | CCCNCCAGAGTCG |
| 1208 | Asn | GTT | GTCTCTGTGGCGCAATCGGTTAGCGCGTTCG |
| | | | GCTGTTAACCGNAAAGGTTGGTGGTTCGAGC |
| | | | CCACCCAGGGACG |
| 1209 | Asp | GTC | TCCTCGTTAGTATAGTGGTGAGTATCCCCGC |
| | | | CTGTCACGCGGGAGACCGGGGTTCGATTCCC |
| | | | CGACGGGGAG |
| 1210 | Cys | GCA | GGGGGTATAGCTCAGNGGGTAGAGCATTTG |
| | | | ACTGCAGATCAAGAGGTCCCCGGTTCAAATC |
| | | | CGGGTGCCCCCT |
| 1211 | Gln | CTG | GGTTCCATGGTGTAATGGTNAGCACTCTGGA |
| | | | CTCTGAATCCAGCGATCCGAGTTCAAGTCTC |
| | | | GGTGGAACCT |
| 1212 | Gln | TTG | GGTCCCATGGTGTAATGGTTAGCACTCTGGA |
| | | | CTTTGAATCCAGCGATCCGAGTTCAAATCTC |
| | | | GGTGGGACCT |
| 1213 | Glu | CTC | TCCCTGGTGGTCTAGTGGTTAGGATTCGGCG |
| | | | CTCTCACCGCCGCGGCCCGGGTTCGATTCCC |
| | | | GGTCAGGGAA |
| 1214 | Glu | TTC | TCCCTGGTGGTCTAGTGGCTAGGATTCGGCG |
| | | | CTTTCACCGCNGCGGCCCGGGTTCGATTCCC |
| | | | GGTCAGGGAA |
| 1215 | Gly | CCC | GCATTGGTGGTTCAGTGGTAGAATTCTCGCC |
| | | | TCCCACGCNGGAGACCCGGGTTCGATTCCCG |
| | | | GCCAATGCA |
| 1216 | Gly | GCC | GCATTGGTGGTTCAGTGGTAGAATTCTCGCC |
| | | | TGCCACGCGGGAGGCCCGGGTTCGATTCCCG |
| | | | GCCAATGCA |
| 1217 | Gly | TCC | GCGTTGGTGGTATAGTGGTGAGCATAGCTGC |
| | | | CTTCCAAGCAGTTGACCCGGGTTCGATTCCC |
| | | | GGCCAACGCA |
| 1218 | Ile | AAT | GGCCGGTTAGCTCAGTTGGTTAGAGCGTGGT |
| | | | GCTAATAACGCCAAGGTCGCGGGTTCGATCC |
| | | | CCGTACGGGCCA |
| 1219 | Ile | TAT | GCTCCAGTGGCGCAATCGGTTAGCGCGCGGT |
| | | | ACTTATAATGCCGAGGTTGTGAGTTCGAGCC |
| | | | TCACCTGGAGCA |
| 1220 | Leu | AAG | GGTAGCGTGGCCGAGCGGTCTAAGGCGCTG |
| | | | GATTAAGGCTCCAGTCTCTTCGGGGGCGTGG |
| | | | GTTCGAATCCCACCGCTGCCA |
| 1221 | Leu | CAA | GTCAGGATGGCCGAGTGGTCNTAAGGCGCC |
| | | | AGACTCAAGTTCTGGTCTCCGNATGGAGGCG |
| | | | TGGGTTCGAATCCCACTTCTGACA |
| 1222 | Leu | CAG | GTCAGGATGGCCGAGCGGTCTAAGGCGCTG |
| | | | CGTTCAGGTCGCAGTCTCCCCTGGAGGCGTG |
| | | | GGTTCGAATCCCACTCCTGACA |
| 1223 | Leu | TAA | ACCAGGATGGCCGAGTGGTTAAGGCGTTGG |
| | | | ACTTAAGATCCAATGGACAGATGTCCGCGTG |
| | | | GGTTCGAACCCCACTCCTGGTA |
| 1224 | Leu | TAG | GGTAGCGTGGCCGAGCGGTCTAAGGCGCTG |
| | | | GATTTAGGCTCCAGTCTCTTCGGNGGCGTGG |
| | | | GTTCGAATCCCACCGCTGCCA |
| 1225 | Lys | CTT | GCCCGGCTAGCTCAGTCGGTAGAGCATGAG |
| | | | ACTCTTAATCTCAGGGTCGTGGGTTCGAGCC |
| | | | CCACGTTGGGCGNNN |
| 1226 | Lys | TTT | GCCTGGATAGCTCAGTCGGTAGAGCATCAG |
| | | | ACTTTTAATCTGAGGGTCCAGGGTTCAAGTC |
| | | | CCTGTTCAGGCG |
| 1227 | Met | CAT | GCCCTCTTAGCGCAGTNGGCAGCGCGTCAGT |
| | | | CTCATAATCTGAAGGTCCTGAGTTCGAGCCT |
| | | | CAGAGAGGGCA |
| 1228 | Phe | GAA | GCCGAAATAGCTCAGTTGGGAGAGCGTTAG |
| | | | ACTGAAGATCNTAAAGGTCCCTGGTTCAATC |
| | | | CCGGGTTTCGGCA |
| 1229 | Pro | AGG | GGCTCGTTGGTCTAGGGGTATGATTCTCGCT |
| | | | TAGGATGCGAGAGGTCCCGGGTTCAAATCC |
| | | | CGGACGAGCCC |
| 1230 | Pro | CGG | GGCTCGTTGGTCTAGGGGTATGATTCTCGCT |
| | | | TCGGGTGCGAGAGGTCCCGGGTTCAAATCCC |
| | | | GGACGAGCCC |
| 1231 | Pro | TGG | GGCTCGTTGGTCTAGGGGTATGATTCTCGCT |
| | | | TTGGGTGCGAGAGGTCCCGGGTTCAAATCCC |
| | | | GGACGAGCCC |
| 1232 | Ser | AGA | GTAGTCGTGGCCGAGTGGTTAAGGCGATGG |
| | | | ACTAGAAATCCATTGGGGTTTCCCCGCGCAG |
| | | | GTTCGAATCCTGCCGACTACG |
| 1233 | Ser | CGA | GCTGTGATGGCCGAGTGGTTAAGGCGTTGG |
| | | | ACTCGAAATCCAATGGGGTCTCCCCGCGCAG |
| | | | GTTCGAATCCTGCTCACAGCG |
| 1234 | Ser | GCT | GACGAGGNNTGGCCGAGTGGTTAAGGCGAT |
| | | | GGACTGCTAATCCATTGTGCTCTGCACGCGT |
| | | | GGGTTCGAATCCCATCCTCGTCG |
| 1235 | Ser | TGA | GTAGTCGTGGCCGAGTGGTTAAGGCGATGG |
| | | | ACTTGAAATCCATTGGGGTCTCCCCGCGCAG |
| | | | GTTCGAATCCTGCCGGCTACG |
| 1236 | Thr | AGT | GGCTCCGTGGCTTAGCTGGTTAAAGCGCCTG |
| | | | TCTAGTAAACAGGAGATCCTGGGTTCGAATC |
| | | | CCAGCGGGGCCT |
| 1237 | Thr | CGT | GGCNCTGTGGCTNAGTNGGNTAAAGCGCCG |
| | | | GTCTCGTAAACCNGGAGATCNTGGGTTCGA |
| | | | ATCCCANCNGGGCCT |
| 1238 | Thr | TGT | GGCTCCATAGCTCAGNGGGTTAGAGCACTG |
| | | | GTCTTGTAAACCAGGGGTCGCGAGTTCAAAT |
| | | | CTCGCTGGGGCCT |
| 1239 | Trp | CCA | GACCTCGTGGCGCAACGGTAGCGCGTCTGA |
| | | | CTCCAGATCAGAAGGTTGCGTGTTCAAATCA |
| | | | CGTCGGGGTCA |
| 1240 | Tyr | GTA | CCTTCGATAGCTCAGCTGGTAGAGCGGAGG |
| | | | ACTGTAGATCCTTAGGTCGCTGGTTCGATTC |
| | | | CGGCTCGAAGGA |
| 1241 | Val | AAC | GTTTCCGTAGTGTAGTGGTTATCACGTTCGC |
| | | | CTAACACGCGAAAGGTCCCCGGTTCGAAAC |
| | | | CGGGCGGAAACA |
| 1242 | Val | CAC | GTTTCCGTAGTGTAGTGGTTATCACGTTCGC |
| | | | CTCACACGCGAAAGGTCCCCGGTTCGAAAC |
| | | | CGGGCGGAAACA |
| 1243 | Val | TAC | GGTTCCATAGTGTAGTGGTTATCACGTCTGC |
| | | | TTTACACGCAGAAGGTCCTGGGTTCGAGCCC |
| | | | CAGTGGAACCA |
| 1244 | iMet | CAT | AGCAGAGTGGCGCAGCGGAAGCGTGCTGGG |
| | | | CCCATAACCCAGAGGTCGATGGATCGAAAC |
| | | | CATCCTCTGCTA |

TABLE 7
Consensus sequence computationally
generated for each isodecoder by aligning
members of the isodecoder family

| SEQ ID | Amino | | |
|---|---|---|---|
| NO. | Acid | Anticodon | Consensus sequence |
| 1245 | Ala | AGC | GGGGAATTAGCTCAAGTGGTAGAGCGCTTGC |
| | | | TTAGCATGCAAGAGGTAGTGGGATCGATGCC |
| | | | CACATTCTCCANNN |
| 1246 | Ala | CGC | GGGGATGTAGCTCAGTGGTAGAGCGCATGCT |
| | | | TCGCATGTATGAGGTCCCGGGTTCGATCCCC |
| | | | GGCATCTCCANNN |
| 1247 | Ala | TGC | GGGGGTGTAGCTCAGTGGTAGAGCGCATGCT |
| | | | TTGCATGTATGAGGCCCCGGGTTCGATCCCC |
| | | | GGCACCTCCANNN |
| 1248 | Arg | ACG | GGGCCAGTGGCGCAATGGATAACGCGTCTGA |
| | | | CTACGGATCAGAAGATTCCAGGTTCGACTCC |
| | | | TGGCTGGCTCGNNN |
| 1249 | Arg | CCG | GGCCGCGTGGCCTAATGGATAAGGCGTCTGA |
| | | | TTCCGGATCAGAAGATTGAGGGTTCGAGTCC |
| | | | CTTCGTGGTCGNNN |
| 1250 | Arg | CCT | GCCCCAGTGGCCTAATGGATAAGGCACTGGC |
| | | | CTCCTAAGCCAGGGATTGTGGGTTCGAGTCC |
| | | | CACCTGGGGTANNN |
| 1251 | Arg | TCG | GACCGCGTGGCCTAATGGATAAGGCGTCTGA |
| | | | CTTCGGATCAGAAGATTGAGGGTTCGAGTCC |
| | | | CTCCGTGGTCGNNN |
| 1252 | Arg | TCT | GGCTCTGTGGCGCAATGGATNAGCGCATTGG |
| | | | ACTTCTAATTCAAAGGTTGCGGGTTCGAGTC |
| | | | CCNCCAGAGTCGNNN |
| 1253 | Asn | GTT | GTCTCTGTGGCGCAATCGGTTAGCGCGTTCG |
| | | | GCTGTTAACCGNAAAGGTTGGTGGTTCGAGC |
| | | | CCACCCAGGGACGNNN |
| 1254 | Asp | GTC | TCCTCGTTAGTATAGTGGTGAGTATCCCCGCC |
| | | | TGTCACGCGGGAGACCGGGGTTCGATTCCCC |
| | | | GACGGGGAGNNN |
| 1255 | Cys | GCA | GGGGGTATAGCTCAGNGGGTAGAGCATTTGA |
| | | | CTGCAGATCAAGAGGTCCCCGGTTCAAATCC |
| | | | GGGTGCCCCCTNNN |
| 1256 | Gln | CTG | GGTTCCATGGTGTAATGGTNAGCACTCTGGA |
| | | | CTCTGAATCCAGCGATCCGAGTTCAAGTCTC |
| | | | GGTGGAACCTNNN |
| 1257 | Gln | TTG | GGTCCCATGGTGTAATGGTTAGCACTCTGGA |
| | | | CTTTGAATCCAGCGATCCGAGTTCAAATCTC |
| | | | GGTGGGACCTNNN |
| 1258 | Glu | CTC | TCCCTGGTGGTCTAGTGGTTAGGATTCGGCG |
| | | | CTCTCACCGCCGCGGCCCGGGTTCGATTCCC |
| | | | GGTCAGGGAANNN |
| 1259 | Glu | TTC | TCCCTGGTGGTCTAGTGGCTAGGATTCGGCG |
| | | | CTTTCACCGCNGCGGCCCGGGTTCGATTCCC |
| | | | GGTCAGGGAANNN |
| 1260 | Gly | CCC | GCATTGGTGGTTCAGTGGTAGAATTCTCGCCT |
| | | | CCCACGCNGGAGACCCGGGTTCGATTCCCGG |
| | | | CCAATGCANNN |
| 1261 | Gly | GCC | GCATTGGTGGTTCAGTGGTAGAATTCTCGCCT |
| | | | GCCACGCGGGAGGCCCGGGTTCGATTCCCGG |
| | | | CCAATGCANNN |
| 1262 | Gly | TCC | GCGTTGGTGGTATAGTGGTGAGCATAGCTGC |
| | | | CTTCCAAGCAGTTGACCCGGGTTCGATTCCC |
| | | | GGCCAACGCANNN |
| 1263 | Ile | AAT | GGCCGGTTAGCTCAGTTGGTTAGAGCGTGGT |
| | | | GCTAATAACGCCAAGGTCGCGGGTTCGATCC |
| | | | CCGTACGGGCCANNN |
| 1264 | Ile | TAT | GCTCCAGTGGCGCAATCGGTTAGCGCGCGGT |
| | | | ACTTATAATGCCGAGGTTGTGAGTTCGAGCC |
| | | | TCACCTGGAGCANNN |
| 1265 | Leu | AAG | GGTAGCGTGGCCGAGCGGTCTAAGGCGCTGG |
| | | | ATTAAGGCTCCAGTCTCTTCGGGGGCGTGGG |
| | | | TTCGAATCCCACCGCTGCCANNN |
| 1266 | Leu | CAA | GTCAGGATGGCCGAGTGGTCNTAAGGCGCCA |
| | | | GACTCAAGTTCTGGTCTCCGNATGGAGGCGT |
| | | | GGGTTCGAATCCCACTTCTGACANNN |
| 1267 | Leu | CAG | GTCAGGATGGCCGAGCGGTCTAAGGCGCTGC |
| | | | GTTCAGGTCGCAGTCTCCCCTGGAGGCGTGG |
| | | | GTTCGAATCCCACTCCTGACANNN |
| 1268 | Leu | TAA | ACCAGGATGGCCGAGTGGTTAAGGCGTTGGA |
| | | | CTTAAGATCCAATGGACAGATGTCCGCGTGG |
| | | | GTTCGAACCCCACTCCTGGTANNN |
| 1269 | Leu | TAG | GGTAGCGTGGCCGAGCGGTCTAAGGCGCTGG |
| | | | ATTTAGGCTCCAGTCTCTTCGGNGGCGTGGG |
| | | | TTCGAATCCCACCGCTGCCANNN |
| 1270 | Lys | CTT | GCCCGGCTAGCTCAGTCGGTAGAGCATGAGA |
| | | | CTCTTAATCTCAGGGTCGTGGGTTCGAGCCCC |
| | | | ACGTTGGGCGNNNNNN |
| 1271 | Lys | TTT | GCCTGGATAGCTCAGTCGGTAGAGCATCAGA |
| | | | CTTTTAATCTGAGGGTCCAGGGTTCAAGTCCC |
| | | | TGTTCAGGCGNNN |
| 1272 | Met | CAT | GCCCTCTTAGCGCAGTNGGCAGCGCGTCAGT |
| | | | CTCATAATCTGAAGGTCCTGAGTTCGAGCCT |
| | | | CAGAGAGGGCANNN |
| 1273 | Phe | GAA | GCCGAAATAGCTCAGTTGGGAGAGCGTTAGA |
| | | | CTGAAGATCNTAAAGGTCCCTGGTTCAATCC |
| | | | CGGGTTTCGGCANNN |
| 1274 | Pro | AGG | GGCTCGTTGGTCTAGGGGTATGATTCTCGCTT |
| | | | AGGATGCGAGAGGTCCCGGGTTCAAATCCCG |
| | | | GACGAGCCCNNN |
| 1275 | Pro | CGG | GGCTCGTTGGTCTAGGGGTATGATTCTCGCTT |
| | | | CGGGTGCGAGAGGTCCCGGGTTCAAATCCCG |
| | | | GACGAGCCCNNN |
| 1276 | Pro | TGG | GGCTCGTTGGTCTAGGGGTATGATTCTCGCTT |
| | | | TGGGTGCGAGAGGTCCCGGGTTCAAATCCCG |
| | | | GACGAGCCCNNN |
| 1277 | Ser | AGA | GTAGTCGTGGCCGAGTGGTTAAGGCGATGGA |
| | | | CTAGAAATCCATTGGGGTTTCCCCGCGCAGG |
| | | | TTCGAATCCTGCCGACTACGNNN |
| 1278 | Ser | CGA | GCTGTGATGGCCGAGTGGTTAAGGCGTTGGA |
| | | | CTCGAAATCCAATGGGGTCTCCCCGCGCAGG |
| | | | TTCGAATCCTGCTCACAGCGNNN |
| 1279 | Ser | GCT | GACGAGGNNTGGCCGAGTGGTTAAGGCGAT |
| | | | GGACTGCTAATCCATTGTGCTCTGCACGCGT |
| | | | GGGTTCGAATCCCATCCTCGTCGNNN |
| 1280 | Ser | TGA | GTAGTCGTGGCCGAGTGGTTAAGGCGATGGA |
| | | | CTTGAAATCCATTGGGGTCTCCCCGCGCAGG |
| | | | TTCGAATCCTGCCGGCTACGNNN |
| 1281 | Thr | AGT | GGCTCCGTGGCTTAGCTGGTTAAAGCGCCTG |
| | | | TCTAGTAAACAGGAGATCCTGGGTTCGAATC |
| | | | CCAGCGGGGCCTNNN |
| 1282 | Thr | CGT | GGCNCTGTGGCTNAGTNGGNTAAAGCGCCGG |
| | | | TCTCGTAAACCNGGAGATCNTGGGTTCGAAT |
| | | | CCCANCNGGGCCTNNN |
| 1283 | Thr | TGT | GGCTCCATAGCTCAGNGGGTTAGAGCACTGG |
| | | | TCTTGTAAACCAGGGGTCGCGAGTTCAAATC |
| | | | TCGCTGGGGCCTNNN |
| 1284 | Trp | CCA | GACCTCGTGGCGCAACGGTAGCGCGTCTGAC |
| | | | TCCAGATCAGAAGGTTGCGTGTTCAAATCAC |
| | | | GTCGGGGTCANNN |
| 1285 | Tyr | GTA | CCTTCGATAGCTCAGCTGGTAGAGCGGAGGA |
| | | | CTGTAGATCCTTAGGTCGCTGGTTCGATTCCG |
| | | | GCTCGAAGGANNN |
| 1286 | Val | AAC | GTTTCCGTAGTGTAGTGGTTATCACGTTCGCC |
| | | | TAACACGCGAAAGGTCCCCGGTTCGAAACCG |
| | | | GGCGGAAACANNN |
| 1287 | Val | CAC | GTTTCCGTAGTGTAGTGGTTATCACGTTCGCC |
| | | | TCACACGCGAAAGGTCCCCGGTTCGAAACCG |
| | | | GGCGGAAACANNN |
| 1288 | Val | TAC | GGTTCCATAGTGTAGTGGTTATCACGTCTGCT |
| | | | TTACACGCAGAAGGTCCTGGGTTCGAGCCCC |
| | | | AGTGGAACCANNN |
| 1289 | iMet | CAT | AGCAGAGTGGCGCAGCGGAAGCGTGCTGGG |
| | | | CCCATAACCCAGAGGTCGATGGATCGAAACC |
| | | | ATCCTCTGCTANNN |

TABLE 8
Score values alignment

| | Candidate | Reference | Match |
|---|---|---|---|
| Row | nucleotide | nucleotide | score |
| 1 | A | A | 1 |
| 2 | T | T | 1 |
| 3 | U | T | 1 |
| 4 | C | C | 1 |
| 5 | G | G | 1 |
| 6 | A | N | 0 |
| 7 | T | N | 0 |
| 8 | C | N | 0 |
| 9 | G | N | 0 |
| 10 | N | A | 0 |
| 11 | N | T | 0 |
| 12 | N | C | 0 |
| 13 | N | G | 0 |
| 14 | N | N | 0 |

### Repeat Expansion Diseases (REDs)

A TREM composition disclosed herein can be used treat a RED, e.g., as described herein. REDs described herein are characterized by the expansion of a nucleic acid sequence, e.g., a RED codon, in the genome. For example, a RED may be a trinucleotide repeat disease (e.g., a disease comprising a RED codon) or a hexanucleotide repeat disease. Non-limiting examples of REDs are listed in Table 9.

In an embodiment, the subject has a RED provided in Table 9. In an embodiment, the cell is associated with, e.g., is obtained from a subject who has, a RED listed in Table 9.

For example, the RED can be chosen from the left column of Table 9. As another example, the RED is chosen from the left column of Table 9 and, in some embodiments, the trinucleotide sequence (e.g., the RED codon) or the hexanucleotide sequence is in a gene chosen from the middle column of Table 9, e.g., any one of the genes provided in the middle column of Table 9. In some embodiments, the RED codon or the hexanucleotide sequence is in a gene corresponding to the RED provided in the left column of Table 9. In some embodiments, the RED codon or the hexanucleotide sequence is a repeat motif chosen from the right column of Table 9. For example, the RED is chosen from the left column of Table 9, the RED codon is in a gene provided in the corresponding, e.g., of the same row, middle column of Table 9, and the RED codon is the repeat motif provided in the corresponding right column of Table 9.

In an embodiment, the RED codon is a repeat motif provided in the right column of Table 9 and the repeat expansion is not in a gene provided in Table 9.

TABLE 9
Exemplary Repeat Expansion Diseases,
Related Genes, and Repeated Motifs

| | | Repeat |
|---|---|---|
| Disease | Gene | Motif |
| Huntington's disease | HTT | CAG |
| Spinocerebellar ataxia type 1 (SCA1) | ATXN1 | CAG |
| Spinocerebellar ataxia type 3 | ATXN3 | CAG |
| Myotonic dystrophy type 1 | DMPK | CTG |
| Oculopharyngodistal myopathy 2 | GIPC1 | CGG |
| X-linked intellectual disability | ARX | GCN |
| Dentatorubropallidoluysian atrophy | ATN1 | CAG |
| Spinal and bulbar muscular atrophy | AR | CAG |
| Spinocerebellar ataxia type 7 | ATXN7 | CAG |
| Spinocerebellar ataxia type 17 | TBP | CAG |
| Pseudoachondroplasia | COMP | GAC |
| Multiple epiphyseal dysplasia | COMP | GAC |
| Cleidocranial dysplasia | RUNX2 | GCN |
| Synpolydactyly 1 | HOXD13 | GCN |
| Spinocerebellar ataxia type 2 | ATXN2 | CAG |
| Glutaminase deficiency | GLS | CAG |
| Jacobsen syndrome | CBL2 | CCG |
| Intellectual disability associated | AFF3 | CGG |
| with fragile site FRA2A | | |
| Fragile X syndrome | FMR1 | CGG |
| Fragile X-associated primary ovarian | FMR1 | CGG |
| insufficiency | | |
| Fragile X-associated tremor/ataxia syndrome | FMR1 | CGG |
| Oculopharyngeal myopathy with | LOC642361, | CGG/ |
| leukoencephalopathy | NUTM2B-AS1 | CCG |
| Spinocerebellar ataxia type 8 | ATXN8OS, | CTG/ |
| | ATXN8b | CAG |
| Huntington disease-like 2 | JPH3 | CTG |
| Blepharophimosis, ptosis and epicanthus inversus | FOXL2 | GCN |
| syndrome | | |
| Congenital central hypoventilation syndrome | PHOX2B | GCN |
| Holoprosencephaly 5 | ZIC2 | GCN |
| X-linked hypopituitarism | SOX3 | GCN |
| Amyotrophic lateral sclerosis | C9ORF72 | GGGGCC, |
| | | GGCCGG, |
| | | GGGCCG, |
| | | CCCCGG, |
| | | CCGGCC |

In another aspect, the present disclosure features methods of treating a disease or disorder in a cell or subject by administration of a TREM (e.g., a TREM described herein) to the cell or subject. Exemplary diseases or disorders include REDs, e.g., a RED provided in Table 9.

In some embodiments, the disease or disorder is a polyglutamine (polyQ) disease. PolyQ diseases are characterized by a repeat expansion of the RED codon CAG, which results in an abnormally large stretch of consecutive glutamine residues, which can trigger protein misfolding and amyloid-like aggregations leading to severe cytotoxicities contributing to neurodegeneration. In some embodiments, the disease or disorder is Huntington's disease. Huntington's disease is a genetic neurodegenerative disease. Common symptoms usually begin between 30-50 years of age and include a lack of coordination and unsteady gait that eventually progress to uncoordinated, involuntary body movements characteristic of chorea. In some embodiments, the Huntington's disease is characterized by a repeat expansion in the HTT gene. In some embodiments, the repeat expansion comprises repeats of the RED codon CAG. In some embodiments, the Huntington's disease is characterized by at least 36 (e.g., at least 37, 38, 39, 40, 45, 50, 100, or more) repeats of the RED codon CAG in the HTT gene. In some embodiments, the Huntington's disease is characterized by between about 36-250 (e.g., between about 37-200, 38-150, 39-100, 40-75, or 50) repeats of the RED codon CAG in the HTT gene.

In some embodiments, the polyQ disease is a spinocerebellar ataxia (SCA) (e.g., SCA type 1 (SCA1), SCA2, SCA3, SCA7, SCA8, and SCA17). SCAs are progressive degenerative genetic diseases characterized by neurological symptoms including dysarthria, hypermetric saccades, and ataxia of gait and stance. Approximately 150,000 people in the United States have a diagnosis of SCA. There is no known effective treatment or cure.

In some embodiments, the polyQ disease is SCA1. SCA1 is an autosomal dominant disorder which typically results in death within 10-30 years of symptom onset. SCA1 is typically diagnosed in subjects between 30-40 years of age. In some embodiments, the SCA1 is characterized by a repeat expansion in the ATXN1 gene. In some embodiments, the repeat expansion comprises repeats of the RED codon CAG. In some embodiments, the SCA1 is characterized by at least 40 (e.g., at least 45, 50, 55, 60, 65, 70, 75, or more) repeats of the RED codon CAG in the ATXN1 gene. SCA1 polyQ toxicity results in degradation in neurons (e.g., Purkinje neurons), for example in the cerebellum and spinal cord.

In some embodiments, the polyQ disease is SCA2. SCA2 is a fatal progressive genetic disorder in which poly Q toxicity results in neurodegeneration, e.g., in the cerebellum, inferior olive, and pons. SCA2 symptoms include ataxia, parkinsonism, and dementia. In some embodiments, the SCA2 is characterized by a repeat expansion in the ATXN2 gene. In some embodiments, the repeat expansion comprises repeats of the RED codon CAG. In some embodiments, the SCA2 is characterized by at least 32 (e.g., at least 33, 34, 35, 40, 45, 50, 55, or more) repeats of the RED codon CAG in the ATXN2 gene.

In some embodiments, the polyQ disease is SCA3. SCA3 is an autosomal dominant disease that causes progressive cerebellar ataxia. SCA3 symptoms include gaze-evoked nystagmus, upper motor neuron degeneration, and slow saccades. In some embodiments, the SCA3 is characterized by a repeat expansion in the ATXN3 gene. In some embodiments, the repeat expansion comprises repeats of the RED codon CAG. In some embodiments, the SCA3 is characterized by at least 45 (e.g., at least 50, 55, 60, 65, 70, 75, 80 or more) repeats of the RED codon CAG in the ATXN3 gene. In some embodiments, the SCA3 is characterized by between about 61-87 (e.g., between about 62-86, 63-85, 64-80, 65-75, or 70) repeats of the RED codon CAG in the ATXN3 gene. SCA3 polyQ toxicity results in neurodegeneration primarily in the hindbrain.

In some embodiments, the polyQ disease is SCA7. SCA7 symptoms include macular degeneration, upper motor neuron degeneration, and slow saccades. In some embodiments, the SCA7 is characterized by a repeat expansion in the ATXN7 gene. In some embodiments, the repeat expansion comprises repeats of the RED codon CAG. In some embodiments, the SCA7 is characterized by at least 11 (e.g., at least 12, 13, 14, 15, 20, 25, 30, 40, 45, 50, or more) repeats of the RED codon CAG in the ATXN7 gene. SCA7 polyQ toxicity results in intranuclear inclusion bodies that cause macular degeneration, e.g., in the retina, and neurodegeneration, e.g., in the cerebellum and brainstem.

In some embodiments, the polyQ disease is SCA8. SCA8 symptoms include horizontal nystagmus, dysarthria, mild aspiration, and ataxia. In some embodiments, the SCA8 is characterized by a repeat expansion in the ATXN8b gene or the ATXN8OS. In some embodiments, the repeat expansion comprises repeats of the RED codons CAG or CTG. In some embodiments, the SCA8 comprises a repeat expansion in the ATXN8b gene, and the repeat expansion comprises repeats of the RED codon CAG. In some embodiments, the SCA8 is characterized by at least 51 (e.g., at least 52, 53, 54, 55, 60, 65, 70, 75, 100, or more) repeats of the RED codon CAG in the ATXN8b gene. In some embodiments, the SCA8 is characterized by between about 71-1300 (e.g., between about 72-1,200, 73-1,100, 74-1,000, 75-750, 100-500, or 300) repeats of the RED codon CAG in the ATXN8b gene. SCA8 polyQ toxicity results in neurodegeneration. In some embodiments, the SCA8 comprises a repeat expansion In the ATXN8OS gene, and the repeat expansion comprises repeats of the RED codon CTG. In some embodiments, the SCA8 is characterized by at least 51 (e.g., at least 52, 53, 54, 55, 60, 65, 70, 75, 100, or more) repeats of the RED codon CTG in the ATXN8OS gene. In some embodiments, the SCA8 is characterized by between about 71-1300 (e.g., between about 72-1,200, 73-1,100, 74-1,000, 75-750, 100-500, or 300) repeats of the RED codon CTG in the ATXN8OS gene. ATXN8OS mRNAs comprising the expanded repeat sequence accumulate as ribonuclear inclusions that colocalize with the RNA-binding protein MBNL1 in cerebellar cortical neurons, indicating that these transcripts can dysregulate gene pathways in the brain similar to the mechanism involved in myotonic dystrophy type 1 (DM1) (Daughters et al. PloS Genet. 5: e1000600, 2009).

In some embodiments, the polyQ disease is SCA17. SCA17 is one of the most heterogeneous forms of autosomal dominant cerebellar ataxias with a large clinical spectrum that can mimic other movement disorders such as Huntington's disease, dystonia, and parkinsonism. In addition to gait and limb ataxia and dysarthria, the symptoms may include parkinsonism, choreic movements, dystonia, epilepsy, cognitive, and psychiatric symptoms. In some embodiments, the SCA17 is characterized by a repeat expansion in the TBP gene. In some embodiments, the SCA17 is characterized by at least 41 (e.g., at least 42, 43, 44, 45, 50, 55, 60, 65, 70, or more) repeats of the RED codon CAG in the TBP gene. SCA17 polyQ toxicity results in neurodegeneration.

In some embodiments, the polyQ disease is dentatorubropallidoluysian atrophy (DRPLA). DRPLA is an autosomal dominant spinocerebellar degeneration disorder also known as Haw River Syndrome and Naito-Oyanagi disease. DRPLA symptoms may include ataxia, choreoathetosis, dementia, seizures, myoclonus, cervical dystonia, corneal endothelial degeneration, autism, and surgery-resistant obstructive sleep apnea. In some embodiments, the DRPLA is characterized by a repeat expansion in the ATN1 gene. In some embodiments, the repeat expansion comprises repeats of the RED codon CAG. In some embodiments, the DRPLA is characterized by at least 36 (e.g., at least 37, 38, 39, 40, 45, 50, or more) repeats of the RED codon CAG in the ATN1 gene. In some embodiments, the DRPLA is characterized by between about 49-88 (e.g., between about 50-87, 55-86, 60-85, 65-80, or 70-75) repeats of the RED codon CAG in the ATN1 gene. DRPLA polyQ toxicity results in neuronal intranuclear inclusions and neuronal degeneration, e.g., in CNS tissue throughout the brain and spinal cord.

In some embodiments, the polyQ disease is spinal and bulbar muscular atrophy (SBMA), a rare, adult-onset, X-linked recessive lower motor neuron disease. SBMA symptoms include dysarthria, dysphagia, muscle denervation, and bulbar and lower motor neuron degeneration. In some embodiments, the SBMA is characterized by a repeat expansion in the AR gene. In some embodiments, the repeat expansion comprises repeats of the RED codon CAG. In some embodiments, the SBMA is characterized by at least 35 (e.g., at least 40, 45, 50, 55, 60, 65, 70, or more) repeats of the RED codon CAG in the AR gene. SBMA polyQ toxicity results in neurodegeneration, e.g., in the anterior horn of the spinal cord and brainstem.

In some embodiments, the polyQ disease is glutaminase deficiency, an autosomal recessive genetic disorder that presents in childhood. Glutaminase deficiency is associated with epilepsy and is characterized by refractory seizures, respiratory failure, brain abnormalities, and death in the neonatal period. In some embodiments, glutaminase deficiency is characterized by a repeat expansion in the GLS gene. In some embodiments, the repeat expansion comprises repeats of the RED codon CAG. In some embodiments, the glutaminase deficiency is characterized by at least 35 (e.g., at least 40, 45, 50, 55, 60, 65, 70, or more) repeats of the RED codon CAG in the GLS gene.

In some embodiments, the RED is fragile X syndrome (FRAXA). FRAXA is an X-linked dominant genetic disorder characterized by mild to moderate intellectual disability. FRAXA symptoms include autism, delayed speech, and hyperactivity. In some embodiments, the FRAXA is characterized by a repeat expansion in the FMR1 gene. In some embodiments, the repeat expansion comprises repeats of the RED codon CGG. In some embodiments, the FRAXA is characterized by at least 54 (e.g., at least 55, 60, 65, 70, 75, 100, or more) repeats of the RED codon CGG in the FMR1 gene. In some embodiments, the FRAXA is characterized by at least 230 (e.g., 240, 250, 300, or more) repeats of the RED codon CGG in the FMR1 gene.

In some embodiments, the RED is fragile X-associated tremor/ataxia syndrome (FXTAS). FXTAS is a late-onset neurodegenerative disorder most frequently diagnosed in male premutation carriers of FRAXA over the age of 50. Symptoms may include cerebellar gait ataxia, action tremor, parkinsonism, cognitive decline, and dysfunction of the autonomic nervous system. In some embodiments, the FXTAS is characterized by a repeat expansion in the FMR1 gene. In some embodiments, the repeat expansion comprises repeats of the RED codon CGG. In some embodiments, the FXTAS is characterized by at least 54 (e.g., at least 55, 60, 65, 70, 75, 100, or more) repeats of the RED codon CGG in the FMR1 gene. In some embodiments, the FXTAS is characterized by between about 55-200 (e.g., between about 60-175, 75-150, or 100-125) repeats of the RED codon CGG in the FMR1 gene.

In some embodiments, the RED is fragile X-associated primary ovarian insufficiency (FXPOI). FXPOI is the most common known genetic cause of ovarian insufficiency for women with a normal chromosome number (46, XX) and accounts for 5-10% of premature ovarian failure. Symptoms may include repeated elevation of follicle stimulating hormone and the loss of menstruation for at least 4-6 months. In some embodiments, the FXPOI is characterized by a repeat expansion in the FMR1 gene. In some embodiments, the repeat expansion comprises repeats of the RED codon CGG. In some embodiments, the FXPOI is characterized by between about 55-199 (e.g., between about 60-175, 75-150, or 100-125) repeats of the RED codon CGG in the FMR1 gene. In some embodiments, the FXPOI is characterized by between about 70-100 (e.g., between about 75-95, 80-90, or 85) repeats of the RED codon CGG in the FMR1 gene.

In some embodiments, the RED is myotonic dystrophy type 1 (DM1). DM1 is an autosomal dominant type of muscular dystrophy causing symptoms including delayed relaxation of muscles after contraction (myotonia), ptosis, hypersomnia, and abnormalities in the electrical activity of the heart, e.g., arrhythmias or conduction blocks. In some embodiments, the DM1 is characterized by a repeat expansion in the DMPK gene. In some embodiments, the repeat expansion comprises repeats of the RED codon CTG. In some embodiments, the DM1 is characterized by at least 35 (e.g., at least 40, 45, 50, 55, 60, 65, or more) repeats of the RED codon CTG in the DMPK gene. In some embodiments, the DM1 is characterized by at least 50 (e.g., at least 100, 150, 200, or more) repeats of the RED codon CTG in the DMPK gene.

In some embodiments, the RED is a disease or disorder characterized by a repeat expansion of the RED codon GCN, where N can be A, C, T, or G, resulting in a poly-alanine (polyA) tract of abnormal length in the resultant protein. Such REDs may be classified as polyA REDs. The abnormally long poly A tracts, similar to polyQ repeat expansions, promote protein aggregations, e.g., intranuclear inclusions, which contribute to cell death and neurodegeneration. In some embodiments, the polyA RED is selected from Blepharophimosis, ptosis, and epicanthus inversus syndrome (BPES), Cleidocranial dysplasia (CCD), Congenital central hypoventilation syndrome (CHS), Holoprosencephaly 5 (HPE), Synpolydactyly 1 (SPD1), and an X-linked intellectual disability (e.g., X-linked hypopituitarism).

In some embodiments, the polyA RED is BPES. BPES is a rare autosomal dominant genetic disease characterized by symptoms including horizontally narrow eyes (blepharophimosis), drooping eyelids (ptosis), and a fold of skin running from the side of the nose to the lower eyelids (epicanthus inversus). Type 1 and Type 2 BPES can be distinguished by the presence (Type 1) or absence (Type 2) of the symptom of premature ovarian insufficiency in females, which often results in menopausal symptoms and infertility in patients as young as 15 years of age. In some embodiments, the BPES is characterized by a repeat expansion in the FOXL2 gene. In some embodiments, the repeat expansion comprises repeats of the RED codon GCN. In some embodiments, the BPES is characterized by at least 15 (e.g., at least 20, 25, 30, 35, 40, 45, 50, or more) repeats of the RED codon GCN in the FOXL2 gene.

In some embodiments, the polyA RED is CCD. CCD is an autosomal dominant birth defect that mostly affects the bones and teeth. The collarbones are typically either poorly developed or absent, which brings the shoulders to form close together, the front of the skull closes abnormally late, resulting in a shorter than average skull, and other symptoms may include a prominent forehead, wide-set eyes, abnormal teeth, scoliosis, decreased bone density, and a flat nose. In some embodiments, the CCD is characterized by a repeat expansion in the RUNX2 gene. In some embodiments, the repeat expansion comprises repeats of the RED codon GCN. In some embodiments, the CCD is characterized by at least 15 (e.g., at least 20, 25, 30, 35, 40, 45, 50, or more) repeats of the RED codon GCN in the RUNX2 gene.

In some embodiments, the polyA RED is CHS. CHS is a congenital sleep-related breathing disorder that causes ineffective breathing, apnea, or respiratory arrest during sleep (mild form) and during wakefulness (severe form) due to a failure of autonomic breathing control. In some embodiments, the CHS is characterized by a repeat expansion in the PHOX2B gene. In some embodiments, the repeat expansion comprises repeats of the RED codon GCN. In some embodiments, the CHS is characterized by at least 15 (e.g., at least 20, 25, 30, 35, 40, 45, 50, or more) repeats of the RED codon GCN in the PHOX2B gene.

In some embodiments, the polyA RED is HPE. HPE is a congenital cephalic disorder in which the prosencephalon fails to develop into two hemispheres, typically between the 18^{th} and 28^{th} day of gestation. HPE symptoms can range from mild (no facial defects, no organ defects, anosmia, or only a single central incisor) to severe (cyclopia). In some embodiments, the HPE is characterized by a repeat expansion in the ZIC2 gene. In some embodiments, the repeat expansion comprises repeats of the RED codon GCN. In some embodiments, the HPE is characterized by at least 7 (e.g., at least 8, 9, 10, or more) repeats of the RED codon GCN in the ZIC2 gene.

In some embodiments, the polyA RED is SPD1. SPD1 is an autosomal dominant disorder characterized by joint presentation of syndactyl and polydactyly. In some embodiments, the SPD1 is characterized by a repeat expansion in the HOXD13 gene. In some embodiments, the repeat expansion comprises repeats of the RED codon GCN. In some embodiments, the SPD1 is characterized by at least 7 (e.g., at least 8, 9, 10, or more) repeats of the RED codon GCN in the HOXD13 gene.

In some embodiments, the polyA RED is an X-linked intellectual disability, e.g., a medical disorder associated with X-linked recessive inheritance that results in intellectual disability. Non-limiting examples of such disorders include Coffin-Lowry syndrome, CCD3× syndrome, MASA syndrome, MECP2 duplication syndrome, mental retardation and microcephaly with pontine and cerebellar hypoplasia, and X-linked alpha thalassemia mental retardation syndrome. In some embodiments, the X-linked intellectual disability is characterized by a repeat expansion in the ARX gene. In some embodiments, the repeat expansion comprises repeats of the RED codon GCN. In some embodiments, the X-linked disability is characterized by at least 7 (e.g., at least 8, 9, 10, or more) repeats of the RED codon GCN in the ARX gene. PolyA repeat expansion in the ARX protein reduces both the concentration and function of ARX protein in cells, which is associated with impairment of the normal development and migration of interneurons. The subsequent deterioration of interneuron function likely underlies neurological problems characteristic of X-linked intellectual disabilities.

In some embodiments, the polyA RED is X-linked hypopituitarism. X-linked hypopituitarism is an X-linked intellectual disability in which the cause of the disease is a mutation in the SOX3 gene, with variable phenotypes including growth hormone deficiency due to hypopituitarism. In some embodiments, the X-linked hypopituitarism is characterized by a repeat expansion in the SOX3 gene. In some embodiments, the repeat expansion comprises repeats of the RED codon GCN. In some embodiments, the X-linked hypopituitarism is characterized by at least 7 (e.g., at least 8, 9, 10, or more) repeats of the RED codon GCN in the SOX3 gene.

In some embodiments, the RED is oculopharyngodistal myopathy 2, an autosomal dominant genetic disorder characterized by progressive muscle weakness. In some embodiments, the oculopharyngodistal myopathy 2 is characterized by a repeat expansion in the GIPC1 gene. In some embodiments, the repeat expansion comprises repeats of the RED codon CGG. In some embodiments, the oculopharyngodistal myopathy 2 is characterized by at least 31 (e.g., at least 32, 33, 34, 35, 40, 45, 50, or more) repeats of the RED codon CGG in the GIPC1 gene. In some embodiments, the oculopharyngodistal myopathy 2 is characterized by at least 60 (e.g., at least 65, 70, 75, 100, or more) repeats of the RED codon CGG in the GIPC1 gene.

In some embodiments, the RED is pseudoachondroplasia (PSACH). PSACH is an autosomal dominant bone growth disorder. Symptoms of PSACH may include a waddling gait, lower limb deformities, and mild to severely short stature. In some embodiments, PSACH is characterized by a repeat expansion in the COMP gene. In some embodiments, the repeat expansion comprises repeats of the RED codon GAC. In some embodiments, the PSACH is characterized by at least 5 (e.g., at least 6, 7, 8, 9 10, or more) repeats of the RED codon GAC in the COMP gene.

In some embodiments, the RED is multiple epiphyseal dysplasia (MED). MED is an autosomal dominant genetic skeletal disorder. Symptoms may include joint pain and fatigue after exercising, small and irregular ossifications centers, and a waddling gait. In some embodiments, MED is characterized by a repeat expansion in the COMP gene. In some embodiments, the repeat expansion comprises repeats of the RED codon GAC. In some embodiments, the MED is characterized by at least 5 (e.g., at least 6, 7, 8, 9 10, or more) repeats of the RED codon GAC in the COMP gene.

In some embodiments, the RED is Jacobsen syndrome, a congenital disorder resulting in symptoms including intellectual disabilities, dysmorphic features, delayed development, heart defects, and other secondary symptoms including eye problems, ear and sinus infections, hearing problems, bone deformities, growth hormone deficiency, gastrointestinal problems, and kidney malfunctions. In some embodiments, the Jacobsen syndrome is characterized by a repeat expansion in the CBL2 gene. In some embodiments, the repeat expansion comprises repeats of the RED codon CCG. In some embodiments, the Jacobsen syndrome is characterized by at least 3 (e.g., at least 4, 5, 10, 15, 20, or more) repeats of the RED codon CCG in the CBL2 gene.

In some embodiments, the RED is intellectual disability associated with fragile site FRA2A. Symptoms may include impaired cognitive function, communication and behavioral problems, and a short attention span. In some embodiments, the intellectual disability associated with fragile site FRA2A is characterized by a repeat expansion in the AFF3 gene. In some embodiments, the repeat expansion comprises repeats of the RED codon CGG. In some embodiments, the intellectual disability associated with fragile site FRA2A is characterized by at least 10 (e.g., at least 15, 20, 25, 50, or more) repeats of the RED codon CGG in the AFF3 gene.

In some embodiments, the RED is oculopharyngeal myopathy with leukoencephalopathy (OPML). OPML symptoms include ptosis, ophthalmoplegia, dysphagia, dysarthria, and limb weakness. In some embodiments, the OPML is characterized by a repeat expansion in the LOC642361 gene. In some embodiments, the repeat expansion comprises repeats of the RED codon CGG. In some embodiments, the OPML is characterized by at least 3 (e.g., at least 4, 5, 10, 15, 20, 25, 50, or more) repeats of the RED codon in the LOC642361 gene. In some embodiments, the OPML is characterized by a repeat expansion in the NUTM2B-AS1 gene. In some embodiments, the repeat expansion comprises repeats of the RED codon CCG. In some embodiments, the OPML is characterized by at least 3 (e.g., at least 4, 5, 10, 15, 20, 25, 50, or more) repeats of the RED codon in the NUTM2B-AS1 gene.

In some embodiments, the RED is Huntington disease-like 2 (HDL2). HDL2 typically presents in midlife with a relentless progressive triad of movement, emotional, and cognitive abnormalities that lead to death within 10-20 years. In some embodiments, the HDL2 is characterized by a repeat expansion in the JPH3 gene. In some embodiments, the repeat expansion comprises repeats of the RED codon CTG. In some embodiments, the HDL2 is characterized by at least 29 (e.g., at least 30, 35, 40, 45, 50, or more) repeats of the RED codon CTG in the JPH3 gene. In some embodiments, the HDL2 is characterized by between about 40-59 (e.g., between about 45-58, 50-57, or 55-56) repeats of the RED codon CTG in the JPH3 gene.

In some embodiments, the RED is amyotrophic lateral sclerosis (ALS). ALS is a neurodegenerative disease that is the most common form of the motor neuron diseases. Symptoms of ALS may include muscle weakness, atrophy, and muscle spasms. In some embodiments, the ALS is characterized by a repeat expansion in the C9ORF72 gene. In some embodiments, the repeat expansion is a hexanucleotide repeat expansion (HRE). In some embodiments, the HRE comprises repeats of the hexanucleotide sequences GGGGCC, GGCCGG, GGGCCG, CCCCGG, or CCGGCC. In some embodiments, the ALS is characterized by at least 3 (e.g., at least 4, 5, 10, 15, 20, 25, 50, 100, 200, or more) repeats of a hexanucleotide sequence selected from GGGGCC, GGCCGG, GGGCCG, CCCCGG, and CCGGCC.

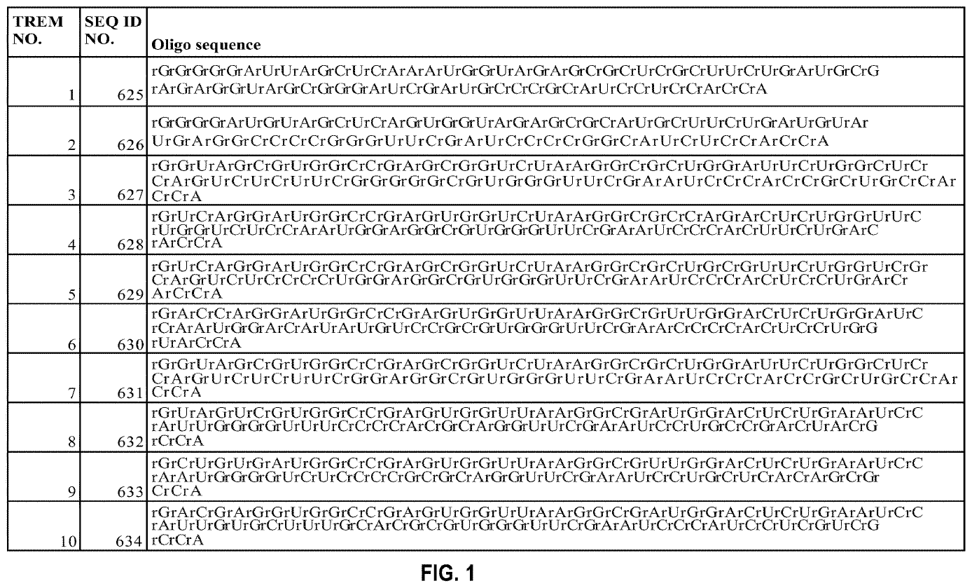

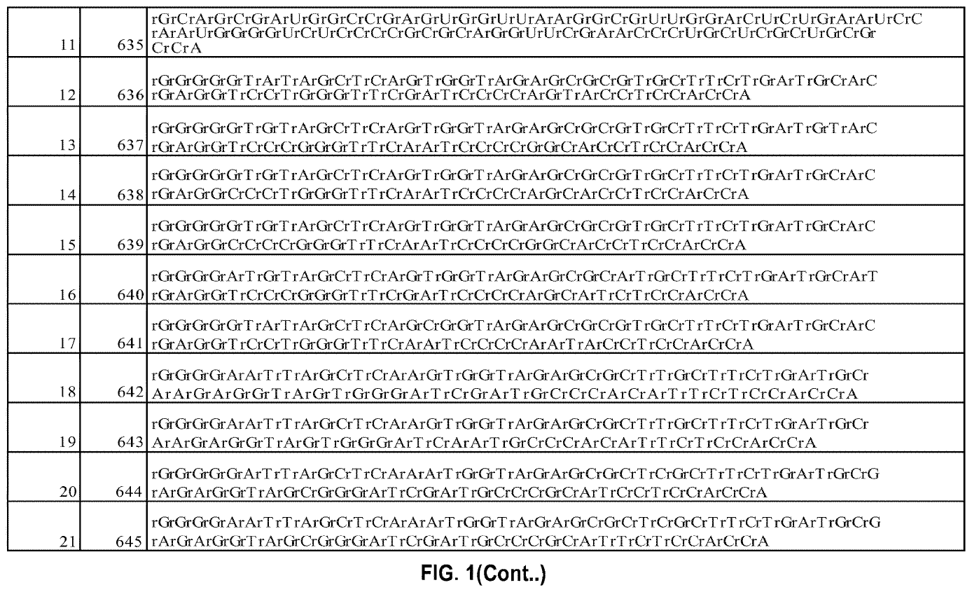

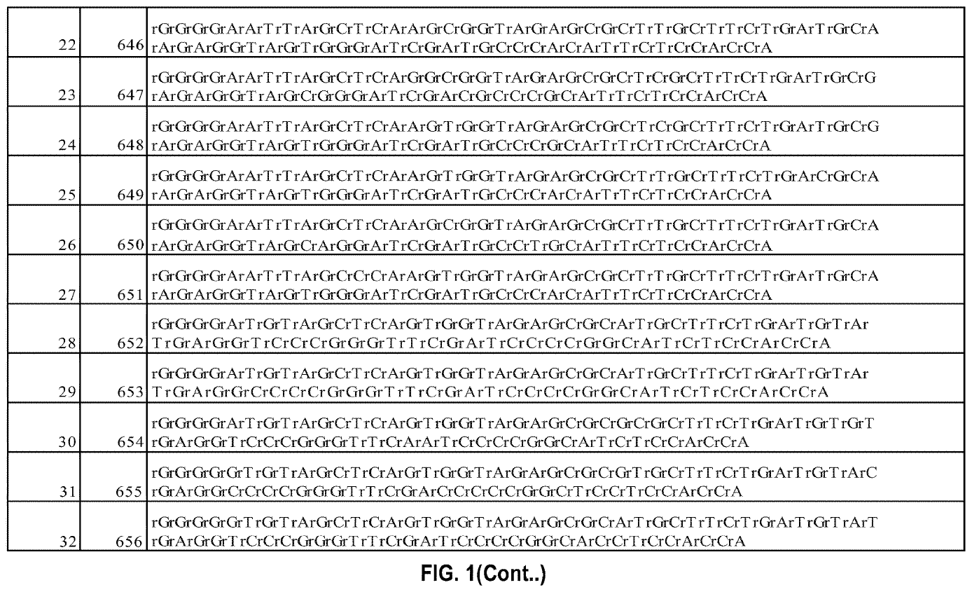

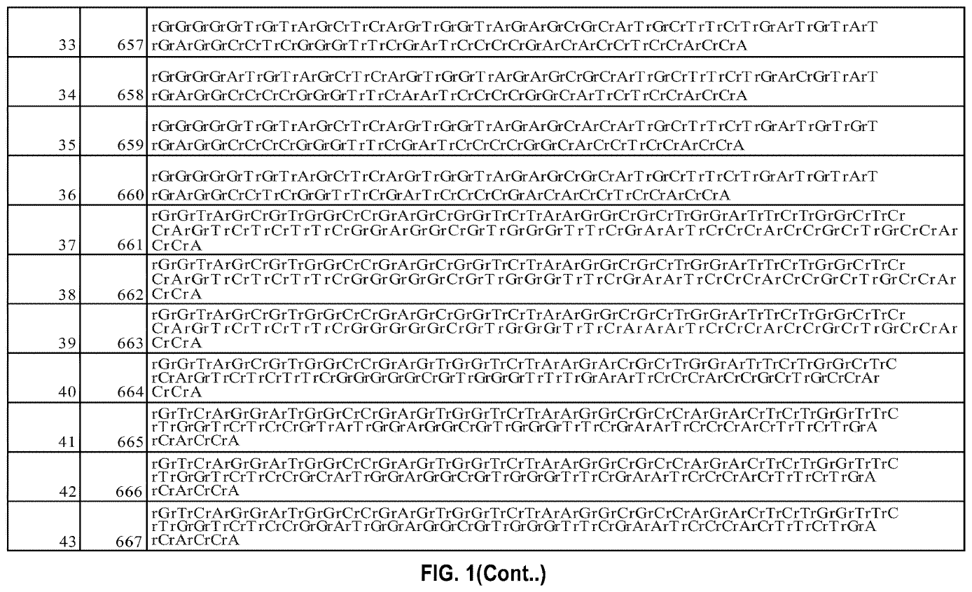

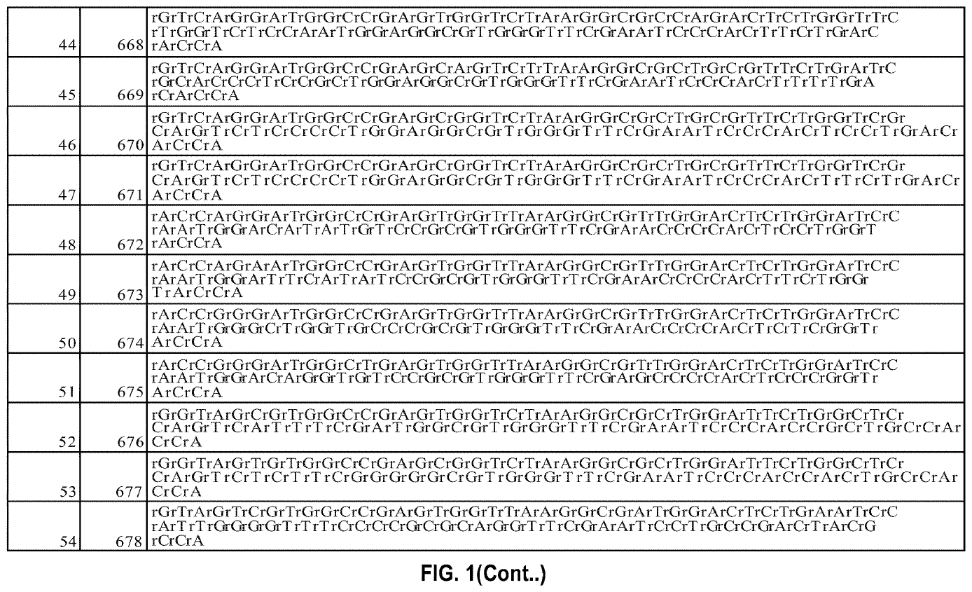

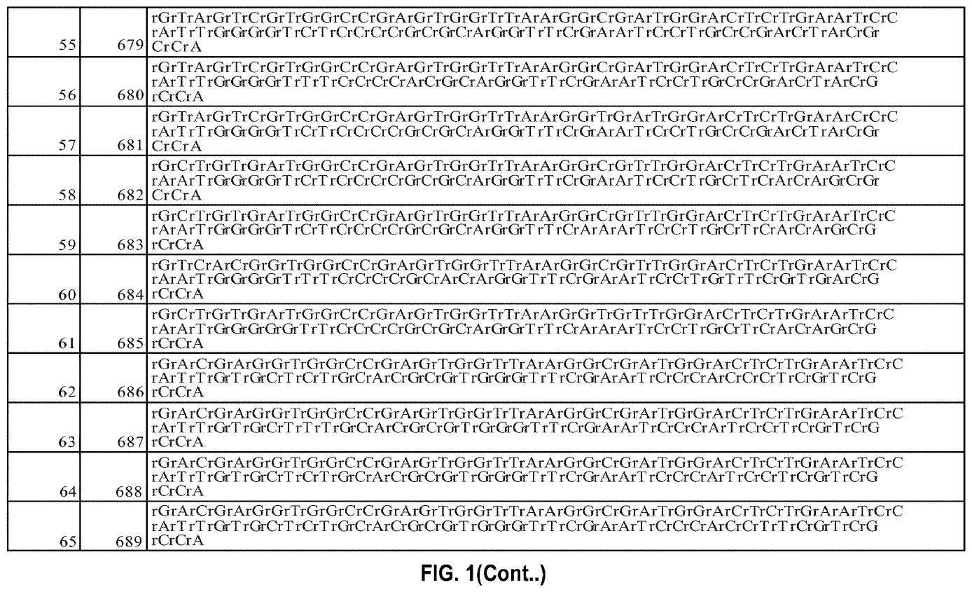

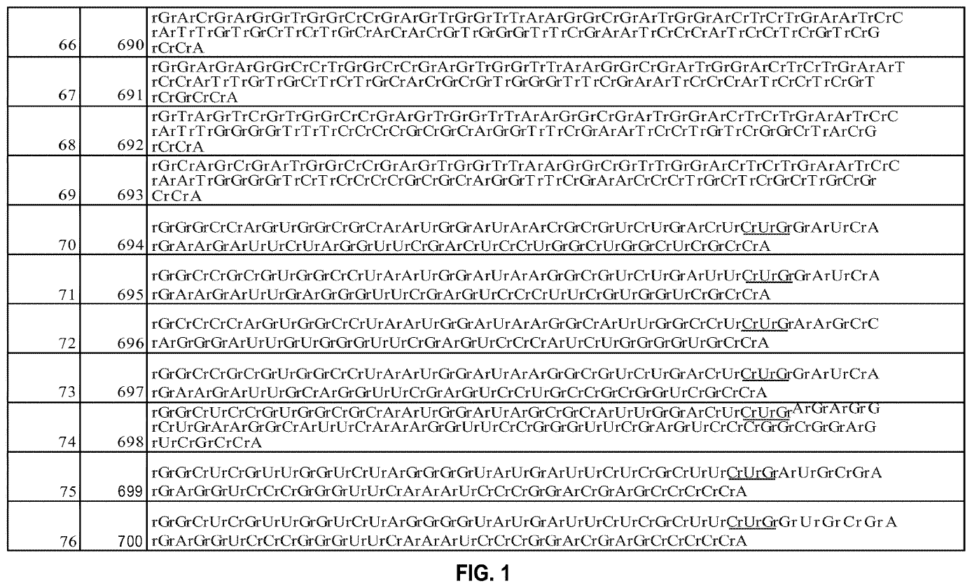

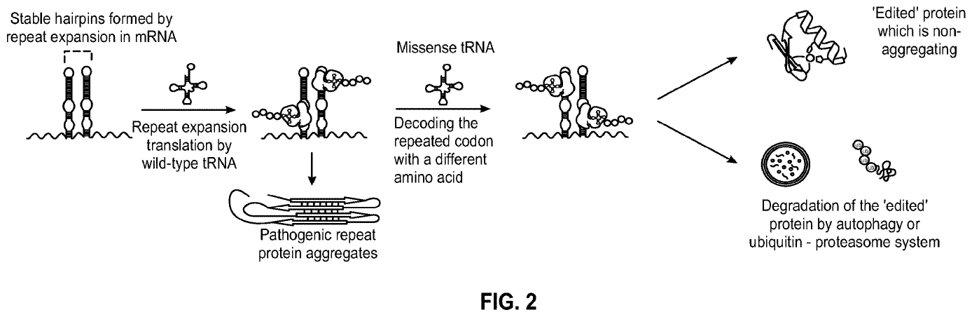

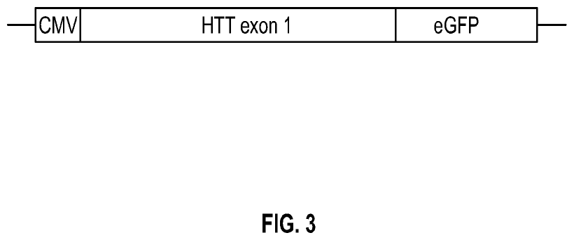

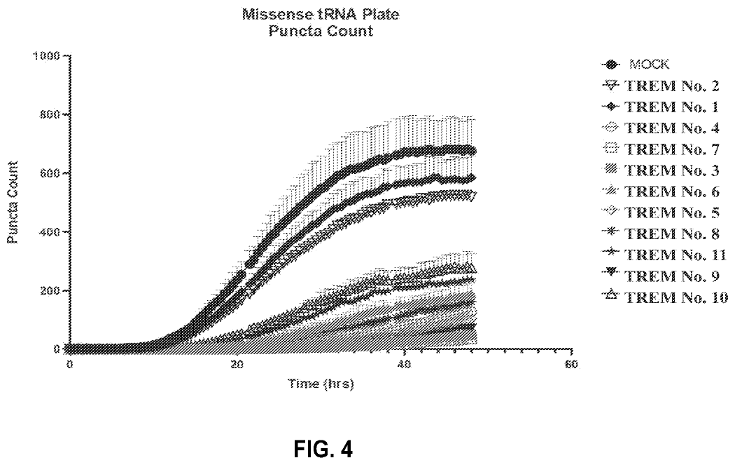

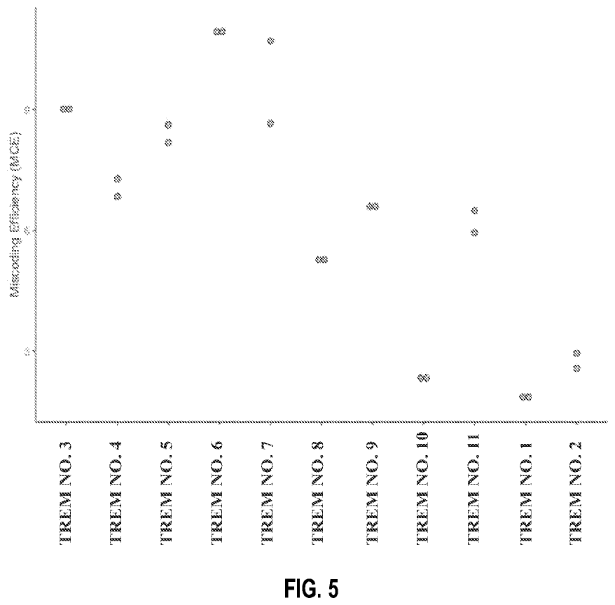

Methods of treating REDs In one aspect, the present disclosure features a method of treating a disease or disorder in a subject, the method comprising administering to the subject a TREM comprising the nucleotide sequence of any one of the TREMs described herein (e.g., a TREM of FIG. 1, e.g., a TREM having a sequence provided by any one of SEQ ID NOs: 622-693). In an embodiment, the disease or disorder is a RED, such as a RED selected from Table 9.

In some embodiments, the method comprises inserting a missense mutation into an ORF of a gene, wherein the missense mutation results in replacing a RED codon with a replacement codon, the method comprising contacting the ORF with a TREM described herein. In some embodiments, the replacement codon corresponds to an amino acid selected from an alanine (Ala), a leucine (Leu), and a serine (Ser). In some embodiments, the amino acid is an Ala, and the replacement codon is selected from: GCT, GCC, GCA, and GCG. In some embodiments, the amino acid is a Leu, and the replacement codon is selected from: TTA, TTG, CTT, CTC, CTA, and CTG. In some embodiments, the amino acid is a Ser, and the replacement codon is selected from: TCT, TCC, TCA, TCG.

For example, in some embodiments, the present disclosure features a method of treating a RED (e.g., Huntington's disease, SCA1, SCA2, SCA3, SCA7, SCA8, SCA17, DRPLA, glutaminase deficiency, and SBMA) comprising a repeat expansion comprising the repeat motif (e.g., RED codon) CAG. In some embodiments, the RED codon is CAG, and the replacement codon corresponds to an amino acid selected from Ala, Leu, and Ser. In some embodiments, the RED codon is CAG, the replacement codon corresponds to Ala, and the replacement codon is selected from: GCT, GCC, GCA, and GCG. In some embodiments, the RED codon is CAG, the replacement codon corresponds to Leu, and the replacement codon is selected from: TTA, TTG, CTT, CTC, CTA, and CTG. In some embodiments, the RED codon is CAG, the replacement codon corresponds to Ser, and the replacement codon is selected from: TCT, TCC, TCA, TCG.

In some embodiments, the present disclosure features a method of treating a RED (e.g., DM1, HDL2, and SCA8) comprising a repeat expansion comprising the repeat motif (e.g., RED codon) CTG. In some embodiments, the RED codon is CTG, and the replacement codon corresponds to an amino acid selected from Ala, Leu, and Ser. In some embodiments, the RED codon is CTG, the replacement codon corresponds to Ala, and the replacement codon is selected from: GCT, GCC, GCA, and GCG. In some embodiments, the RED codon is CTG, the replacement codon corresponds to Leu, and the replacement codon is selected from: TTA, TTG, CTT, CTC, CTA, and CTG. In some embodiments, the RED codon is CTG, the replacement codon corresponds to Ser, and the replacement codon is selected from: TCT, TCC, TCA, TCG.

In some embodiments, the present disclosure features a method of treating a RED (e.g., FRAXA, FXPOI, FXTAS, intellectual disability associated with fragile site FRA2A, oculopharyngodistal myopathy 2, and OPML) comprising a repeat expansion comprising the repeat motif (e.g., RED codon) CGG. In some embodiments, the RED codon is CGG, and the replacement codon corresponds to an amino acid selected from Ala, Leu, and Ser. In some embodiments, the RED codon is CGG, the replacement codon corresponds to Ala, and the replacement codon is selected from: GCT, GCC, GCA, and GCG. In some embodiments, the RED codon is CGG, the replacement codon corresponds to Leu, and the replacement codon is selected from: TTA, TTG, CTT, CTC, CTA, and CTG. In some embodiments, the RED codon is CGG, the replacement codon corresponds to Ser, and the replacement codon is selected from: TCT, TCC, TCA, TCG.

In some embodiments, the present disclosure features a method of treating a RED (e.g., Jacobsen syndrome and OPML) comprising a repeat expansion comprising the repeat motif (e.g., RED codon) CCG. In some embodiments, the RED codon is CCG, and the replacement codon corresponds to an amino acid selected from Ala, Leu, and Ser. In some embodiments, the RED codon is CCG, the replacement codon corresponds to Ala, and the replacement codon is selected from: GCT, GCC, GCA, and GCG. In some embodiments, the RED codon is CCG, the replacement codon corresponds to Leu, and the replacement codon is selected from: TTA, TTG, CTT, CTC, CTA, and CTG. In some embodiments, the RED codon is CCG, the replacement codon corresponds to Ser, and the replacement codon is selected from: TCT, TCC, TCA, TCG.

In some embodiments, the present disclosure features a method of treating a RED (e.g., MED and PSACH) comprising a repeat expansion comprising the repeat motif (e.g., RED codon) GAC. In some embodiments, the RED codon is GAC, and the replacement codon corresponds to an amino acid selected from Ala, Leu, and Ser. In some embodiments, the RED codon is GAC, the replacement codon corresponds to Ala, and the replacement codon is selected from: GCT, GCC, GCA, and GCG. In some embodiments, the RED codon is GAC, the replacement codon corresponds to Leu, and the replacement codon is selected from: TTA, TTG, CTT, CTC, CTA, and CTG. In some embodiments, the RED codon is GAC, the replacement codon corresponds to Ser, and the replacement codon is selected from: TCT, TCC, TCA, TCG.

In some embodiments, the present disclosure features a method of treating a RED (e.g., BPES, CCD, CHS, HPE, SPD1, X-linked hypopituitarism, and X-linked intellectual disability) comprising a repeat expansion comprising the repeat motif (e.g., RED codon) GCN, where N can be A, C, T, or G. In some embodiments, the RED codon is GCN, and the replacement codon corresponds to an amino acid selected from Ala, Leu, and Ser. In some embodiments, the RED codon is GCN, the replacement codon corresponds to Ala, and the replacement codon is selected from: GCT, GCC, GCA, and GCG. In some embodiments, the RED codon is GCN, the replacement codon corresponds to Leu, and the replacement codon is selected from: TTA, TTG, CTT, CTC, CTA, and CTG. In some embodiments, the RED codon is GCN, the replacement codon corresponds to Ser, and the replacement codon is selected from: TCT, TCC, TCA, TCG.

In another aspect, the method comprises inserting a missense mutation into an ORF of a gene, wherein the missense mutation results in replacing a 1^{st} or 2^{nd} trinucleotide Sequence within a hexanucleotide repeat with a replacement codon, the method comprising contacting the ORF with a TREM described herein (e.g., a TREM of FIG. 1, e.g., a TREM having a Sequence provided by any one of SEQ ID NOs: 622-693). In an embodiment, the disease or disorder is a RED, such as a RED selected from Table 9. In some embodiments, the RED is ALS, and the repeat expansion sequence comprises a hexanucleotide sequence selected from: GGGGCC, GGCCGG, GGGCCG, CCCCGG, and CCGGCC, wherein the 1^{st} trinucleotide Sequence comprises the 1^{st} three nucleotides in the sequence and the 2^{nd} trinucleotide sequence comprises the 2^{nd} three nucleotides in the sequence. For example, in some embodiments, (a) the hexanucleotide sequence is GGGGCC, the 1^{st} trinucleotide sequence is GGG, and the 2^{nd} trinucleotide sequence is GCC; and (b) the replacement codon replaces the 1^{st} trinucleotide sequence (e.g., GGG). In some embodiments, (a) the hexanucleotide sequence is GGGGCC, the 1^{st} trinucleotide sequence is GGG, and the 2^{nd} trinucleotide sequence is GCC; and (b) the replacement codon replaces the 2^{nd} trinucleotide sequence (e.g., GCC). In some embodiments, the replacement codon corresponds to an amino acid selected from Ala, Leu, and Ser. In some embodiments, the replacement codon corresponds to Ala, and the replacement codon is selected from: GCT, GCC, GCA, and GCG. In some embodiments, the replacement codon corresponds to Leu, and the replacement codon is selected from: TTA, TTG, CTT, CTC, CTA, and CTG. In some embodiments, the replacement codon corresponds to Ser, and the replacement codon is selected from: TCT, TCC, TCA, TCG.

In some embodiments, the method comprises reducing aggregation of a protein associated with repeat expansion (e.g., a protein associated with a RED codon, e.g., HTT, SCA1, SCA2, SCA3, SCA7, SCA8, SCA17, DRPLA, glutaminase deficiency, and SBMA), by administering a missense TREM to a sample (e.g., a cell) or a subject. In some embodiments, the method comprises reducing aggregation of a protein associated with repeat expansion by at least 1 (e.g., at least 2, 3, 4, 5, 10, or more) fold. For example, in some embodiments, the method comprises reducing aggregation of a protein associated with repeat expansion by at least 2 fold. In some embodiments, the method comprises reducing aggregation of a protein associated with repeat expansion by at least 3 fold. In some embodiments, the method comprises reducing aggregation of a protein associated with repeat expansion by at least 4 fold. In some embodiments, the method comprises reducing aggregation of a protein associated with repeat expansion by at least 5 fold. In some embodiments, the method comprises reducing aggregation of a protein associated with repeat expansion by at least 10 fold. In some embodiments, the method comprises reducing aggregation of a protein associated with repeat expansion by more than 10 fold.

In some embodiments, the method comprises reducing aggregation of a protein associated with repeat expansion by at least 1% (e.g., at least 5%, 10%, 25%, 50%, 75%, or more) fold. For example, in some embodiments, the method comprises reducing aggregation of a protein associated with repeat expansion by 5%. In some embodiments, the method comprises reducing aggregation of a protein associated with repeat expansion by at least 10%. In some embodiments, the method comprises reducing aggregation of a protein associated with repeat expansion by at least 25%. In some embodiments, the method comprises reducing aggregation of a protein associated with repeat expansion by at least 50%. In some embodiments, the method comprises reducing aggregation of a protein associated with repeat expansion by at least 75%. In some embodiments, the method comprises reducing aggregation of a protein associated with repeat expansion by more than 75%.

In some embodiments, the method comprises reducing aggregation of a protein associated with repeat expansion within 2 (e.g., within 3, 4, 5, 6, 10, 12, 24, 48, 72, 96, or more) hours. For example, in some embodiments, the method comprises reducing aggregation of a protein associated with repeat expansion within 3 hours. In some embodiments, the method comprises reducing aggregation of a protein associated with repeat expansion within 4 hours. In some embodiments, the method comprises reducing aggregation of a protein associated with repeat expansion within 5 hours. In some embodiments, the method comprises reducing aggregation of a protein associated with repeat expansion within 6 hours. In some embodiments, the method comprises reducing aggregation of a protein associated with repeat expansion within 10 hours. In some embodiments, the method comprises reducing aggregation of a protein associated with repeat expansion within 12 hours. In some embodiments, the method comprises reducing aggregation of a protein associated with repeat expansion within 24 hours. In some embodiments, the method comprises reducing aggregation of a protein associated with repeat expansion within 48 hours. In some embodiments, the method comprises reducing aggregation of a protein associated with repeat expansion within 72 hours. In some embodiments, the method comprises reducing aggregation of a protein associated with repeat expansion within 96 hours. In some embodiments, the method comprises reducing aggregation of a protein associated with repeat expansion after 96 hours.

In some embodiments, the method comprises reducing aggregation of a protein associated with repeat expansion after 1 (e.g., after 2, 3, 4, 5, 6, 7, 14, 21, or more) day. For example, in some embodiments, the method comprises reducing aggregation of a protein associated with repeat expansion after 2 days. In some embodiments, the method comprises reducing aggregation of a protein associated with repeat expansion after 3 days. In some embodiments, the method comprises reducing aggregation of a protein associated with repeat expansion after 4 days. In some embodiments, the method comprises reducing aggregation of a protein associated with repeat expansion after 5 days. In some embodiments, the method comprises reducing aggregation of a protein associated with repeat expansion after 6 days. In some embodiments, the method comprises reducing aggregation of a protein associated with repeat expansion after 7 days. In some embodiments, the method comprises reducing aggregation of a protein associated with repeat expansion after 14 days. In some embodiments, the method comprises reducing aggregation of a protein associated with repeat expansion after 21 days. In some embodiments, the method comprises reducing aggregation of a protein associated with repeat expansion after more than 21 days. In some embodiments, the reducing aggregation of a protein associated with repeat expansion comprises administering one or more Leu encoding missense TREMs.

In some embodiments, the reducing aggregation of a protein associated with repeat expansion comprises administering one or more Ser encoding missense TREMs.

In some embodiments, the reducing aggregation of a protein associated with repeat expansion comprises administering one or more Ala encoding missense TREMs.

### Method of Making TREMs, TREM Core Fragments, and TREM Fragments

In vitro methods for synthesizing oligonucleotides are known in the art and can be used to make a TREM, a TREM core fragment or a TREM fragment disclosed herein. For example, a TREM, TREM core fragment or TREM fragment can be synthesized using solid state synthesis or liquid phase synthesis.

In an embodiment, a TREM, a TREM core fragment or a TREM fragment made according to an in vitro synthesis method disclosed herein has a different modification profile compared to a TREM expressed and isolated from a cell, or compared to a naturally occurring tRNA.

An exemplary method for making a modified TREM is provided in Example 1. The method provided in Example 1 can also be used to make a synthetic TREM core fragment or synthetic TREM fragment. Additional synthetic methods are disclosed in Hartsel S A et al., (2005) Oligonucleotide Synthesis, 033-050, the entire contents of which are hereby incorporated by reference.

### TREM Composition

In an embodiment, a TREM composition, e.g., a TREM pharmaceutical composition, comprises a pharmaceutically acceptable excipient. Exemplary excipients include those provided in the FDA Inactive Ingredient Database (https://www.accessdata.fda.gov/scripts/cder/iig/index. Cfm).

In an embodiment, a TREM composition, e.g., a TREM pharmaceutical composition, comprises at least 1, 2, 3, 4, 5, 6, 7, 8, 9, 10, 15, 20, 30, 40, 50, 60, 70, 80, 90, 100 or 150 grams of TREM, TREM core fragment or TREM fragment. In an embodiment, a TREM composition, e.g., a TREM pharmaceutical composition, comprises at least 1, 2, 3, 4, 5, 6, 7, 8, 9, 10, 15, 20, 30, 40, 50 or 100 milligrams of TREM, TREM core fragment or TREM fragment.

In an embodiment, a TREM composition, e.g., a TREM pharmaceutical composition, is at least 10, 20, 30, 40, 50, 60, 70, 80, 90, 95 or 99% dry weight TREMs, TREM core fragments or TREM fragments.

In an embodiment, a TREM composition comprises at least 1×10⁶ TREM molecules, at least 1×10⁷ TREM molecules, at least 1×10⁸ TREM molecules or at least 1×10⁹ TREM molecules.

In an embodiment, a TREM composition comprises at least 1×10⁶ TREM core fragment molecules, at least 1×10⁷ TREM core fragment molecules, at least 1×10⁸ TREM core fragment molecules or at least 1×10⁹ TREM core fragment molecules.

In an embodiment, a TREM composition comprises at least 1×10⁶ TREM fragment molecules, at least 1×10⁷ TREM fragment molecules, at least 1×10⁸ TREM fragment molecules or at least 1×10⁹ TREM fragment molecules.

In an embodiment, a TREM composition produced by any of the methods of making disclosed herein can be charged with an amino acid using an in vitro charging reaction as known in the art.

In an embodiment, a TREM composition comprise one or more species of TREMs, TREM core fragments, or TREM fragments. In an embodiment, a TREM composition comprises a single species of TREM, TREM core fragment, or TREM fragment. In an embodiment, a TREM composition comprises a first TREM, TREM core fragment, or TREM fragment species and a second TREM, TREM core fragment, or TREM fragment species. In an embodiment, the TREM composition comprises X TREM, TREM core fragment, or TREM fragment species, wherein x=2, 3, 4, 5, 6, 7, 8, 9, or 10.

In an embodiment, the TREM, TREM core fragment, or TREM fragment has at least 70, 75, 80, 85, 90, or 95, or has 100%, identity with a sequence encoded by a nucleic acid in Table 1.

In an embodiment, the TREM comprises a consensus sequence provided herein.

A TREM composition can be formulated as a liquid composition, as a lyophilized composition or as a frozen composition.

In some embodiments, a TREM composition can be formulated to be suitable for pharmaceutical use, e.g., a pharmaceutical TREM composition. In an embodiment, a pharmaceutical TREM composition is substantially free of materials and/or reagents used to separate and/or purify a TREM, TREM core fragment, or TREM fragment.

In some embodiments, a TREM composition can be formulated with water for injection. In some embodiments, a TREM composition formulated with water for injection is suitable for pharmaceutical use, e.g., comprises a pharmaceutical TREM composition.

### TREM Characterization

A TREM, TREM core fragment, or TREM fragment, or a TREM composition, e.g., a pharmaceutical TREM composition, produced by any of the methods disclosed herein can be assessed for a characteristic associated with the TREM, TREM core fragment, or TREM fragment or the TREM composition, such as purity, sterility, concentration, structure, or functional activity of the TREM, TREM core fragment, or TREM fragment. Any of the above-mentioned characteristics can be evaluated by providing a value for the characteristic, e.g., by evaluating or testing the TREM, TREM core fragment, or TREM fragment, or the TREM composition, or an intermediate in the production of the TREM composition. The value can also be compared with a standard or a reference value. Responsive to the evaluation, the TREM composition can be classified, e.g., as ready for release, meets production standard for human trials, complies with ISO standards, complies with cGMP standards, or complies with other pharmaceutical standards. Responsive to the evaluation, the TREM composition can be subjected to further processing, e.g., it can be divided into aliquots, e.g., into single or multi-dosage amounts, disposed in a container, e.g., an end-use vial, packaged, shipped, or put into commerce. In embodiments, in response to the evaluation, one or more of the characteristics can be modulated, processed or re-processed to optimize the TREM composition. For example, the TREM composition can be modulated, processed or re-processed to (i) increase the purity of the TREM composition; (ii) decrease the amount of fragments in the composition; (iii) decrease the amount of endotoxins in the composition; (iv) increase the in vitro translation activity of the composition; (v) increase the TREM concentration of the composition; or (vi) inactivate or remove any viral contaminants present in the composition, e.g., by reducing the pH of the composition or by filtration.

In an embodiment, the TREM, TREM core fragment, or TREM fragment (e.g., TREM composition or an intermediate in the production of the TREM composition) has a purity of at least 30%, 40%, 50%, 60%, 70%, 80%, 85%, 90%, 91%, 92%, 93%, 94%, 95%, 96%, 97%, 98%, or 99%, i.e., by mass.

In an embodiment, the TREM (e.g., TREM composition or an intermediate in the production of the TREM composition) has less than 0.1%, 0.5%, 1%, 2%, 3%, 4%, 5%, 6%, 7%, 8%, 9%, 10%, 15%, 20%, 25% TREM fragments relative to full length TREMs.

In an embodiment, the TREM, TREM core fragment, or TREM fragment (e.g., TREM composition or an intermediate in the production of the TREM composition) has low levels or absence of endotoxins, e.g., a negative result as measured by the Limulus amebocyte lysate (LAL) test.

In an embodiment, the TREM, TREM core fragment, or TREM fragment (e.g., TREM composition or an intermediate in the production of the TREM composition) has in-vitro translation activity.

In an embodiment, the TREM, TREM core fragment, or TREM fragment (e.g., TREM composition or an intermediate in the production of the TREM composition) has a TREM concentration of at least 0.1 ng/mL, 0.5 ng/mL, 1 ng/mL, 5 ng/ml, 10 ng/ml, 50 ng/ml, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL.

In an embodiment, the TREM, TREM core fragment, or TREM fragment (e.g., TREM composition or an intermediate in the production of the TREM composition) is sterile, e.g., the composition or preparation supports the growth of fewer than 100 viable microorganisms as tested under aseptic conditions, the composition or preparation meets the standard of USP <71>, and/or the composition or preparation meets the standard of USP <85>.

In an embodiment, the TREM, TREM core fragment, or TREM fragment (e.g., TREM composition or an intermediate in the production of the TREM composition) has an undetectable level of viral contaminants, e.g., no viral contaminants. In an embodiment, any viral contaminant, e.g., residual virus, present in the composition is inactivated or removed. In an embodiment, any viral contaminant, e.g., residual virus, is inactivated, e.g., by reducing the pH of the composition. In an embodiment, any viral contaminant, e.g., residual virus, is removed, e.g., by filtration or other methods known in the field.

### TREM Administration

Any TREM composition or pharmaceutical composition described herein can be administered to a cell, tissue or subject, e.g., by direct administration to a cell, tissue and/or an organ in vitro, ex-vivo or in vivo. In-vivo administration may be via, e.g., by local, systemic and/or parenteral routes, for example intravenous, subcutaneous, intraperitoneal, intrathecal, intramuscular, ocular, nasal, urogenital, intradermal, dermal, enteral, intravitreal, intracerebral, intrathecal, or epidural.

### Vectors and Carriers

In some embodiments, the TREM, TREM core fragment, or TREM fragment or TREM composition described herein, is delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, or an adenovirus. In some embodiments, the system or components of the system are delivered to cells with a viral-like particle or a virosome. In some embodiments, the delivery uses more than one virus, viral-like particle or virosome.

### Carriers

A TREM, a TREM composition or a pharmaceutical TREM composition described herein may comprise, may be formulated with, or may be delivered in, a carrier.

### Viral Vectors

The carrier may be a viral vector (e.g., a viral vector comprising a sequence encoding a TREM, a TREM core fragment or a TREM fragment). The viral vector may be administered to a cell or to a subject (e.g., a human subject or animal model) to deliver a TREM, a TREM core fragment or a TREM fragment, a TREM composition or a pharmaceutical TREM composition.

A viral vector may be systemically or locally administered (e.g., injected). Viral genomes provide a rich source of vectors that can be used for the efficient delivery of exogenous genes into a mammalian cell. Viral genomes are known in the art as useful vectors for delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference. In some embodiments, the system or components of the system are delivered to cells with a viral-like particle or a virosome.

### Cell and Vesicle-Based Carriers

A TREM, a TREM core fragment or a TREM fragment, a TREM composition or a pharmaceutical TREM composition described herein can be administered to a cell in a vesicle or other membrane-based carrier.

In embodiments, a TREM, a TREM core fragment or a TREM fragment, or TREM composition, or pharmaceutical TREM composition described herein is administered in or via a cell, vesicle or other membrane-based carrier. In one embodiment, the TREM, TREM core fragment, TREM fragment, or TREM composition or pharmaceutical TREM composition can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi: 10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi: 10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

### Lipid Nanoformulations/Lipid-Based Carriers

Lipid nanoparticles are another example of a carrier that provides a biocompatible and biodegradable delivery system for the TREM, TREM core fragment, TREM fragment, or TREM composition or pharmaceutical TREM composition described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi: 10.3390/nano7060122.

Exemplary lipid nanoparticles are disclosed in International Application PCT/US2014/053907, the entire contents of which are hereby incorporated by reference. For example, an LNP described in paragraphs [403-406] or [410-413] of PCT/US2014/053907 can be used as a carrier for the TREM, TREM core fragment, TREM fragment, or TREM composition or pharmaceutical TREM composition described herein.

Additional exemplary lipid nanoparticles are disclosed in U.S. Pat. No. 10,562,849 the entire contents of which are hereby incorporated by reference. For example, an LNP of formula (I) as described in columns 1-3 of U.S. Pat. No. 10,562,849 can be used as a carrier for the TREM, TREM core fragment, TREM fragment, or TREM composition or pharmaceutical TREM composition described herein.

Lipids that can be used in nanoparticle formations (e.g., lipid nanoparticles) include, for example those described in Table 4 of WO2019217941, which is incorporated by reference, e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), incorporated herein by reference.

### Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N-dimethyl-N,N-dioctacyl ammonium bromide (DDAB) and chloride DDAC), N-(1-(2,3-dioleyloxy) propyl)-N,N,N-trimethylammonium chloride (DOTMA), 3β-[N—(N,N-dimethylaminoethyl) carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy) propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy) propane (CLinDMA), 2-[5′-(cholest-5-en-3-beta-oxy)-3′-oxapentoxy)-3-dimethyl-1-(cis, cis-9′,12′-octadecadienoxy) propane (CpLin DMA), N,N-Dimethyl-3,4-dioleyloxybenzylamine (DMOBA), and the cationic lipids described in e.g. Martin et al., Current Pharmaceutical Design, pages 1-394, which is herein incorporated by reference in its entirety. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, which is incorporated by reference.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

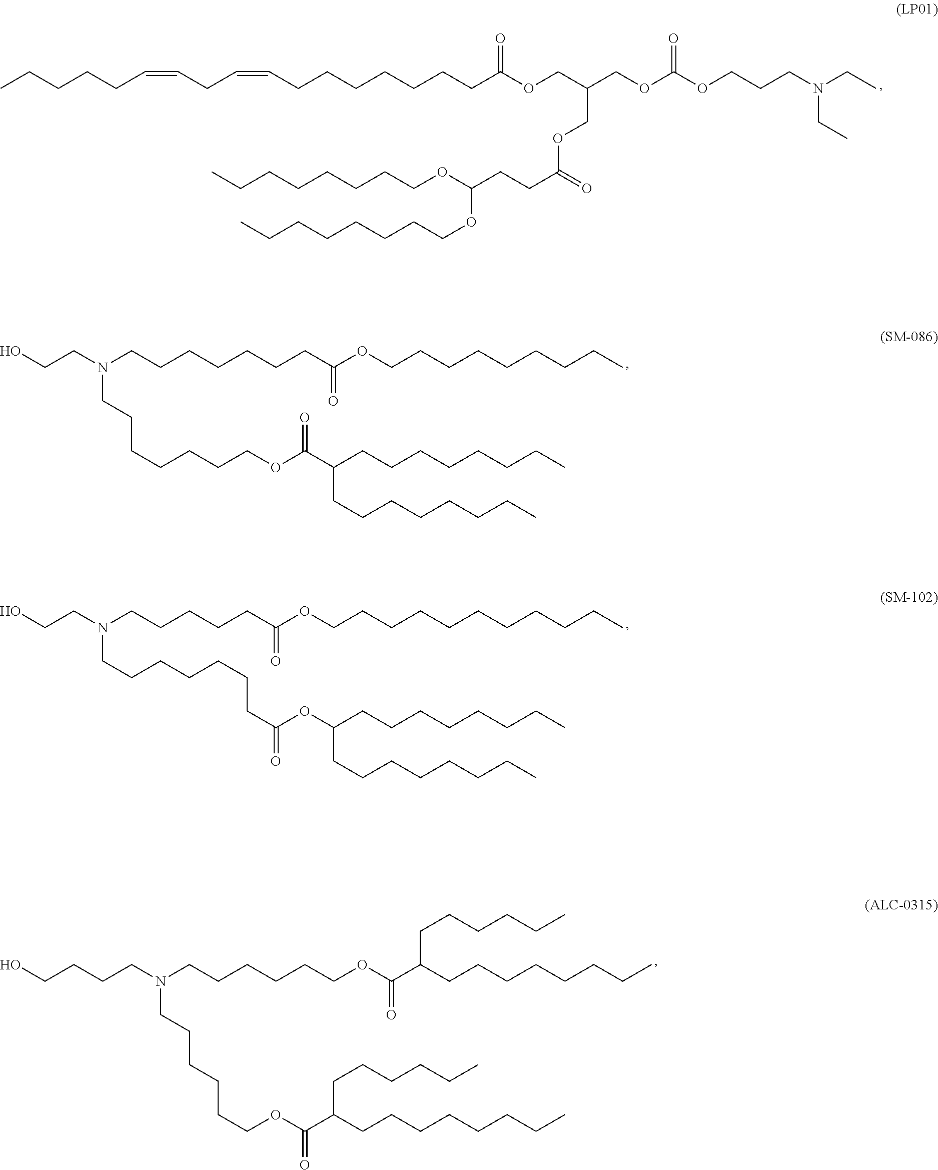

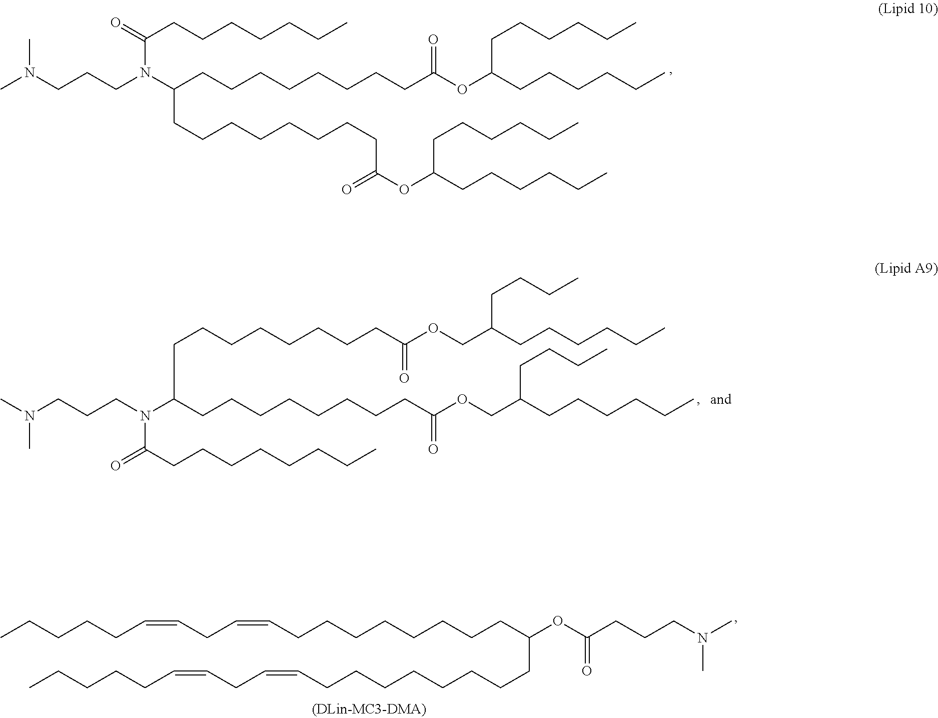

(see WO 2017/004143A1, which is incorporated herein by reference in its entirety).

In some embodiments, the lipid-based carrier (or lipid nanoformulation) further comprises one or more compounds described by WO 2021/113777 (e.g., a lipid of Formula (3) such as a lipid of Table 3 of WO 2021/113777), which is incorporated herein by reference in its entirety.

In one embodiment, the ionizable lipid is a lipid disclosed in Hou, X., et al. Nat Rev Mater 6, 1078-1094 (2021). https://doi.org/10.1038/s41578-021-00358-0 (e.g., L319, C12-200, and DLin-MC3-DMA), (which is incorporated by reference herein in its entirety).

Examples of other ionizable lipids that can be used in lipid-based carrier (or lipid nanoformulation) include, without limitation, one or more of the following formulas: X of US 2016/0311759; I of US20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y—Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; III-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-O13 or 503-O13 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, all of which are incorporated herein by reference in their entirety.

In some embodiments, the lipid-based carrier (or lipid nanoformulation) further includes biodegradable ionizable lipids, for instance, (9Z,12Z)-3-((4,4-bis(octyloxy) butanoyl)oxy)-2-((((3-(diethylamino) propoxy) carbonyl)oxy)methyl) propyl octadeca-9,12-dienoate, also called 3-((4,4-bis(octyloxy) butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, which are incorporated herein by reference in their entirety.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA) described herein includes,

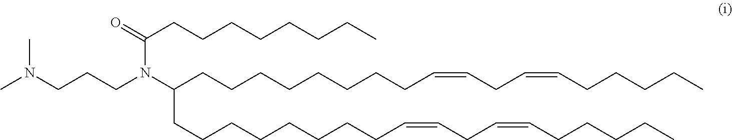 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a TREM composition described herein to the liver and/or hepatocyte cells.

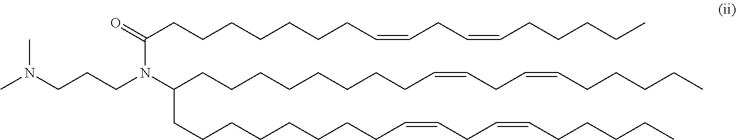 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a TREM composition described herein to the liver and/or hepatocyte cells.

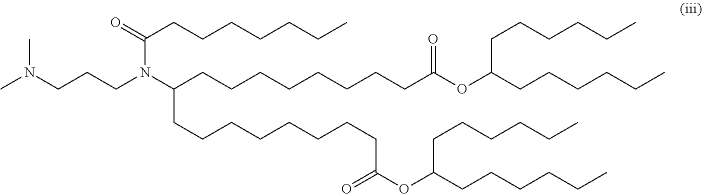 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a TREM composition described herein to the liver and/or hepatocyte cells.

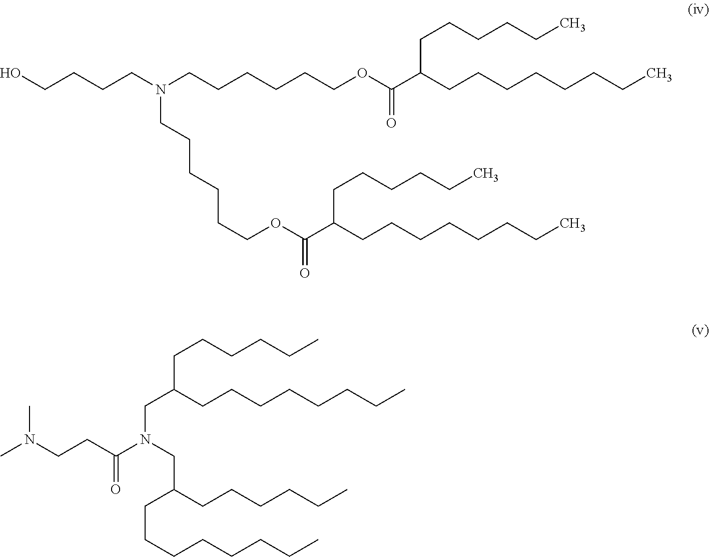

In some embodiments an LNP comprising Formula (v) is used to deliver a TREM composition described herein to the liver and/or hepatocyte cells.

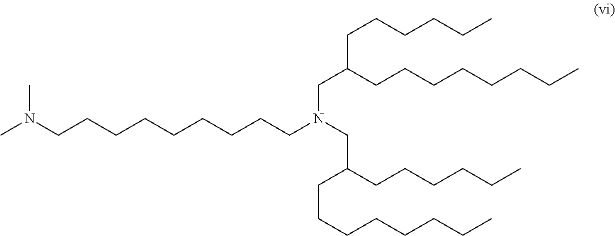 [SMILES: CCCCCCCCC(CCCCCC)CN(CCCCCCCCCN(C)C)CC(CCCCCC)CCCCCCCC]

In some embodiments an LNP comprising Formula (vi) is used to deliver a TREM composition described herein to the liver and/or hepatocyte cells.

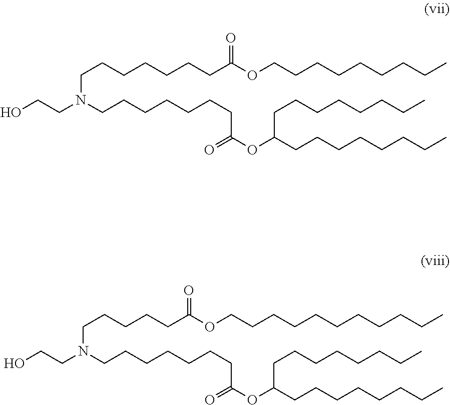 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a TREM composition described herein to the liver and/or hepatocyte cells.

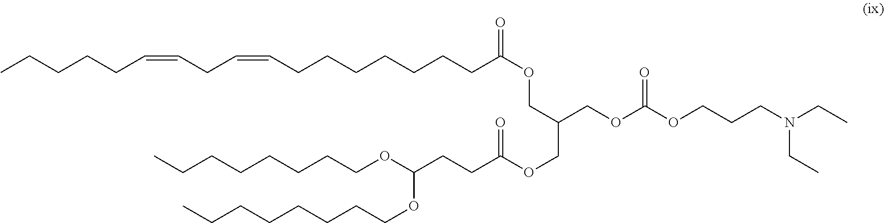 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a TREM composition described herein to the liver and/or hepatocyte cells.

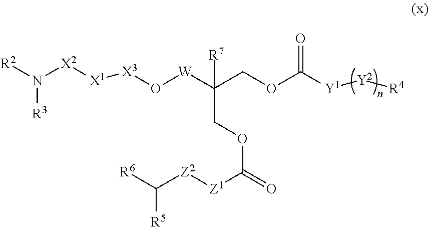

wherein X¹ is O, NR¹, or a direct bond, X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

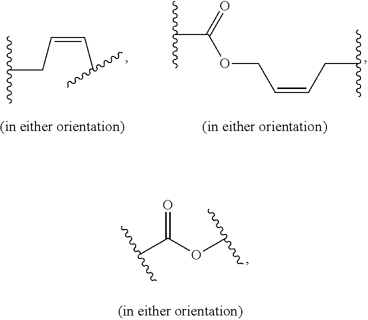 [SMILES: */C=C\C* ; *C/C=C\COC(*)=O ; *OC(*)=O]

n is 0 to 3, R⁴ is C1-15 alkyl, Z¹ is C1-6 alkylene or a direct bond, Z² is

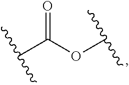 [SMILES: *OC(*)=O]

(in either orientation) or absent, provided that if Z¹ is a direct bond, Z² is absent; R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is; R⁴ is

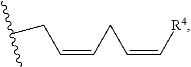

linear C5 alkyl, Z¹ is C2 alkylene, Z² is absent, W is methylene, and R⁷ is H, then R⁵ and R⁶ are not Cx alkoxy.

In some embodiments an LNP comprising Formula (xii) is used to deliver a TREM composition described herein to the liver and/or hepatocyte cells.

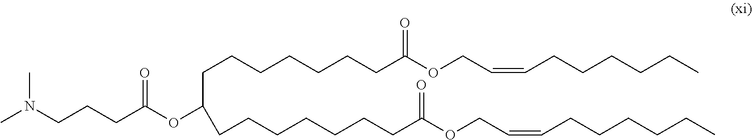 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a TREM composition described herein to the liver and/or hepatocyte cells.

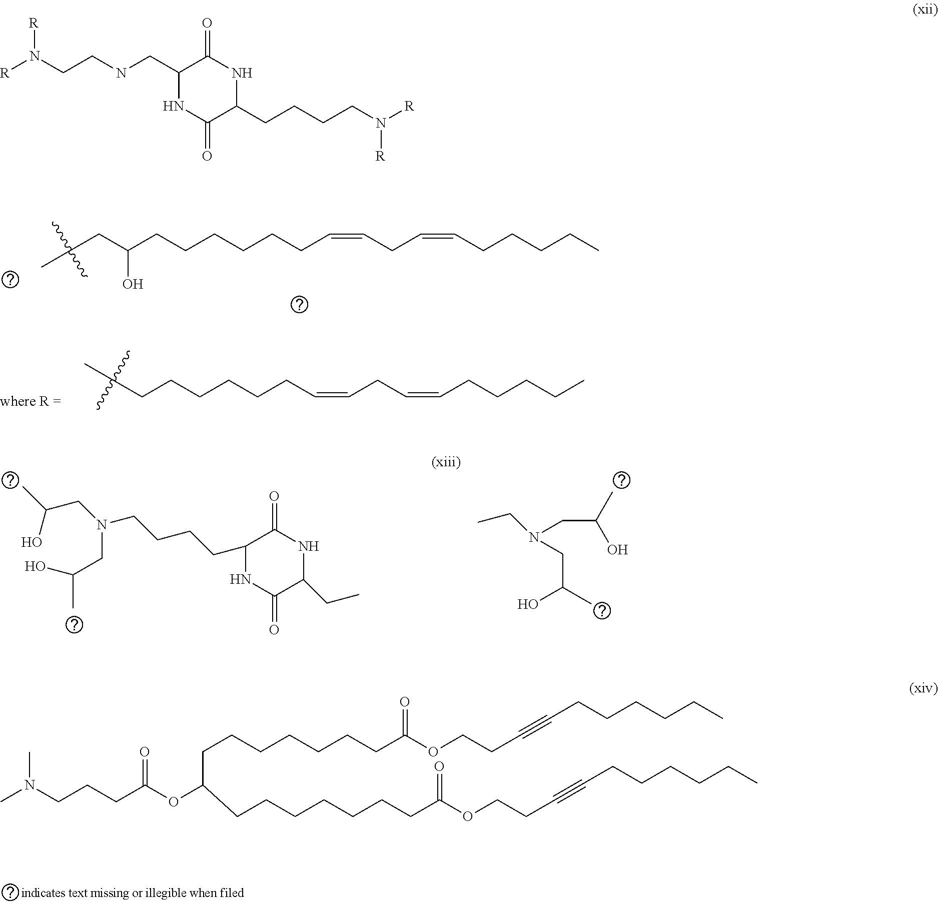

In some embodiments an LNP comprises a compound of Formula (xiii) and a compound of Formula (xiv).

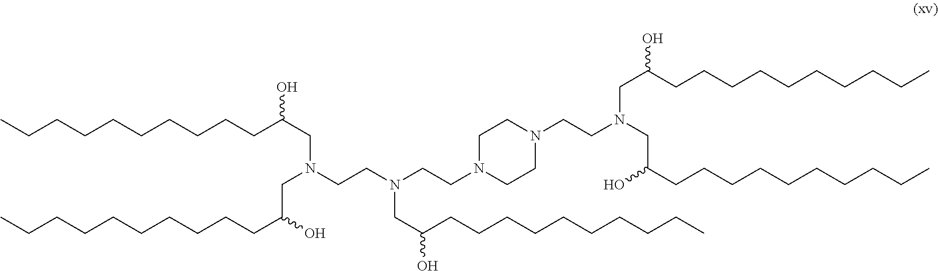 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, an LNP comprising Formula (xv) is used to deliver a TREM composition described herein to the liver and/or hepatocyte cells.

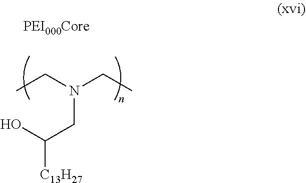

In some embodiments an LNP comprising a formulation of Formula (xvi) is used to deliver a TREM composition described herein to the lung endothelial cells.

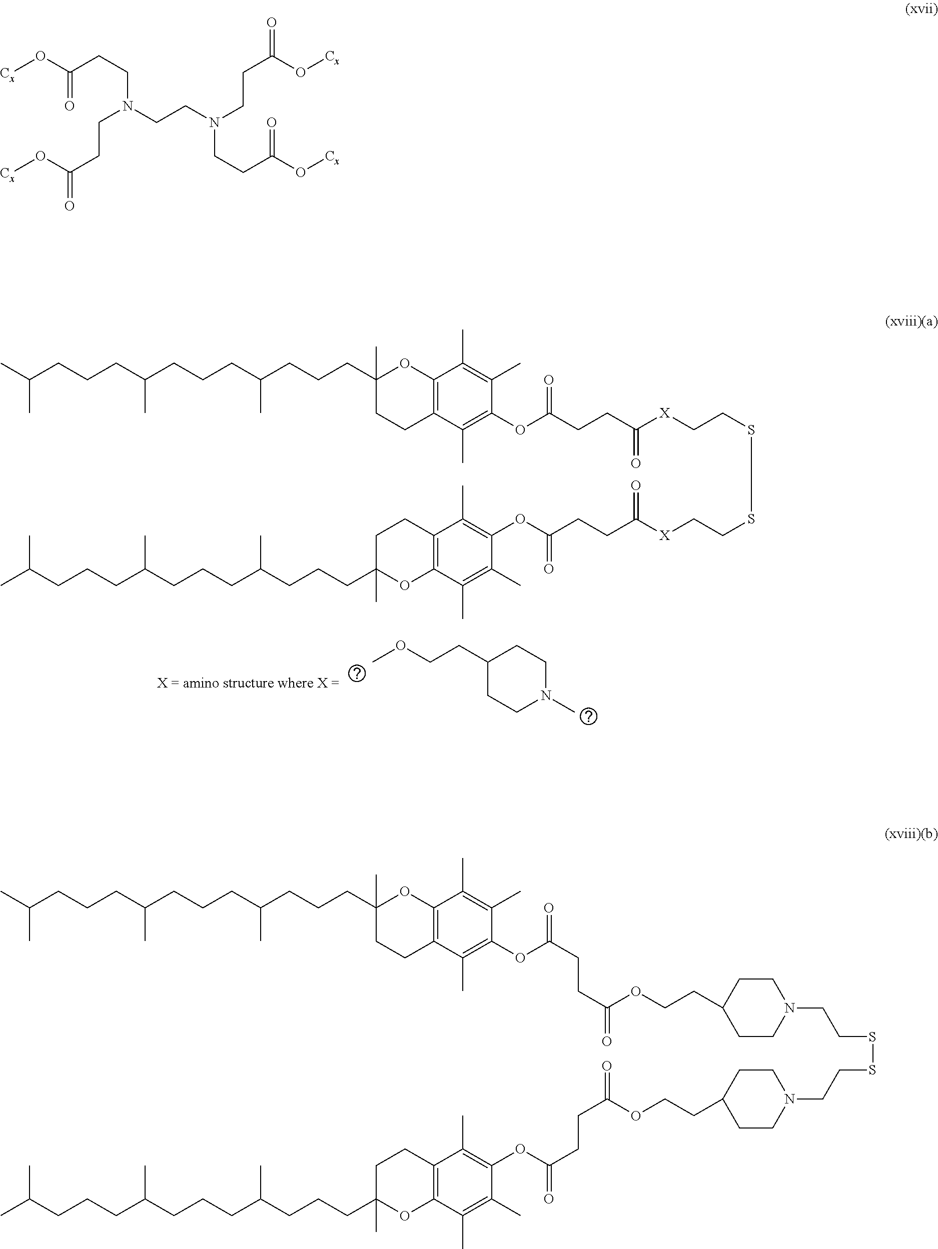

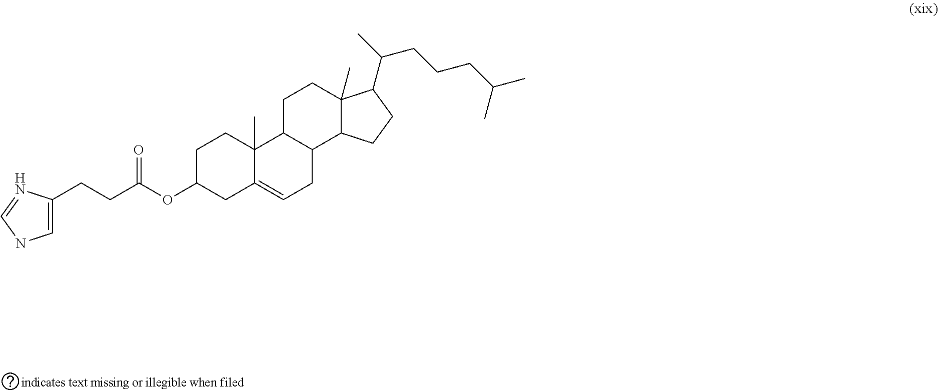

In some embodiments, a lipid compound used to form lipid nanoparticles for the delivery of compositions described herein, e.g., a TREM described herein is made by one of the following reactions:

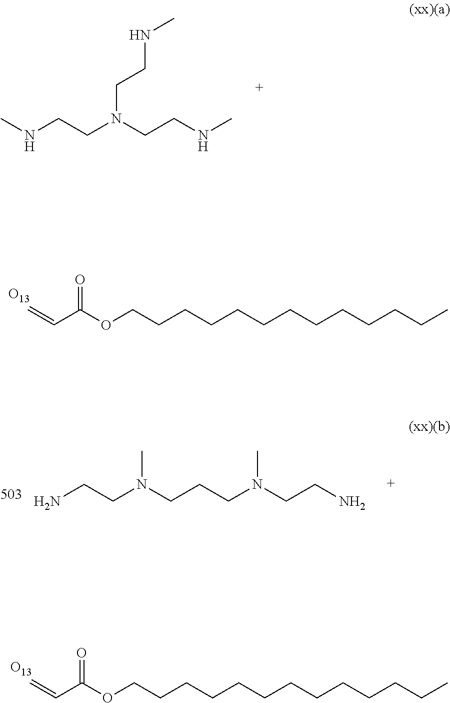

In some embodiments, a composition described herein (e.g., TREM composition) is provided in an LNP that comprises an ionizable lipid. In some embodiments, the ionizable lipid is heptadecan-9-yl 8-((2-hydroxyethyl) (6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z, 12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)-butanoyl)oxy) heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl) (2-hydroxydodecyl)amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10,13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a TREM described herein, encapsulated within or associated with the lipid nanoparticle. In some embodiments, the TREM is co-formulated with the cationic lipid. The TREM may be adsorbed to the surface of an LNP, e.g., an LNP comprising a cationic lipid. In some embodiments, the TREM may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of a TREM.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y—Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,3 IZ)-heptatriaconta-6,9,28,3 1-tetraen-19-yl-4-(dimethylamino)butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

### Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, which is incorporated herein by reference. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, which are incorporated herein by reference in their entirety.

In one embodiment, the lipid-based carrier (or lipid nanoformulation) further comprises one or more non-cationic lipid that is oleic acid or a compound of Formula I, II, or IV of US 2018/0028664, which is incorporated herein by reference in its entirety.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

### Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipid in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, which are incorporated herein by reference in their entirety. Additional exemplary sterols include phytosterols, including those described in Eygeris et al. (2020), Nano Lett. 2020; 20 (6): 4543-4549, incorporated herein by reference.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4-hydroxy)-butyl ether. Exemplary cholesterol derivatives are described in WO 2009/127060 and US 2010/0130588, each of which is incorporated herein by reference in its entirety.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

### Polymers and Polyethylene Glycol (PEG)-Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy(Polyethylene glycol)-(mPEG 350 PE); 1,2-Diacyl-sn-Glycero-3-Phosphoethanolamine-N-[Methoxy(Polyethylene glycol)-550] (mPEG 550 PE); 1,2-Diacyl-sn-Glycero-3-Phosphoethanolamine-N-[Methoxy(Polyethylene glycol)-750] (mPEG 750 PE); 1,2-Diacyl-sn-Glycero-3-Phosphoethanolamine-N-[Methoxy(Polyethylene glycol)-1000] (mPEG 1000 PE); 1,2-Diacyl-sn-Glycero-3-Phosphoethanolamine-N-[Methoxy(Polyethylene glycol)-2000] (mPEG 2000 PE); 1,2-Diacyl-sn-Glycero-3-Phosphoethanolamine-N-[Methoxy(Polyethylene glycol)-(mPEG 3000 PE); 1,2-Diacyl-sn-Glycero-3-Phosphoethanolamine-N-[Methoxy(Polyethylene glycol)-5000] (mPEG 5000 PE); N-Acyl-Sphingosine-1-[Succinyl(Methoxy Polyethylene Glycol) 750] (mPEG 750 Ceramide); N-Acyl-Sphingosine-1-[Succinyl(Methoxy Polyethylene Glycol) 2000] (mPEG 2000 Ceramide); and N-Acyl-Sphingosine-1-[Succinyl(Methoxy Polyethylene Glycol) 5000] (mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, which is incorporated herein by reference in its entirety). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2,3-di(tetradecanoyloxy) propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (which is herein incorporated by reference in its entirety), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, all of which are incorporated herein by reference in their entirety.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, both of which are incorporated herein by reference in their entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

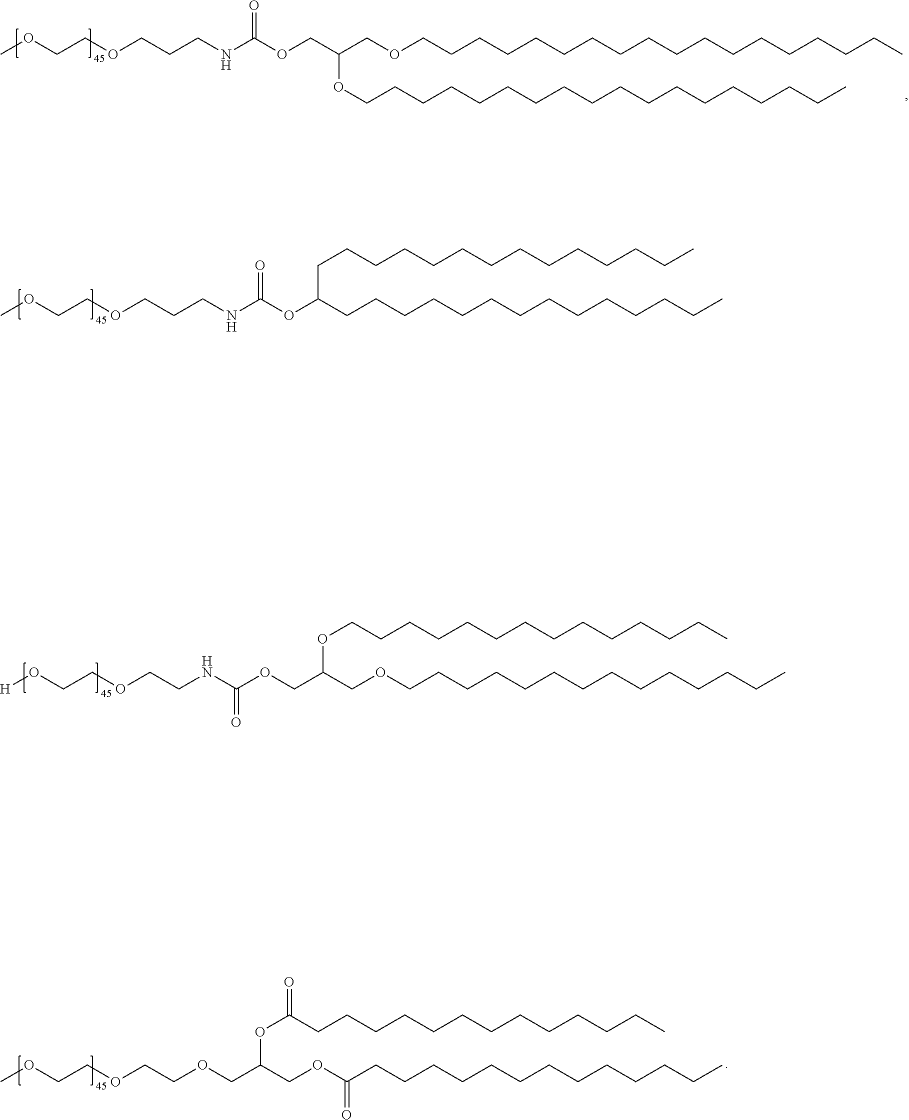

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, which is incorporated herein by reference in its entirety.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289:1128-1133 (1999); Gabizon et al., J Controlled Release 53:275-279 (1998); Adlakha Hutcheon et al., Nat Biotechnol 17:775-779 (1999); and Koning et al., Biochim Biophys Acta 1420:153-167 (1999), which are incorporated herein by reference in their entirety.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3 [beta]-oxy) carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy (polyethylene glycol)-2000]. In some embodiments, the PEG-lipid comprises PEG-DMG, 1,2-dimyristoyl-sn-glycero-3-phosphoethanolamine-N-[methoxy (polyethylene glycol)-2000]. In some embodiments, the PEG-lipid comprises a structure selected from:

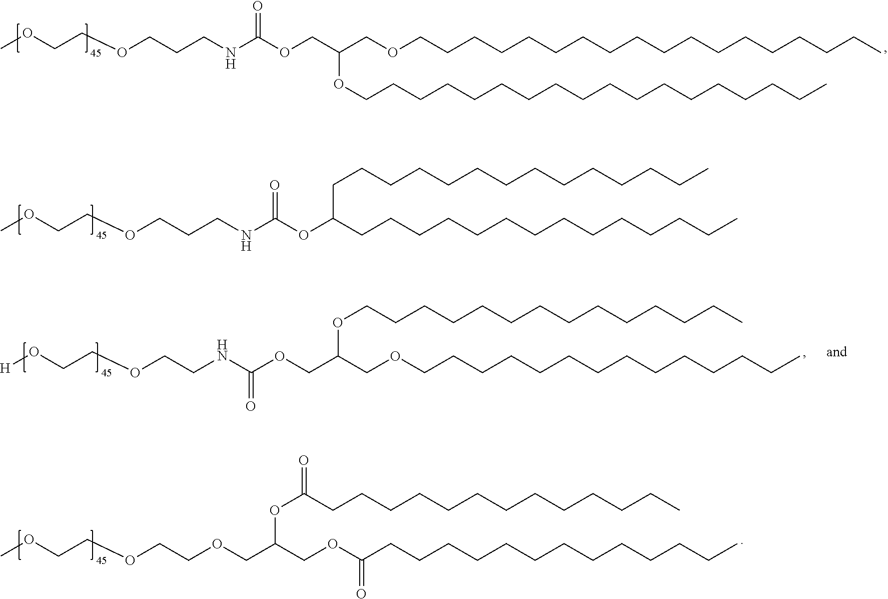

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

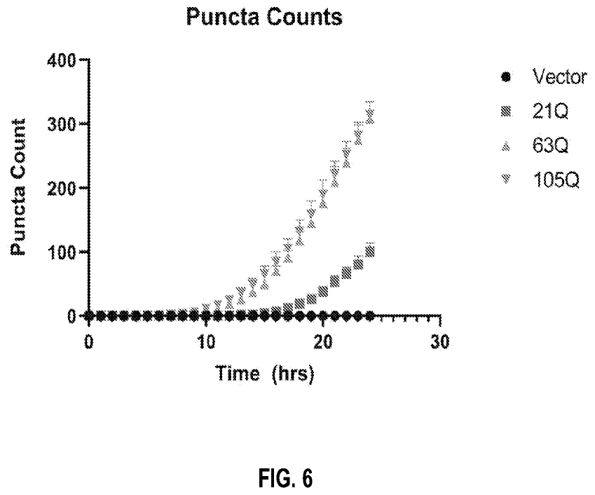

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18 (7): 1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15 (4): 313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy) butanoyl)oxy)-2-((((3-(diethylamino)propoxy)carbonyl)oxy)methyl)propyl (9Z,12Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a TREM describes the amount of TREM that is encapsulated or otherwise associated with a LNP after preparation, relative to the initial amount provided. The encapsulation efficiency is desirably high (e.g., close to 100%). The encapsulation efficiency may be measured, for example, by comparing the amount of TREM in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free TREM in a solution. For the lipid nanoparticles described herein, the encapsulation efficiency of a TREM may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51 (34): 8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exosomes can also be used as drug delivery vehicles for the TREM, TREM core fragment, TREM fragment, or TREM compositions or pharmaceutical TREM composition described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a TREM, TREM core fragment, TREM fragment, or TREM composition, or pharmaceutical TREM composition described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111 (28): 10131-10136; U.S. Pat. No. 9,644,180; Huang et al. 2017. Nature Communications 8:423; Shi et al. 2014. Proc Natl Acad Sci USA. 111 (28): 10131-10136.

Fusosome compositions, e.g., as described in WO2018208728, can also be used as carriers to deliver the TREM, TREM core fragment, TREM fragment, or TREM composition, or pharmaceutical TREM composition described herein.

Virosomes and virus-like particles (VLPs) can also be used as carriers to deliver a TREM, TREM core fragment, TREM fragment, or TREM composition, or pharmaceutical TREM composition described herein to targeted cells.

Plant nanovesicles, e.g., as described in WO2011097480A1, WO2013070324A1, or WO2017004526A1 can also be used as carriers to deliver the TREM, TREM core fragment, TREM fragment, or TREM composition, or pharmaceutical TREM composition described herein.

### Percentages of Lipid Nanoformulation Components

In some embodiments, the lipid-based carrier (or lipid nanoformulation) comprises one of more of the compounds described herein, optionally a non-cationic lipid (e.g., a phospholipid), a sterol, a neutral lipid, and optionally conjugated lipid (e.g., a PEGylated lipid) that inhibits aggregation of particles. In some embodiments, the lipid-based carrier (or lipid nanoformulation) further comprises a TREM (e.g., a TREM described herein). The amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid components, provided that the total mol % of the lipid component does not exceed 100%.

In some embodiments, the lipid-based carrier (or lipid nanoformulation) comprises about 25-100 mol % of the ionizable lipid including the lipid compounds described herein, about 0-50 mol % phospholipid, about 0-50 mol % sterol, and about 0-10 mol % PEGylated lipid.

In some embodiments, the lipid-based carrier comprises a TREM (e.g., a TREM described herein) that is formulated in a lipid nanoparticle, wherein the lipid nanoparticle comprises about 25-100 mol % of the ionizable lipid including the lipid compounds described herein, about 0-50 mol % phospholipid, about 0-50 mol % sterol, and about 0-10 mol % PEGylated lipid. In some embodiments, the encapsulation efficiency of the payload may be at least 70%.

In one embodiment, the lipid-based carrier (or lipid nanoformulation) comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier comprises a TREM (e.g., a TREM described herein) that is formulated in a lipid nanoparticle, wherein the lipid nanoparticle comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid. In some embodiments, the encapsulation efficiency of the payload may be at least 70%.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, the lipid-based carrier comprises a TREM (e.g., a TREM described herein) that is formulated in a lipid nanoparticle, wherein the lipid nanoparticle comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid. In some embodiments, the encapsulation efficiency of the payload may be at least 70%.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier comprises a TREM (e.g., a TREM described herein) that is formulated in a lipid nanoparticle, wherein the lipid nanoparticle comprises molar ratios of ionizable lipid/sterol/phospholipid (or another structural lipid)/PEG-lipid/additional components in the following ranges: ionizable lipid (25-100%); phospholipid (DSPC) (0-40%); sterol (0-50%); and PEG lipid (0-5%). In some embodiments, the encapsulation efficiency of the payload may be at least 70%.

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid).

In some embodiments, the lipid-based carrier comprises a TREM (e.g., a TREM described herein) that is formulated in a lipid nanoparticle, wherein the lipid nanoparticle comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid). In some embodiments, the encapsulation efficiency of the payload may be at least 70%.

In some embodiments, the lipid-based carrier (or lipid nanoformulation) comprises (i) a TREM (e.g., a TREM described herein); (ii) a cationic lipid comprising from 50 mol % to 65 mol % of the total lipid present in the lipid-based carrier; (iii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iv) a conjugated lipid comprising 0.5 mol % to 2 mol % of the total lipid present in the particle.

In some embodiments, the lipid-based carrier (or lipid nanoformulation) comprises (i) a TREM (e.g., a TREM described herein); (ii) a cationic lipid comprising from 50 mol % to 85 mol % of the total lipid present in the lipid-based carrier; (iii) a non-cationic lipid comprising from 13 mol % to 49.5 mol % of the total lipid present in the lipid-based carrier; and (d) a conjugated lipid comprising from 0.5 mol % to 2 mol % of the total lipid present in the lipid-based carrier.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the payload (e.g., an encapsulated therapeutic agent such as a TREM (e.g., a TREM described herein) can be varied as desired. For example, the total lipid components to the payload (mass or weight) ratio can be from about 10:1 to about 30:1. In some embodiments, the total lipid components to the payload ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the payload can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation's) overall lipid content can range from about 5 mg/ml to about 30 mg/mL. Nitrogen: phosphate ratios (N: P ratio) is evaluated at values between 0.1 and 100.

The efficiency of encapsulation of a payload such as a protein and/or nucleic acid, describes the amount of protein and/or nucleic acid that is encapsulated or otherwise associated with a lipid nanoformulation (e.g., liposome or LNP) after preparation, relative to the initial amount provided. The encapsulation efficiency is desirably high (e.g., at least 70%, 80%, 90%, 95%, close to 100%). The encapsulation efficiency may be measured, for example, by comparing the amount of protein or nucleic acid in a solution containing the liposome or LNP before and after breaking up the liposome or LNP with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid-based carrier (or lipid nanoformulation) described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 70%. In some embodiments, the encapsulation efficiency may be at least 80%. In some embodiments, the encapsulation efficiency may be at least 90%. In some embodiments, the encapsulation efficiency may be at least 95%.

Delivery without a Carrier

A TREM, a TREM core fragment or a TREM fragment, a TREM composition or a pharmaceutical TREM composition described herein can be administered to a cell without a carrier, e.g., via naked delivery of the TREM, a TREM core fragment or a TREM fragment, a TREM composition or a pharmaceutical TREM composition.

In some embodiments, naked delivery as used herein refers to delivery without a carrier. In some embodiments, delivery without a carrier, e.g., naked delivery, comprises delivery with a moiety, e.g., a targeting peptide.

In some embodiments, a TREM, a TREM core fragment or a TREM fragment, or TREM composition, or pharmaceutical TREM composition described herein is delivered to a cell without a carrier, e.g., via naked delivery. In some embodiments, the delivery without a carrier, e.g., naked delivery, comprises delivery with a moiety, e.g., a targeting peptide.

### Use of TREMs

A TREM composition (e.g., a pharmaceutical TREM composition described herein) can modulate a function in a cell, tissue or subject. In embodiments, a TREM composition (e.g., a pharmaceutical TREM composition) described herein is contacted with a cell or tissue, or administered to a subject in need thereof, in an amount and for a time sufficient to modulate (increase or decrease) one or more of the following parameters: adaptor function (e.g., cognate or non-cognate adaptor function), e.g., the rate, efficiency, robustness, and/or specificity of initiation or elongation of a polypeptide chain; ribosome binding and/or occupancy; regulatory function (e.g., gene silencing or signaling); cell fate; mRNA stability; protein stability; protein transduction; protein compartmentalization. A parameter may be modulated, e.g., by at least 5% (e.g., at least 10%, 15%, 20%, 25%, 30%, 40%, 50%, 60%, 70%, 80%, 90%, 100%, 150%, 200% or more) compared to a reference tissue, cell or subject (e.g., a healthy, wild-type or control cell, tissue or subject).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### ENUMERATED EMBODIMENTS

The disclosure herein is further presented as a non-limiting list of numbered embodiments.

1. A method for inserting a missense mutation into an open reading frame (ORF) of a gene, wherein the missense mutation results in replacing a repeat expansion disease (RED) codon with a replacement codon,
the method comprising contacting the ORF with a tRNA effector molecule (TREM) comprising a sequence of Formula (A):

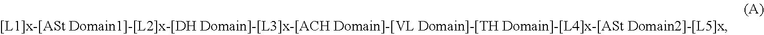

wherein:
independently, [L1] and [VL Domain], are optional and x=0 or 1, thereby inserting the missense mutation into the ORF of the gene.
   2. The method of embodiment 1, wherein the RED codon comprises CAG, CTG, CGG, GAC, or CCG.
   3. The method of embodiment 2, wherein the RED codon is CAG.
   4. The method of embodiment 2, wherein the RED codon is CTG.
   5. The method of embodiment 2, wherein the RED codon is CGG.
   6. The method of embodiment 2, wherein the RED codon is GAC.
   7. The method of embodiment 2, wherein the RED codon is CCG.
   8. The method of any one of the preceding embodiments, wherein the ORF comprises at least 5, 10, 15, 20, 25, 30, 35, 40, 45, 50, 55, 60, 65, 70, 75, or more copies (e.g., consecutive copies) of the RED codon.
   9. The method of embodiment 8, wherein the ORF comprises at least 5 or more copies (e.g., consecutive copies) of the RED codon.
   10. The method of embodiment 8, wherein the ORF comprises at least 10 or more copies (e.g., consecutive copies) of the RED codon.
   11. The method of embodiment 8, wherein the ORF comprises at least 15 or more copies (e.g., consecutive copies) of the RED codon.
   12. The method of embodiment 8, wherein the ORF comprises at least 20 or more copies (e.g., consecutive copies) of the RED codon.
   13. The method of embodiment 8, wherein the ORF comprises at least 25 or more copies (e.g., consecutive copies) of the RED codon.
   14. The method of embodiment 8, wherein the ORF comprises at least 30 or more copies (e.g., consecutive copies) of the RED codon.
   15. The method of embodiment 8, wherein the ORF comprises at least 35 or more copies (e.g., consecutive copies) of the RED codon.
   16. The method of embodiment 8, wherein the ORF comprises at least 40 or more copies (e.g., consecutive copies) of the RED codon.
   17. The method of embodiment 8, wherein the ORF comprises at least 45 or more copies (e.g., consecutive copies) of the RED codon.
   18. The method of embodiment 8, wherein the ORF comprises at least 50 or more copies (e.g., consecutive copies) of the RED codon.
   19. The method of embodiment 8, wherein the ORF comprises at least 55 or more copies (e.g., consecutive copies) of the RED codon.
   20. The method of embodiment 8, wherein the ORF comprises at least 60 or more copies (e.g., consecutive copies) of the RED codon.
   21. The method of embodiment 8, wherein the ORF comprises at least 65 or more copies (e.g., consecutive copies) of the RED codon.
   22. The method of embodiment 8, wherein the ORF comprises at least 70 or more copies (e.g., consecutive copies) of the RED codon.
   23. The method of embodiment 8, wherein the ORF comprises at least 75 or more copies (e.g., consecutive copies) of the RED codon.
   24. The method of any one of the preceding embodiments, wherein the ORF comprises at least 10 copies (e.g., consecutive copies) of the RED codon.
   25. The method of any one of the preceding embodiments, wherein the gene is selected from HTT, ATXN1, ATXN3, DMPK, GIPC1, ARX, ATN1, AR, ATXN7, TBP, COMP, RUNX2, HOXD13, ATXN2, GLS, CBL2, AFF3, FMR1, ATXN8OS, ATXN8b, LOC642361, NUTM2b-AS1, JPH3, FOXL2, PHOX2B, ZIC2, SOX3, and C9orf72.
   26. The method of embodiment 25, wherein the gene is HTT.
   27. The method of embodiment 25, wherein the gene is ATXN1.
   28 The method of embodiment 25, wherein the gene is ATXN3.
   29 The method of embodiment 25, wherein the gene is DMPK.
   30 The method of embodiment 25, wherein the gene is GIPC1.
   31. The method of embodiment 25, wherein the gene is ARX.
   32. The method of embodiment 25, wherein the gene is ATN1.
   33. The method of embodiment 25, wherein the gene is AR.
   34. The method of embodiment 25, wherein the gene is ATXN7.
   35 The method of embodiment 25, wherein the gene is TBP.
   36. The method of embodiment 25, wherein the gene is COMP.
   37 The method of embodiment 25, wherein the gene is RUNX2.
   38. The method of embodiment 25, wherein the gene is HOXD13.
   39. The method of embodiment 25, wherein the gene is ATXN2.
   40. The method of embodiment 25, wherein the gene is GLS.
   41. The method of embodiment 25, wherein the gene is CBL2.
   42. The method of embodiment 25, wherein the gene is AFF3.
   43. The method of embodiment 25, wherein the gene is FMR1.
   44. The method of embodiment 25, wherein the gene is ATXN8OS.
   45. The method of embodiment 25, wherein the gene is ATXN8b.
   46. The method of embodiment 25, wherein the gene is LOC642361.
   47. The method of embodiment 25, wherein the gene is NUTM2b-AS1.
   48. The method of embodiment 25, wherein the gene is JPH3.
   49. The method of embodiment 25, wherein the gene is FOXL2.
   50. The method of embodiment 25, wherein the gene is PHOX2B.
   51. The method of embodiment 25, wherein the gene is ZIC2.
   52. The method of embodiment 25, wherein the gene is SOX3.
   53. The method of embodiment 25, wherein the gene is C9orf72.
   54. The method of any one of the preceding embodiments, wherein the gene is selected from HTT, ATXN1, ATXN3, ATN1, AR, ATXN7, TBP, ATXN2, and GLS.
   55. The method of embodiment 54, wherein the gene is HTT.
   56. The method of embodiment 54, wherein the gene is ATXN1.
   57. The method of embodiment 54, wherein the gene is ATXN3.
   58. The method of embodiment 54, wherein the gene is ATN1.
   59. The method of embodiment 54, wherein the gene is AR.
   60. The method of embodiment 54, wherein the gene is ATXN7.
   61. The method of embodiment 54, wherein the gene is TBP.
   62. The method of embodiment 54, wherein the gene is ATXN2.
   63. The method of embodiment 54, wherein the gene is GLS.
   64. The method of any one of the preceding embodiments, wherein the replacement codon is a codon that encodes for leucine, alanine, or serine.
   65. The method of embodiment 64, wherein the replacement codon is a codon that encodes for leucine.
   66. The method of embodiment 64, wherein the replacement codon is a codon that encodes for alanine.
   67. The method of embodiment 64, wherein the replacement codon is a codon that encodes for serine.
   68. The method of any one of the preceding embodiments, wherein the replacement codon is selected from TTA, TTG, TCT, TCC, TCA, TCG, CTT, CTC, CTA, CTG, AGT, AGC, GCT, GCC, GCA, and GCG.
   69. The method of embodiment 68, wherein the replacement codon is TTA.
   70. The method of embodiment 68, wherein the replacement codon is TTG.
   71. The method of embodiment 68, wherein the replacement codon is TCT.
   72. The method of embodiment 68, wherein the replacement codon is TCC.
   73. The method of embodiment 68, wherein the replacement codon is TCA.
   74. The method of embodiment 68, wherein the replacement codon is TCG.
   75. The method of embodiment 68, wherein the replacement codon is CTT.
   76. The method of embodiment 68, wherein the replacement codon is CTC.
   77. The method of embodiment 68, wherein the replacement codon is CTA.
   78. The method of embodiment 68, wherein the replacement codon is CTG.
   79. The method of embodiment 68, wherein the replacement codon is AGT.
   80. The method of embodiment 68, wherein the replacement codon is AGC.
   81. The method of embodiment 68, wherein the replacement codon is GCT.
   82. The method of embodiment 68, wherein the replacement codon is GCC.
   83 The method of embodiment 68, wherein the replacement codon is GCA.
   84. The method of embodiment 68, wherein the replacement codon is GCG.
   85. The method of any one of the preceding embodiments, wherein the TREM comprises a TREM having at least 70%, 75%, 80%, 85%, 90%, 95%, or 99% sequence identity with the nucleotide sequence of a TREM provided in FIG. 1.
   86. The method of embodiment 85, wherein the TREM comprises a TREM having at least 70% sequence identity with the nucleotide sequence of a TREM provided in FIG. 1.
   87. The method of embodiment 85, wherein the TREM comprises a TREM having at least 75% sequence identity with the nucleotide sequence of a TREM provided in FIG. 1.
   88. The method of embodiment 85, wherein the TREM comprises a TREM having at least 80% sequence identity with the nucleotide sequence of a TREM provided in FIG. 1.
   89. The method of embodiment 85, wherein the TREM comprises a TREM having at least 85% sequence identity with the nucleotide sequence of a TREM provided in FIG. 1.
   90. The method of embodiment 85, wherein the TREM comprises a TREM having at least 90% sequence identity with the nucleotide sequence of a TREM provided in FIG. 1.
   91. The method of embodiment 85, wherein the TREM comprises a TREM having at least 95% sequence identity with the nucleotide sequence of a TREM provided in FIG. 1.
   92. The method of embodiment 85, wherein the TREM comprises a TREM having at least 99% sequence identity with the nucleotide sequence of a TREM provided in FIG. 1.
   93. The method of any one of the preceding embodiments, wherein the TREM comprises a nucleotide sequence that differs by 2, 3, 4, 5, 6, 7, 8, 9, 10, 11, 12, 13, 14, 15, 16, 17, 18, 19, or 20 nucleotides from the nucleotide sequence of a TREM provided in FIG. 1.
   94. The method of any one of the preceding embodiments, wherein the TREM comprises a nucleotide sequence that differs by 10, 15, 20, 25, 30, 35 or 40 nucleotides from the nucleotide sequence of a TREM provided in FIG. 1.
   95. The method of any one of the preceding embodiments, wherein the TREM comprises a nucleotide sequence that differs by more than 5 nucleotides from the nucleotide sequence of a TREM provided in FIG. 1.
   96. The method of any one of the preceding embodiments, wherein the TREM comprises a nucleotide sequence that differs by more than 10 nucleotides from the nucleotide sequence of a TREM provided in FIG. 1.
   97. The method of any one of the preceding embodiments, wherein the TREM comprises a nucleotide sequence that differs by no more than 1, 2, 3, 4, 5, 6, 7, 8, 9, or 10 nucleotides from the nucleotide sequence of a TREM provided in FIG. 1.
   98. The method of any one of the preceding embodiments, wherein the TREM comprises a nucleotide sequence that differs by no more than 10, 15, 20, 25, 30, 35 or 40 nucleotides from the nucleotide sequence of a TREM provided in FIG. 1.
   99. The method of any one of the preceding embodiments, wherein the TREM comprises a nucleotide sequence that differs by no more than 1 nucleotide from the nucleotide sequence of a TREM provided in FIG. 1.
   100. The method of any one of the preceding embodiments, wherein the TREM comprises a nucleotide sequence that differs by no more than 5 nucleotides from the nucleotide sequence of a TREM provided in FIG. 1.
   101. The method of any one of the preceding embodiments, wherein the TREM comprises a nucleotide sequence that differs by no more than 10 nucleotides from the nucleotide sequence of a TREM provided in FIG. 1.
   102. The method of any one of the preceding embodiments, wherein the replacement of a RED codon with a replacement codon results in a reduction of a symptom of a disease, disorder, or condition.
   103. The method of embodiment 102, wherein the disease, disorder, or condition is a repeat expansion disease, e.g., from a disease listed in Table 9.
   104. The method of embodiment 23, wherein the repeat expansion disease is selected from Huntington's disease, spinocerebellar ataxia type 1, spinocerebellar ataxia type 2, spinocerebellar ataxia type 3, and myotonic dystrophy type 1.
   105. The method of embodiment 104, wherein the repeat expansion disease is Huntington's disease.
   106. The method of embodiment 104, wherein the repeat expansion disease is spinocerebellar ataxia type 1.
   107. The method of embodiment 104, wherein the repeat expansion disease is spinocerebellar ataxia type 2.
   108. The method of embodiment 104, wherein the repeat expansion disease is spinocerebellar ataxia type 3.
   109. The method of embodiment 104, wherein the repeat expansion disease is myotonic dystrophy type 1.
   110. The method of any one of the preceding embodiments, wherein the TREM is capable of inserting a RED codon into the ORF of a gene, e.g., with at least 50%, 55%, 60%, 65%, 70%, 75%, 80%, 85%, 90%, 95%, 99%, 99.9% efficiency, e.g., compared to a reference TREM.
   111. The method of embodiment 110, wherein the TREM is capable of inserting a RED codon into the ORF of a gene, e.g., with at least 50% efficiency, e.g., compared to a reference TREM.
   112. The method of embodiment 110, wherein the TREM is capable of inserting a RED codon into the ORF of a gene, e.g., with at least 55% efficiency, e.g., compared to a reference TREM.
   113. The method of embodiment 110, wherein the TREM is capable of inserting a RED codon into the ORF of a gene, e.g., with at least 60% efficiency, e.g., compared to a reference TREM.
   114. The method of embodiment 110, wherein the TREM is capable of inserting a RED codon into the ORF of a gene, e.g., with at least 65% efficiency, e.g., compared to a reference TREM.
   115. The method of embodiment 110, wherein the TREM is capable of inserting a RED codon into the ORF of a gene, e.g., with at least 70% efficiency, e.g., compared to a reference TREM.
   116. The method of embodiment 110, wherein the TREM is capable of inserting a RED codon into the ORF of a gene, e.g., with at least 75% efficiency, e.g., compared to a reference TREM.
   117. The method of embodiment 110, wherein the TREM is capable of inserting a RED codon into the ORF of a gene, e.g., with at least 80% efficiency, e.g., compared to a reference TREM.
   118. The method of embodiment 110, wherein the TREM is capable of inserting a RED codon into the ORF of a gene, e.g., with at least 85% efficiency, e.g., compared to a reference TREM.
   119. The method of embodiment 110, wherein the TREM is capable of inserting a RED codon into the ORF of a gene, e.g., with at least 90% efficiency, e.g., compared to a reference TREM.
   120. The method of embodiment X, wherein the TREM is capable of inserting a RED codon into the ORF of a gene, e.g., with at least 95% efficiency, e.g., compared to a reference TREM.
   121. The method of embodiment 110, wherein the TREM is capable of inserting a RED codon into the ORF of a gene, e.g., with at least 99% efficiency, e.g., compared to a reference TREM.
   122. The method of embodiment 110, wherein the TREM is capable of inserting a RED codon into the ORF of a gene, e.g., with at least 99.9% efficiency, e.g., compared to a reference TREM.
   123. The method of any one of the preceding embodiments, wherein the TREM is capable of modulating a functional parameter.
   124. The method of embodiment 123, wherein the modulating comprises improving a functional parameter.
   125. The method of embodiment 124, wherein the improving comprises an improvement of about 5%, 10%, 15%, 20%, 25%, 30%, 35%, 40%, 45%, 50%, 55%, 60%, 65%, 70%, 75%, 80%, 85%, 90%, 95%, or greater, e.g., compared with a reference standard.
   126. The method of embodiment 125, wherein the improving comprises an improvement of about 5% or greater, e.g., compared with a reference standard.
   127. The method of embodiment 125, wherein the improving comprises an improvement of about 10% or greater, e.g., compared with a reference standard.
   128. The method of embodiment 125, wherein the improving comprises an improvement of about 15% or greater, e.g., compared with a reference standard.
   129. The method of embodiment 125, wherein the improving comprises an improvement of about 20% or greater, e.g., compared with a reference standard.
   130. The method of embodiment 125, wherein the improving comprises an improvement of about 25% or greater, e.g., compared with a reference standard.
   131. The method of embodiment 125, wherein the improving comprises an improvement of about 30% or greater, e.g., compared with a reference standard.
   132. The method of embodiment 125, wherein the improving comprises an improvement of about 35% or greater, e.g., compared with a reference standard.
   133. The method of embodiment 125, wherein the improving comprises an improvement of about 40% or greater, e.g., compared with a reference standard.
   134. The method of embodiment 125, wherein the improving comprises an improvement of about 45% or greater, e.g., compared with a reference standard.
   135. The method of embodiment 125, wherein the improving comprises an improvement of about 50% or greater, e.g., compared with a reference standard.
   136. The method of embodiment 125, wherein the improving comprises an improvement of about 55% or greater, e.g., compared with a reference standard.
   137. The method of embodiment 125, wherein the improving comprises an improvement of about 60% or greater, e.g., compared with a reference standard.
   138. The method of embodiment 125, wherein the improving comprises an improvement of about 65% or greater, e.g., compared with a reference standard.
   139. The method of embodiment 125, wherein the improving comprises an improvement of about 70% or greater, e.g., compared with a reference standard.
   140. The method of embodiment 125, wherein the improving comprises an improvement of about 75% or greater, e.g., compared with a reference standard.
   141. The method of embodiment 125, wherein the improving comprises an improvement of about 80% or greater, e.g., compared with a reference standard.
   142. The method of embodiment 125, wherein the improving comprises an improvement of about 85% or greater, e.g., compared with a reference standard.
   143. The method of embodiment 125, wherein the improving comprises an improvement of about 90% or greater, e.g., compared with a reference standard.
   144. The method of embodiment 125, wherein the improving comprises an improvement of about 95% or greater, e.g., compared with a reference standard.
   145. The method of embodiment 124, wherein the modulating comprises reducing a functional parameter.
   146. The method of embodiment 145, wherein the reducing comprises a reduction of about 5%, 10%, 15%, 20%, 25%, 30%, 35%, 40%, 45%, 50%, 55%, 60%, 65%, 70%, 75%, 80%, 85%, 90%, 95%, or greater, e.g., compared with a reference standard.
   147. The method of embodiment 146, wherein the reducing comprises a reduction of about 5% or greater, e.g., compared with a reference standard.
   148. The method of embodiment 146, wherein the reducing comprises a reduction of about 10% or greater, e.g., compared with a reference standard.
   149. The method of embodiment 146, wherein the reducing comprises a reduction of about 15% or greater, e.g., compared with a reference standard.
   150. The method of embodiment 146, wherein the reducing comprises a reduction of about 20% or greater, e.g., compared with a reference standard.
   151. The method of embodiment 146, wherein the reducing comprises a reduction of about 25% or greater, e.g., compared with a reference standard.
   152. The method of embodiment 146, wherein the reducing comprises a reduction of about 30% or greater, e.g., compared with a reference standard.
   153. The method of embodiment 146, wherein the reducing comprises a reduction of about 35% or greater, e.g., compared with a reference standard.
   154. The method of embodiment 146, wherein the reducing comprises a reduction of about 40% or greater, e.g., compared with a reference standard.
   155. The method of embodiment 146, wherein the reducing comprises a reduction of about 45% or greater, e.g., compared with a reference standard.
   156. The method of embodiment 146, wherein the reducing comprises a reduction of about 50% or greater, e.g., compared with a reference standard.
   157. The method of embodiment 146, wherein the reducing comprises a reduction of about 55% or greater, e.g., compared with a reference standard.
   158. The method of embodiment 146, wherein the reducing comprises a reduction of about 60% or greater, e.g., compared with a reference standard.
   159. The method of embodiment 146, wherein the reducing comprises a reduction of about 65% or greater, e.g., compared with a reference standard.
   160. The method of embodiment 146, wherein the reducing comprises a reduction of about 70% or greater, e.g., compared with a reference standard.
   170. The method of embodiment X, wherein the reducing comprises a reduction of about 75% or greater, e.g., compared with a reference standard.
   171. The method of embodiment 146, wherein the reducing comprises a reduction of about 80% or greater, e.g., compared with a reference standard.
   172. The method of embodiment 146, wherein the reducing comprises a reduction of about 85% or greater, e.g., compared with a reference standard.
   173. The method of embodiment 146, wherein the reducing comprises a reduction of about 90% or greater, e.g., compared with a reference standard.
   174. The method of embodiment 146, wherein the reducing comprises a reduction of about 95% or greater, e.g., compared with a reference standard.
   175. The method of any one of embodiments 27-31, wherein the functional parameter is an expression parameter or a signaling parameter.
   176. The method of embodiment 175, wherein the expression parameter is selected from:
(a) protein translation;
(b) expression level (e.g., of polypeptide or protein, or mRNA);
(c) post-translational modification of polypeptide or protein;
(d) folding (e.g., of polypeptide or protein, or mRNA),
(e) structure (e.g., of polypeptide or protein, or mRNA),
(f) transduction (e.g., of polypeptide or protein),
(g) compartmentalization (e.g., of polypeptide or protein, or mRNA),
(h) incorporation (e.g., of polypeptide or protein, or mRNA) into a supermolecular structure, e.g., incorporation into a membrane, proteasome, or ribosome,
(i) incorporation into a multimeric polypeptide, e.g., a homo or heterodimer, and/or
(j) stability.
   177. The method of embodiment 176, wherein the expression parameter is protein translation.
   178. The method of embodiment 176, wherein the expression parameter is expression level.
   179. The method of embodiment 176, wherein the expression parameter is post-translational modification of polypeptide or protein.
   180. The method of embodiment 176, wherein the expression parameter is folding.
   181. The method of embodiment 176, wherein the expression parameter is structure.
   182. The method of embodiment 176, wherein the expression parameter is transduction.
   183. The method of embodiment 176, wherein the expression parameter is compartmentalization.
   184. The method of embodiment 176, wherein the expression parameter is incorporation into a supermolecular structure.
   185. The method of embodiment 176, wherein the expression parameter is incorporation into a multimeric polypeptide.
   186. The method of embodiment 176, wherein the expression parameter is stability.
   187. The method of embodiment 175, wherein the signaling parameter is selected from:
(1) modulation of a signaling pathway, e.g., a cellular signaling pathway which is downstream or upstream of the protein encoded by the endogenous ORF having a first sequence; (2) cell fate modulation;
(3) ribosome occupancy modulation;
(4) protein translation modulation;
(5) mRNA stability modulation;
(6) protein folding and structure modulation;
(7) protein transduction or compartmentalization modulation; and/or
(8) protein stability modulation.
   188. The method of embodiment 187, wherein the signaling parameter is modulation of a signaling pathway, e.g., a cellular signaling pathway which is downstream or upstream of the protein encoded by the endogenous ORF having a first sequence.
   189. The method of embodiment 187, wherein the signaling parameter is cell fate modulation.
   190. The method of embodiment 187, wherein the signaling parameter is ribosome occupancy modulation.
   191. The method of embodiment 187, wherein the signaling parameter is protein translation modulation.
   192. The method of embodiment 187, wherein the signaling parameter is mRNA stability modulation.
   193. The method of embodiment 187, wherein the signaling parameter is protein folding and structure modulation.
   194. The method of embodiment 187, wherein the signaling parameter is protein transduction or compartmentalization modulation.
   195. The method of embodiment 187, wherein the signaling parameter is protein stability modulation.
   196. The method of any one of the preceding embodiments, wherein the TREM comprises a non-naturally occurring modification.
   197. The method of embodiment 196, wherein the non-naturally occurring modification is present on the 2′-position of a nucleotide sugar or within the internucleotide region (e.g., a backbone modification).
   198. The method of any one of the preceding embodiments, wherein the non-naturally occurring modification is selected from a 2′-O-methyl(2-OMe), 2′-halo (e.g., 2′F or 2′ Cl), 2′-O-methoxyethyl(2′MOE), or 2′-deoxy modification.
   199. The method of embodiment 198, wherein the non-naturally occurring modification is 2′-O-methyl(2-OMe).
   200. The method of embodiment 198, wherein the non-naturally occurring modification is 2′-halo (e.g., 2′F or 2′Cl).
   201. The method of embodiment 198, wherein the non-naturally occurring modification is 2′-O-methoxyethyl(2′MOE).
   202. The method of embodiment 198, wherein the non-naturally occurring modification is 2′-deoxy modification.
   203. The method of any one of the preceding embodiments, wherein the non-naturally occurring modification is a phosphorothioate modification.
   204. The method of any one of the preceding embodiments, wherein the TREM comprises 1, 2, 3, 4, 5, 6, 7, 8, 9, 10 or more non-naturally occurring modifications.
   205. The method of any one of the preceding embodiments, wherein the TREM comprises 5, 10, 15, 20, 25, 30, 35, 40, 45, or more non-naturally occurring modifications.
   206. The method of any one of the preceding embodiments, wherein the TREM has a sequence selected from a sequence provided in FIG. 1.
   207. The method of any one of the preceding embodiments, wherein the TREM comprises a sequence selected from: TREM NO. 3 (SEQ ID NO: 627), TREM NO. 4 (SEQ ID NO: 628), TREM NO. 5 (SEQ ID NO: 629), TREM NO. 6 (SEQ ID NO: 630), TREM NO. 7 (SEQ ID NO: 631), TREM NO. 9 (SEQ ID NO: 633), and TREM NO. 11 (SEQ ID NO: 635).
   208. The method of embodiment 207, wherein the TREM comprises a sequence of TREM NO. 3 (SEQ ID NO: 627).
   209. The method of embodiment 207, wherein the TREM comprises a sequence of TREM NO. 4 (SEQ ID NO: 628).
   210. The method of embodiment 207, wherein the TREM comprises a sequence of TREM NO. 5 (SEQ ID NO: 629).
   211. The method of embodiment 207, wherein the TREM comprises a sequence of TREM NO. 6 (SEQ ID NO: 630).
   212. The method of embodiment 207, wherein the TREM comprises a sequence of TREM NO. 7 (SEQ ID NO: 631).
   213. The method of embodiment 207, wherein the TREM comprises a sequence of TREM NO. 9 (SEQ ID NO: 633).
   214. The method of embodiment 207, wherein the TREM comprises a sequence of TREM NO. 11 (SEQ ID NO: 635).
   215. The method of any one of the preceding embodiments, wherein the non-naturally occurring modification is present at a nucleotide position which corresponds to one or more of nucleotides 1-76, according to the CtNS.
   216. A TREM having the nucleotide sequence of a TREM provided in FIG. 1 (e.g., a TREM having any one of SEQ ID NOs.: 625-693).
   217. The TREM of embodiment 216, wherein the TREM comprises a nucleotide sequence that differs by 2, 3, 4, 5, 6, 7, 8, 9, 10, 11, 12, 13, 14, 15, 16, 17, 18, 19, or 20 nucleotides from the nucleotide sequence of a TREM provided in FIG. 1.
   218. The TREM of any one of the preceding embodiments, wherein the TREM comprises a nucleotide sequence that differs by 10, 15, 20, 25, 30, 35 or 40 nucleotides from the nucleotide sequence of a TREM provided in FIG. 1.
   219. The TREM of any one of the preceding embodiments, wherein the TREM comprises a nucleotide sequence that differs by more than 5 nucleotides from the nucleotide sequence of a TREM provided in FIG. 1.
   220. The TREM of any one of the preceding embodiments, wherein the TREM comprises a nucleotide sequence that differs by more than 10 nucleotides from the nucleotide sequence of a TREM provided in FIG. 1.

221. The TREM of any one of the preceding embodiments, wherein the TREM comprises a nucleotide sequence that differs by no more than 1, 2, 3, 4, 5, 6, 7, 8, 9, or 10 nucleotides from the nucleotide sequence of a TREM provided in FIG. 1.

222. The TREM of embodiment 221, wherein the TREM comprises a nucleotide sequence that differs by no more than 1 nucleotide from the nucleotide sequence of a TREM provided in FIG. 1.
223. The TREM of embodiment 221, wherein the TREM comprises a nucleotide sequence that differs by no more than 2 nucleotides from the nucleotide sequence of a TREM provided in FIG. 1.
224. The TREM of embodiment 221, wherein the TREM comprises a nucleotide sequence that differs by no more than 3 nucleotides from the nucleotide sequence of a TREM provided in FIG. 1.
225. The TREM of embodiment 221, wherein the TREM comprises a nucleotide sequence that differs by no more than 4 nucleotides from the nucleotide sequence of a TREM provided in FIG. 1.
226. The TREM of embodiment 221, wherein the TREM comprises a nucleotide sequence that differs by no more than 5 nucleotides from the nucleotide sequence of a TREM provided in FIG. 1.
227. The TREM of embodiment 221, wherein the TREM comprises a nucleotide sequence that differs by no more than 6 nucleotides from the nucleotide sequence of a TREM provided in FIG. 1.
228. The TREM of embodiment 221, wherein the TREM comprises a nucleotide sequence that differs by no more than 7 nucleotides from the nucleotide sequence of a TREM provided in FIG. 1.
229. The TREM of embodiment 221, wherein the TREM comprises a nucleotide sequence that differs by no more than 8 nucleotides from the nucleotide sequence of a TREM provided in FIG. 1.
230. The TREM of embodiment 221, wherein the TREM comprises a nucleotide sequence that differs by no more than 9 nucleotides from the nucleotide sequence of a TREM provided in FIG. 1.
231. The TREM of embodiment 221, wherein the TREM comprises a nucleotide sequence that differs by no more than 10 nucleotides from the nucleotide sequence of a TREM provided in FIG. 1.
232. The TREM of any one of the preceding embodiments, wherein the TREM comprises a nucleotide sequence that differs by no more than 10, 15, 20, 25, 30, 35 or 40 nucleotides from the nucleotide sequence of a TREM provided in FIG. 1.
233. The TREM of embodiment 232, wherein the TREM comprises a nucleotide sequence that differs by no more than 10 nucleotides from the nucleotide sequence of a TREM provided in FIG. 1.
234. The TREM of embodiment 232, wherein the TREM comprises a nucleotide sequence that differs by no more than 15 nucleotides from the nucleotide sequence of a TREM provided in FIG. 1.
235. The TREM of embodiment 232, wherein the TREM comprises a nucleotide sequence that differs by no more than 20 nucleotides from the nucleotide sequence of a TREM provided in FIG. 1.
236. The TREM of embodiment 232, wherein the TREM comprises a nucleotide sequence that differs by no more than 25 nucleotides from the nucleotide sequence of a TREM provided in FIG. 1.
237. The TREM of embodiment 232, wherein the TREM comprises a nucleotide sequence that differs by no more than 30 nucleotides from the nucleotide sequence of a TREM provided in FIG. 1.
238. The TREM of embodiment 232, wherein the TREM comprises a nucleotide sequence that differs by no more than 35 nucleotides from the nucleotide sequence of a TREM provided in FIG. 1.
239. The TREM of embodiment 232, wherein the TREM comprises a nucleotide sequence that differs by no more than 40 nucleotides from the nucleotide sequence of a TREM provided in FIG. 1.
240. The TREM of any one of the preceding embodiments, wherein the TREM comprises a nucleotide sequence that differs by no more than 1 nucleotide from the nucleotide sequence of a TREM provided in FIG. 1.
241. The TREM of any one of the preceding embodiments, wherein the TREM comprises a nucleotide sequence that differs by no more than 5 nucleotides from the nucleotide sequence of a TREM provided in FIG. 1.
242. The TREM of any one of the preceding embodiments, wherein the TREM comprises a nucleotide sequence that differs by no more than 10 nucleotides from the nucleotide sequence of a TREM provided in FIG. 1.
243. The TREM of any one of the preceding embodiments, wherein the TREM is capable of inserting a missense mutation into an open reading frame (ORF) of a gene, wherein the missense mutation results in replacing a repeat expansion disease (RED) codon with a replacement codon.
244. The TREM of embodiment 243, wherein the RED codon comprises CAG, CGG, GAC, CCG, or CTG.
245. The TREM of embodiment 244, wherein the RED codon comprises CAG.
246. The TREM of embodiment 244, wherein the RED codon comprises CGG.
247. The TREM of embodiment 244, wherein the RED codon comprises GAC.
248. The TREM of embodiment 244, wherein the RED codon comprises CCG.
249. The TREM of embodiment 244, wherein the RED codon comprises CTG.
250. The TREM of any one of the preceding, wherein the RED codon is selected from CAG, CTG, CGG, GAC, CCG, and CTG.
251. The TREM of embodiment 250, wherein the RED codon is CTG.
252. The TREM of embodiment 250, wherein the RED codon is CGG.
253. The TREM of embodiment 250, wherein the RED codon is GAC.
254. The TREM of embodiment 250, wherein the RED codon is CCG.
255. The TREM of any one of the preceding embodiments, wherein the RED codon is CAG.
256. The TREM of any one of the preceding embodiments, wherein the ORF comprises at least 5, 10, 15, 20, 25, 30, 35, 40, 45, 50, 55, 60, 65, 70, 75, or more copies (e.g., consecutive copies) of the RED codon.
257. The TREM of embodiment 256, wherein the ORF comprises at least 5 or more copies (e.g., consecutive copies) of the RED codon.
258. The TREM of embodiment 256, wherein the ORF comprises at least 10 or more copies (e.g., consecutive copies) of the RED codon.
259. The TREM of embodiment 256, wherein the ORF comprises at least 15 or more copies (e.g., consecutive copies) of the RED codon.
260. The TREM of embodiment 256, wherein the ORF comprises at least 20 or more copies (e.g., consecutive copies) of the RED codon.
261. The TREM of embodiment 256, wherein the ORF comprises at least 25 or more copies (e.g., consecutive copies) of the RED codon.
262. The TREM of embodiment 256, wherein the ORF comprises at least 30 or more copies (e.g., consecutive copies) of the RED codon.
263. The TREM of embodiment 256, wherein the ORF comprises at least 35 or more copies (e.g., consecutive copies) of the RED codon.
264. The TREM of embodiment 256, wherein the ORF comprises at least 40 or more copies (e.g., consecutive copies) of the RED codon.
265. The TREM of embodiment 256, wherein the ORF comprises at least 45 or more copies (e.g., consecutive copies) of the RED codon.
266. The TREM of embodiment 256, wherein the ORF comprises at least 50 or more copies (e.g., consecutive copies) of the RED codon.
267. The TREM of embodiment 256, wherein the ORF comprises at least 55 or more copies (e.g., consecutive copies) of the RED codon.
268. The TREM of embodiment 256, wherein the ORF comprises at least 60 or more copies (e.g., consecutive copies) of the RED codon.
269. The TREM of embodiment 256, wherein the ORF comprises at least 65 or more copies (e.g., consecutive copies) of the RED codon.
270. The TREM of embodiment 256, wherein the ORF comprises at least 70 or more copies (e.g., consecutive copies) of the RED codon.
271. The TREM of embodiment 256, wherein the ORF comprises at least 75 or more copies (e.g., consecutive copies) of the RED codon.
272. The TREM of any one of the preceding embodiments, wherein the ORF comprises at least 10 copies (e.g., consecutive copies) of the RED codon.
273. The TREM of any one of the preceding embodiments, wherein the gene is selected from HTT, ATXN1, ATXN3, DMPK, GIPC1, ARX, ATN1, AR, ATXN7, TBP, COMP, RUNX2, HOXD13, ATXN2, GLS, CBL2, AFF3, FMR1, ATXN8OS, ATXN8b, LOC642361, NUTM2b-AS1, JPH3, FOXL2, PHOX2B, ZIC2, SOX3, and C9orf72.
274. The TREM of embodiment 273, wherein the gene is HTT.
275. The TREM of embodiment 273, wherein the gene is ATXN1.
276. The TREM of embodiment 273, wherein the gene is ATXN3.
277. The TREM of embodiment 273, wherein the gene is DMPK.
278. The TREM of embodiment 273, wherein the gene is GIPC1.
279. The TREM of embodiment 273, wherein the gene is ARX.
280. The TREM of embodiment 273, wherein the gene is ATN1.
281. The TREM of embodiment 273, wherein the gene is AR.
282. The TREM of embodiment 273, wherein the gene is ATXN7.
283. The TREM of embodiment 273, wherein the gene is TBP.
284. The TREM of embodiment 273, wherein the gene is COMP.
285. The TREM of embodiment 273, wherein the gene is RUNX2.
286. The TREM of embodiment 273, wherein the gene is HOXD13.
287. The TREM of embodiment 273, wherein the gene is ATXN2.
288. The TREM of embodiment 273, wherein the gene is GLS.
289. The TREM of embodiment 273, wherein the gene is CBL2.
290. The TREM of embodiment 273, wherein the gene is AFF3.
291. The TREM of embodiment 273, wherein the gene is FMR1.
292. The TREM of embodiment 273, wherein the gene is ATXN8OS.
293. The TREM of embodiment 273, wherein the gene is ATXN8b.
294. The TREM of embodiment 273, wherein the gene is LOC642361.
295. The TREM of embodiment 273, wherein the gene is NUTM2b-AS1.
296. The TREM of embodiment 273, wherein the gene is JPH3.
297. The TREM of embodiment 273, wherein the gene is FOXL2.
298. The TREM of embodiment 273, wherein the gene is PHOX2B.
299. The TREM of embodiment 273, wherein the gene is ZIC2.
300. The TREM of embodiment 273, wherein the gene is SOX3.
301. The TREM of embodiment 273, wherein the gene is C9orf72.
302. The TREM of embodiment 273, wherein the gene is selected from HTT, ATXN1, ATXN3, ATN1, AR, ATXN7, TBP, ATXN2, and GLS.
303. The TREM of embodiment 302, wherein the gene is HTT.
304. The TREM of embodiment 302, wherein the gene is ATXN1.
305. The TREM of embodiment 302, wherein the gene is ATXN3.
306. The TREM of embodiment 302, wherein the gene is ATN1.
307. The TREM of embodiment 302, wherein the gene is AR.
308. The TREM of embodiment 302, wherein the gene is ATXN7.
309. The TREM of embodiment 302, wherein the gene is TBP.
310. The TREM of embodiment 302, wherein the gene is ATXN2.
311. The TREM of embodiment 302, wherein the gene is GLS.
312. The TREM of any one of the preceding embodiments, wherein the replacement codon is a codon that encodes for leucine, alanine, or serine.
313. The TREM of embodiment 312, wherein the replacement codon is a codon that encodes for leucine.
314. The TREM of embodiment 312, wherein the replacement codon is a codon that encodes for alanine.
315. The TREM of embodiment 312, wherein the replacement codon is a codon that encodes for serine.
316. The TREM of any one of the preceding embodiments, wherein the replacement codon is selected from TTA, TTG, TCT, TCC, TCA, TCG, CTT, CTC, CTA, CTG, AGT, AGC, GCT, GCC, GCA, and GCG.
317. The TREM of any one of embodiment 316, wherein the replacement codon is TTA.
318. The TREM of any one of embodiment 316, wherein the replacement codon is TTG.
319. The TREM of any one of embodiment 316, wherein the replacement codon is TCT.
320. The TREM of any one of embodiment 316, wherein the replacement codon is TCC.
321. The TREM of any one of embodiment 316, wherein the replacement codon is TCA.
322. The TREM of any one of embodiment 316, wherein the replacement codon is TCG.
323. The TREM of any one of embodiment 316, wherein the replacement codon is CTT.
324. The TREM of any one of embodiment 316, wherein the replacement codon is CTC.
325. The TREM of any one of embodiment 316, wherein the replacement codon is CTA.
326. The TREM of any one of embodiment 316, wherein the replacement codon is CTG.
327. The TREM of any one of embodiment 316, wherein the replacement codon is AGT.
328. The TREM of any one of embodiment 316, wherein the replacement codon is AGC.
329. The TREM of any one of embodiment 316, wherein the replacement codon is GCT.
330. The TREM of any one of embodiment 316, wherein the replacement codon is GCC.
331. The TREM of any one of embodiment 316, wherein the replacement codon is GCA.
332. The TREM of any one of embodiment 316, wherein the replacement codon is GCG.
333. A pharmaceutical composition comprising a TREM of any one of the preceding embodiments.
334. The pharmaceutical composition of embodiment 333, further comprising a pharmaceutically acceptable component, e.g., an excipient.
335. A lipid nanoparticle formulation comprising a TREM of any one of the preceding embodiments.
336. A lipid nanoparticle formulation comprising a pharmaceutical composition of embodiment 333 or 334.
337. A method of treating a subject having a repeat expansion disorder comprising administering to the subject a TREM, TREM core fragment, or TREM fragment described herein (e.g., a TREM of any one of embodiments 44-64), thereby treating the subject having repeat expansion disease.
338. The method of embodiment 337, wherein the repeat expansion disorder is selected from a disease listed in Table 9.
339. The method of embodiment 338, wherein the repeat expansion disease is selected from Huntington's disease, spinocerebellar ataxia type 1, spinocerebellar ataxia type 2, spinocerebellar ataxia type 3, and myotonic dystrophy type 1.
340. The method of embodiment 339, wherein the repeat expansion disease is Huntington's disease.
341. The method of embodiment 339, wherein the repeat expansion disease is spinocerebellar ataxia type 1.
342. The method of embodiment 339, wherein the repeat expansion disease is spinocerebellar ataxia type 2.
343. The method of embodiment 339, wherein the repeat expansion disease is spinocerebellar ataxia type 3.
344. The method of embodiment 339, wherein the repeat expansion disease is myotonic dystrophy type 1.

### EXAMPLES

Table of Contents for Examples

| | |
|---|---|
| Example 1 | Synthesis of modified TREMs |
| Example 2 | HPLC and MS analysis of modified TREMs |
| Example 3 | Analysis of modified TREMs via anion-exchange HPLC |
| Example 4 | Analysis of TREMs via PAGE Purification and Analysis |
| Example 5 | Deprotection of synthesized TREM |
| Example 6 | TREMs for removal of protein aggregates in Repeat Expansion Diseases |
| | (REDs) |
| Example 7 | Effect of missense TREMs on HTT protein aggregation in HEK293 cells |
| Example 8 | Missense TREM induce basal autophagy in Hek293 cells |

### Example 1: Synthesis of Modified TREMs

Generally, TREM molecules (e.g., modified TREMs) may be chemically synthesized and purified by HPLC according to standard solid phase synthesis methods using phosphoramidite chemistry. (see, e.g., Scaringe S. et al. (2004) Curr Protoc Nucleic Acid Chem, 2.10.1-2.10.16; Usman N. et al. (1987) J. Am. Chem. Soc, 109, 7845-7854). Individually modified TREM molecules containing one or more 2′-methoxy (2′OMe), 2′-fluoro (2′F), 2′-methoxyethyl (2′-MOE), or phosphorothioate (PS) modifications were prepared according to phosphoramidite technology on solid phase used in oligonucleotide synthesis. For clarity, the arginine non-cognate TREM molecule named TREM-Arg-TGA contains the sequence of ARG-UCU-TREM body but with the anticodon sequence corresponding to UCA instead of UCU (i.e., SEQ ID NO: 622). Similarly, a serine non-cognate TREM molecule named TREM-Ser-TAG contains the sequence of SER-GCU-TREM body but with the anticodon sequence corresponding to CUA instead of GCU (i.e., SEQ ID NO: 623). A glutamine non-cognate TREM molecule named TREM-Gln-TAA contains the sequence of GLN-CUG-TREM body but with the anticodon sequence corresponding to UUA instead of CUG (i.e., SEQ ID NO: 624).

To make the 2′OMe modified TREMs, the following 2′-O-methyl phosphoramidites were used: (5′-O-dimethoxytrityl-N6-(benzoyl)-2′-O-methyl-adenosine-3′-O-(2-cyanoethyl-N,N-diisopropy-lamino)phosphoramidite, 5′-O-dimethoxy-trityl-N4-(acetyl)-2′-O-methyl-cytidine-3′-O-(2-cyanoethyl-N,N-diisopropyl-amino)phosphoramidite, (5′-O-dimethoxytrityl-N2-(isobutyryl)-2′-O-methyl-guanosine-3′-O-(2-cyano-ethyl-N,N-diisopropylamino)-phosphoramidite, and 5′-O-dimethoxy-trityl-2′-O-methyluridine-3′-O-(2-cyanoethyl-N,N-diisopropylamino)phosphoramidite. To make the 2′-deoxy and 2′-F modified TREMs, analogous 2′-deoxy and 2′-fluoro-phosphoramidites with the same protecting groups as the 2′-O-methyl RNA amidites were used. To make the 2′-MOE modified TREMs, the following 2′-MOE-phosphoramidites were used: 5′-O-(4,4′-Dimethoxytrityl)-2′-O-methoxyethyl-N6-benzoyl-adenosine-3′-O-[(2-cyanoethyl)-(N,N-diisopropyl)]-phosphoramidite, 5′-O-(4,4′-Dimethoxytrityl)-2′-O-methoxyethyl-5-methyl-N4-benzoyl-cytidine-3′-O-[(2-cyanoethyl)-(N,N-diisopropyl)]-phosphoramidite, 5′-O-(4,4′-Dimethoxytrityl)-2′-O-methoxyethyl-N2-isobutyryl-guanosine-3′-O-[(2-cyanoethyl)-(N,N-diisopropyl)]-phosphoramidite, 5′-O-(4,4′-Dimethoxytrityl)-2′-O-methoxyethyl-5-methyl-uridine-3′-O-[(2-cyanoethyl)-(N,N-diisopropyl)]-phosphoramidite.

During the oligonucleotide synthesis via this phosphoramidites approach, the phosphorothioate was introduced by oxidizing the phosphite triester using a sulfur transfer reagent, such as tetraethylthiuram disulfide (TETD), bis(O,O-diisopropoxy phosphinothioyl) disulfide (Stec's reagent), 3H-1,2-benzodithiol-3-one-1,1,-dioxide (Beaucage reagent), phenylacetyl disulfide (PADS), 3-ethoxy-1,2,4-dithiazoline-5-one (EDITH), 1,2-dithiazole-5-thione (xanthane hydride or ADTT), 3-((dimethylamino-methylidene)amino)-3H-1,2,4-dithiazole-3-thione (DDTT), dimethylthiuram disulfide (DTD), 3-phenyl-1,2,4-dithiazoline-5-one (PolyOrg Sulfa or POS).

A series of TREMs, some of which contain non-naturally occurring modifications, were synthesized according to this procedure. The sequences of each of these TREMs are provided in FIG. 1 (e.g., SEQ ID NOs: 622-693), wherein r: ribonucleotide; m: 2′-OMe; *: PS linkage; f: 2′-fluoro; moe: 2′-moe; d: deoxyribonucleotide; 5MeC: 5-methylcytosine. Thus, for example, mA represents 2′-O-methyl adenosine, moe5MeC represents 2′-MOE nucleotide with 5-methylcytosine nucleobase, and dA represents an adenosine deoxyribonucleotide.

### Example 2: HPLC and MS Analysis of Modified TREMs

Chemically modified TREM molecules may be analyzed by HPLC, for example, to evaluate the purity and homogeneity of the compositions. A Waters Aquity UPLC system using a Waters BEH C18 column (2.1 mm×50 mm×1.7 μm) may be used for this analysis. Samples may be prepared by dissolving 0.5 nmol of the TREM in 75 μL of water and injecting 2 μL of the solution. The buffers used may be 50 mM dimethylhexylammonium acetate with 10% CH₃CN (acetonitrile) as buffer A and ₅₀ mM dimethylhexylammonium acetate with 75% CH₃CN as buffer B (gradient 25-75% buffer B over 5 mins), with a flow rate of 0.5 mL/min at 60° C. ESI-LCMS data for the chemically modified TREMs may be acquired on a Thermo Ultimate 3000-LTQ-XL mass spectrometer.

A series of singly and multiply modified TREMs were synthesized according to the protocol outlined in Example 1. The calculated and detected molecular weights for each sequence were determined as outlined herein.

### Example 3: Analysis of Modified TREMs Via Anion-Exchange HPLC

This example describes the quality control of a synthesized TREM via anion-exchange HPLC. Using the Dionex DNA-Pac-PA-100 column, a gradient is employed using HPLC buffer A and HPLC buffer B. 0.5 ODUs of a sample that has been dissolved in H₂O or Tris buffer, pH 7.5 is injected onto the gradient. The gradient employed is based on oligonucleotide length and can be applied according to Table 10. The parameters provided in Table 11 can be used to program a linear gradient on the HPLC analyzer.

TABLE 10
Oligonucleotide length and gradient percentages

| Length | Gradient |
|---|---|
| (bases) | (% B) |
| 0-5 | 0-30 |
| 6-10 | 10-40 |
| 11-16 | 20-50 |
| 17-32 | 30-60 |
| 33-50 | 40-70 |
| >50 | 50-80 |

TABLE 11
Parameters for a linear gradient on HPLC analyzer

| Time | Flow | % Buffer | % Buffer |
|---|---|---|---|
| (min) | (mL/min) | A | B |
| 0 | 1.5 | 100 | 0 |
| 1 | 1.5 | 100 | 0 |
| 3 | 1.5 | 70a | 30a |
| 15 | 1.5 | 40a | 60a |
| 15.5 | 2.5 | 0 | 100 |
| 17 | 2.5 | 0 | 100 |
| 17.25 | 2.5 | 100 | 0 |
| 23 | 2.5 | 100 | 0 |
| s23.1 | 1.5 | 100 | 0 |
| 24 | 1.5 | 100 | 0 |
| 25 | 0.1 | 100 | 0 |

### Example 4: Analysis of TREMs Via PAGE Purification and Analysis

This example describes the quality control of a synthesized TREM via PAGE Purification and Analysis. Gel purification and analysis of 2′-ACE protected RNA follows standard protocols for denaturing PAGE (Ellington and Pollard (1998) In Current Protocols in Molecular Biology, Chanda, V). Briefly, the 2′-ACE protected oligo is resuspended in 200 mL of gel loading buffer. Invitrogen™ NuPAGE™ 4-12% Bis-Tris Gels or similar gel is prepared in gel apparatus. Samples are loaded and gel ran at 50-120 W, maintaining the apparatus at 40° C. When complete, the gel is exposed to ultraviolet (UV) light at 254 nm to visualize the purity of the RNA using UV shadowing. If necessary, the desired gel band is excised with a clean razor blade. The gel slice is crushed and 0.3M NaOAc elution buffer is added to the gel particles, and soaked overnight. The mixture is decanted and filtered through a Sephadex column such as Nap-10 or Nap-25.

### Example 5: Deprotection of Synthesized TREM

This example describes the deprotection of a TREM made according to an in vitro synthesis method. The 2′-protecting groups are removed using 100 mM acetic acid, pH 3.8. The formic acid and ethylene glycol byproducts are removed by incubating at 60° C. for 30 min followed by lyophilization or SpeedVac-ing to dryness. After this final deprotection step, the oligonucleotides are ready for use.

### Example 6: A Method to Reduce Protein Aggregates in Repeat Expansion Diseases (REDs) Using Missense tRNAs

The TREMs described herein were designed and tested using a mass spectrometry based assay called MS-READ. To test the feasibility of repeat expansion disease correction using missense TREMs described herein, reporters containing exon 1 of the HTT mRNA with different number of CAG repeats were designed and transfected with missense TREMs in human cell lines. The change in polyQ aggregate formation was monitored using live cell fluorescence microscopy and western blotting of cell lysate. Several Leu and Ser encoding missense TREMs were identified that decreased the number of polyQ protein aggregates in a reporter-based system and can be engineered to be more active in in vitro and in vivo models of Huntington's disease.

### Missense RNA Design and Activity Measurement

For missense tRNAs, the anticodon of the wild type tRNA was mutated to 5′-CTG-3′ to decode the CAG codon. Ala, Leu and Ser tRNA scaffolds were selected for missense mutation incorporation as the charging of these TREMs is independent of the anticodon. The mutated oligo sequences were cloned into the plko. 1 mCherry vector downstream of the H1 promoter and upstream of the poly(T) termination sequence for plasmid-based tRNA expression.

Table 13 summarizes the sequences of missense TREMs expressed from plasmids. The anticodon has been underlined and is complementary to the CAG codon. No modifications were introduced to the tRNA scaffold.

Next, the activity of engineered missense TREMs was measured using MS-READ. Briefly, a MS-READ reporter containing a CAG codon in the insert site was co-transfected with engineered missense tRNAs, and the identity of the amino acid in the insert site was measured using liquid chromatography mass spectrometry (LC-MS). Table 13 also summarizes the readout of MS-READ activity data (based on percentage of on-target missense incorporation) for engineered missense TREMs that decode the CAG codon with a non-Gln amino acid. “++” indicates missense TREM miscoding efficiency of greater than 5%, while “+” indicated missense TREM miscoding efficiency of less than 5%.

TABLE 13
Missense TREM activity

| | | SEQ | TREM |
|---|---|---|---|
| tRNA | Activity | ID NO. | NO. |
| Leu-AAG-CTG | ++ | 627 | 3 |
| Leu-CAA-CTG | ++ | 628 | 4 |
| Leu-CAG-CTG | ++ | 629 | 5 |
| Leu-TAA-CTG | ++ | 630 | 6 |
| Leu-TAG-CTG | ++ | 631 | 7 |
| Ser-AGA-CTG | + | 632 | 8 |
| Ser-CGA-CTG | ++ | 633 | 9 |
| Ser-GCT-CTG | + | 634 | 10 |
| Ser-TGA-CTG | ++ | 635 | 11 |
| Ala-AGC-CTG | + | 625 | 1 |
| Ala-CGC-CTG | + | 626 | 2 |

### HTT Reporter Design for Repeat Expansion Modeling

HTT mRNA exon 1 was appended upstream of the eGFP CDS and cloned into the pcDNA 3.4 plasmid downstream of the CMV promoter (FIG. 2). The length of the CAG repeat in exon 1 was kept at 21 CAGs (wild-type), 63 CAGs (pathogenic) or 105 CAGs (severe form of Huntington's disease).

### HTT Protein Aggregation Detection and Measurement

U87 cells were grown in Eagle's minimum essential medium supplemented with 10% fetal bovine serum (FBS) and 1x-L-Glutamine. HTT reporter was transfected in U87 cells seeded in appropriate cell culture plate. The GFP signal was visualized on a Sartorius IncuCyte® over 2-3 days to monitor aggregate formation. For western blotting, cells were lysed with Sigma CelLytic™ MT and the insoluble fraction containing the polyQ aggregates was separated from the soluble fraction using centrifugation. The insoluble fraction was treated with DNase I in buffer to shear chromatin and solubilize the fraction, and boiled in 2% SDS to disrupt protein aggregates. Both the soluble and insoluble fractions were resolved on 4-12% Bis-Tris gels in Bolt MOPS-SDS buffer, and the blots were stained with primary antibodies against GFP, polyQ protein, and markers for off-target effect detection and loading control.

For aggregation disruption, equal amounts of HTT reporter plasmid and missense tRNA plasmids were transfected in HEK293 (FIG. 3) or U87 cells and aggregation was measured as described above.

### Protein Aggregate Quantification

Images were analyzed using the IncuCyte® Basic Analysis Software (IncuCyte 2022A, Sartorius). Phase images were processed using the AI-driven Confluence Analysis module. Growth curves were generated from percentage confluence data obtained from phase images spanning the duration of the experiment. To identify puncta in experiments using HEK293 cells, fluorescent images were processed using a surface fit background subtraction, with a threshold setting of 300 green calibrated units (GCU). A maximum eccentricity filter of 0.85 was set to eliminate irregular non-circular objects that are unlikely to be true puncta. Graphs show standard error. Histograms of puncta area were generated using the IncuCyte® Basic Analysis Software. To identify puncta in experiments using U87 cells, fluorescent images were processed using a surface fit background subtraction.

### Western Blotting for Endogenous Protein and Autophagy Markers

For western blotting, cells were lysed with CelLytic™ MT and the insoluble fraction containing cellular debris was removed by centrifugation. Protein lysate was quantified and separated on a 4-12% Bis-Tris gel in 1x MES-SDS or 1x MOPS-SDS buffer depending on the protein being resolved. Blots were probed with appropriate primary and secondary antibodies against the indicated proteins.

### Cell Viability Measurement in Transfected Cells

Cell viability was measured using CellTiter-Glo luminescent cell viability assay (Promega) per the manufacturer's protocol. Luminescence values were captured using standard settings of a Varioskan LUX multimode microplate reader.

### Autophagy Inhibition Using Small Molecule Inhibitor

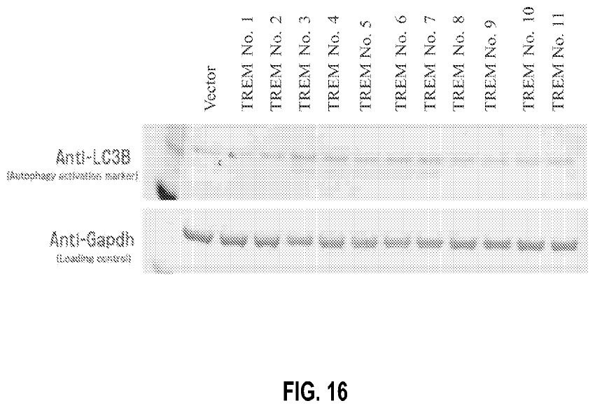

Bafilomycin A (Tocris) was used for autophagy inhibition for desired duration of experiment. A non-toxic dose of 50 nM Bafilomycin A was identified using dilution curves and was used for all experiments in Hek293 cells. LC3B cleavage was assessed by immunoblotting (FIG. 16) and was subsequently used a marker for active autophagosome formation.

### Activity of Missense TREMs Using MS-READ

The miscoding efficiency of missense tRNAs was measured using MS-READ. The MS-READ reporter containing a CAG codon in the insert position was co-transfected in U87 cells along with missense TREM plasmids. Substitution of the expected Gln residue in the reporter with other non-cognate amino acids was measured using mass spectrometry. Missense TREMs that decode the CAG codon with Leucine had the highest miscoding efficiency, followed by Serine missense TREMs. Alanine missense TREMs had the lowest miscoding efficiency on this reporter, suggesting that the Alanine tRNA anticodon may not be easily replaced with other anticodon sequences without affecting its activity.

### HTT Protein Aggregate Measurement Using Fluorescence Microscopy

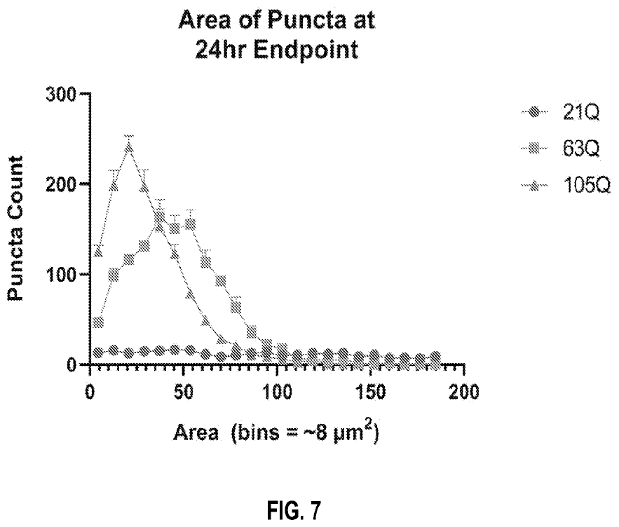

To validate the expression of HTT reporter plasmids and observe formation of protein aggregates, reporter plasmids were transfected in Hek293 cells and imaged using phase and GFP channels. Both 63Q and 105Q repeat expansion reporters which contain the CAG repeat length above the pathogenic threshold formed puncta in Hek293 cells starting at ~15 hours post-transfection which kept increasing over time. In contrast, the 21Q wild type reporter did not form any visible aggregates (FIGS. 6, 7). This suggests that HTT reporters recapitulate the aggregation phenotype observed in previous Huntington's disease models. Example 7: Effect of exemplary missense TREMs on HTT protein aggregation in HEK293 cells

To determine the effect of missense TREMs on HTT reporters, HTT 63Q reporter was co-transfected in Hek293 cells along with missense TREM plasmids and aggregate formation was measured using GFP fluorescence imaging. A decrease in GFP aggregate formation was observed in cells transfected with Leu or Ser missense tRNA but not with Ala missense tRNA (FIG. 7). The extent of aggregation reduction correlated positively with the miscoding efficiency of these tRNAs, suggesting that Leu and Ser missense tRNAs have sufficient activity to induce miscoding at the CAG repeat in the HTT 63Q reporter.

### Effect of Missense TREMs on ATXN7 Protein Aggregation in Hek293 Cells

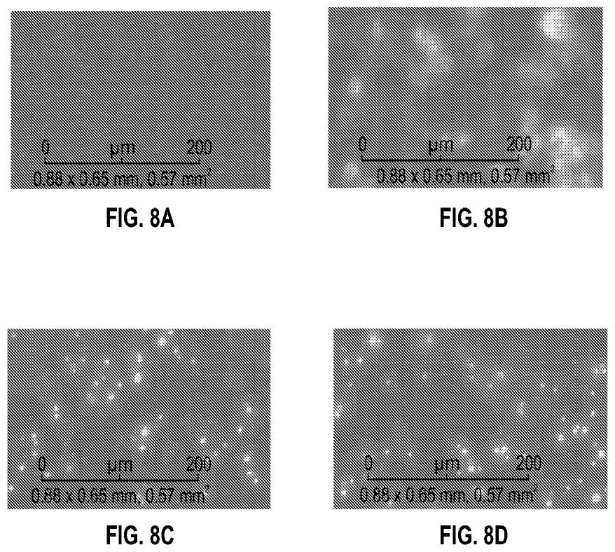

To determine the effect of missense TREMs on ATXN7 reporters, ATXN7 10Q or 52Q reporter was co-transfected in Hek293 cells along with missense TREM plasmids and aggregate formation was measured using GFP fluorescence imaging (FIG. 8). Delayed aggregate formation of ATXN7 52Q reporter was observed compared to HTT 63Q and HTT 105Q reporters, suggesting that ATXN7 reporters have different kinetic rate of protein aggregation in Hek293 cells. ATXN7 10Q reporter did not form any aggregates as expected. As observed with HTT reporters, transfection with Leu or Ser missense TREMs led to a strong reduction in the number of visible GFP aggregates in Hek293 cells compared to empty vector. Ala missense TREMs also did not lead to any change in ATXN7 52Q reporter aggregation. This suggests that ATXN7 protein aggregation can be reduced by the miscoding activity of Leu and Ser missense TREMs.

### Effect of Missense TREMs on ATXN3 Protein Aggregation in Hek293 Cells

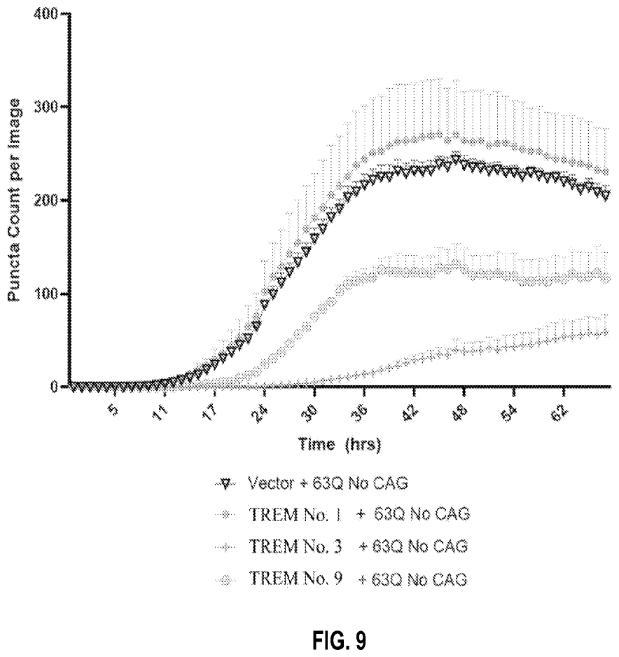

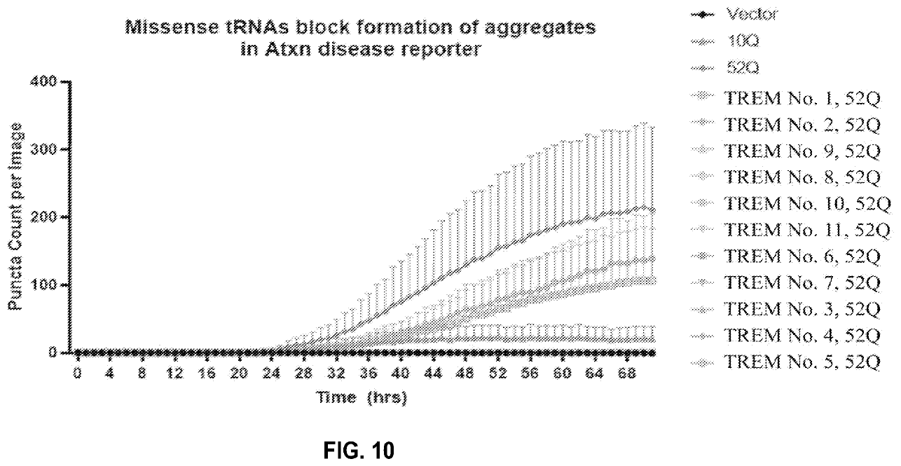

To determine the effect of missense TREMs on ATXN3 reporters, ATXN3 24Q or 74Q reporter was co-transfected in Hek293 cells along with missense TREM plasmids and aggregate formation was measured using GFP fluorescence imaging (FIG. 8). A similar kinetic rate of aggregate formation of ATXN3 74Q reporter was observed compared to HTT 63Q and HTT 105Q reporters in Hek293 cells (FIG. 9). ATXN3 24Q reporter did not form any aggregates as expected. A strong decrease in ATXN3 74Q reporter aggregation was observed in Leu or Ser missense TREM transfected cells but there was no significant reduction in protein aggregation in Ala missense TREM transfected cells (FIG. 10). None of the missense tRNAs had any effect on Hek293 cell growth.

Data from HTT, ATXN3 and ATXN7 repeat aggregation models suggests three main conclusions. First, HTT, ATXN3 and ATXN7 reporters form aggregates in cells if the CAG repeat length is above the pathogenic threshold as described in patient cohorts. Second, protein aggregates decrease in cells expressing Leu or Ser missense TREMs, but not Ala missense TREMs nor overexpression of wild type Leu, Ser and Ala tRNAs which cannot decode the CAG codon. Third, the efficacy of missense TREMs on protein aggregation correlates positively with their miscoding efficacy, suggesting that more active missense TREMs lead to a stronger reduction in protein aggregation by disrupting more of the polyQ peptide repeat in aggregating protein with non-Gln amino acids such as Leu or Ser.

### Missense TREM Expression has No Effect on Cell Viability in Hek293 or U87 Cells

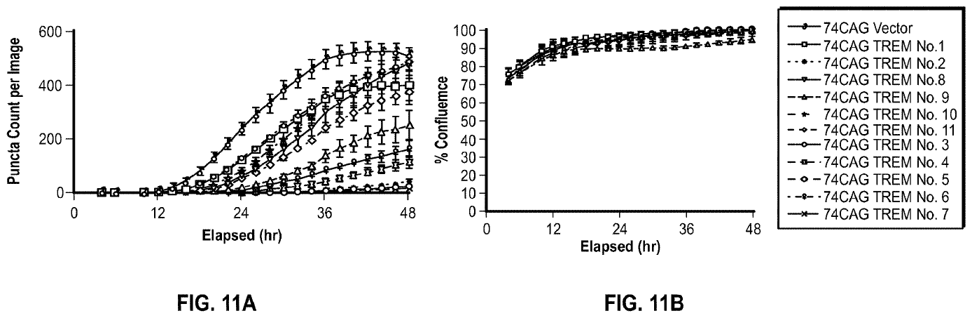

One consequence of the expression of missense TREMs in human cells could be the off-target editing of endogenous proteins containing Gln residues with other amino acids which could lead to global proteome disruption and deleterious effect on cell health. To determine the effect of missense TREMs on cell health, Hek293 or U87 cells were transfected with missense TREM plasmid and cell viability was measured after 48 hours of transfection (FIG. 11). No significant differences in cell viability in either cell type were observed compared to empty vector transfection. This suggests that while missense TREM expression may lead to off-targeting of endogenous proteins containing Gln residues, the overall viability and growth of cells is not affected.

### Missense TREMs Reduce Protein Aggregation Through Degradation of the Repeat Expansion Protein

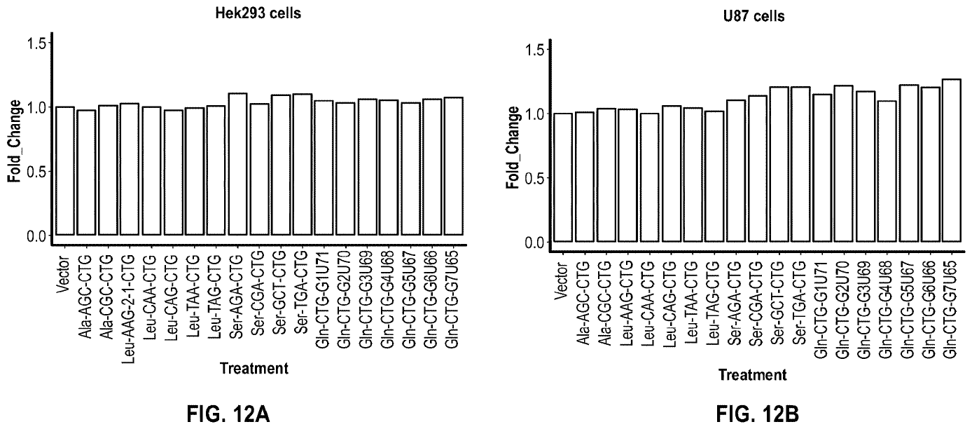

To investigate the mechanism of action of missense TREMs on repeat expanded proteins, cell lysate from control or missense TREM transfected Hek293 cells was analyzed using western blotting. A smear pattern under the HTT 63Q polyQ protein band was observed in missense TREMs transfected cells (FIG. 12), which suggests degradation of the repeat expansion protein, presumably caused by editing of the protein sequence and incorporation of non-Gln amino acids in the polyQ repeat.

Missense TREMs do not Affect the Levels of Endogenous Proteins that Contain polyQ Repeats

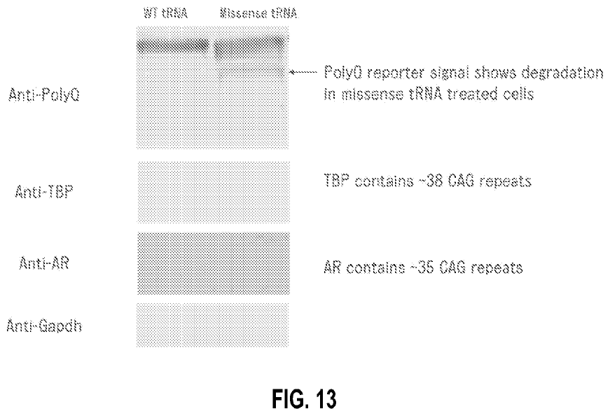

Since no effect of missense TREMs on cell viability was observed, the levels of endogenous proteins that contain polyQ repeats in their sequence were measured by assessing the levels of TBP, which contains ~38 CAG repeats in its sequence and is an important component of the RNA pol II transcription machinery, and AR, which contains 35 CAG repeats and is expressed at intermediate levels in human kidney cells (FIG. 13). There were no significant changes in the expression of these two proteins in missense tRNA transfected cells compared to empty vector transfected cells. This suggests that editing of endogenous proteins by missense TREMs under these expression conditions is not sufficient to perturb their levels, which could be due to cellular protein quality control pathways that remove toxic and non-functional proteins from cellular proteome.

### Serine Missense TREMs Reduce Protein Aggregation Via the Autophagy Pathway

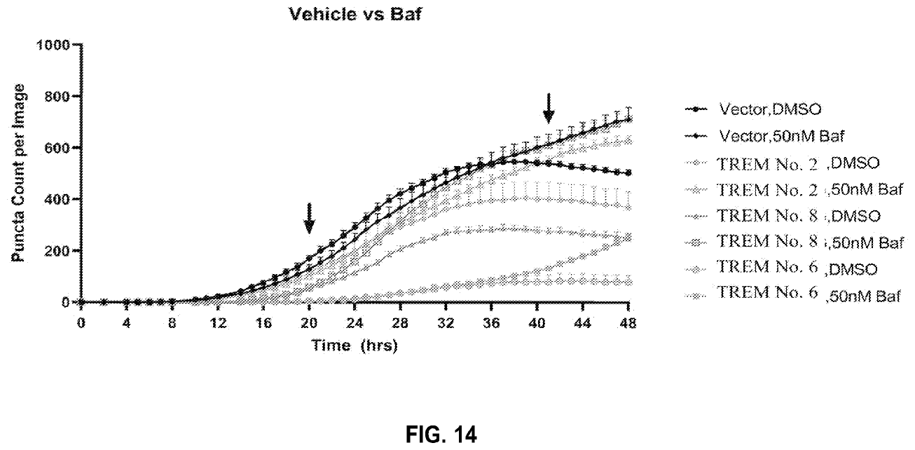

Autophagy is a cellular process responsible for degradation of cellular components such as proteins and RNA in the lysosome and is often dysregulated in neurodegenerative diseases and cancer. Autophagy also plays a role in the clearance of toxic and non-functional proteins from cells. To investigate whether autophagy plays a role in the removal of HTT 63Q polyQ protein aggregates from missense TREM transfected cells, autophagy was inhibited using a small molecule inhibitor Bafilomycin A1 (BafA1), which inhibits the vacuolar ATPase responsible for the degradation of protein cargo in autophagosomes. BafA1 treatment ~20 hours post-transfection led to an increase in protein aggregates in Ser missense tRNA transfected cells compared to DMSO treatment. However, BafA1 treatment had no effect on Leu missense TREM transfected cells, which could be due to the already low levels of visible protein aggregates in these cells (FIG. 14). In contrast, Ala missense TREM transfected cells showed no effect of autophagy inhibition on protein aggregation.

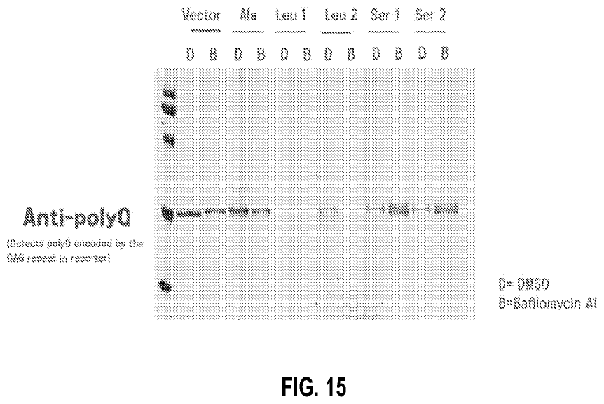

To analyze the effect of autophagy inhibition on polyQ protein accumulation, levels of HTT 63Q polyQ protein from the insoluble fraction of the cellular lysate were visualized using western blotting. BafA1 treatment in empty vector or Ala missense TREM transfected cells had no effect on polyQ protein levels (FIG. 15). The levels of polyQ protein in two different Leu missense TREM transfected cells were very low to undetectable using western blotting, consistent with the observed decrease in polyQ protein aggregates in these cells. However, cells transfected with two difference Ser missense TREMs had increased levels of polyQ protein under BafA1 treatment; hence, more polyQ protein accumulates in BafA1 treated cells after Ser missense TREM transfection.

The effect of BafA1 on protein aggregation suggests that cellular autophagy regulates polyQ protein aggregation levels in Hek293 cells, and combined with editing of protein Sequence with missense TREMs belonging to the Ser and possibly the Leu families, reduce the levels of visible protein aggregates significantly. Thus, activation of cellular autophagy combined with missense TREM expression leads to a synergistic decrease in protein aggregation in polyQ diseases, e.g., Huntington's disease.

### Example 8: Missense TREMs Induce Basal Autophagy in Hek293 Cells

PolyQ protein clearance by autophagy could be due to basal autophagy in growing cultured cells or due to specific induction of autophagy upon missense TREM treatment. To investigate these two possibilities, Hek293 cells were transfected with different missense tRNA plasmids and visualized the expression of LC3B protein using western blotting (FIG. 16). LC3B is cleaved and processed to a shorter protein upon autophagic induction and is commonly used as a marker of active autophagy in cells. LC3B cleavage was observed in Leu missense TREM transfected cells but on in Ala or Ser missense TREM transfected cells. This observation was consistent with the strong decrease in visible protein aggregates in Leu missense TREM transfected cells as well absence of detectable polyQ protein in the insoluble fraction of cell lysate. Leu missense TREMs decrease polyQ protein aggregates through induction of basal autophagy and presumably via the editing of polyQ protein to insert Leu in the polyQ tract.

Autophagy induction has been hypothesized to be a potential therapeutic strategy for the treatment of neurodegenerative diseases such as Parkinson's disease and Alzheimer's disease and for many types of cancers. Our data suggests that beyond polyQ disease correction, autophagy induction using missense TREMs could be a therapeutic strategy in other disease to ameliorate the accumulation of toxic proteins in cells. Further, in cancer cells, increasing the activity of missense TREMs to induce more widespread global proteome perturbation could lead to the expression of neoepitopes in cancer cells and the clearance of tumor cells through cellular immune activation pathways.

What is claimed is:

1. A method for inserting a missense mutation into an open reading frame (ORF) of a gene, wherein the missense mutation results in replacing a repeat expansion disease (RED) codon with a replacement codon,
the method comprising contacting the ORF with a tRNA effector molecule (TREM) comprising a sequence of Formula (A): 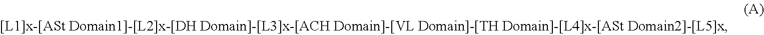
wherein:
independently, [L1] and [VL Domain], are optional and x=0 or 1,
thereby inserting the missense mutation into the ORF of the gene.

2. The method of claim 1, wherein the RED codon comprises CAG, CTG, CGG, GAC, CCG, or CTG.

3. The method of claim 1, wherein the RED codon is selected from CAG, CTG, CGG, GAC, CCG, and CTG.

4. The method of any one of the preceding claims, wherein the RED codon is CAG.

5. The method of any one of the preceding claims, wherein the ORF comprises at least 5, 10, 15, 20, 25, 30, 35, 40, 45, 50, 55, 60, 65, 70, 75, or more copies (e.g., consecutive copies) of the RED codon.

6. The method of any one of the preceding claims, wherein the ORF comprises at least 10 copies (e.g., consecutive copies) of the RED codon.

7. The method of any one of the preceding claims, wherein the gene is selected from HTT, ATXN1, ATXN3, DMPK, GIPC1, ARX, ATN1, AR, ATXN7, TBP, COMP, RUNX2, HOXD13, ATXN2, GLS, CBL2, AFF3, FMR1, ATXN8OS, ATXN8b, LOC642361, NUTM2b-AS1, JPH3, FOXL2, PHOX2B, ZIC2, SOX3, and C9orf72.

8. The method of any one of the preceding claims, wherein the gene is selected from HTT, ATXN1, ATXN3, ATN1, AR, ATXN7, TBP, ATXN2, and GLS.

9. The method of any one of the preceding claims, wherein the gene is HTT.

10. The method of any one of the preceding claims, wherein the replacement codon is a codon that encodes for leucine, alanine, or serine.

11. The method of any one of the preceding claims, wherein the replacement codon is selected from TTA, TTG, TCT, TCC, TCA, TCG, CTT, CTC, CTA, CTG, AGT, AGC, GCT, GCC, GCA, and GCG.

12. The method of any one of the preceding claims, wherein the TREM comprises a TREM having at least 70%, 75%, 80%, 85%, 90%, 95%, or 99% sequence identity with the nucleotide sequence of a TREM provided in FIG. 1.

13. The method of any one of the preceding claims, wherein the TREM comprises a nucleotide sequence that differs by 2, 3, 4, 5, 6, 7, 8, 9, 10, 11, 12, 13, 14, 15, 16, 17, 18, 19, or 20 nucleotides from the nucleotide sequence of a TREM provided in FIG. 1.

14. The method of any one of the preceding claims, wherein the TREM comprises a nucleotide sequence that differs by 10, 15, 20, 25, 30, 35 or 40 nucleotides from the nucleotide sequence of a TREM provided in FIG. 1.

15. The method of any one of the preceding claims, wherein the TREM comprises a nucleotide sequence that differs by more than 5 nucleotides from the nucleotide sequence of a TREM provided in FIG. 1.

16. The method of any one of the preceding claims, wherein the TREM comprises a nucleotide sequence that differs by more than 10 nucleotides from the nucleotide sequence of a TREM provided in FIG. 1.

17. The method of any one of the preceding claims, wherein the TREM comprises a nucleotide sequence that differs by no more than 1, 2, 3, 4, 5, 6, 7, 8, 9, or 10 nucleotides from the nucleotide sequence of a TREM provided in FIG. 1.

18. The method of any one of the preceding claims, wherein the TREM comprises a nucleotide sequence that differs by no more than 10, 15, 20, 25, 30, 35 or 40 nucleotides from the nucleotide sequence of a TREM provided in FIG. 1.

19. The method of any one of the preceding claims, wherein the TREM comprises a nucleotide sequence that differs by no more than 1 nucleotide from the nucleotide sequence of a TREM provided in FIG. 1.

20. The method of any one of the preceding claims, wherein the TREM comprises a nucleotide sequence that differs by no more than 5 nucleotides from the nucleotide sequence of a TREM provided in FIG. 1.

21. The method of any one of the preceding claims, wherein the TREM comprises a nucleotide sequence that differs by no more than 10 nucleotides from the nucleotide sequence of a TREM provided in FIG. 1.

22. The method of any one of the preceding claims, wherein the replacement of a RED codon with a replacement codon results in a reduction of a symptom of a disease, disorder, or condition.

23. The method of claim 22, wherein the disease, disorder, or condition is a repeat expansion disease, e.g., from a disease listed in Table 9.

24. The method of claim 23, wherein the repeat expansion disease is selected from Huntington's disease, spinocerebellar ataxia type 1, spinocerebellar ataxia type 2, spinocerebellar ataxia type 3, and myotonic dystrophy type 1.

25. The method of any one of claims 23-24, wherein the repeat expansion disease is Huntington's disease.

26. The method of any one of the preceding claims, wherein the TREM is capable of inserting a RED codon into the ORF of a gene, e.g., with at least 50%, 55%, 60%, 65%, 70%, 75%, 80%, 85%, 90%, 95%, 99%, 99.9% efficiency, e.g., compared to a reference TREM.

27. The method of any one of the preceding claims, wherein the TREM is capable of modulating a functional parameter.

28. The method of claim 27, wherein the modulating comprises improving a functional parameter.

29. The method of claim 28, wherein the improving comprises an improvement of about 5%, 10%, 15%, 20%, 25%, 30%, 35%, 40%, 45%, 50%, 55%, 60%, 65%, 70%, 75%, 80%, 85%, 90%, 95%, or greater, e.g., compared with a reference standard.

30. The method of claim 29, wherein the modulating comprises reducing a functional parameter.

31. The method of claim 30, wherein the reducing comprises a reduction of about 5%, 10%, 15%, 20%, 25%, 30%, 35%, 40%, 45%, 50%, 55%, 60%, 65%, 70%, 75%, 80%, 85%, 90%, 95%, or greater, e.g., compared with a reference standard.

32. The method of any one of claims 27-31, wherein the functional parameter is an expression parameter or a signaling parameter.

33. The method of claim 32, wherein the expression parameter is selected from:
(a) protein translation;
(b) expression level (e.g., of polypeptide or protein, or mRNA);
(c) post-translational modification of polypeptide or protein;
(d) folding (e.g., of polypeptide or protein, or mRNA),
(e) structure (e.g., of polypeptide or protein, or mRNA),
(f) transduction (e.g., of polypeptide or protein),
(g) compartmentalization (e.g., of polypeptide or protein, or mRNA),
(h) incorporation (e.g., of polypeptide or protein, or mRNA) into a supermolecular structure, e.g., incorporation into a membrane, proteasome, or ribosome,
(i) incorporation into a multimeric polypeptide, e.g., a homo or heterodimer, and/or
(j) stability.

34. The method of claim 32, wherein the signaling parameter is selected from:
(1) modulation of a signaling pathway, e.g., a cellular signaling pathway which is downstream or upstream of the protein encoded by the endogenous ORF having a first sequence;
(2) cell fate modulation;
(3) ribosome occupancy modulation;
(4) protein translation modulation;
(5) mRNA stability modulation;
(6) protein folding and structure modulation;
(7) protein transduction or compartmentalization modulation; and/or
(8) protein stability modulation.

35. The method of any one of the preceding claims, wherein the TREM comprises a non-naturally occurring modification.

36. The method of claim 35, wherein the non-naturally occurring modification is present on the 2′-position of a nucleotide sugar or within the internucleotide region (e.g., a backbone modification).

37. The method of any one of claims 35-36, wherein the non-naturally occurring modification is selected from a 2′-O-methyl(2-OMe), 2′-halo (e.g., 2′F or 2′Cl), 2′-O-methoxyethyl(2′MOE), or 2′-deoxy modification.

38. The method of any one of claims 35-37, wherein the non-naturally occurring modification is a phosphorothioate modification.

39. The method of any one of claims 35-38, wherein the TREM comprises 1, 2, 3, 4, 5, 6, 7, 8, 9, 10 or more non-naturally occurring modifications.

40. The method of any one of claims 22-26, wherein the TREM comprises 5, 10, 15, 20, 25, 30, 35, 40, 45, or more non-naturally occurring modifications.

41. The method of any one of the preceding claims, wherein the TREM has a sequence selected from a sequence provided in FIG. 1.

42. The method of any one of the preceding claims, wherein the TREM comprises a sequence selected from: TREM NO. 3 (SEQ ID NO: 627), TREM NO. 4 (SEQ ID NO: 628), TREM NO. 5 (SEQ ID NO: 629), TREM NO. 6 (SEQ ID NO: 630), TREM NO. 7 (SEQ ID NO: 631, TREM NO. 9 (SEQ ID NO: 633), and TREM NO. 11 (SEQ ID NO: 635).

43. The method of any one of the preceding claims, wherein the non-naturally occurring modification is present at a nucleotide position which corresponds to one or more of nucleotides 1-76, according to the CtNS.

44. A TREM having the nucleotide sequence of a TREM provided in FIG. 1 (e.g., a TREM having any one of SEQ ID NOs.: 625-693).

45. The TREM of claim 44, wherein the TREM comprises a nucleotide sequence that differs by 2, 3, 4, 5, 6, 7, 8, 9, 10, 11, 12, 13, 14, 15, 16, 17, 18, 19, or 20 nucleotides from the nucleotide sequence of a TREM provided in FIG. 1.

46. The TREM of any one of claims 44-45, wherein the TREM comprises a nucleotide sequence that differs by 10, 15, 20, 25, 30, 35 or 40 nucleotides from the nucleotide sequence of a TREM provided in FIG. 1.

47. The TREM of any one of claims 44-46, wherein the TREM comprises a nucleotide sequence that differs by more than 5 nucleotides from the nucleotide sequence of a TREM provided in FIG. 1.

48. The TREM of any one of claims 44-47, wherein the TREM comprises a nucleotide sequence that differs by more than 10 nucleotides from the nucleotide sequence of a TREM provided in FIG. 1.

49. The TREM of any one of claims 44-48, wherein the TREM comprises a nucleotide sequence that differs by no more than 1, 2, 3, 4, 5, 6, 7, 8, 9, or 10 nucleotides from the nucleotide sequence of a TREM provided in FIG. 1.

50. The TREM of any one of claims 44-49, wherein the TREM comprises a nucleotide sequence that differs by no more than 10, 15, 20, 25, 30, 35 or 40 nucleotides from the nucleotide sequence of a TREM provided in FIG. 1.

51. The TREM of any one of claims 44-50, wherein the TREM comprises a nucleotide sequence that differs by no more than 1 nucleotide from the nucleotide sequence of a TREM provided in FIG. 1.

52. The TREM of any one of claims 44-51, wherein the TREM comprises a nucleotide sequence that differs by no more than 5 nucleotides from the nucleotide sequence of a TREM provided in FIG. 1.

53. The TREM of any one of claims 44-52, wherein the TREM comprises a nucleotide sequence that differs by no more than 10 nucleotides from the nucleotide sequence of a TREM provided in FIG. 1.

54. The TREM of any one of claims 44-53, wherein the TREM is capable of inserting a missense mutation into an open reading frame (ORF) of a gene, wherein the missense mutation results in replacing a repeat expansion disease (RED) codon with a replacement codon.

55. The TREM of claim 54, wherein the RED codon comprises CAG, CTG, CGG, GAC, CCG, or CTG.

56. The TREM of any one of claims 54-55, wherein the RED codon is selected from CAG, CTG, CGG, GAC, CCG, and CTG.

57. The TREM of any one of claims 54-56, wherein the RED codon is CAG.

58. The TREM of any one of claims 54-57, wherein the ORF comprises at least 5, 10, 15, 20, 25, 30, 35, 40, 45, 50, 55, 60, 65, 70, 75, or more copies (e.g., consecutive copies) of the RED codon.

59. The TREM of any one of claims 54-58, wherein the ORF comprises at least 10 copies (e.g., consecutive copies) of the RED codon.

60. The TREM of any one of claims 54-59, wherein the gene is selected from HTT, ATXN1, ATXN3, DMPK, GIPC1, ARX, ATN1, AR, ATXN7, TBP, COMP, RUNX2, HOXD13, ATXN2, GLS, CBL2, AFF3, FMR1, ATXN8OS, ATXN8b, LOC642361, NUTM2b-AS1, JPH3, FOXL2, PHOX2B, ZIC2, SOX3, and C9orf72.

61. The TREM of any one of claims 54-60, wherein the gene is selected from HTT, ATXN1, ATXN3, ATN1, AR, ATXN7, TBP, ATXN2, and GLS.

62. The TREM of any one of claims 54-61, wherein the gene is HTT.

63. The TREM of any one of claims 54-62, wherein the replacement codon is a codon that encodes for leucine, alanine, or serine.

64. The TREM of any one of claims 54-63, wherein the replacement codon is selected from TTA, TTG, TCT, TCC, TCA, TCG, CTT, CTC, CTA, CTG, AGT, AGC, GCT, GCC, GCA, and GCG.

65. A pharmaceutical composition comprising a TREM of any one of claims 44-64.

66. The pharmaceutical composition of claim 65, further comprising a pharmaceutically acceptable component, e.g., an excipient.

67. A lipid nanoparticle formulation comprising a TREM of any one of claims 44-64.

68. A lipid nanoparticle formulation comprising a pharmaceutical composition of claim 67.

69. A method of treating a subject having a repeat expansion disorder comprising administering to the subject a TREM, TREM core fragment, or TREM fragment described herein (e.g., a TREM of any one of claims 44-64), thereby treating the subject having repeat expansion disease.

70. The method of claim 69, wherein the repeat expansion disorder is selected from a disease listed in Table 9.

71. The method of claim 69, wherein the repeat expansion disease is selected from Huntington's disease, spinocerebellar ataxia type 1, spinocerebellar ataxia type 2, spinocerebellar ataxia type 3, and myotonic dystrophy type 1.
